SEQUENCE LISTING (152 sequences)

SEQ ID NO: 1 - DNA, 81 bp, Mus musculus
  source 1..81: mol_type = genomic DNA
ttctgggcccttgtggtggttgccggagtgctgttttgctatgggctcctggttaccgttgccctttgtgtgatttggacc

SEQ ID NO: 2 - protein, 27 aa, Mus musculus
FWALVVVAGVLFCYGLLVTVALCVIWT

SEQ ID NO: 3 - DNA, 81 bp, Homo sapiens
  source 1..81: mol_type = genomic DNA
ttttgggtattggtagtggtgggcggagttttagcctgctacagcctcctggtaacagtggcttttatcatcttttgggtg

SEQ ID NO: 4 - protein, 27 aa, Homo sapiens
FWVLVVVGGVLACYSLLVTVAFIIFWV

SEQ ID NO: 5 - DNA, 729 bp, Homo sapiens
  source 1..729: mol_type = genomic DNA
cagcgggctttagtcttgcggcgtaaacgtaaaagaatgacagatccaactcgcaggttcttcaaagtgacccccccacctgggtccggaccgcagaaccaatatgggaatgtcctgtctctgcctacgcctacaagtggactgggtagggctcagaggtgggctgccggtctcggcggaactgcgccatcttacggaaatccctcctccgacgttcaggcagacggggccctggggtctcgatccccgcctggtgttggaccagaagaggaagagggcgagggctacgaagagcccgactccgaagaggacagtgagttttacgagaacgacagcaacctggggcaggatcagctgtcacaggatggctcaggatatgaaaaccctgaggacgagcctttggggcctgaagatgaggactccttttctaatgcagagtcatatgagaatgaggacgaagaattgactcaacccgtggcaagaacaatggatttcctcagtccacacgggagtgcatgggacccctccagagaggctactagcctcggttctcaaagctatgaggacatgaggggtattctgtacgcagcgcctcagttgaggtccatccgcggccagccaggcccaaaccatgaggaagatgccgattcttacgaaaacatggacaaccccgatggtcctgaccccgcatgggggggcggcgggaggatgggcacctggtctactcgc

SEQ ID NO: 6 - protein, 243 aa, Homo sapiens
QRALVLRRKRKRMTDPTRRFFKVTPPPGSGPQNQYGNVLSLPTPTSGLGRAQRWAAGLGGTAPSYGNPSSDVQADGALGSRSPPGVGPEEEEGEGYEEPDSEEDSEFYENDSNLGQDQLSQDGSGYENPEDEPLGPEDEDSFSNAESYENEDEELTQPVARTMDFLSPHGSAWDPSREATSLGSQSYEDMRGILYAAPQLRSIRGQPGPNHEEDADSYENMDNPDGPDPAWGGGGRMGTWSTR

SEQ ID NO: 7 - DNA, 186 bp, Homo sapiens
  source 1..186: mol_type = genomic DNA
aagaaggttgcaaaaaaacctactaataaggctccccatcctaagcaagagccccaagaaattaactttcccgatgatcttccgggttctaacacggcagccccggtgcaggagaccctgcatggttgtcaacccgtcactcaggaggacgggaaagagtctcgtatctccgtccaggagagacag

SEQ ID NO: 8 - protein, 62 aa, Homo sapiens
KKVAKKPTNKAPHPKQEPQEINFPDDLPGSNTAAPVQETLHGCQPVTQEDGKESRISVQERQ

SEQ ID NO: 9 - DNA, 330 bp, Homo sapiens
  source 1..330: mol_type = genomic DNA
aagaaggttgcaaaaaaacctactaataaggctccccatcctaagcaagagccccaagaaattaactttcccgatgatcttccgggttctaacacggcagccccggtgcaggagaccctgcatggttgtcaacccgtcactcaggaggacgggaaagagtctcgtatctccgtccaggagagacaggacaaggacgatagtaaagcagggatggaggaggaccatacatacgagggactggatatcgatcagacagccacgtacgaagacattgtgacactgagaactggcgaggtgaagtggtcagtgggagaacatccggggcaggaa

SEQ ID NO: 10 - protein, 110 aa, Homo sapiens
KKVAKKPTNKAPHPKQEPQEINFPDDLPGSNTAAPVQETLHGCQPVTQEDGKESRISVQERQDKDDSKAGMEEDHTYEGLDIDQTATYEDIVTLRTGEVKWSVGEHPGQE

SEQ ID NO: 11 - DNA, 312 bp, Homo sapiens
  source 1..312: mol_type = genomic DNA
aagaaggttgcaaaaaaacctactaataaggctccccatcctaagcaagagccccaagaaattaactttcccgatgatcttccgggttctaacacggcagccccggtgcaggagaccctgcatggttgtcaacccgtcactcaggaggacgggaaagagtctcgtatctccgtccaggagagacagaaaagaggccgaaaaaagctgctgtacatcttcaaacaacccttcatgcgacctgttcagacgacacaggaggaggacggctgcagctgtaggtttcccgaagaagaggagggaggatgcgaactt

SEQ ID NO: 12 - protein, 104 aa, Homo sapiens
KKVAKKPTNKAPHPKQEPQEINFPDDLPGSNTAAPVQETLHGCQPVTQEDGKESRISVQERQKRGRKKLLYIFKQPFMRPVQTTQEEDGCSCRFPEEEEGGCEL

SEQ ID NO: 13 - DNA, 126 bp, Homo sapiens
  source 1..126: mol_type = genomic DNA
aaaagaggccgaaaaaagctgctgtacatcttcaaacaacccttcatgcgacctgttcagacgacacaggaggaggacggctgcagctgtaggtttcccgaagaagaggagggaggatgcgaactt

SEQ ID NO: 14 - protein, 42 aa, Homo sapiens
KRGRKKLLYIFKQPFMRPVQTTQEEDGCSCRFPEEEEGGCEL

SEQ ID NO: 15 - DNA, 414 bp, Homo sapiens
  source 1..414: mol_type = genomic DNA
aagaaggttgcaaaaaaacctactaataaggctccccatcctaagcaagagccccaagaaattaactttcccgatgatcttccgggttctaacacggcagccccggtgcaggagaccctgcatggttgtcaacccgtcactcaggaggacgggaaagagtctcgtatctccgtccaggagagacagcgcaaaaaacgtataagcgcaaactctacagatccagtaaaagccgcgcaattcgagcctcccggccgccagatgattgcaatacggaaacgtcaactggaggaaactaataatgactatgagacggccgacggtggatacatgacccttaatccccgcgcgccaaccgacgatgataagaacatatatctgacgctcccccctaacgatcacgttaacagtaataat

SEQ ID NO: 16 - protein, 138 aa, Homo sapiens
KKVAKKPTNKAPHPKQEPQEINFPDDLPGSNTAAPVQETLHGCQPVTQEDGKESRISVQERQRKKRISANSTDPVKAAQFEPPGRQMIAIRKRQLEETNNDYETADGGYMTLNPRAPTDDDKNIYLTLPPNDHVNSNN

SEQ ID NO: 17 - DNA, 228 bp, Homo sapiens
  source 1..228: mol_type = genomic DNA
cgcaaaaaacgtataagcgcaaactctacagatccagtaaaagccgcgcaattcgagcctcccggccgccagatgattgcaatacggaaacgtcaactggaggaaactaataatgactatgagacggccgacggtggatacatgacccttaatccccgcgcgccaaccgacgatgataagaacatatatctgacgctcccccctaacgatcacgttaacagtaataat

SEQ ID NO: 18 - protein, 76 aa, Homo sapiens
RKKRISANSTDPVKAAQFEPPGRQMIAIRKRQLEETNNDYETADGGYMTLNPRAPTDDDKNIYLTLPPNDHVNSNN

SEQ ID NO: 19 - DNA, 681 bp, Homo sapiens
  source 1..681: mol_type = genomic DNA
atggcggcgggcgggcccggcgccggaagcgccgcgccagtctcatctacgtccagtctgccactggctgccctgaacatgagagtgagacgccgtttatccctcttcctgaatgtgcggacccaggtcgccgctgattggaccgccctggccgaagagatggactttgaatacttggaaatcagacagctggaaacacaggcagacccaaccgggagactgcttgacgcctggcagggacgcccaggggcaagtgttggtcggttactggagcttttaactaagttgggccgcgatgacgtgctgttggagttaggacccagtatcgaggaggattgtcagaaatacatcttgaaacagcagcaggaggaggcggaaaagcccctgcaggtggcggccgttgacagcagtgtacccagaacagctgagctggccggcatcacaaccctggatgatcccctgggccacatgcctgagaggttcgacgctttcataaagaaggttgcaaaaaaacctactaataaggctccccatcctaagcaagagccccaagaaattaactttcccgatgatcttccgggttctaacacggcagccccggtgcaggagaccctgcatggttgtcaacccgtcactcaggaggacgggaaagagtctcgtatctccgtccaggagagacag

SEQ ID NO: 20 - protein, 227 aa, Homo sapiens
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFIKKVAKKPTNKAPHPKQEPQEINFPDDLPGSNTAAPVQETLHGCQPVTQEDGKESRISVQERQ

SEQ ID NO: 21 - DNA, 495 bp, Homo sapiens
  source 1..495: mol_type = genomic DNA
atggcggcgggcgggcccggcgccggaagcgccgcgccagtctcatctacgtccagtctgccactggctgccctgaacatgagagtgagacgccgtttatccctcttcctgaatgtgcggacccaggtcgccgctgattggaccgccctggccgaagagatggactttgaatacttggaaatcagacagctggaaacacaggcagacccaaccgggagactgcttgacgcctggcagggacgcccaggggcaagtgttggtcggttactggagcttttaactaagttgggccgcgatgacgtgctgttggagttaggacccagtatcgaggaggattgtcagaaatacatcttgaaacagcagcaggaggaggcggaaaagcccctgcaggtggcggccgttgacagcagtgtacccagaacagctgagctggccggcatcacaaccctggatgatcccctgggccacatgcctgagaggttcgacgctttcata

SEQ ID NO: 22 - protein, 165 aa, Homo sapiens
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFI

SEQ ID NO: 23 - DNA, 183 bp, Homo sapiens
  source 1..183: mol_type = genomic DNA
aggaaacgatggcagaacgagaagctcgggttggatgccggggatgaatatgaagatgaaaacctttatgaaggcctgaacctggacgactgctccatgtatgaggacatctcccggggcctccagggcacctaccaggatgtgggcagcctcaacataggagatgtccagctggagaagccg

SEQ ID NO: 24 - protein, 61 aa, Homo sapiens
RKRWQNEKLGLDAGDEYEDENLYEGLNLDDCSMYEDISRGLQGTYQDVGSLNIGDVQLEKP

SEQ ID NO: 25 - DNA, 147 bp, Homo sapiens
  source 1..147: mol_type = genomic DNA
ctggacaaggatgacagcaaggctggcatggaggaagatcacacctacgagggcctggacattgaccagacagccacctatgaggacatagtgacgctgcggacaggggaagtgaagtggtctgtaggtgagcacccaggccaggag

SEQ ID NO: 26 - protein, 49 aa, Homo sapiens
LDKDDSKAGMEEDHTYEGLDIDQTATYEDIVTLRTGEVKWSVGEHPGQE

SEQ ID NO: 27 - DNA, 165 bp, Homo sapiens
  source 1..165: mol_type = genomic DNA
ttcgtgcctgtgttcctcccagctaagcccactaccacccccgctccaaggccgcccacgcccgctcctactattgctagtcagcctttaagtttacgacccgaagcttgcaggcccgccgccggcggcgctgtgcacaccagggggcttgattttgcctgcgac

SEQ ID NO: 28 - protein, 55 aa, Homo sapiens
FVPVFLPAKPTTTPAPRPPTPAPTIASQPLSLRPEACRPAAGGAVHTRGLDFACD

SEQ ID NO: 29 - DNA, 189 bp, synthetic construct
  source 1..189: mol_type = other DNA
ggcgctggtagtggcggtaactggagccaccctcaatttgagaagggcgggtcaggcggatcaggtggtagtggtgggtccaactggagccatccgcaatttgaaaagggcggaagcggcggttccggcggttcaggcggtagcaactggtcacatccgcaatttgagaaaggcgggtcaggcggcggg

SEQ ID NO: 30 - protein, 63 aa, synthetic construct
GAGSGGNWSHPQFEKGGSGGSGGSGGSNWSHPQFEKGGSGGSGGSGGSNWSHPQFEKGGSGGG

SEQ ID NO: 31 - DNA, 903 bp, Homo sapiens
  source 1..903: mol_type = genomic DNA
cccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccgagctgcaactggaggagagctgtgcggaggcgcaggacggggagctggacgggctgtggacgaccatcaccatcttcatcacactcttcctgttaagcgtgtgctacagtgccaccgtcaccttcttcaaggtgaagtggatcttctcctcggtggtggacctgaagcagaccatcatccccgactacaggaacatgatcggacagggggcctga

SEQ ID NO: 32 - protein, 300 aa, Homo sapiens
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPELQLEESCAEAQDGELDGLWTTITIFITLFLLSVCYSATVTFFKVKWIFSSVVDLKQTIIPDYRNMIGQGA

SEQ ID NO: 33 - DNA, 726 bp, synthetic construct
  source 1..726: mol_type = other DNA
gaggttcaacttgttcaatctggggcagaagtgaagaagcccggggcatctgtgaaagtatcatgcaaaacatccggctatacgtttaccgaatacaccattcactgggtcagacaggctcccggtcaaagcctcgaatggatgggaaatattaaccctaacaatggcggaaccacatataatcagaaattccaaggccgagtgacgataactgtcgataagagtacgtccacagcttacatggaactcagctctttgagatccgaagacactgcagtttattattgtgcagctggatggaacttcgactattggggacaagggactcttgttacggtgtccagtggcaaaccaggtagtggtaaacccggaagcggcaagcccgggagcggtaaacctggtagcgacatcgtcatgactcaaagccctgactcactcgccgtgagcctgggagagcgtgcaacgctatcttgtcgggcctctcaggatgtcggaactgctgtagactggtatcaacagaaacctgaccaatcaccaaaactcctgatttattgggcctcaacacgtcacacaggagtgccagataggttcacaggtagtggcagtggaactgattttactttgacaattagcagcctgcaagccgaagatgtagccgtttacttctgtcaacaatataactcatacccactaacgttcggtgccgggacgaaggtagagattaaa

SEQ ID NO: 34 - protein, 242 aa, synthetic construct
EVQLVQSGAEVKKPGASVKVSCKTSGYTFTEYTIHWVRQAPGQSLEWMGNINPNNGGTTYNQKFQGRVTITVDKSTSTAYMELSSLRSEDTAVYYCAAGWNFDYWGQGTLVTVSSGKPGSGKPGSGKPGSGKPGSDIVMTQSPDSLAVSLGERATLSCRASQDVGTAVDWYQQKPDQSPKLLIYWASTRHTGVPDRFTGSGSGTDFTLTISSLQAEDVAVYFCQQYNSYPLTFGAGTKVEIK

SEQ ID NO: 35 - DNA, 735 bp, synthetic construct
  source 1..735: mol_type = other DNA
gaagtccaattggttgaaagcggtggtggactcgtcaaacctggcggtagccttaaactttcatgtgccgcaagcggcttcacgtttagtaactatgctatgagttgggtccgccaaagtccagaaaagcgcctcgaatgggtggcggagatctctggaggaggaacatatacatattatccagacaccatgaccggtaggtttacaatctcaagagacaacgctaagaacaccctgtacctggaaatgtcaagcctgagatcagaagatacggccatgtattattgtacgcgcctactcgactattggggtcaaggaacttccgtgacggtgtcaagcggaggaggtgggagcggaggaggcggaagtggcggtggtggctctggtggcggtggaagtgatatagtgatgacgcaagctgccttttcaaaccctgttactttggggactagcgcatcaatctcctgtaggtccagcaaatctttgctgcacagtaatggaatcacctatcttttctggtatttgcaaaagcctgggcagagcccgcaactgctgatctatcaaatgtcaaatcttgcttccggagttccagaccgcttctcaagttccgggtccggcactgattttaccttgagaatttctagggtcgaagctgaagacgtcggtgtctattattgcgcgcaaaaccttgagcttccatacaccttcggggggggcacaaaacttgagatcaag

SEQ ID NO: 36 - protein, 245 aa, synthetic construct
EVQLVESGGGLVKPGGSLKLSCAASGFTFSNYAMSWVRQSPEKRLEWVAEISGGGTYTYYPDTMTGRFTISRDNAKNTLYLEMSSLRSEDTAMYYCTRLLDYWGQGTSVTVSSGGGGSGGGGSGGGGSGGGGSDIVMTQAAFSNPVTLGTSASISCRSSKSLLHSNGITYLFWYLQKPGQSPQLLIYQMSNLASGVPDRFSSSGSGTDFTLRISRVEAEDVGVYYCAQNLELPYTFGGGTKLEIK

SEQ ID NO: 37 - DNA, 759 bp, synthetic construct
  source 1..759: mol_type = other DNA
cagtctgtgttgacgcagccgccctcagtgtctgcggccccaggacagagggtcaccatctcctgctctggaaccaggtccaacattgggagtgattatgtttcctggtaccaacacctcccaggaacagcccccaaactcctcgtttatggcgataatctgcgaccctcagggattcctgaccgattctctgcctccaagtctggcacgtcagccaccctgggcatcaccggactccagactggggacgaggccgattattactgcggcacatgggattacaccctgaatggtgtggtgttcggcggagggaccaagctgaccgtcctaggttctagaggtggtggtggtagcggcggcggcggctctggtggtggtggatccctcgagatggcccaggtgcagctggtggagtctgggggaggcttggtacagcctggggggtccctgagactctcctgtgcagcctctggattcacctttagcagctatgccatgagctgggtccgccaggctccagggaaggggctggagtgggtctcagttatttatagcggtggtagtagcacatactatgcagactccgtgaagggccggttcaccatctccagagataattccaagaacacgctgtatctgcaaatgaacagcctgagagccgaggacacggccgtatattactgtgcgcgcacttcttacctgaaccatggtgattactggggtcaaggtactctggtgaccgtgtctagcgccgctgca

SEQ ID NO: 38 - protein, 253 aa, synthetic construct
QSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSAAA

SEQ ID NO: 39 - DNA, 1704 bp, synthetic construct
  source 1..1704: mol_type = other DNA
gaggttcaacttgttcaatctggggcagaagtgaagaagcccggggcatctgtgaaagtatcatgcaaaacatccggctatacgtttaccgaatacaccattcactgggtcagacaggctcccggtcaaagcctcgaatggatgggaaatattaaccctaacaatggcggaaccacatataatcagaaattccaaggccgagtgacgataactgtcgataagagtacgtccacagcttacatggaactcagctctttgagatccgaagacactgcagtttattattgtgcagctggatggaacttcgactattggggacaagggactcttgttacggtgtccagtggcaaaccaggtagtggtaaacccggaagcggcaagcccgggagcggtaaacctggtagcgacatcgtcatgactcaaagccctgactcactcgccgtgagcctgggagagcgtgcaacgctatcttgtcgggcctctcaggatgtcggaactgctgtagactggtatcaacagaaacctgaccaatcaccaaaactcctgatttattgggcctcaacacgtcacacaggagtgccagataggttcacaggtagtggcagtggaactgattttactttgacaattagcagcctgcaagccgaagatgtagccgtttacttctgtcaacaatataactcatacccactaacgttcggtgccgggacgaaggtagagattaaattcgtgcctgtgttcctcccagctaagcccactaccacccccgctccaaggccgcccacgcccgctcctactattgctagtcagcctttaagtttacgacccgaagcttgcaggcccgccgccggcggcgctgtgcacaccagggggcttgattttgcctgcgacttttgggtattggtagtggtgggcggagttttagcctgctacagcctcctggtaacagtggcttttatcatcttttgggtgcagcgggctttagtcttgcggcgtaaacgtaaaagaatgacagatccaactcgcaggttcttcaaagtgacccccccacctgggtccggaccgcagaaccaatatgggaatgtcctgtctctgcctacgcctacaagtggactgggtagggctcagaggtgggctgccggtctcggcggaactgcgccatcttacggaaatccctcctccgacgttcaggcagacggggccctggggtctcgatccccgcctggtgttggaccagaagaggaagagggcgagggctacgaagagcccgactccgaagaggacagtgagttttacgagaacgacagcaacctggggcaggatcagctgtcacaggatggctcaggatatgaaaaccctgaggacgagcctttggggcctgaagatgaggactccttttctaatgcagagtcatatgagaatgaggacgaagaattgactcaacccgtggcaagaacaatggatttcctcagtccacacgggagtgcatgggacccctccagagaggctactagcctcggttctcaaagctatgaggacatgaggggtattctgtacgcagcgcctcagttgaggtccatccgcggccagccaggcccaaaccatgaggaagatgccgattcttacgaaaacatggacaaccccgatggtcctgaccccgcatgggggggcggcgggaggatgggcacctggtctactcgctag

SEQ ID NO: 40 - protein, 567 aa, synthetic construct
EVQLVQSGAEVKKPGASVKVSCKTSGYTFTEYTIHWVRQAPGQSLEWMGNINPNNGGTTYNQKFQGRVTITVDKSTSTAYMELSSLRSEDTAVYYCAAGWNFDYWGQGTLVTVSSGKPGSGKPGSGKPGSGKPGSDIVMTQSPDSLAVSLGERATLSCRASQDVGTAVDWYQQKPDQSPKLLIYWASTRHTGVPDRFTGSGSGTDFTLTISSLQAEDVAVYFCQQYNSYPLTFGAGTKVEIKFVPVFLPAKPTTTPAPRPPTPAPTIASQPLSLRPEACRPAAGGAVHTRGLDFACDFWVLVVVGGVLACYSLLVTVAFIIFWVQRALVLRRKRKRMTDPTRRFFKVTPPPGSGPQNQYGNVLSLPTPTSGLGRAQRWAAGLGGTAPSYGNPSSDVQADGALGSRSPPGVGPEEEEGEGYEEPDSEEDSEFYENDSNLGQDQLSQDGSGYENPEDEPLGPEDEDSFSNAESYENEDEELTQPVARTMDFLSPHGSAWDPSREATSLGSQSYEDMRGILYAAPQLRSIRGQPGPNHEEDADSYENMDNPDGPDPAWGGGGRMGTWSTR

SEQ ID NO: 41 - DNA, 1122 bp, synthetic construct
  source 1..1122: mol_type = other DNA
gaggttcaacttgttcaatctggggcagaagtgaagaagcccggggcatctgtgaaagtatcatgcaaaacatccggctatacgtttaccgaatacaccattcactgggtcagacaggctcccggtcaaagcctcgaatggatgggaaatattaaccctaacaatggcggaaccacatataatcagaaattccaaggccgagtgacgataactgtcgataagagtacgtccacagcttacatggaactcagctctttgagatccgaagacactgcagtttattattgtgcagctggatggaacttcgactattggggacaagggactcttgttacggtgtccagtggcaaaccaggtagtggtaaacccggaagcggcaagcccgggagcggtaaacctggtagcgacatcgtcatgactcaaagccctgactcactcgccgtgagcctgggagagcgtgcaacgctatcttgtcgggcctctcaggatgtcggaactgctgtagactggtatcaacagaaacctgaccaatcaccaaaactcctgatttattgggcctcaacacgtcacacaggagtgccagataggttcacaggtagtggcagtggaactgattttactttgacaattagcagcctgcaagccgaagatgtagccgtttacttctgtcaacaatataactcatacccactaacgttcggtgccgggacgaaggtagagattaaattcgtgcctgtgttcctcccagctaagcccactaccacccccgctccaaggccgcccacgcccgctcctactattgctagtcagcctttaagtttacgacccgaagcttgcaggcccgccgccggcggcgctgtgcacaccagggggcttgattttgcctgcgacttttgggtattggtagtggtgggcggagttttagcctgctacagcctcctggtaacagtggcttttatcatcttttgggtgctggacaaggatgacagcaaggctggcatggaggaagatcacacctacgagggcctggacattgaccagacagccacctatgaggacatagtgacgctgcggacaggggaagtgaagtggtctgtaggtgagcacccaggccaggagtga

SEQ ID NO: 42 - protein, 373 aa, synthetic construct
EVQLVQSGAEVKKPGASVKVSCKTSGYTFTEYTIHWVRQAPGQSLEWMGNINPNNGGTTYNQKFQGRVTITVDKSTSTAYMELSSLRSEDTAVYYCAAGWNFDYWGQGTLVTVSSGKPGSGKPGSGKPGSGKPGSDIVMTQSPDSLAVSLGERATLSCRASQDVGTAVDWYQQKPDQSPKLLIYWASTRHTGVPDRFTGSGSGTDFTLTISSLQAEDVAVYFCQQYNSYPLTFGAGTKVEIKFVPVFLPAKPTTTPAPRPPTPAPTIASQPLSLRPEACRPAAGGAVHTRGLDFACDFWVLVVVGGVLACYSLLVTVAFIIFWVLDKDDSKAGMEEDHTYEGLDIDQTATYEDIVTLRTGEVKWSVGEHPGQE

SEQ ID NO: 43 - DNA, 1305 bp, synthetic construct
  source 1..1305: mol_type = other DNA
gaggttcaacttgttcaatctggggcagaagtgaagaagcccggggcatctgtgaaagtatcatgcaaaacatccggctatacgtttaccgaatacaccattcactgggtcagacaggctcccggtcaaagcctcgaatggatgggaaatattaaccctaacaatggcggaaccacatataatcagaaattccaaggccgagtgacgataactgtcgataagagtacgtccacagcttacatggaactcagctctttgagatccgaagacactgcagtttattattgtgcagctggatggaacttcgactattggggacaagggactcttgttacggtgtccagtggcaaaccaggtagtggtaaacccggaagcggcaagcccgggagcggtaaacctggtagcgacatcgtcatgactcaaagccctgactcactcgccgtgagcctgggagagcgtgcaacgctatcttgtcgggcctctcaggatgtcggaactgctgtagactggtatcaacagaaacctgaccaatcaccaaaactcctgatttattgggcctcaacacgtcacacaggagtgccagataggttcacaggtagtggcagtggaactgattttactttgacaattagcagcctgcaagccgaagatgtagccgtttacttctgtcaacaatataactcatacccactaacgttcggtgccgggacgaaggtagagattaaattcgtgcctgtgttcctcccagctaagcccactaccacccccgctccaaggccgcccacgcccgctcctactattgctagtcagcctttaagtttacgacccgaagcttgcaggcccgccgccggcggcgctgtgcacaccagggggcttgattttgcctgcgacttttgggtattggtagtggtgggcggagttttagcctgctacagcctcctggtaacagtggcttttatcatcttttgggtgaagaaggttgcaaaaaaacctactaataaggctccccatcctaagcaagagccccaagaaattaactttcccgatgatcttccgggttctaacacggcagccccggtgcaggagaccctgcatggttgtcaacccgtcactcaggaggacgggaaagagtctcgtatctccgtccaggagagacaggacaaggacgatagtaaagcagggatggaggaggaccatacatacgagggactggatatcgatcagacagccacgtacgaagacattgtgacactgagaactggcgaggtgaagtggtcagtgggagaacatccggggcaggaataa

SEQ ID NO: 44 - protein, 434 aa, synthetic construct
EVQLVQSGAEVKKPGASVKVSCKTSGYTFTEYTIHWVRQAPGQSLEWMGNINPNNGGTTYNQKFQGRVTITVDKSTSTAYMELSSLRSEDTAVYYCAAGWNFDYWGQGTLVTVSSGKPGSGKPGSGKPGSGKPGSDIVMTQSPDSLAVSLGERATLSCRASQDVGTAVDWYQQKPDQSPKLLIYWASTRHTGVPDRFTGSGSGTDFTLTISSLQAEDVAVYFCQQYNSYPLTFGAGTKVEIKFVPVFLPAKPTTTPAPRPPTPAPTIASQPLSLRPEACRPAAGGAVHTRGLDFACDFWVLVVVGGVLACYSLLVTVAFIIFWVKKVAKKPTNKAPHPKQEPQEINFPDDLPGSNTAAPVQETLHGCQPVTQEDGKESRISVQERQDKDDSKAGMEEDHTYEGLDIDQTATYEDIVTLRTGEVKWSVGEHPGQE

SEQ ID NO: 45 - DNA, 1287 bp, synthetic construct
  source 1..1287: mol_type = other DNA
gaggttcaacttgttcaatctggggcagaagtgaagaagcccggggcatctgtgaaagtatcatgcaaaacatccggctatacgtttaccgaatacaccattcactgggtcagacaggctcccggtcaaagcctcgaatggatgggaaatattaaccctaacaatggcggaaccacatataatcagaaattccaaggccgagtgacgataactgtcgataagagtacgtccacagcttacatggaactcagctctttgagatccgaagacactgcagtttattattgtgcagctggatggaacttcgactattggggacaagggactcttgttacggtgtccagtggcaaaccaggtagtggtaaacccggaagcggcaagcccgggagcggtaaacctggtagcgacatcgtcatgactcaaagccctgactcactcgccgtgagcctgggagagcgtgcaacgctatcttgtcgggcctctcaggatgtcggaactgctgtagactggtatcaacagaaacctgaccaatcaccaaaactcctgatttattgggcctcaacacgtcacacaggagtgccagataggttcacaggtagtggcagtggaactgattttactttgacaattagcagcctgcaagccgaagatgtagccgtttacttctgtcaacaatataactcatacccactaacgttcggtgccgggacgaaggtagagattaaattcgtgcctgtgttcctcccagctaagcccactaccacccccgctccaaggccgcccacgcccgctcctactattgctagtcagcctttaagtttacgacccgaagcttgcaggcccgccgccggcggcgctgtgcacaccagggggcttgattttgcctgcgacttttgggtattggtagtggtgggcggagttttagcctgctacagcctcctggtaacagtggcttttatcatcttttgggtgaagaaggttgcaaaaaaacctactaataaggctccccatcctaagcaagagccccaagaaattaactttcccgatgatcttccgggttctaacacggcagccccggtgcaggagaccctgcatggttgtcaacccgtcactcaggaggacgggaaagagtctcgtatctccgtccaggagagacagaaaagaggccgaaaaaagctgctgtacatcttcaaacaacccttcatgcgacctgttcagacgacacaggaggaggacggctgcagctgtaggtttcccgaagaagaggagggaggatgcgaactttaa

SEQ ID NO: 46 - protein, 428 aa, synthetic construct
EVQLVQSGAEVKKPGASVKVSCKTSGYTFTEYTIHWVRQAPGQSLEWMGNINPNNGGTTYNQKFQGRVTITVDKSTSTAYMELSSLRSEDTAVYYCAAGWNFDYWGQGTLVTVSSGKPGSGKPGSGKPGSGKPGSDIVMTQSPDSLAVSLGERATLSCRASQDVGTAVDWYQQKPDQSPKLLIYWASTRHTGVPDRFTGSGSGTDFTLTISSLQAEDVAVYFCQQYNSYPLTFGAGTKVEIKFVPVFLPAKPTTTPAPRPPTPAPTIASQPLSLRPEACRPAAGGAVHTRGLDFACDFWVLVVVGGVLACYSLLVTVAFIIFWVKKVAKKPTNKAPHPKQEPQEINFPDDLPGSNTAAPVQETLHGCQPVTQEDGKESRISVQERQKRGRKKLLYIFKQPFMRPVQTTQEEDGCSCRFPEEEEGGCEL

SEQ ID NO: 47 - protein, 462 aa, synthetic construct
EVQLVQSGAEVKKPGASVKVSCKTSGYTFTEYTIHWVRQAPGQSLEWMGNINPNNGGTTYNQKFQGRVTITVDKSTSTAYMELSSLRSEDTAVYYCAAGWNFDYWGQGTLVTVSSGKPGSGKPGSGKPGSGKPGSDIVMTQSPDSLAVSLGERATLSCRASQDVGTAVDWYQQKPDQSPKLLIYWASTRHTGVPDRFTGSGSGTDFTLTISSLQAEDVAVYFCQQYNSYPLTFGAGTKVEIKFVPVFLPAKPTTTPAPRPPTPAPTIASQPLSLRPEACRPAAGGAVHTRGLDFACDFWVLVVVGGVLACYSLLVTVAFIIFWVKKVAKKPTNKAPHPKQEPQEINFPDDLPGSNTAAPVQETLHGCQPVTQEDGKESRISVQERQRKKRISANSTDPVKAAQFEPPGRQMIAIRKRQLEETNNDYETADGGYMTLNPRAPTDDDKNIYLTLPPNDHVNSNN

SEQ ID NO: 48 - DNA, 1656 bp, synthetic construct
  source 1..1656: mol_type = other DNA
gaggttcaacttgttcaatctggggcagaagtgaagaagcccggggcatctgtgaaagtatcatgcaaaacatccggctatacgtttaccgaatacaccattcactgggtcagacaggctcccggtcaaagcctcgaatggatgggaaatattaaccctaacaatggcggaaccacatataatcagaaattccaaggccgagtgacgataactgtcgataagagtacgtccacagcttacatggaactcagctctttgagatccgaagacactgcagtttattattgtgcagctggatggaacttcgactattggggacaagggactcttgttacggtgtccagtggcaaaccaggtagtggtaaacccggaagcggcaagcccgggagcggtaaacctggtagcgacatcgtcatgactcaaagccctgactcactcgccgtgagcctgggagagcgtgcaacgctatcttgtcgggcctctcaggatgtcggaactgctgtagactggtatcaacagaaacctgaccaatcaccaaaactcctgatttattgggcctcaacacgtcacacaggagtgccagataggttcacaggtagtggcagtggaactgattttactttgacaattagcagcctgcaagccgaagatgtagccgtttacttctgtcaacaatataactcatacccactaacgttcggtgccgggacgaaggtagagattaaattcgtgcctgtgttcctcccagctaagcccactaccacccccgctccaaggccgcccacgcccgctcctactattgctagtcagcctttaagtttacgacccgaagcttgcaggcccgccgccggcggcgctgtgcacaccagggggcttgattttgcctgcgacttttgggtattggtagtggtgggcggagttttagcctgctacagcctcctggtaacagtggcttttatcatcttttgggtgatggcggcgggcgggcccggcgccggaagcgccgcgccagtctcatctacgtccagtctgccactggctgccctgaacatgagagtgagacgccgtttatccctcttcctgaatgtgcggacccaggtcgccgctgattggaccgccctggccgaagagatggactttgaatacttggaaatcagacagctggaaacacaggcagacccaaccgggagactgcttgacgcctggcagggacgcccaggggcaagtgttggtcggttactggagcttttaactaagttgggccgcgatgacgtgctgttggagttaggacccagtatcgaggaggattgtcagaaatacatcttgaaacagcagcaggaggaggcggaaaagcccctgcaggtggcggccgttgacagcagtgtacccagaacagctgagctggccggcatcacaaccctggatgatcccctgggccacatgcctgagaggttcgacgctttcataaagaaggttgcaaaaaaacctactaataaggctccccatcctaagcaagagccccaagaaattaactttcccgatgatcttccgggttctaacacggcagccccggtgcaggagaccctgcatggttgtcaacccgtcactcaggaggacgggaaagagtctcgtatctccgtccaggagagacagtga

SEQ ID NO: 49 - protein, 551 aa, synthetic construct
EVQLVQSGAEVKKPGASVKVSCKTSGYTFTEYTIHWVRQAPGQSLEWMGNINPNNGGTTYNQKFQGRVTITVDKSTSTAYMELSSLRSEDTAVYYCAAGWNFDYWGQGTLVTVSSGKPGSGKPGSGKPGSGKPGSDIVMTQSPDSLAVSLGERATLSCRASQDVGTAVDWYQQKPDQSPKLLIYWASTRHTGVPDRFTGSGSGTDFTLTISSLQAEDVAVYFCQQYNSYPLTFGAGTKVEIKFVPVFLPAKPTTTPAPRPPTPAPTIASQPLSLRPEACRPAAGGAVHTRGLDFACDFWVLVVVGGVLACYSLLVTVAFIIFWVMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFIKKVAKKPTNKAPHPKQEPQEINFPDDLPGSNTAAPVQETLHGCQPVTQEDGKESRISVQERQ

SEQ ID NO: 50 - DNA, 1158 bp, synthetic construct
  source 1..1158: mol_type = other DNA
gaggttcaacttgttcaatctggggcagaagtgaagaagcccggggcatctgtgaaagtatcatgcaaaacatccggctatacgtttaccgaatacaccattcactgggtcagacaggctcccggtcaaagcctcgaatggatgggaaatattaaccctaacaatggcggaaccacatataatcagaaattccaaggccgagtgacgataactgtcgataagagtacgtccacagcttacatggaactcagctctttgagatccgaagacactgcagtttattattgtgcagctggatggaacttcgactattggggacaagggactcttgttacggtgtccagtggcaaaccaggtagtggtaaacccggaagcggcaagcccgggagcggtaaacctggtagcgacatcgtcatgactcaaagccctgactcactcgccgtgagcctgggagagcgtgcaacgctatcttgtcgggcctctcaggatgtcggaactgctgtagactggtatcaacagaaacctgaccaatcaccaaaactcctgatttattgggcctcaacacgtcacacaggagtgccagataggttcacaggtagtggcagtggaactgattttactttgacaattagcagcctgcaagccgaagatgtagccgtttacttctgtcaacaatataactcatacccactaacgttcggtgccgggacgaaggtagagattaaattcgtgcctgtgttcctcccagctaagcccactaccacccccgctccaaggccgcccacgcccgctcctactattgctagtcagcctttaagtttacgacccgaagcttgcaggcccgccgccggcggcgctgtgcacaccagggggcttgattttgcctgcgacttttgggtattggtagtggtgggcggagttttagcctgctacagcctcctggtaacagtggcttttatcatcttttgggtgaggaaacgatggcagaacgagaagctcgggttggatgccggggatgaatatgaagatgaaaacctttatgaaggcctgaacctggacgactgctccatgtatgaggacatctcccggggcctccagggcacctaccaggatgtgggcagcctcaacataggagatgtccagctggagaagccgtga

SEQ ID NO: 51 - protein, 385 aa, synthetic construct
EVQLVQSGAEVKKPGASVKVSCKTSGYTFTEYTIHWVRQAPGQSLEWMGNINPNNGGTTYNQKFQGRVTITVDKSTSTAYMELSSLRSEDTAVYYCAAGWNFDYWGQGTLVTVSSGKPGSGKPGSGKPGSGKPGSDIVMTQSPDSLAVSLGERATLSCRASQDVGTAVDWYQQKPDQSPKLLIYWASTRHTGVPDRFTGSGSGTDFTLTISSLQAEDVAVYFCQQYNSYPLTFGAGTKVEIKFVPVFLPAKPTTTPAPRPPTPAPTIASQPLSLRPEACRPAAGGAVHTRGLDFACDFWVLVVVGGVLACYSLLVTVAFIIFWVRKRWQNEKLGLDAGDEYEDENLYEGLNLDDCSMYEDISRGLQGTYQDVGSLNIGDVQLEKP

SEQ ID NO: 52 - DNA, 1122 bp, synthetic construct; identical to SEQ ID NO: 41
  source 1..1122: mol_type = other DNA

SEQ ID NO: 53 - protein, 373 aa, synthetic construct; identical to SEQ ID NO: 42

SEQ ID NO: 54 - DNA, 1143 bp, synthetic construct
  source 1..1143: mol_type = other DNA
gaggttcaacttgttcaatctggggcagaagtgaagaagcccggggcatctgtgaaagtatcatgcaaaacatccggctatacgtttaccgaatacaccattcactgggtcagacaggctcccggtcaaagcctcgaatggatgggaaatattaaccctaacaatggcggaaccacatataatcagaaattccaaggccgagtgacgataactgtcgataagagtacgtccacagcttacatggaactcagctctttgagatccgaagacactgcagtttattattgtgcagctggatggaacttcgactattggggacaagggactcttgttacggtgtccagtggcaaaccaggtagtggtaaacccggaagcggcaagcccgggagcggtaaacctggtagcgacatcgtcatgactcaaagccctgactcactcgccgtgagcctgggagagcgtgcaacgctatcttgtcgggcctctcaggatgtcggaactgctgtagactggtatcaacagaaacctgaccaatcaccaaaactcctgatttattgggcctcaacacgtcacacaggagtgccagataggttcacaggtagtggcagtggaactgattttactttgacaattagcagcctgcaagccgaagatgtagccgtttacttctgtcaacaatataactcatacccactaacgttcggtgccgggacgaaggtagagattaaaggcgctggtagtggcggtaactggagccaccctcaatttgagaagggcgggtcaggcggatcaggtggtagtggtgggtccaactggagccatccgcaatttgaaaagggcggaagcggcggttccggcggttcaggcggtagcaactggtcacatccgcaatttgagaaaggcgggtcaggcggcgggttttgggctctcgtggtggtggctggagtgcttttctgctatggcctgctggtaaccgtggccctttgtgtaatctggaccgataaagacgatggaaaagccgggatggaagaagaccatacctacgaggggctcaatattgatcaaaccgccacgtatgaagacattgtaacactgcgcacaggtgaggtcaagtggtccgtcggtgaacacccaggacaagaataa

SEQ ID NO: 55 - protein, 380 aa, synthetic construct
EVQLVQSGAEVKKPGASVKVSCKTSGYTFTEYTIHWVRQAPGQSLEWMGNINPNNGGTTYNQKFQGRVTITVDKSTSTAYMELSSLRSEDTAVYYCAAGWNFDYWGQGTLVTVSSGKPGSGKPGSGKPGSGKPGSDIVMTQSPDSLAVSLGERATLSCRASQDVGTAVDWYQQKPDQSPKLLIYWASTRHTGVPDRFTGSGSGTDFTLTISSLQAEDVAVYFCQQYNSYPLTFGAGTKVEIKGAGSGGNWSHPQFEKGGSGGSGGSGGSNWSHPQFEKGGSGGSGGSGGSNWSHPQFEKGGSGGGFWALVVVAGVLFCYGLLVTVALCVIWTDKDDGKAGMEEDHTYEGLNIDQTATYEDIVTLRTGEVKWSVGEHPGQE

SEQ ID NO: 56 - DNA, 1152 bp, synthetic construct
  source 1..1152: mol_type = other DNA
gaagtccaattggttgaaagcggtggtggactcgtcaaacctggcggtagccttaaactttcatgtgccgcaagcggcttcacgtttagtaactatgctatgagttgggtccgccaaagtccagaaaagcgcctcgaatgggtggcggagatctctggaggaggaacatatacatattatccagacaccatgaccggtaggtttacaatctcaagagacaacgctaagaacaccctgtacctggaaatgtcaagcctgagatcagaagatacggccatgtattattgtacgcgcctactcgactattggggtcaaggaacttccgtgacggtgtcaagcggaggaggtgggagcggaggaggcggaagtggcggtggtggctctggtggcggtggaagtgatatagtgatgacgcaagctgccttttcaaaccctgttactttggggactagcgcatcaatctcctgtaggtccagcaaatctttgctgcacagtaatggaatcacctatcttttctggtatttgcaaaagcctgggcagagcccgcaactgctgatctatcaaatgtcaaatcttgcttccggagttccagaccgcttctcaagttccgggtccggcactgattttaccttgagaatttctagggtcgaagctgaagacgtcggtgtctattattgcgcgcaaaaccttgagcttccatacaccttcggggggggcacaaaacttgagatcaagggcgctgggagcggcgggaattggagtcatccacaattcgaaaagggtgggtccggcggcagtggtggaagcggcgggagtaactggtcacatccccagtttgagaaaggcggtagtggtggcagcggcggtagtggtggcagtaattggagccatccccaattcgaaaagggcggttccggcggcggattttgggctcttgttgtggtggccggagtattgttttgctatggcctgctcgttacagtggcattgtgcgtaatttggactgataaagacgacggcaaagccgggatggaagaagatcacacctatgaggggcttaatatagatcaaacagccacatatgaagatattgtgactctaaggactggagaggttaaatggagtgtgggtgagcatccaggacaagaataa

SEQ ID NO: 57 - protein, 383 aa, synthetic construct
EVQLVESGGGLVKPGGSLKLSCAASGFTFSNYAMSWVRQSPEKRLEWVAEISGGGTYTYYPDTMTGRFTISRDNAKNTLYLEMSSLRSEDTAMYYCTRLLDYWGQGTSVTVSSGGGGSGGGGSGGGGSGGGGSDIVMTQAAFSNPVTLGTSASISCRSSKSLLHSNGITYLFWYLQKPGQSPQLLIYQMSNLASGVPDRFSSSGSGTDFTLRISRVEAEDVGVYYCAQNLELPYTFGGGTKLEIKGAGSGGNWSHPQFEKGGSGGSGGSGGSNWSHPQFEKGGSGGSGGSGGSNWSHPQFEKGGSGGGFWALVVVAGVLFCYGLLVTVALCVIWTDKDDGKAGMEEDHTYEGLNIDQTATYEDIVTLRTGEVKWSVGEHPGQE

SEQ ID NO: 58 - DNA, 1191 bp, synthetic construct
  source 1..1191: mol_type = other DNA
cagtctgtgttgacgcagccgccctcagtgtctgcggccccaggacagagggtcaccatctcctgctctggaaccaggtccaacattgggagtgattatgtttcctggtaccaacacctcccaggaacagcccccaaactcctcgtttatggcgataatctgcgaccctcagggattcctgaccgattctctgcctccaagtctggcacgtcagccaccctgggcatcaccggactccagactggggacgaggccgattattactgcggcacatgggattacaccctgaatggtgtggtgttcggcggagggaccaagctgaccgtcctaggttctagaggtggtggtggtagcggcggcggcggctctggtggtggtggatccctcgagatggcccaggtgcagctggtggagtctgggggaggcttggtacagcctggggggtccctgagactctcctgtgcagcctctggattcacctttagcagctatgccatgagctgggtccgccaggctccagggaaggggctggagtgggtctcagttatttatagcggtggtagtagcacatactatgcagactccgtgaagggccggttcaccatctccagagataattccaagaacacgctgtatctgcaaatgaacagcctgagagccgaggacacggccgtatattactgtgcgcgcacttcttacctgaaccatggtgattactggggtcaaggtactctggtgaccgtgtctagcgccgctgcattcgtgcctgtgttcctcccagctaagcccactaccacccccgctccaaggccgcccacgcccgctcctactattgctagtcagcctttaagtttacgacccgaagcttgcaggcccgccgccggcggcgctgtgcacaccagggggcttgattttgcctgcgacttttgggtattggtagtggtgggcggagttttagcctgctacagcctcctggtaacagtggcttttatcatcttttgggtgaggaaacgatggcagaacgagaagctcgggttggatgccggggatgaatatgaagatgaaaacctttatgaaggcctgaacctggacgactgctccatgtatgaggacatctcccggggcctccagggcacctaccaggatgtgggcagcctcaacataggagatgtccagctggagaagccgtga

SEQ ID NO: 59 - protein, 396 aa, synthetic construct
QSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSAAAFVPVFLPAKPTTTPAPRPPTPAPTIASQPLSLRPEACRPAAGGAVHTRGLDFACDFWVLVVVGGVLACYSLLVTVAFIIFWVRKRWQNEKLGLDAGDEYEDENLYEGLNLDDCSMYEDISRGLQGTYQDVGSLNIGDVQLEKP

SEQ ID NO: 60 - DNA, 1155 bp, synthetic construct
  source 1..1155: mol_type = other DNA
cagtctgtgttgacgcagccgccctcagtgtctgcggccccaggacagagggtcaccatctcctgctctggaaccaggtccaacattgggagtgattatgtttcctggtaccaacacctcccaggaacagcccccaaactcctcgtttatggcgataatctgcgaccctcagggattcctgaccgattctctgcctccaagtctggcacgtcagccaccctgggcatcaccggactccagactggggacgaggccgattattactgcggcacatgggattacaccctgaatggtgtggtgttcggcggagggaccaagctgaccgtcctaggttctagaggtggtggtggtagcggcggcggcggctctggtggtggtggatccctcgagatggcccaggtgcagctggtggagtctgggggaggcttggtacagcctggggggtccctgagactctcctgtgcagcctctggattcacctttagcagctatgccatgagctgggtccgccaggctccagggaaggggctggagtgggtctcagttatttatagcggtggtagtagcacatactatgcagactccgtgaagggccggttcaccatctccagagataattccaagaacacgctgtatctgcaaatgaacagcctgagagccgaggacacggccgtatattactgtgcgcgcacttcttacctgaaccatggtgattactggggtcaaggtactctggtgaccgtgtctagcgccgctgcattcgtgcctgtgttcctcccagctaagcccactaccacccccgctccaaggccgcccacgcccgctcctactattgctagtcagcctttaagtttacgacccgaagcttgcaggcccgccgccggcggcgctgtgcacaccagggggcttgattttgcctgcgacttttgggtattggtagtggtgggcggagttttagcctgctacagcctcctggtaacagtggcttttatcatcttttgggtgctggacaaggatgacagcaaggctggcatggaggaagatcacacctacgagggcctggacattgaccagacagccacctatgaggacatagtgacgctgcggacaggggaagtgaagtggtctgtaggtgagcacccaggccaggagtga

SEQ ID NO: 61 - protein, 384 aa, synthetic construct
QSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSAAAFVPVFLPAKPTTTPAPRPPTPAPTIASQPLSLRPEACRPAAGGAVHTRGLDFACDFWVLVVVGGVLACYSLLVTVAFIIFWVLDKDDSKAGMEEDHTYEGLDIDQTATYEDIVTLRTGEVKWSVGEHPGQE

SEQ ID NO: 62 - DNA, 1653 bp, synthetic construct
  source 1..1653: mol_type = other DNA
cagtctgtgttgacgcagccgccctcagtgtctgcggccccaggacagagggtcaccatctcctgctctggaaccaggtccaacattgggagtgattatgtttcctggtaccaacacctcccaggaacagcccccaaactcctcgtttatggcgataatctgcgaccctcagggattcctgaccgattctctgcctccaagtctggcacgtcagccaccctgggcatcaccggactccagactggggacgaggccgattattactgcggcacatgggattacaccctgaatggtgtggtgttcggcggagggaccaagctgaccgtcctaggttctagaggtggtggtggtagcggcggcggcggctctggtggtggtggatccctcgagatggcccaggtgcagctggtggagtctgggggaggcttggtacagcctggggggtccctgagactctcctgtgcagcctctggattcacctttagcagctatgccatgagctgggtccgccaggctccagggaaggggctggagtgggtctcagttatttatagcggtggtagtagcacatactatgcagactccgtgaagggccggttcaccatctccagagataattccaagaacacgctgtatctgcaaatgaacagcctgagagccgaggacacggccgtatattactgtgcgcgcacttcttacctgaaccatggtgattactggggtcaaggtactctggtgaccgtgtctagccccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccgagctgcaactggaggagagctgtgcggaggcgcaggacggggagctggacgggctgtggacgaccatcaccatcttcatcacactcttcctgttaagcgtgtgctacagtgccaccgtcaccttcttcaaggtgaagtggatcttctcctcggtggtggacctgaagcagaccatcatccccgactacaggaacatgatcggacagggggcctga

SEQ ID NO: 63 - protein, 550 aa, synthetic construct
QSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPELQLEESCAEAQDGELDGLWTTITIFITLFLLSVCYSATVTFFKVKWIFSSVVDLKQTIIPDYRNMIGQGA

SEQ ID NO: 64 - DNA, 648 bp, synthetic construct
  source 1..648: mol_type = other DNA
cagtctgtgttgacgcagccgccctcagtgtctgcggccccaggacagagggtcaccatctcctgctctggaaccaggtccaacattgggagtgattatgtttcctggtaccaacacctcccaggaacagcccccaaactcctcgtttatggcgataatctgcgaccctcagggattcctgaccgattctctgcctccaagtctggcacgtcagccaccctgggcatcaccggactccagactggggacgaggccgattattactgcggcacatgggattacaccctgaatggtgtggtgttcggcggagggaccaagctgaccgtcctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 65 - protein, 216 aa, synthetic construct
QSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 66 - DNA, 1554 bp, synthetic construct
  source 1..1554: mol_type = other DNA
caggtgcagctggtggagtctgggggaggcttggtacagcctggggggtccctgagactctcctgtgcagcctctggattcacctttagcagctatgccatgagctgggtccgccaggctccagggaaggggctggagtgggtctcagttatttatagcggtggtagtagcacatactatgcagactccgtgaagggccggttcaccatctccagagataattccaagaacacgctgtatctgcaaatgaacagcctgagagccgaggacacggccgtatattactgtgcgcgcacttcttacctgaaccatggtgattactggggtcaaggtactctggtgaccgtgtctagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccgagctgcaactggaggagagctgtgcggaggcgcaggacggggagctggacgggctgtggacgaccatcaccatcttcatcacactcttcctgttaagcgtgtgctacagtgccaccgtcaccttcttcaaggtgaagtggatcttctcctcggtggtggacctgaagcagaccatcatccccgactacaggaacatgatcggacagggggcctga

SEQ ID NO: 67 - protein, 517 aa, synthetic construct
QVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPELQLEESCAEAQDGELDGLWTTITIFITLFLLSVCYSATVTFFKVKWIFSSVVDLKQTIIPDYRNMIGQGA

SEQ ID NO: 68 - DNA, 327 bp, synthetic construct
  source 1..327: mol_type = other DNA
atgagactttcaacagcaacactcctcctgttgctggcttcatgtctgagccctggtcatggtattttggaggcccactatacaaatctcaaatgtcggtgttcaggcgtaatatccaccgtagtcggcctgaacattatcgataggattcaggttacaccccccgggaacggatgtcctaagaccgaggtggtgatttggaccaagatgaagaaggtcatttgtgtgaacccacgggctaaatggctgcagcgtcttttgcgacacgtgcagtccaagagcttgtccagcacacctcaggccccagttagcaagcgacgtgcagcc

SEQ ID NO: 69 - protein, 109 aa, synthetic construct
MRLSTATLLLLLASCLSPGHGILEAHYTNLKCRCSGVISTVVGLNIIDRIQVTPPGNGCPKTEVVIWTKMKKVICVNPRAKWLQRLLRHVQSKSLSSTPQAPVSKRRAA

SEQ ID NO: 70 - DNA, 696 bp, synthetic construct
  source 1..696: mol_type = other DNA
atgacagtgctggcccccgcgtggtctcccaatagctcactcctcctcttgctgctactgctcagcccatgcctcaggggcacccccgattgttacttcagccacagcccaatctcctccaacttcaaagtgaaatttagggaactgaccgaccacctgctgaaagattatcctgtgactgtggcagtgaacctgcaagacgaaaagcattgtaaggcgctatggagcctctttcttgcccaacgatggattgagcaactcaaaactgtagccggaagcaaaatgcagacgctactggaggacgtgaatactgagattcacttcgttaccagttgtactttccagccactgccagagtgtctcaggtttgtgcagactaatatcagccacctgctgaaggatacttgcacccagctcctggctctcaagccttgtataggcaaggcttgtcaaaattttagcaggtgtctcgaagtccagtgccagccagattcatccacactgctgccgccccgaagccctatcgcactcgaagcgacagagttgccagagcctcgtcccagacagcttctgctgctgctacttctgctgctgccgctaactctggtgctacttgctgccgcctggggcctcagatggcaacgcgccagacgccgaggcgaactccaccctggggtgccactgccatcccaccca

SEQ ID NO: 71 - protein, 232 aa, synthetic construct
MTVLAPAWSPNSSLLLLLLLLSPCLRGTPDCYFSHSPISSNFKVKFRELTDHLLKDYPVTVAVNLQDEKHCKALWSLFLAQRWIEQLKTVAGSKMQTLLEDVNTEIHFVTSCTFQPLPECLRFVQTNISHLLKDTCTQLLALKPCIGKACQNFSRCLEVQCQPDSSTLLPPRSPIALEATELPEPRPRQLLLLLLLLLPLTLVLLAAAWGLRWQRARRRGELHPGVPLPSHP

SEQ ID NO: 72 - DNA, 342 bp, synthetic construct
  source 1..342: mol_type = other DNA
atgcgactcttgttgttgacttttctcggagtgtgctgcctgacaccctgggtcgtagagggagttggcactgaagtactagaagagtcctcctgcgttaacctgcagacacagcggctcccagtccagaaaattaagacctacattatatgggaaggagcaatgcgagcggtgatttttgtgaccaagaggggtctcaagatttgcgcggaccctgaggccaagtgggtcaaagcagctattaagacagtagacggaagagcctccaccaggaagaatatggcagaaactgtaccgaccggtgcgcagcggtcaacatctaccgcaatcacactcaccggc

SEQ ID NO: 73 - protein, 60 aa, synthetic construct
MRAVIFVTKRGLKICADPEAKWVKAAIKTVDGRASTRKNMAETVPTGAQRSTSTAITLTG

SEQ ID NO: 74 - DNA, 1533 bp, synthetic construct
  source 1..1533: mol_type = other DNA
atgagcaagggccttctcctgctgtggctagtaactgaattgtggtggttgtacctgacacctgccgctagtgaggacaccatcattggtttccttgggcagcccgtcaccctcccttgccattacctaagctggagccagtcacggaactctatgtgctggggaaaggggtcatgccctaattccaagtgcaacgccgagctgttgcgcacggacggcaccagaataatctcaagaaagtccaccaagtatacgctgctcggcaaggtgcaattcggtgaagtgagcttgaccataagtaacaccaaccgcggtgactccggagtttattgttgcaggatcgaagtgccaggctggtttaacgacgtgaagaaaaacgtgcggctggaactgaggagggcaactacgaccaagaaaccaacaaccacgacgagacctaccaccactccttacgtgacaaccacgacaccggagctgttgccaactaccgtcatgacaacatctgtgttgccaactaccacccccccccaaacgctcgcgacaactgccttttccacagccgttaccacatgtccttccaccaccccaggctctttttctcaagaaactaccaagggatcagcttttaccaccgagtctgaaactctcccagcaagtaatcactcacagcggtcaatgatgaccatcagcacagacatcgctgtcttgagacctactggcagcaatccaggcattctgccctccacttcacagctgactacccaaaagactacactaaccaccagcgaaagtctgcagaaaactacaaagagccatcaaataaactcccggcagactcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 75 - protein, 511 aa, synthetic construct
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 76 - DNA, 927 bp, synthetic construct
  source 1..927: mol_type = other DNA
atggatcagcatacactggacgtggaagatacagccgatgccagacaccctgctggaacgtcctgtcccagcgacgctgccctgctcagagacaccgggctgctcgcagatgctgctctgctgagtgataccgttcggccaactaacgcggccctacccacagatgccgcatatcccgcggtaaatgtcagggaccgggaagctgcctggccaccggccctcaatttctgctctagacatccgaaactgtacggtctggtcgcactggtactgctgctacttatagcagcttgtgttcccatatttacccgcactgaacccagacccgctctcactattacaacttcaccaaacttgggcacacgtgaaaacaatgcagatcaggttacccctgtaagtcatattggatgccccaacaccacacaacagggaagtccggtgtttgcaaaactccttgctaagaatcaggcttcactgtgtaacactactcttaattggcactcacaagacggggccgggagtagctatctcagccaaggtctccgctatgaagaagataagaaagagttggtggtggacagcccaggactctactacgtcttcctggagctaaaactaagccccacttttactaacactggacataaggtccaaggttgggtgtccctcgtacttcaagctaaaccccaggtggacgacttcgataacctggcgttgacagttgagctctttccttgctctatggaaaataagctcgtggatcggagctggtctcaactgttgctgcttaaagccggtcatcgtctgtctgttggactacgcgcatacttgcatggagcccaggacgcatatcgtgattgggaactgagctacccgaataccactagctttggactatttcttgttaaaccagataatccttgggag

SEQ ID NO: 77 - protein, 309 aa, synthetic construct
MDQHTLDVEDTADARHPAGTSCPSDAALLRDTGLLADAALLSDTVRPTNAALPTDAAYPAVNVRDREAAWPPALNFCSRHPKLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 78 - DNA, 705 bp, synthetic construct
  source 1..705: mol_type = other DNA
atggagagcgtagtgcaacccagcgtatttgtggtggatggacagaccgacatcccattcagacgcttggaacagaaccaccgaagaaggcggtgcggcaccgtccaggtgtccctcgctctcgtgctgctgcttggtgctggcctcgcaacacaagggtggtttcttttgagactccatcaacgcttgggagacatagtggcccacctgcctgatggtgggaagggctcttggcaggaccagcgatcacaccaggctaaccccgccgctcacctgacaggggcgaatgccagcttgatcggaataggtgggccgctgctgtgggaaactaggcttggacttgcctttctgagagggcttacataccatgacggagccctcgtaacaatggagcctggttattactacgtgtacagtaaggtgcagctttctggagtcgggtgtccccaggggctggctaacggactgcccatcactcatggactatacaaacgcacatccagatatcctaaagagctggaactgttggtgtcccgtaggagcccgtgtggcagggccaactcttcccgtgtgtggtgggactcctcttttctgggcggcgtggtccatctggaagctggtgaggaagtcgtcgtaagagtacctggaaaccgtctggttcgcccccgcgatggcaccaggtcctacttcggagctttcatggta

SEQ ID NO: 79 - protein, 235 aa, synthetic construct
MESVVQPSVFVVDGQTDIPFRRLEQNHRRRRCGTVQVSLALVLLLGAGLATQGWFLLRLHQRLGDIVAHLPDGGKGSWQDQRSHQANPAAHLTGANASLIGIGGPLLWETRLGLAFLRGLTYHDGALVTMEPGYYYVYSKVQLSGVGCPQGLANGLPITHGLYKRTSRYPKELELLVSRRSPCGRANSSRVWWDSSFLGGVVHLEAGEEVVVRVPGNRLVRPRDGTRSYFGAFMV

SEQ ID NO: 80 - DNA, 1821 bp, synthetic construct
  source 1..1821: mol_type = other DNA
atgtgcccacagaaactcacaatttcttggttcgcaatcgtcctgctggtgtcacccctgatggcaatgtgggagttggaaaaggatgtatacgtcgtcgaggtcgactggacacctgacgctccgggtgaaactgtcaacctcacttgcgatactcctgaagaggacgacatcacgtggacgagcgaccagcgacatggagtgatagggtctggcaagacgcttactatcacggttaaggaatttctcgacgcagggcagtacacatgtcacaagggcggcgagactctgagccactcccatttgctgctgcacaagaaggagaatggtatctggtctaccgaaatcctgaagaattttaagaacaagacttttctgaaatgcgaggccccaaattattccggacgtttcacttgcagttggctcgttcaaagaaatatggacttgaaatttaacattaaatccagctcttcatctcctgacagcagggccgtaacttgtggaatggcttcattgtcagctgagaaagttacgcttgaccaaagggattatgagaaatacagcgtgagttgccaggaagatgtgacatgtccaacggcagaggaaacgttgccaattgagctcgctttggaagctcgtcaacaaaacaagtatgaaaactatagtactagcttcttcatacgggacatcatcaaaccagatccacctaagaatttgcagatgaagcctctgaagaattcacaagtcgaggtatcctgggaatacccagattcatggtccactcctcatagttactttagcctgaaattctttgtacgcatacagcggaagaaggagaaaatgaaggagacggaagaaggctgcaatcagaaaggcgcttttcttgttgaaaagacgagcactgaggttcaatgcaaaggcgggaatgtatgtgttcaagcccaagataggtattataatagctcctgctctaagtgggcttgcgtaccatgcagagttagaagtggctcaacctcaggctccggaaaacctggttccggtgaaggttccacaaaagggcgtgtgattcctgtgtccggcccagctaggtgtctctcccagtcacggaatctcctgaaaaccacggatgacatggtaaagacagctagggagaaactcaagcactactcctgcacagctgaggatatcgatcatgaggacatcaccagggaccagacatccactctgaaaacttgcctgcctttggaactccacaagaacgaatcttgtctggcaacgcgtgaaacgagttctactacaagagggtcctgtcttccccctcaaaagacaagccttatgatgaccttgtgtctcggtagcatttatgaggacctaaagatgtatcaaaccgagtttcaggctatcaatgcagcgctccagaatcataaccatcagcagatcattcttgacaaaggaatgctcgtggccattgatgaactaatgcagagcctaaaccacaatggcgagactcttcgacagaaaccgcctgtgggcgaggccgatccatatagagtcaaaatgaaactgtgtattctcctgcatgcatttagtactcgtgtagtgactattaacagagtgatgggttacctttcctcagctaatacacttgtcctctttggcgctgggttcggcgccgtcataacggttgttgtcatcgtggtaataatcaagtgcttttgcaagcacaggtcttgttttcgcaggaatgaagcctctagagaaacaaataattcactgacctttggccccgaagaagctcttgcagagcaaacggtgtttctc

SEQ ID NO: 81 - protein, 607 aa, synthetic construct
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGSTSGSGKPGSGEGSTKGRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSANTLVLFGAGFGAVITVVVIVVIIKCFCKHRSCFRRNEASRETNNSLTFGPEEALAEQTVFL

SEQ ID NO: 82 - DNA, 1716 bp, synthetic construct
  source 1..1716: mol_type = other DNA
atgtgtcagtcacgctatcttctcttccttgctactctggccttgctcaatcacttgtcccttgctcgtgtgattcctgtgtccggcccagctaggtgtctctcccagtcacggaatctcctgaaaaccacggatgacatggtaaagacagctagggagaaactcaagcactactcctgcacagctgaggatatcgatcatgaggacatcaccagggaccagacatccactctgaaaacttgcctgcctttggaactccacaagaacgaatcttgtctggcaacgcgtgaaacgagttctactacaagagggtcctgtcttccccctcaaaagacaagccttatgatgaccttgtgtctcggtagcatttatgaggacctaaagatgtatcaaaccgagtttcaggctatcaatgcagcgctccagaatcataaccatcagcagatcattcttgacaaaggaatgctcgtggccattgatgaactaatgcagagcctaaaccacaatggcgagactcttcgacagaaaccgcctgtgggcgaggccgatccatatagagtcaaaatgaaactgtgtattctcctgcatgcatttagtactcgtgtagtgactattaacagagtgatgggttacctttcctcagctggaagcggcgccaccaacttctccctgctgaagcaggccggcgacgtggaggagaaccccggccccatgtgcccacagaaactcacaatttcttggttcgcaatcgtcctgctggtgtcacccctgatggcaatgtgggagttggaaaaggatgtatacgtcgtcgaggtcgactggacacctgacgctccgggtgaaactgtcaacctcacttgcgatactcctgaagaggacgacatcacgtggacgagcgaccagcgacatggagtgatagggtctggcaagacgcttactatcacggttaaggaatttctcgacgcagggcagtacacatgtcacaagggcggcgagactctgagccactcccatttgctgctgcacaagaaggagaatggtatctggtctaccgaaatcctgaagaattttaagaacaagacttttctgaaatgcgaggccccaaattattccggacgtttcacttgcagttggctcgttcaaagaaatatggacttgaaatttaacattaaatccagctcttcatctcctgacagcagggccgtaacttgtggaatggcttcattgtcagctgagaaagttacgcttgaccaaagggattatgagaaatacagcgtgagttgccaggaagatgtgacatgtccaacggcagaggaaacgttgccaattgagctcgctttggaagctcgtcaacaaaacaagtatgaaaactatagtactagcttcttcatacgggacatcatcaaaccagatccacctaagaatttgcagatgaagcctctgaagaattcacaagtcgaggtatcctgggaatacccagattcatggtccactcctcatagttactttagcctgaaattctttgtacgcatacagcggaagaaggagaaaatgaaggagacggaagaaggctgcaatcagaaaggcgcttttcttgttgaaaagacgagcactgaggttcaatgcaaaggcgggaatgtatgtgttcaagcccaagataggtattataatagctcctgctctaagtgggcttgcgtaccatgcagagttagaagt

SEQ ID NO: 83 - protein, 572 aa, synthetic construct
MCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGSGATNFSLLKQAGDVEENPGPMCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 84 - DNA, 570 bp, synthetic construct
  source 1..570: mol_type = other DNA
atggccaggctttgcgcttttctcgtcatgctgatcgtcatgagttactggtccatttgcagcctcggatgtgatctgccccacacctacaacctgcgcaacaaacgagctctcaaagtgttggcccaaatgaggcggttgcccttcctttcctgtctcaaagacaggcaagattttggatttccactagagaaagtagacaatcaacagatacagaaagctcaagctatccccgtgttgagggacttgactcaacagacgttgaatctatttactagcaaggccagctctgctgcttggaatgccacccttcttgactcattttgcaatgacctacatcaacaactgaatgatctccaaacatgtttgatgcagcaggtaggtgtccaagaacccccgcttactcaggaagacgcccttctggctgtccgcaagtactttcacagaatcacagtgtacctgcgcgaaaagaaacactccccctgcgcttgggaagtggtcagggccgaggtttggcgagccctgagtagctccgtcaatctccttcctcggttgtccgaggagaaagag

SEQ ID NO: 85 - protein, 190 aa, synthetic construct
MARLCAFLVMLIVMSYWSICSLGCDLPHTYNLRNKRALKVLAQMRRLPFLSCLKDRQDFGFPLEKVDNQQIQKAQAIPVLRDLTQQTLNLFTSKASSAAWNATLLDSFCNDLHQQLNDLQTCLMQQVGVQEPPLTQEDALLAVRKYFHRITVYLREKKHSPCAWEVVRAEVWRALSSSVNLLPRLSEEKE

SEQ ID NO: 86 - DNA, 918 bp, synthetic construct
  source 1..918: mol_type = other DNA
atggcttgcaactgtcagctcatgcaagatactcccctgcttaagtttccctgccctagactcattctcctcttcgtccttctcattcgcctaagccaggtgagttccgatgtggatgaacaactgagtaaatctgtcaaggataaagttctgctcccatgccgctacaatagcccccatgaggacgagtccgaagataggatttactggcagaaacatgataaggtggtgctatccgtcattgccggtaaattgaaggtgtggcccgaatataagaatagaaccctgtatgacaacacaacttatagcctaatcatcctcggtctcgtactgagcgaccgaggtacttactcatgcgttgtgcagaagaaggagcgcggaacatacgaagtcaagcaccttgcattggtgaaattgtcaataaaagctgacttttcaactcctaatattactgaatcaggtaacccttccgcagacactaaaagaattacatgcttcgcctctggcgggtttcccaaaccacggttctcttggctagagaatgggagagaacttccaggtatcaatacaaccatctctcaagacccagaatcagaactgtacaccatctccagccaactcgatttcaataccacaagaaatcatacaataaaatgtctgataaagtacggagatgcacatgtctctgaagatttcacatgggagaaaccaccagaggacccgccagacagcaagaatacacttgtcctctttggcgctgggttcggcgccgtcataacggttgttgtcatcgtggtaataatcaagtgcttttgcaagcacaggtcttgttttcgcaggaatgaagcctctagagaaacaaataattcactgacctttggccccgaagaagctcttgcagagcaaacggtgtttctc

SEQ ID NO: 87 - protein, 306 aa, synthetic construct
MACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFL

SEQ ID NO: 88 - DNA, 780 bp, synthetic construct
  source 1..780: mol_type = other DNA
atgatcgaaacttattcccaaccctcaccgcgctcagtagcaactggcctaccagccagcatgaagatattcatgtacctcttgactgtattcttgatcacgcaaatgattggtagtgttttgttcgccgtttatctccacaggcgcctggataaagttgaagaagaggttaatctccatgaagacttcgtgttcattaagaaactcaaaagatgtaacaaaggtgagggatctctgtctcttctgaactgtgaggagatgcgacggcaattcgaggacctcgtaaaagacataactctcaacaaagaagagaagaaagaaaactctttcgagatgcaacggggcgacgaggaccctcaaattgccgcacatgtcgtttctgaagcgaattccaatgccgcgtccgtgctccagtgggcgaagaagggatactacacgatgaagagcaaccttgtgatgcttgaaaatggcaagcagctcacagttaaacgcgagggactctactatgtatacacccaagtgaccttttgttccaaccgggagccaagtagccaacgcccgttcatcgttgggctgtggctcaagccttcttcagggagtgaacgaatccttctcaaggcagccaacacgcattccagcagccaactgtgtgagcaacaatccgtgcatcttggcggggtctttgagctgcaagcgggcgcctctgtgttcgtgaatgttaccgaagccagccaggttatccaccgcgtgggtttcagtagttttggcctgctcaagctg

SEQ ID NO: 89 - protein, 260 aa, synthetic construct
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 90 - DNA, 438 bp, synthetic construct
  source 1..438: mol_type = other DNA
atggagcgtactctggtctgccttgttgtgatattcttggggacagttgcacacaaatcatcaccccaaggaccggatagactcctcatacgcctgcgccatctgattgacattgtcgagcagttgaagatttatgagaacgacctggaccctgaactattgagcgcgcctcaagacgtcaaagggcattgcgagcatgctgcatttgcatgttttcagaaagctaagctcaaaccaagtaatcccggtaacaataaaacattcatcatcgacctggtggcccaactaagacgccggttgccggcgcgccggggtggtaagaaacagaaacatattgctaaatgcccctcttgcgactcttacgagaaaaggacacctaaggaattcctcgaacgattgaaatggttgttgcagaagatgatccatcaacatctgagc

SEQ ID NO: 91 - protein, 146 aa, synthetic construct [sequence omitted]

SEQ ID NO: 92 - DNA, 399 bp, synthetic construct
  source 1..399: mol_type = other DNA
atggcacaaatgatgacactgtccctacttagtctagttctagctttgtgtattccctggactcaaggcagtgacggaggaggacaagactgctgcctcaaatattctcaaaagaaaatcccttattctatagtccgaggttaccgtaagcaagaaccgagtctaggttgtcctatccccgcaatcctctttctaccacggaaacatagcaaaccagaattgtgcgccaacccagaagagggttgggtccaaaatttgatgaggcgccttgaccaaccaccggccccgggtaaacaatcaccggggtgtcggaagaataggggtacatccaaatccgggaagaaagggaaggggagtaagggctgtaagagaacggaacaaactcaacctagcagaggt

SEQ ID NO: 93 - protein, 133 aa, synthetic construct
MAQMMTLSLLSLVLALCIPWTQGSDGGGQDCCLKYSQKKIPYSIVRGYRKQEPSLGCPIPAILFLPRKHSKPELCANPEEGWVQNLMRRLDQPPAPGKQSPGCRKNRGTSKSGKKGKGSKGCKRTEQTQPSRG

SEQ ID NO: 94 - DNA, 1224 bp, synthetic construct
  source 1..1224: mol_type = other DNA
atggaaaccgacacattgctcctctgggttctccttctatgggtccccggttccaccggagatatccaaatgacacaatcacccagcagcctgcctgcctctctgggcgaccgcgttaccatcaattgtcaagcttcccaagatataagtaattatctcaactggtaccagcaaaagcccggtaaagcgcctaaattgctgatttattatactaataaactcgcagatggagttcctagtagattttctggttcagggagtggacgggactccagttttaccatatcaagtctggaatccgaggatatcggcagctactattgccagcaatattataattacccttggacttttggacccgggactaaacttgagatcaaaagaggcggaggaggcagtggtggtggtggatcaggcggcggtggtagtgaggtacaactcgtggaatcaggcggcggactggtccaacccggcaagagccttaaactctcttgtgaggccagtggatttacattcagcggttatggaatgcactgggtgagacaagctcccggcaggggcctagaatcagtggcgtacatcaccagctcatcaataaacattaaatacgctgatgcagtcaagggccggtttactgtatcccgcgacaacgctaagaatcttctctttctgcaaatgaacatacttaagagcgaggatactgccatgtattattgtgcccgcttcgattgggataagaattattggggacaaggcaccatggttaccgttagtagtccaaacatcacatcaaataatagcaaccccgtggaaggggacgactctgtttcactcacctgtgattcctataccgatcctgataatatcaactatctatggtctcgtaacggtgaaagtctcagcgaaggcgaccggttgaaactctccgaaggtaacagaacccttacgcttctgaacgtcacccggaacgataccgggccctatgtttgcgaaactaggaaccctgttagcgtgaatcgtagcgaccctttctccctaaataatactctagtgctattcggagcgggattcggtgccgtcatcacagtagtcgttattgtagtcattattaaatgcttttgtaaacataggtcttgcttcagaagaaatgaggccagccgtgaaactaataattccctgacctttgggcccgaagaagctttggctgaacagactgtgtttctc

SEQ ID NO: 95 - protein, 408 aa, synthetic construct
METDTLLLWVLLLWVPGSTGDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKRGGGGSGGGGSGGGGSEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSPNITSNNSNPVEGDDSVSLTCDSYTDPDNINYLWSRNGESLSEGDRLKLSEGNRTLTLLNVTRNDTGPYVCETRNPVSVNRSDPFSLNNTLVLFGAGFGAVITVVVIVVIIKCFCKHRSCFRRNEASRETNNSLTFGPEEALAEQTVFL

SEQ ID NO: 96 - DNA, 420 bp, synthetic construct
  source 1..420: mol_type = other DNA
atggttcttctcaggagcctcttcatcctgcaagtactagtacggatggggctaacttacaacttttctaactgcaacttcacgtcaattacgaaaatatattgtaacataatttttcatgacctgactggagatttgaaaggggctaagttcgagcaaatcgaggactgtgagagcaagccagcttgtctcctgaaaatcgagtactatactctcaatcctatccctggctgcccttcactccccgacaaaacatttgcccggagaacaagagaagccctcaatgaccactgcccaggctaccctgaaactgagagaaatgacggtactcaggaaatggcacaagaagtccaaaacatctgcctgaatcaaacctcacaaattctaagattgtggtattccttcatgcaatctccagaa

SEQ ID NO: 97 - protein, 140 aa, synthetic construct
MVLLRSLFILQVLVRMGLTYNFSNCNFTSITKIYCNIIFHDLTGDLKGAKFEQIEDCESKPACLLKIEYYTLNPIPGCPSLPDKTFARRTREALNDHCPGYPETERNDGTQEMAQEVQNICLNQTSQILRLWYSFMQSPE

SEQ ID NO: 98 - DNA, 423 bp, synthetic construct
  source 1..423: mol_type = other DNA
atgtggctgcagaatttacttttcctgggcattgtggtctacagcctctcagcacccacccgctcacccatcactgtcacccggccttggaagcatgtagaggccatcaaagaagccctgaacctcctggatgacatgcctgtcacgttgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccaggccaaaaa

SEQ ID NO: 99 - protein, 141 aa, synthetic construct
MWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 100 - DNA, 465 bp, synthetic construct
  source 1..465: mol_type = other DNA
atgaacgctacacactgcatcttggctttgcagctcttcctcatggctgtttctggctgttactgccacggcacagtcattgaaagcctagaaagtctgaataactattttaactcaagtggcatagatgtggaagaaaagagtctcttcttggatatctggaggaactggcaaaaggatggtgacatgaaaatcctgcagagccagattatctctttctacctcagactctttgaagtcttgaaagacaatcaggccatcagcaacaacataagcgtcattgaatcacacctgattactaccttcttcagcaacagcaaggcgaaaaaggatgcattcatgagtattgccaagtttgaggtcaacaacccacaggtccagcgccaagcattcaatgagctcatccgagtggtccaccagctgttgccggaatccagcctcaggaagcggaaaaggagtcgctgc

SEQ ID NO: 101 - protein, 155 aa, synthetic construct
MNATHCILALQLFLMAVSGCYCHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRC

SEQ ID NO: 102 - DNA, 462 bp, synthetic construct
  source 1..462: mol_type = other DNA
atgttccatgtttcttttagatatatctttggaattcctccactgatccttgttctgctgcctgtcacatcatctgagtgccacattaaagacaaagaaggtaaagcatatgagagtgtactgatgatcagcatcgatgaattggacaaaatgacaggaactgatagtaattgcccgaataatgaaccaaacttttttagaaaacatgtatgtgatgatacaaaggaagctgcttttctaaatcgtgctgctcgcaagttgaagcaatttcttaaaatgaatatcagtgaagaattcaatgtccacttactaacagtatcacaaggcacacaaacactggtgaactgcacaagtaaggaagaaaaaaacgtaaaggaacagaaaaagaatgatgcatgtttcctaaagagactactgagagaaataaaaacttgttggaataaaattttgaagggcagtata

SEQ ID NO: 103 - protein, 154 aa, synthetic construct
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 104 - DNA, 966 bp, synthetic construct
  source 1..966: mol_type = other DNA
atgcagctaaagtgtccctgttttgtgtccttgggaaccaggcagcctgtttggaagaagctccatgtttctagcgggttcttttctggtcttggtctgttcttgctgctgttgagcagcctctgtgctgcctctgcagagactgaagtcggtgcaatggtgggcagcaatgtggtgctcagctgcattgacccccacagacgccatttcaacttgagtggtctgtatgtctattggcaaatcgaaaacccagaagtttcggtgacttactacctgccttacaagtctccagggatcaatgtggacagttcctacaagaacaggggccatctgtccctggactccatgaagcagggtaacttctctctgtacctgaagaatgtcacccctcaggatacccaggagttcacatgccgggtatttatgaatacagccacagagttagtcaagatcttggaagaggtggtcaggctgcgtgtggcagcaaacttcagtacacctgtcatcagcacctctgatagctccaacccgggccaggaacgtacctacacctgcatgtccaagaatggctacccagagcccaacctgtattggatcaacacaacggacaatagcctaatagacacggctctgcagaataacactgtctacttgaacaagttgggcctgtatgatgtaatcagcacattaaggctcccttggacatctcgtggggatgttctgtgctgcgtagagaatgtggctctccaccagaacatcactagcattagccaggcagaaagtttcactggaaataacacaaagaacccacaggaaacccacaataatgagttaaaagtccttgtccccgtccttgctgtactggcggcagcggcattcgtttccttcatcatatacagacgcacgcgtccccaccgaagctatacaggacccaagactgtacagcttgaacttacagaccacgcc

SEQ ID NO: 105 - protein, 320 aa, synthetic construct
MQLKCPCFVSLGTRQPVWKKLHVSSGFFSGLGLFLLLLSSLCAASAETEVGAMVGSNVVLSCIDPHRRHFNLSGLYVYWQIENPEVSVTYYLPYKSPGINVDSSYKNRGHLSLDSMKQGNFSLYLKNVTPQDTQEFTCRVFMNTATELVKILEEVVRLRVAANFSTPVISTSDSSNPGQERTYTCMSKNGYPEPNLYWINTTDNSLIDTALQNNTVYLNKLGLYDVISTLRLPWTSRGDVLCCVENVALHQNITSISQAESFTGNNTKNPQETHNNELKVLVPVLAVLAAAAFVSFIIYRRTRPHRSYTGPKTVQLELTD

SEQ ID NO: 106 - DNA, 909 bp, synthetic construct
  source 1..909: mol_type = other DNA
atgtggcccttggcggcggcgctgttgctgggctcctgctgctgcggttcagctcaactactgtttagtaacgtcaactccatagagttcacttcatgcaatgaaactgtggtcatcccttgcatcgtccgtaatgtggaggcgcaaagcaccgaagaaatgtttgtgaagtggaagttgaacaaatcgtatattttcatctatgatggaaataaaaatagcactactacagatcaaaactttaccagtgcaaaaatctcagtctcagacttaatcaatggcattgcctctttgaaaatggataagcgcgatgccatggtgggaaactacacttgcgaagtgacagagttatccagagaaggcaaaacagttatagagctgaaaaaccgcacggtttcgtggttttctccaaatgaaaagatcctcattgttattttcccaattttggctatactcctgttctggggaaagtttggtattttaacactcaaatataaatccagccatacgaataagagaatcattctgctgctcgttgccgggctggtgctcacagtcatcgtggttgttggagccatccttctcatcccaggagaaaagcccgtgaagaatgcttctggacttggcctcattgtaatctctacggggatattaatactacttcagtacaatgtgtttatgacagcttttggaatgacctctttcaccattgccatattgatcactcaagtgctgggctacgtccttgctttggtcgggctgtgtctctgcatcatggcatgtgagccagtgcacggcccccttttgatttcaggtttggggatcatagctctagcagaactacttggattagtttatatgaagtttgtcgcttccaaccagaggactatccaacctcctaggaatagg

SEQ ID NO: 107 - protein, 303 aa, synthetic construct
MWPLAAALLLGSCCCGSAQLLFSNVNSIEFTSCNETVVIPCIVRNVEAQSTEEMFVKWKLNKSYIFIYDGNKNSTTTDQNFTSAKISVSDLINGIASLKMDKRDAMVGNYTCEVTELSREGKTVIELKNRTVSWFSPNEKILIVIFPILAILLFWGKFGILTLKYKSSHTNKRIILLLVAGLVLTVIVVVGAILLIPGEKPVKNASGLGLIVISTGILILLQYNVFMTAFGMTSFTIAILITQVLGYVLALVGLCLCIMACEPVHGPLLISGLGIIALAELLGLVYMKFVASNQRTIQPPRNR

SEQ ID NO: 108 - DNA, 1161 bp, synthetic construct
  source 1..1161: mol_type = other DNA
atggcagcagcagtaacttggatacctctcctggcaggtctcctggcaggactgagggacaccaaggcccagcagacaactttacacctacttgtgggtcgtgtgtttgtgcatcctttggaacatgccaccttcctgcgccttccagaacacgttgcggtgccacccactgtccgactcacctaccacgctcacctccagggacatccagacctgcccaggtggctgcactacacacagcgcagtccctataaccctggcttcctctacggctcccccactccagaagatcgtgggtaccaagtcatcgaggtcacagcctacaatcgagacagttttgacaccactagacagaggctgctgctgctgattggggaccccgaaggtccccggttgccataccaagctgagttcctggtgcgcagccatgatgtggaggaggtgctgcccaccacacctgccaaccgcttcctcaccgccttggggggactgtgggagccaggagagcttcagctgctcaacatcacttccgccttggaccggggaggccgagtccctcttcctattgagggacggaaggaaggggtatacattaaggtaggctctgccacacccttctccacctgcctgaagatggtggcgtcgcccgacagctatgcccgttgtgcccagggacagcctccactactgtcctgctacgacactttggcaccccacttccgcgttgactggtgcaatgtgtctctggtagacaagtcagtacccgagcccctggatgaggtacctactccaggcgatgggatcttggagcacgacccgttcttctgcccacccactgaagccacagaccgagacttcctgacagatgccttggtgaccctcttggtgcctttgttggtggctctgctgcttactctgttgctggcttacatcatgtgctttcggcgtgaaggacggctgaagagagacatggccacctctgacatccagatgtttcaccactgttccatccatgggaatacagaagagcttcggcagatggcagccagccgagaggtgccccggcctctttccaccttgcccatgtttaatgttcgtacaggagagcggttacctccccgagtagacagcgcacagatgcctcttatcctggaccagcac

SEQ ID NO: 109 - protein, 387 aa, synthetic construct
MAAAVTWIPLLAGLLAGLRDTKAQQTTLHLLVGRVFVHPLEHATFLRLPEHVAVPPTVRLTYHAHLQGHPDLPRWLHYTQRSPYNPGFLYGSPTPEDRGYQVIEVTAYNRDSFDTTRQRLLLLIGDPEGPRLPYQAEFLVRSHDVEEVLPTTPANRFLTALGGLWEPGELQLLNITSALDRGGRVPLPIEGRKEGVYIKVGSATPFSTCLKMVASPDSYARCAQGQPPLLSCYDTLAPHFRVDWCNVSLVDKSVPEPLDEVPTPGDGILEHDPFFCPPTEATDRDFLTDALVTLLVPLLVALLLTLLLAYIMCFRREGRLKRDMATSDIQMFHHCSIHGNTEELRQMAASREVPRPLSTLPMFNVRTGERLPPRVDSAQMPLILDQH

SEQ ID NO: 110 - DNA, 627 bp, synthetic construct
  source 1..627: mol_type = other DNA
atgtggaaatggatactgacacattgtgcctcagcctttccccacctgccgggctgctgttgctgcttcttgttgctctttttggtgtcttcgttccctgtcacctgccaagctcttggtcaggacatggtgtcacaggaggccaccaactgctcttcttcctcctcgtccttctcctctccttccagtgcgggaaggcatgtgcggagctacaatcacctccaaggagatgtccgctggagaaggctgttctccttcaccaagtactttctcacgattgagaagaacggcaaggtcagcgggaccaagaatgaagactgtccgtacagtgtcctggagataacatcagtggaaatcggagttgttgccgtcaaagccatcaacagcaactattacttagccatgaacaagaaggggaaactctatggctcaaaagagtttaacaacgactgtaagctgaaagagagaatagaggaaaatggatacaacacctatgcatcttttaactggcagcacaatggcaggcaaatgtatgtggcattgaatggaaaaggagctcccaggagaggacaaaaaacaagaaggaaaaacacctctgctcacttcctccccatgacgatccaaaca

SEQ ID NO: 111 - protein, 209 aa, synthetic construct
MWKWILTHCASAFPHLPGCCCCFLLLFLVSSFPVTCQALGQDMVSQEATNCSSSSSSFSSPSSAGRHVRSYNHLQGDVRWRRLFSFTKYFLTIEKNGKVSGTKNEDCPYSVLEITSVEIGVVAVKAINSNYYLAMNKKGKLYGSKEFNNDCKLKERIEENGYNTYASFNWQHNGRQMYVALNGKGAPRRGQKTRRKNTSAHFLPMTIQT

SEQ ID NO: 112 - DNA, 5853 bp, synthetic construct
  source 1..5853: mol_type = other DNA
atgcctcctctgccactggaacacagacccaggcagcagcctggtgcctccgtgctggttcggtacttcatgatcccctgcaacatctgcttgatcctcttggctacttctacgttgggctttgcggtgctgcttttcctcagcaactacaaacctgggatccacttcacagcagcgccttctatgcctcctgatgtgtgcaggggaatgttatgtggctttggtgctgtgtgtgaacctagtgttgaggatccaggccgggcctcctgtgtgtgcaagaagaatgtctgccctgctatggtagctcctgtgtgtggctcagatgcttccacctatagcaacgagtgtgagctacagcgtgcacagtgcaaccagcaacggcgcatccgcctactccgccaagggccatgtgggtcccgggacccctgtgccaatgtgacctgcagtttcggtagtacctgtgtaccttcagccgatggacagaccgcctcgtgtctgtgtcctacaacctgctttggggcccctgatggcacagtgtgtggcagtgatggtgttgactaccctagtgagtgccagctgctccgtcatgcctgtgccaaccaggagcacatctttaagaagttcgatggtccttgtgacccctgccagggcagcatgtcagacctgaatcatatttgccgggtgaacccacgtacacggcacccagaaatgcttctgcggcctgagaactgccccgcccaacacacacctatctgtggagatgatggggtcacctatgaaaacgactgtgtcatgagccgtataggtgcagcccgtggcctgcttctccagaaagtgcgctctggtcaatgccagactcgagaccagtgcccggagacctgccagtttaactctgtatgcctgtcccgccgcggccgtccccactgttcctgcgatcgcgtcacctgtgatggggcttacaggccagtgtgtgcccaggatgggcacacgtatgacaatgactgttggcgccaacaggccgagtgtcgacaacagcagaccattccccccaagcaccagggcccgtgtgaccagaccccatccccgtgccgtggagcgcagtgtgcatttggggcaacatgcacagtgaagaatgggaaagctgtgtgcgagtgccagcgggtgtgctcgggcggctacgatcctgtgtgcggcagtgatggtgtcacttacggcagtgtgtgcgagctggaatccatggcctgtacccttgggcgggaaatccgagtggcccgcagaggaccgtgtgaccgatgtgggcagtgccggtttggatccttgtgcgaggtggagactggacgctgtgtgtgcccctctgagtgtgtggagtcagcccagcccgtatgtggctctgacggacacacatatgctagtgaatgtgagctgcatgtccacgcctgtacacaccagatcagcctatacgtggcctcagccggacactgccagacctgtggagaaacagtttgtacctttggggctgtgtgctcagctggacagtgtgtatgtccccgttgtgagcaccccccacctggccctgtgtgcggcagtgatggcgtcacctacctcagtgcctgtgagctccgagaggctgcctgtcagcagcaggtacaaattgaggaggcccgtgcagggccgtgtgagccggctgagtgtggctcagggggctctgggtctggggaagacaatgcgtgtgagcaggagctgtgtcggcagcatggtggtgtctgggatgaggactcagaagacgggccgtgtgtctgtgactttagttgccagagtgtccttaaaagcccagtgtgtggctcagatggagtcacctatagcacggagtgccatctgaagaaggccagatgtgaagcgcggcaagagctgtacgtcgctgctcagggagcctgccggggccctaccttggctccactgctacctatggcctccccacactgtgcccaaaccccctatggctgctgccaggacaatgtcactgctgcccagggtgtgggcttggctggctgtcccagcacctgccattgcaacccacacggctcctatagcggcacttgtgacccagtcacagggcagtgctcctgccgaccaggtgtaggaggcctcaggtgtgatcgctgtgagcctggcttctggaacttccgtggcattgtcaccgatggacatagtggttgcactccctgcagctgtgaccctcggggtgctgtaagagatgactgtgagcagatgactggattgtgttcctgtagacctggtgtggctggtcccaagtgtgggcagtgtccagatggtcaagccctgggccatcttggctgtgaagcagatcccacaacaccagtgacttgtgtggaaatgcactgtgagtttggcgcctcctgcgtagaggaggctggttttgcccagtgtgtctgcccaactctcacatgtccagaggctaactctaccaaggtctgtggatcagatggtgtcacatacggcaatgaatgccagctgaagaccattgcctgccgccagcgtctggacatctccattcagagtcttggtccatgccgggagagtgttgctcctggggtttcccctacatctgcatctatgaccaccccaaggcatatcctgagcaggacactggcgtctccccacagcagccttcctctgtctcccagcactactgcccatgattggcccaccccattacccacatcacctcagaccgtagtcggcacccccaggagcactgcagccacaccctctgatgtggccagtcttgctacagcgatcttcagggaatctggcagcaccaacggcagtggcgatgaggagctcagtggcgatgaggaggccagtgggggcgggtctgggggacttgagcccccggtgggcagcgttgtggtgacccacgggccacccatcgagagggcttcctgttacaactcacctttgggctgctgctcagatggcaagacaccctcactggactcagaaggctccaactgtccagctaccaaggcattccagggcgtgctggagcttgagggggtcgagggacaggaactgttctacacaccagagatggctgaccccaagtcagagttgtttggggagactgcaaggagcattgagagcacgctggacgacctgttccggaattcggatgttaagaaggacttctggagcatccgcctacgggaactggggcctggcaaattagtccgtgccattgtggatgttcactttgaccccaccacagccttccaggcaccagatgtgggtcaggccttgctccaacagatccaggtatccaggccgtgggccctggcagtgaggaggcctctgcgggagcatgtgcgattcttggactttgactggtttcccactttttttacgggagctgcaacaggaaccacagctgctgtggccacagccagagccaccactgtgagccgactgtctgcctcttctgtcaccccacgagtctaccccagttacaccagccggcctgttggcagaactacggcaccgctaaccactcgccggccaccaaccactaccgccagtattgaccgacctcggactccaggcccgcaacggcccccaaagtcctgtgattcccagccttgcctccacggaggtacctgccaggacctggattctggcaagggtttcagctgcagctgtactgcaggcagggctggcactgtctgtgagaaagtgcagctcccctctgtgccagcttttaagggccactccttcttggccttccccaccctccgagcctaccacacgctgcgtctggcactagaattccgggcgctggagacagagggactgctgctctacaatggcaatgcacgtggcaaagatttcctggctctggctctgttggatggtcatgtacagttcaggttcgacacgggctcagggccggcggtgctaacaagcttagtgccagtggaaccgggacggtggcaccgcctcgagttgtcacggcattggcggcagggcacactttctgtggatggcgaggctcctgttgtaggtgaaagtccgagtggcactgatggcctcaacttggacacgaagctctatgtgggtggtctcccagaagaacaagttgccacggtgcttgatcggacctctgtgggcatcggcctgaaaggatgcattcgtatgttggacatcaacaaccagcagctggagctgagcgattggcagagggctgtggttcaaagctctggtgtgggggaatgcggagaccatccctgctcacctaacccctgccatggcggggccctctgccaggccctggaggctggcgtgttcctctgtcagtgcccacctggccgctttggcccaacttgtgcagatgaaaagaacccctgccaaccgaacccctgccacgggtcagccccctgccatgtgctttccaggggtggggccaagtgtgcgtgccccctgggacgcagtggttccttctgtgagacagtcctggagaatgctggctcccggcccttcctggctgactttaatggcttctcctacctggaactgaaaggcttgcacaccttcgagagagacctaggggagaagatggcgctggagatggtgttcttggctcgtgggcccagtggcttactcctctacaatgggcagaagacggatggcaagggggactttgtatccctggccctgcataaccggcacctagagttccgctatgaccttggcaagggggctgcaatcatcaggagcaaagagcccatagccctgggcacctgggttagggtattcctggaacgaaatggccgcaagggtgcccttcaagtgggtgatgggccccgtgtgctaggggaatctccgaaatcccgcaaggtcccgcacaccatgctcaacctcaaggagcccctctatgtggggggagctcctgacttcagcaagctggctcggggcgctgcagtggcctccggctttgatggtgccatccagctggtgtctctaagaggccatcagctgctgactcaggagcatgtgttgcgggcagtagatgtagcgccttttgcaggccacccttgtacccaggccgtggacaacccctgccttaatgggggctcctgtatcccgagggaagccacttatgagtgcctgtgtcctgggggcttctctgggctgcactgcgagaaggggatagttgagaagtcagtgggggacctagaaacactggcctttgatgggcggacctacatcgagtacctcaatgctgtgactgagagcgagctgaccaatgagatcccagcccccgaaactctggattcccgggcccttttcagtgagaaagcgctgcagagcaaccactttgagctgagcttacgcactgaggccacgcaggggctggtgctgtggattggaaaggttggagaacgtgcagactacatggctctggccattgtggatgggcacctacaactgagctatgacctaggctcccagccagttgtgctgcgctccactgtgaaggtcaacaccaaccgctggcttcgagtcagggctcacagggagcacagggaaggttcccttcaggtgggcaatgaagcccctgtgactggctcttccccgctgggtgccacacaattggacacagatggagccctgtggcttggaggcctacagaagcttcctgtggggcaggctctccccaaggcctatggcacgggttttgtgggctgtctgcgggacgtggtagtgggccatcgccagctgcatctgctggaggacgctgtcaccaaaccagagctaagaccctgccccactctctga

SEQ ID NO: 113 - protein, 1949 aa, synthetic construct
MPPLPLEHRPRQQPGASVLVRYFMIPCNICLILLATSTLGFAVLLFLSNYKPGIHFTAAPSMPPDVCRGMLCGFGAVCEPSVEDPGRASCVCKKNVCPAMVAPVCGSDASTYSNECELQRAQCNQQRRIRLLRQGPCGSRDPCANVTCSFGSTCVPSADGQTASCLCPTTCFGAPDGTVCGSDGVDYPSECQLLRHACANQEHIFKKFDGPCDPCQGSMSDLNHICRVNPRTRHPEMLLRPENCPAQHTPICGDDGVTYENDCVMSRIGAARGLLLQKVRSGQCQTRDQCPETCQFNSVCLSRRGRPHCSCDRVTCDGAYRPVCAQDGHTYDNDCWRQQAECRQQQTIPPKHQGPCDQTPSPCRGAQCAFGATCTVKNGKAVCECQRVCSGGYDPVCGSDGVTYGSVCELESMACTLGREIRVARRGPCDRCGQCRFGSLCEVETGRCVCPSECVESAQPVCGSDGHTYASECELHVHACTHQISLYVASAGHCQTCGETVCTFGAVCSAGQCVCPRCEHPPPGPVCGSDGVTYLSACELREAACQQQVQIEEARAGPCEPAECGSGGSGSGEDNACEQELCRQHGGVWDEDSEDGPCVCDFSCQSVLKSPVCGSDGVTYSTECHLKKARCEARQELYVAAQGACRGPTLAPLLPMASPHCAQTPYGCCQDNVTAAQGVGLAGCPSTCHCNPHGSYSGTCDPVTGQCSCRPGVGGLRCDRCEPGFWNFRGIVTDGHSGCTPCSCDPRGAVRDDCEQMTGLCSCRPGVAGPKCGQCPDGQALGHLGCEADPTTPVTCVEMHCEFGASCVEEAGFAQCVCPTLTCPEANSTKVCGSDGVTYGNECQLKTIACRQRLDISIQSLGPCRESVAPGVSPTSASMTTPRHILSRTLASPHSSLPLSPSTTAHDWPTPLPTSPQTVVGTPRSTAATPSDVASLATAIFRESGSTNGSGDEELSGDEEASGGGSGGLEPPVGSVVVTHGPPIERASCYNSPLGCCSDGKTPSLDSEGSNCPATKAFQGVLELEGVEGQELFYTPEMADPKSELFGETARSIESTLDDLFRNSDVKKDFWSIRLRELGPGKLVRAIVDVHFDPTTAFQAPDVGQALLQQIQVSRPWALAVRRPLREHVRFLDFDWFPTFFTGAATGTTAAVATARATTVSRLSASSVTPRVYPSYTSRPVGRTTAPLTTRRPPTTTASIDRPRTPGPQRPPKSCDSQPCLHGGTCQDLDSGKGFSCSCTAGRAGTVCEKVQLPSVPAFKGHSFLAFPTLRAYHTLRLALEFRALETEGLLLYNGNARGKDFLALALLDGHVQFRFDTGSGPAVLTSLVPVEPGRWHRLELSRHWRQGTLSVDGEAPVVGESPSGTDGLNLDTKLYVGGLPEEQVATVLDRTSVGIGLKGCIRMLDINNQQLELSDWQRAVVQSSGVGECGDHPCSPNPCHGGALCQALEAGVFLCQCPPGRFGPTCADEKNPCQPNPCHGSAPCHVLSRGGAKCACPLGRSGSFCETVLENAGSRPFADFNGFSYLELKGLHTFERDLGEKMALEMVFLARGPSGLLLYNGQKTDGKGDFVSLALHNRHLEFRYDLGKGAAIIRSKEPIALGTWVRVFLERNGRKGALQVGDGPRVLGESPKSRKVPHTMLNLKEPLYVGGAPDFSKLARGAAVASGFDGAIQLVSLRGHQLLTQEHVLRAVDVAPFAGHPCTQAVDNPCLNGGSCIPREATYECLCPGGFSGLHCEKGIVEKSVGDLETLAFDGRTYIEYLNAVTESELTNEIPAPETLDSRALFSEKALQSNHFELSLRTEATQGLVLWIGKVGERADYMALAIVDGHLQLSYDLGSQPVVLRSTVKVNTNRWLRVRAHREHREGSLQVGNEAPVTGSSPLGATQLDTDGALWLGGLQKLPVGQALPKAYGTGFVGCLRDVVVGHRQLHLLEDAVTKPELRPCPTL

SEQ ID NO: 114 - DNA, 537 bp, synthetic construct
  source 1..537: mol_type = other DNA
atgcctggctcagcactgctatgctgcctgctcttactgactggcatgaggatcagcaggggccagtacagccgggaagacaataactgcacccacttcccagtcggccagagccacatgctcctagagctgcggactgccttcagccaggtgaagactttctttcaaacaaaggaccagctggacaacatactgctaaccgactccttaatgcaggactttaagggttacttgggttgccaagccttatcggaaatgatccagttttacctggtagaagtgatgccccaggcagagaagcatggcccagaaatcaaggagcatttgaattccctgggtgagaagctgaagaccctcaggatgcggctgaggcgctgtcatcgatttctcccctgtgaaaataagagcaaggcagtggagcaggtgaagagtgattttaataagctccaagaccaaggtgtctacaaggccatgaatgaatttgacatcttcatcaactgcatagaagcatacatgatgatcaaaatgaaaagctaa

SEQ ID NO: 115 - protein, 178 aa, synthetic construct
MPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 116 - DNA, 501 bp, synthetic construct
  source 1..501: mol_type = other DNA
atggcagcccccagcggaggcttctggactgcggtggtcctggcggccgcagcgctgaaattggccgccgctgtgtccgagcccaccaccgtgccatttgacgtgaggcccggaggggtcgtgcattcgttctcccaggacgtaggacccgggaacaagtttacatgtacattcacctacgcttcccaaggagggaccaacgagcaatggcagatgagcctggggacaagtgaagacagccagcactttacctgtaccatctggaggccccaggggaaatcctacctctacttcacacagttcaaggctgagttgcgaggtgctgagatcgagtatgccatggcctactccaaagccgcatttgagagagagagtgatgtccccctgaaaagtgaggagtttgaagtgaccaagacagcagtgtctcacaggcctggggccttcaaagctgagctctccaagctggtgatcgtagccaaggcggcacgctcggagctgtga

SEQ ID NO: 117 - protein, 166 aa, synthetic construct
MAAPSGGFWTAVVLAAAALKLAAAVSEPTTVPFDVRPGGVVHSFSQDVGPGNKFTCTFTYASQGGTNEQWQMSLGTSEDSQHFTCTIWRPQGKSYLYFTQFKAELRGAEIEYAMAYSKAAFERESDVPLKSEEFEVTKTAVSHRPGAFKAELSKLVIVAKAARSEL

SEQ ID NO: 118 - DNA, 2412 bp, synthetic construct
  source 1..2412: mol_type = other DNA
cagtctgtgttgacgcagccgccctcagtgtctgcggccccaggacagagggtcaccatctcctgctctggaaccaggtccaacattgggagtgattatgtttcctggtaccaacacctcccaggaacagcccccaaactcctcgtttatggcgataatctgcgaccctcagggattcctgaccgattctctgcctccaagtctggcacgtcagccaccctgggcatcaccggactccagactggggacgaggccgattattactgcggcacatgggattacaccctgaatggtgtggtgttcggcggagggaccaagctgaccgtcctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttcaggcgccggatctggtggaaactggagtcatccccaattcgagaagggcggaagcggtgggagtggcgggtccggtggaagcaactggtcacacccacaattcgagaaaggcggttctggcggatctggtggatctggcggaagtaactggtctcatcctcaattcgaaaagggcggaagcggtggcggcaggctaggtggaggctcagtgcaggtgcagctggtggagtctgggggaggcttggtacagcctggggggtccctgagactctcctgtgcagcctctggattcacctttagcagctatgccatgagctgggtccgccaggctccagggaaggggctggagtgggtctcagttatttatagcggtggtagtagcacatactatgcagactccgtgaagggccggttcaccatctccagagataattccaagaacacgctgtatctgcaaatgaacagcctgagagccgaggacacggccgtatattactgtgcgcgcacttcttacctgaaccatggtgattactggggtcaaggtactctggtgaccgtgtctagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccgagctgcaactggaggagagctgtgcggaggcgcaggacggggagctggacgggctgtggacgaccatcaccatcttcatcacactcttcctgttaagcgtgtgctacagtgccaccgtcaccttcttcaaggtgaagtggatcttctcctcggtggtggacctgaagcagaccatcatccccgactacaggaacatgatcggacagggggcctga

SEQ ID NO: 119 - protein, 803 aa, synthetic construct
QSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECSGAGSGGNWSHPQFEKGGSGGSGGSGGSNWSHPQFEKGGSGGSGGSGGSNWSHPQFEKGGSGGGRLGGGSVQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPELQLEESCAEAQDGELDGLWTTITIFITLFLLSVCYSATVTFFKVKWIFSSVVDLKQTIIPDYRNMIGQGA

SEQ ID NO: 120 - DNA, 645 bp, synthetic construct
  source 1..645: mol_type = other DNA
cagtctgtgttgacgcagccgccctcagtgtctgcggccccaggacagagggtcaccatctcctgctctggaaccaggtccaacattgggagtgattatgtttcctggtaccaacacctcccaggaacagcccccaaactcctcgtttatggcgataatctgcgaccctcagggattcctgaccgattctctgcctccaagtctggcacgtcagccaccctgggcatcaccggactccagactggggacgaggccgattattactgcggcacatgggattacaccctgaatggtgtggtgttcggcggagggaccaagctgaccgtcctatcttcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaaccacaagcccagcaacaccaaggtggacaagagagtggagcccaagagctgc

SEQ ID NO: 121 - protein, 215 aa, synthetic construct
QSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 122 - DNA, 1566 bp, synthetic construct
  source 1..1566: mol_type = other DNA
caggtgcagctggtggagtctgggggaggcttggtacagcctggggggtccctgagactctcctgtgcagcctctggattcacctttagcagctatgccatgagctgggtccgccaggctccagggaaggggctggagtgggtctcagttatttatagcggtggtagtagcacatactatgcagactccgtgaagggccggttcaccatctccagagataattccaagaacacgctgtatctgcaaatgaacagcctgagagccgaggacacggccgtatattactgtgcgcgcacttcttacctgaaccatggtgattactggggtcaaggtactctggtgaccgtgtctagcgcctccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccgagctgcaactggaggagagctgtgcggaggcgcaggacggggagctggacgggctgtggacgaccatcaccatcttcatcacactcttcctgttaagcgtgtgctacagtgccaccgtcaccttcttcaaggtgaagtggatcttctcctcggtggtggacctgaagcagaccatcatccccgactacaggaacatgatcggacagggggcctga

SEQ ID NO: 123 - protein, 521 aa, synthetic construct
QVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPELQLEESCAEAQDGELDGLWTTITIFITLFLLSVCYSATVTFFKVKWIFSSVVDLKQTIIPDYRNMIGQGA

SEQ ID NO: 124 - DNA, 486 bp, synthetic construct
  source 1..486: mol_type = other DNA
atgaaaattttgaaaccatatatgaggaatacatccatctcgtgctacttgtgtttccttctaaacagtcactttttaactgaggctggcattcatgtcttcattttgggctgtgtcagtgtaggtctccctaaaacagaggccaactggatagatgtaagatatgacctggagaaaattgaaagccttattcaatctattcatattgacaccactttatacactgacagtgactttcatcccagttgcaaagttactgcaatgaactgctttctcctggaattgcaggttattttacatgagtacagtaacatgactcttaatgaaacagtaagaaacgtgctctaccttgcaaacagcactctgtcttctaacaagaatgtagcagaatctggctgcaaggaatgtgaggagctggaggagaaaaccttcacagagtttttgcaaagctttatacgcattgtccaaatgttcatcaacacgtcc

SEQ ID NO: 125 - protein, 162 aa, synthetic construct
MKILKPYMRNTSISCYLCFLLNSHFLTEAGIHVFILGCVSVGLPKTEANWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS

SEQ ID NO: 126 - DNA, 1185 bp, synthetic construct
  source 1..1185: mol_type = other DNA
cagtctgtgttgacgcagccgccctcagtgtctgcggccccaggacagagggtcaccatctcctgctctggaaccaggtccaacattgggagtgattatgtttcctggtaccaacacctcccaggaacagcccccaaactcctcgtttatggcgataatctgcgaccctcagggattcctgaccgattctctgcctccaagtctggcacgtcagccaccctgggcatcaccggactccagactggggacgaggccgattattactgcggcacatgggattacaccctgaatggtgtggtgttcggcggagggaccaagctgaccgtcctaggttctagaggtggtggtggtagcggcggcggcggctctggtggtggtggatccctcgagatggcccaggtgcagctggtggagtctgggggaggcttggtacagcctggggggtccctgagactctcctgtgcagcctctggattcacctttagcagctatgccatgagctgggtccgccaggctccagggaaggggctggagtgggtctcagttatttatagcggtggtagtagcacatactatgcagactccgtgaagggccggttcaccatctccagagataattccaagaacacgctgtatctgcaaatgaacagcctgagagccgaggacacggccgtatattactgtgcgcgcacttcttacctgaaccatggtgattactggggtcaaggtactctggtgaccgtgtctagcgccgctgcagtggtccccgtgctgcagaaagttaatagcaccaccactaaacctgtcctgaggactcctagtccagtgcacccaacagggaccagtcagccacagagaccggaagactgcagaccaagaggttcagtgaagggaaccggcctggatttcgcctgcgatttttgggccctggtcgtcgtcgcaggagttttgttttgctatggactgctcgtcacagttgctttgtgtgttatctggacaaggaaacggtggcaaaatgagaagtttggggtggacatgccagatgactatgaagatgaaaatctctatgagggcctgaaccttgatgactgttctatgtatgaggacatctccaggggactccagggcacctaccaggatgtgggcaacctccacattggagatgcccagctggaaaagccatga

SEQ ID NO: 127 - protein, 394 aa, synthetic construct
QSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSAAAVVPVLQKVNSTTTKPVLRTPSPVHPTGTSQPQRPEDCRPRGSVKGTGLDFACDFWALVVVAGVLFCYGLLVTVALCVIWTRKRWQNEKFGVDMPDDYEDENLYEGLNLDDCSMYEDISRGLQGTYQDVGNLHIGDAQLEKP

SEQ ID NO: 128 - DNA, 1152 bp, synthetic construct
  source 1..1152: mol_type = other DNA
gaggttcaacttgttcaatctggggcagaagtgaagaagcccggggcatctgtgaaagtatcatgcaaaacatccggctatacgtttaccgaatacaccattcactgggtcagacaggctcccggtcaaagcctcgaatggatgggaaatattaaccctaacaatggcggaaccacatataatcagaaattccaaggccgagtgacgataactgtcgataagagtacgtccacagcttacatggaactcagctctttgagatccgaagacactgcagtttattattgtgcagctggatggaacttcgactattggggacaagggactcttgttacggtgtccagtggcaaaccaggtagtggtaaacccggaagcggcaagcccgggagcggtaaacctggtagcgacatcgtcatgactcaaagccctgactcactcgccgtgagcctgggagagcgtgcaacgctatcttgtcgggcctctcaggatgtcggaactgctgtagactggtatcaacagaaacctgaccaatcaccaaaactcctgatttattgggcctcaacacgtcacacaggagtgccagataggttcacaggtagtggcagtggaactgattttactttgacaattagcagcctgcaagccgaagatgtagccgtttacttctgtcaacaatataactcatacccactaacgttcggtgccgggacgaaggtagagattaaagtggtccccgtgctgcagaaagttaatagcaccaccactaaacctgtcctgaggactcctagtccagtgcacccaacagggaccagtcagccacagagaccggaagactgcagaccaagaggttcagtgaagggaaccggcctggatttcgcctgcgatttttgggccctggtcgtcgtcgcaggagttttgttttgctatggactgctcgtcacagttgctttgtgtgttatctggacaaggaaacggtggcaaaatgagaagtttggggtggacatgccagatgactatgaagatgaaaatctctatgagggcctgaaccttgatgactgttctatgtatgaggacatctccaggggactccagggcacctaccaggatgtgggcaacctccacattggagatgcccagctggaaaagccatga

SEQ ID NO: 129 - protein, 383 aa, synthetic construct
EVQLVQSGAEVKKPGASVKVSCKTSGYTFTEYTIHWVRQAPGQSLEWMGNINPNNGGTTYNQKFQGRVTITVDKSTSTAYMELSSLRSEDTAVYYCAAGWNFDYWGQGTLVTVSSGKPGSGKPGSGKPGSGKPGSDIVMTQSPDSLAVSLGERATLSCRASQDVGTAVDWYQQKPDQSPKLLIYWASTRHTGVPDRFTGSGSGTDFTLTISSLQAEDVAVYFCQQYNSYPLTFGAGTKVEIKVVPVLQKVNSTTTKPVLRTPSPVHPTGTSQPQRPEDCRPRGSVKGTGLDFACDFWALVVVAGVLFCYGLLVTVALCVIWTRKRWQNEKFGVDMPDDYEDENLYEGLNLDDCSMYEDISRGLQGTYQDVGNLHIGDAQLEKP

SEQ ID NO: 130 - DNA, 1344 bp, synthetic construct
  source 1..1344: mol_type = other DNA
atgtgtcagtcacgctatcttctcttccttgctactctggccttgctcaatcacttgtcccttgctcgtgtgattcctgtgtccggcccagctaggtgtctctcccagtcacggaatctcctgaaaaccacggatgacatggtaaagacagctagggagaaactcaagcactactcctgcacagctgaggatatcgatcatgaggacatcaccagggaccagacatccactctgaaaacttgcctgcctttggaactccacaagaacgaatcttgtctggcaacgcgtgaaacgagttctactacaagagggtcctgtcttccccctcaaaagacaagccttatgatgaccttgtgtctcggtagcatttatgaggacctaaagatgtatcaaaccgagtttcaggctatcaatgcagcgctccagaatcataaccatcagcagatcattcttgacaaaggaatgctcgtggccattgatgaactaatgcagagcctaaaccacaatggcgagactcttcgacagaaaccgcctgtgggcgaggccgatccatatagagtcaaaatgaaactgtgtattctcctgcatgcatttagtactcgtgtagtgactattaacagagtgatgggttacctttcctcagctcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatag

SEQ ID NO: 131 - protein, 447 aa, synthetic construct
MCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 132 - DNA, 1704 bp, synthetic construct
  source 1..1704: mol_type = other DNA
atgtgcccacagaaactcacaatttcttggttcgcaatcgtcctgctggtgtcacccctgatggcaatgtgggagttggaaaaggatgtatacgtcgtcgaggtcgactggacacctgacgctccgggtgaaactgtcaacctcacttgcgatactcctgaagaggacgacatcacgtggacgagcgaccagcgacatggagtgatagggtctggcaagacgcttactatcacggttaaggaatttctcgacgcagggcagtacacatgtcacaagggcggcgagactctgagccactcccatttgctgctgcacaagaaggagaatggtatctggtctaccgaaatcctgaagaattttaagaacaagacttttctgaaatgcgaggccccaaattattccggacgtttcacttgcagttggctcgttcaaagaaatatggacttgaaatttaacattaaatccagctcttcatctcctgacagcagggccgtaacttgtggaatggcttcattgtcagctgagaaagttacgcttgaccaaagggattatgagaaatacagcgtgagttgccaggaagatgtgacatgtccaacggcagaggaaacgttgccaattgagctcgctttggaagctcgtcaacaaaacaagtatgaaaactatagtactagcttcttcatacgggacatcatcaaaccagatccacctaagaatttgcagatgaagcctctgaagaattcacaagtcgaggtatcctgggaatacccagattcatggtccactcctcatagttactttagcctgaaattctttgtacgcatacagcggaagaaggagaaaatgaaggagacggaagaaggctgcaatcagaaaggcgcttttcttgttgaaaagacgagcactgaggttcaatgcaaaggcgggaatgtatgtgttcaagcccaagataggtattataatagctcctgctctaagtgggcttgcgtaccatgcagagttagaagtcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatag

SEQ ID NO: 133 - protein, 567 aa, synthetic construct
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 134 - DNA, 1344 bp, synthetic construct
  source 1..1344: mol_type = other DNA
atgtgtcagtcacgctatcttctcttccttgctactctggccttgctcaatcacttgtcccttgctcgtgtgattcctgtgtccggcccagctaggtgtctctcccagtcacggaatctcctgaaaaccacggatgacatggtaaagacagctagggagaaactcaagcactactcctgcacagctgaggatatcgatcatgaggacatcaccagggaccagacatccactctgaaaacttgcctgcctttggaactccacaagaacgaatcttgtctggcaacgcgtgaaacgagttctactacaagagggtcctgtcttccccctcaaaagacaagccttatgatgaccttgtgtctcggtagcatttatgaggacctaaagatgtatcaaaccgagtttcaggctatcaatgcagcgctccagaatcataaccatcagcagatcattcttgacaaaggaatgctcgtggccattgatgaactaatgcagagcctaaaccacaatggcgagactcttcgacagaaaccgcctgtgggcgaggccgatccatatagagtcaaaatgaaactgtgtattctcctgcatgcatttagtactcgtgtagtgactattaacagagtgatgggttacctttcctcagctcccagagggcccacaatcaagccctgtcctccatgcaaatgcccagcacctaacgctgccggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcggagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 135 - protein, 447 aa, synthetic construct
MCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 136 - DNA, 1704 bp, synthetic construct
  source 1..1704: mol_type = other DNA
atgtgcccacagaaactcacaatttcttggttcgcaatcgtcctgctggtgtcacccctgatggcaatgtgggagttggaaaaggatgtatacgtcgtcgaggtcgactggacacctgacgctccgggtgaaactgtcaacctcacttgcgatactcctgaagaggacgacatcacgtggacgagcgaccagcgacatggagtgatagggtctggcaagacgcttactatcacggttaaggaatttctcgacgcagggcagtacacatgtcacaagggcggcgagactctgagccactcccatttgctgctgcacaagaaggagaatggtatctggtctaccgaaatcctgaagaattttaagaacaagacttttctgaaatgcgaggccccaaattattccggacgtttcacttgcagttggctcgttcaaagaaatatggacttgaaatttaacattaaatccagctcttcatctcctgacagcagggccgtaacttgtggaatggcttcattgtcagctgagaaagttacgcttgaccaaagggattatgagaaatacagcgtgagttgccaggaagatgtgacatgtccaacggcagaggaaacgttgccaattgagctcgctttggaagctcgtcaacaaaacaagtatgaaaactatagtactagcttcttcatacgggacatcatcaaaccagatccacctaagaatttgcagatgaagcctctgaagaattcacaagtcgaggtatcctgggaatacccagattcatggtccactcctcatagttactttagcctgaaattctttgtacgcatacagcggaagaaggagaaaatgaaggagacggaagaaggctgcaatcagaaaggcgcttttcttgttgaaaagacgagcactgaggttcaatgcaaaggcgggaatgtatgtgttcaagcccaagataggtattataatagctcctgctctaagtgggcttgcgtaccatgcagagttagaagtcccagagggcccacaatcaagccctgtcctccatgcaaatgcccagcacctaacgctgccggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcggagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 137 - protein, 567 aa, synthetic construct
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 138 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 139 - DNA, 342 bp, synthetic construct
  source 1..342: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 140 - protein, 570 aa, synthetic construct
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPELQLEESCAEAQDGELDGLWTTITIFITLFLLSVCYSATVTFFKVKWIFSSVVDLKQTIIPDYRNMIGQGA

SEQ ID NO: 141 - DNA, 1713 bp, synthetic construct
  source 1..1713: mol_type = other DNA
atggaaaccgatacactgctgctgtgggtgctgctgctgtgggtgccaggatctaccggtcagtctgtgttgacgcagccgccctcagtgtctgcggccccaggacagagggtcaccatctcctgctctggaaccaggtccaacattgggagtgattatgtttcctggtaccaacacctcccaggaacagcccccaaactcctcgtttatggcgataatctgcgaccctcagggattcctgaccgattctctgcctccaagtctggcacgtcagccaccctgggcatcaccggactccagactggggacgaggccgattattactgcggcacatgggattacaccctgaatggtgtggtgttcggcggagggaccaagctgaccgtcctaggttctagaggtggtggtggtagcggcggcggcggctctggtggtggtggatccctcgagatggcccaggtgcagctggtggagtctgggggaggcttggtacagcctggggggtccctgagactctcctgtgcagcctctggattcacctttagcagctatgccatgagctgggtccgccaggctccagggaaggggctggagtgggtctcagttatttatagcggtggtagtagcacatactatgcagactccgtgaagggccggttcaccatctccagagataattccaagaacacgctgtatctgcaaatgaacagcctgagagccgaggacacggccgtatattactgtgcgcgcacttcttacctgaaccatggtgattactggggtcaaggtactctggtgaccgtgtctagccccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccgagctgcaactggaggagagctgtgcggaggcgcaggacggggagctggacgggctgtggacgaccatcaccatcttcatcacactcttcctgttaagcgtgtgctacagtgccaccgtcaccttcttcaaggtgaagtggatcttctcctcggtggtggacctgaagcagaccatcatccccgactacaggaacatgatcggacagggggcctga

SEQ ID NO: 142 - protein, 236 aa, synthetic construct
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 143 - DNA, 711 bp, synthetic construct
  source 1..711: mol_type = other DNA
atggaaaccgatacactgctgctgtgggtgctgctgctgtgggtgccaggatctaccggtcagtctgtgttgacgcagccgccctcagtgtctgcggccccaggacagagggtcaccatctcctgctctggaaccaggtccaacattgggagtgattatgtttcctggtaccaacacctcccaggaacagcccccaaactcctcgtttatggcgataatctgcgaccctcagggattcctgaccgattctctgcctccaagtctggcacgtcagccaccctgggcatcaccggactccagactggggacgaggccgattattactgcggcacatgggattacaccctgaatggtgtggtgttcggcggagggaccaagctgaccgtcctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttcatag

SEQ ID NO: 144 - protein, 537 aa, synthetic construct
MVFTPQILGLMLFWISASRGQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPELQLEESCAEAQDGELDGLWTTITIFITLFLLSVCYSATVTFFKVKWIFSSVVDLKQTIIPDYRNMIGQGA

SEQ ID NO: 145 - DNA, 1614 bp, synthetic construct
  source 1..1614: mol_type = other DNA
atggtgtttacaccgcaaatattggggctcatgcttttctggatcagtgcaagcaggggacaggtgcagctggtggagtctgggggaggcttggtacagcctggggggtccctgagactctcctgtgcagcctctggattcacctttagcagctatgccatgagctgggtccgccaggctccagggaaggggctggagtgggtctcagttatttatagcggtggtagtagcacatactatgcagactccgtgaagggccggttcaccatctccagagataattccaagaacacgctgtatctgcaaatgaacagcctgagagccgaggacacggccgtatattactgtgcgcgcacttcttacctgaaccatggtgattactggggtcaaggtactctggtgaccgtgtctagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccgagctgcaactggaggagagctgtgcggaggcgcaggacggggagctggacgggctgtggacgaccatcaccatcttcatcacactcttcctgttaagcgtgtgctacagtgccaccgtcaccttcttcaaggtgaagtggatcttctcctcggtggtggacctgaagcagaccatcatccccgactacaggaacatgatcggacagggggcctga

SEQ ID NO: 146 - protein, 404 aa, synthetic construct
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSAAAFVPVFLPAKPTTTPAPRPPTPAPTIASQPLSLRPEACRPAAGGAVHTRGLDFACDFWVLVVVGGVLACYSLLVTVAFIIFWVLDKDDSKAGMEEDHTYEGLDIDQTATYEDIVTLRTGEVKWSVGEHPGQE

SEQ ID NO: 147 - DNA, 1215 bp, synthetic construct
  source 1..1215: mol_type = other DNA
atggaaaccgatacactgctgctgtgggtgctgctgctgtgggtgccaggatctaccggtcagtctgtgttgacgcagccgccctcagtgtctgcggccccaggacagagggtcaccatctcctgctctggaaccaggtccaacattgggagtgattatgtttcctggtaccaacacctcccaggaacagcccccaaactcctcgtttatggcgataatctgcgaccctcagggattcctgaccgattctctgcctccaagtctggcacgtcagccaccctgggcatcaccggactccagactggggacgaggccgattattactgcggcacatgggattacaccctgaatggtgtggtgttcggcggagggaccaagctgaccgtcctaggttctagaggtggtggtggtagcggcggcggcggctctggtggtggtggatccctcgagatggcccaggtgcagctggtggagtctgggggaggcttggtacagcctggggggtccctgagactctcctgtgcagcctctggattcacctttagcagctatgccatgagctgggtccgccaggctccagggaaggggctggagtgggtctcagttatttatagcggtggtagtagcacatactatgcagactccgtgaagggccggttcaccatctccagagataattccaagaacacgctgtatctgcaaatgaacagcctgagagccgaggacacggccgtatattactgtgcgcgcacttcttacctgaaccatggtgattactggggtcaaggtactctggtgaccgtgtctagcgccgctgcattcgtgcctgtgttcctcccagctaagcccactaccacccccgctccaaggccgcccacgcccgctcctactattgctagtcagcctttaagtttacgacccgaagcttgcaggcccgccgccggcggcgctgtgcacaccagggggcttgattttgcctgcgacttttgggtattggtagtggtgggcggagttttagcctgctacagcctcctggtaacagtggcttttatcatcttttgggtgctggacaaggatgacagcaaggctggcatggaggaagatcacacctacgagggcctggacattgaccagacagccacctatgaggacatagtgacgctgcggacaggggaagtgaagtggtctgtaggtgagcacccaggccaggagtga

SEQ ID NO: 148 - protein, 416 aa, synthetic construct
METDTLLLWVLLLWVPGSTGQSVLTQPPSVSAAPGQRVTISCSGTRSNIGSDYVSWYQHLPGTAPKLLVYGDNLRPSGIPDRFSASKSGTSATLGITGLQTGDEADYYCGTWDYTLNGVVFGGGTKLTVLGSRGGGGSGGGGSGGGGSLEMAQVQLVESGGGLVQPGGSLRLSCAASGFTFSSYAMSWVRQAPGKGLEWVSVIYSGGSSTYYADSVKGRFTISRDNSKNTLYLQMNSLRAEDTAVYYCARTSYLNHGDYWGQGTLVTVSSAAAFVPVFLPAKPTTTPAPRPPTPAPTIASQPLSLRPEACRPAAGGAVHTRGLDFACDFWVLVVVGGVLACYSLLVTVAFIIFWVRKRWQNEKLGLDAGDEYEDENLYEGLNLDDCSMYEDISRGLQGTYQDVGSLNIGDVQLEKP

SEQ ID NO: 149 - DNA, 1251 bp, synthetic construct
  source 1..1251: mol_type = other DNA
atggaaaccgatacactgctgctgtgggtgctgctgctgtgggtgccaggatctaccggtcagtctgtgttgacgcagccgccctcagtgtctgcggccccaggacagagggtcaccatctcctgctctggaaccaggtccaacattgggagtgattatgtttcctggtaccaacacctcccaggaacagcccccaaactcctcgtttatggcgataatctgcgaccctcagggattcctgaccgattctctgcctccaagtctggcacgtcagccaccctgggcatcaccggactccagactggggacgaggccgattattactgcggcacatgggattacaccctgaatggtgtggtgttcggcggagggaccaagctgaccgtcctaggttctagaggtggtggtggtagcggcggcggcggctctggtggtggtggatccctcgagatggcccaggtgcagctggtggagtctgggggaggcttggtacagcctggggggtccctgagactctcctgtgcagcctctggattcacctttagcagctatgccatgagctgggtccgccaggctccagggaaggggctggagtgggtctcagttatttatagcggtggtagtagcacatactatgcagactccgtgaagggccggttcaccatctccagagataattccaagaacacgctgtatctgcaaatgaacagcctgagagccgaggacacggccgtatattactgtgcgcgcacttcttacctgaaccatggtgattactggggtcaaggtactctggtgaccgtgtctagcgccgctgcattcgtgcctgtgttcctcccagctaagcccactaccacccccgctccaaggccgcccacgcccgctcctactattgctagtcagcctttaagtttacgacccgaagcttgcaggcccgccgccggcggcgctgtgcacaccagggggcttgattttgcctgcgacttttgggtattggtagtggtgggcggagttttagcctgctacagcctcctggtaacagtggcttttatcatcttttgggtgaggaaacgatggcagaacgagaagctcgggttggatgccggggatgaatatgaagatgaaaacctttatgaaggcctgaacctggacgactgctccatgtatgaggacatctcccggggcctccagggcacctaccaggatgtgggcagcctcaacataggagatgtccagctggagaagccgtga

SEQ ID NO: 150 - DNA, 1389 bp, synthetic construct
  source 1..1389: mol_type = other DNA
gaggttcaacttgttcaatctggggcagaagtgaagaagcccggggcatctgtgaaagtatcatgcaaaacatccggctatacgtttaccgaatacaccattcactgggtcagacaggctcccggtcaaagcctcgaatggatgggaaatattaaccctaacaatggcggaaccacatataatcagaaattccaaggccgagtgacgataactgtcgataagagtacgtccacagcttacatggaactcagctctttgagatccgaagacactgcagtttattattgtgcagctggatggaacttcgactattggggacaagggactcttgttacggtgtccagtggcaaaccaggtagtggtaaacccggaagcggcaagcccgggagcggtaaacctggtagcgacatcgtcatgactcaaagccctgactcactcgccgtgagcctgggagagcgtgcaacgctatcttgtcgggcctctcaggatgtcggaactgctgtagactggtatcaacagaaacctgaccaatcaccaaaactcctgatttattgggcctcaacacgtcacacaggagtgccagataggttcacaggtagtggcagtggaactgattttactttgacaattagcagcctgcaagccgaagatgtagccgtttacttctgtcaacaatataactcatacccactaacgttcggtgccgggacgaaggtagagattaaattcgtgcctgtgttcctcccagctaagcccactaccacccccgctccaaggccgcccacgcccgctcctactattgctagtcagcctttaagtttacgacccgaagcttgcaggcccgccgccggcggcgctgtgcacaccagggggcttgattttgcctgcgacttttgggtattggtagtggtgggcggagttttagcctgctacagcctcctggtaacagtggcttttatcatcttttgggtgaagaaggttgcaaaaaaacctactaataaggctccccatcctaagcaagagccccaagaaattaactttcccgatgatcttccgggttctaacacggcagccccggtgcaggagaccctgcatggttgtcaacccgtcactcaggaggacgggaaagagtctcgtatctccgtccaggagagacagcgcaaaaaacgtataagcgcaaactctacagatccagtaaaagccgcgcaattcgagcctcccggccgccagatgattgcaatacggaaacgtcaactggaggaaactaataatgactatgagacggccgacggtggatacatgacccttaatccccgcgcgccaaccgacgatgataagaacatatatctgacgctcccccctaacgatcacgttaacagtaataattaa

SEQ ID NO: 151 - DNA, 2217 bp, Mus musculus
  source 1..2217: mol_type = genomic DNA
atggccgggaccgtgcgcaccgcgtgcttggtggtggcgatgctgctcagcttggacttcccgggacaggcgcagcccccgccgccgccgccggacgccacctgtcaccaagtccgctccttcttccagagactgcagcccggactcaagtgggtgccagaaactcccgtgccaggatcagatttgcaagtatgtctccctaagggcccaacatgctgctcaagaaagatggaagaaaaataccaactaacagcacgattgaacatggaacagctgcttcagtctgcaagtatggagctcaagttcttaattattcagaatgctgcggttttccaagaggcctttgaaattgttgttcgccatgccaagaactacaccaatgccatgttcaagaacaactacccaagcctgactccacaagcttttgagtttgtgggtgaatttttcacagatgtgtctctctacatcttgggttctgacatcaatgtagatgacatggtcaatgaattgtttgacagcctgtttccagtcatctatacccagctaatgaacccaggcctgcctgattcagccttggacatcaatgagtgcctccgaggagcaagacgtgacctgaaagtatttgggaatttccccaagcttattatgacccaggtttccaagtcactgcaagtcactaggatcttccttcaggctctgaatcttggaattgaagtgatcaacacaactgatcacctgaagttcagtaaggactgtggccgaatgctcaccagaatgtggtactgctcttactgccagggactgatgatggttaaaccctgtggcggttactgcaatgtggtcatgcaaggctgtatggcaggtgtggtggagattgacaagtactggagagaatacattctgtcccttgaagaacttgtgaatggcatgtacagaatctatgacatggagaacgtactgcttggtctcttttcaacaatccatgattctatccagtatgtccagaagaatgcaggaaagctgaccaccactattggcaagttatgtgcccattctcaacaacgccaatatagatctgcttattatcctgaagatctctttattgacaagaaagtattaaaagttgctcatgtagaacatgaagaaaccttatccagccgaagaagggaactaattcagaagttgaagtctttcatcagcttctatagtgctttgcctggctacatctgcagccatagccctgtggcggaaaacgacaccctttgctggaatggacaagaactcgtggagagatacagccaaaaggcagcaaggaatggaatgaaaaaccagttcaatctccatgagctgaaaatgaagggccctgagccagtggtcagtcaaattattgacaaactgaagcacattaaccagctcctgagaaccatgtctatgcccaaaggtagagttctggataaaaacctggatgaggaagggtttgaaagtggagactgcggtgatgatgaagatgagtgcattggaggctctggtgatggaatgataaaagtgaagaatcagctccgcttccttgcagaactggcctatgatctggatgtggatgatgcgcctggaaacagtcagcaggcaactccgaaggacaacgagataagcacctttcacaacgagaaaccaccagaggacccgccagacagcaagaatacacttgtcctctttggcgctgggttcggcgccgtcataacggttgttgtcatcgtggtaataatcaagtgcttttgcaagcacaggtcttgttttcgcaggaatgaagcctctagagaaacaaataattcactgacctttggccccgaagaagctcttgcagagcaaacggtgtttctcatgctcgtgttgttgcctgacgaagtgtcaggccttgaacagttggaatcaatcatcaattttgaaaaactgaccgagtggacatctagcaacgttatggaggagcgaaagataaaagtgtatctgcctagaatgaagatggaggaaaagtataacctgacttccgtcctgatggccatggggatcaccgatgttttcagcagctccgctaaccttagcggcatttccagcgctgagagtctgaagatttctcaagcagtgcacgctgcccacgccgagattaatgaggctgggcgcgaagtagtcggctcagcagaggcaggcgtcgatgcagcatag

SEQ ID NO: 152 - protein, 738 aa, Mus musculus
MAGTVRTACLVVAMLLSLDFPGQAQPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSMPKGRVLDKNLDEEGFESGDCGDDEDECIGGSGDGMIKVKNQLRFLAELAYDLDVDDAPGNSQQATPKDNEISTFHNEKPPEDPPDSKNTLVLFGAGFGAVITVVVIVVIIKCFCKHRSCFRRNEASRETNNSLTFGPEEALAEQTVFLMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAA

(19) United States
(12) Patent Application Publication
(10) Pub. No.: US 2023/0137343 A1
(43) Pub. Date: May 4, 2023

(54) METHODS AND COMPOSITIONS FOR ENHANCING ACTIVITY OF T CELLS WITH MODIFIED B CELLS

(71) Applicant: WALKING FISH THERAPEUTICS, INC., South San Francisco, CA (US)

(72) Inventors: Kathleen BOYLE, South San Francisco, CA (US); Hangil PARK, South San Francisco, CA (US); Srinivas KOTHAKOTA, South San Francisco, CA (US); Mark SELBY, South San Francisco, CA (US); Thomas BRENNAN, South San Francisco, CA (US); Lewis T. WILLIAMS, South San Francisco, CA (US); Rosa Romano, South San Francisco, CA (US); Weijie Lan, South San Francisco, CA (US)

(21) Appl. No.: 17/938,545

(22) Filed: Oct. 6, 2022

Related U.S. Application Data
(60) Provisional application No. 63/252,989, filed on Oct. 6, 2021.

(51) Int. Cl. A61K 35/17; A61K 45/06; A61P 35/00; A61P 37/02

(52) U.S. Cl. CPC A61K 35/17; A61K 45/06; A61P 35/00; A61P 37/02

(57) ABSTRACT

The present invention relates to methods and combination therapies for enhancing the activity and function of non-B cell immune cells (such as T cells) using genetically modified B cells. These methods and combinations can be used, for example, for the treatment of a variety of diseases and disorders, including cancer, heart disease, inflammatory disease, muscle wasting disease, neurological disease, and the like.

### BACKGROUND OF THE INVENTION

B cells, also known as B lymphocytes, are a type of white blood cell responsible for, among other things, helping the body resist infection and diseases. They are part of our adaptive immune system, and are capable of various immune responses, for example, secreting antibodies in response to a recognized antigen. Additionally, B cells are capable of presenting antigens, and can also secrete cytokines.

Many B cells mature into plasma cells that produce antibodies (proteins) capable of fighting off infections. Other B cells mature into memory B cells. All plasma cells descended from a single B cell produce the same antibody that is directed against the antigen that stimulated it to mature. The same principle holds with memory B cells. Thus, all plasma cells and memory cells “remember” the stimulus that led to their formation. The B cell, or B lymphocyte, is not thymus-dependent, has a short lifespan, and is responsible for the production of immunoglobulins. See e.g., https://www.medicinenet.com/script/main/art.asp?articlekey=2413.

B cells express a surface version of antibodies that are antigen specific. B cells can thus recognize antigens on cells, leading to uptake of the antigen. The B cell receptor structure provides that upon antigen encounter, the B cell becomes activated and further, the antigen is internalized. The antigen is routed to a degradation compartment whereby the antigen is proteolytically processed into fragments, some of which associate with MHC II precursor molecules prior to mobilizing to the cell surface. Peptide-loaded MHC II molecules can be recognized by CD4⁺ T cells, leading to activation of T cells in an antigen-specific manner.

B cells appear to be associated with patient outcomes in the treatment of cancer. For example, the presence of tertiary lymphoid structures (TLSs) is associated with better patient outcomes. See, e.g., Helmink, B.A., et al., Nature, 2020, 577(7791), 549-555; Petitprez F et al., Nature, 2020, 577(7791), 556-560. TLSs are aggregates of immune cells (mostly T and B cells) that arise in response to immunological stimuli. While TLSs that surround tumor cells include B cells, the role of B cells in antitumor responses have been unclear. B cells found in tumors can produce inhibitory factors that hinder the function of immune cells. See, e.g., Kessel, A., et al., Autoimmun Rev., 2012, 11(9), 670-677; Khan, A.R., et al., Nature Commun., 2015, 6, 5997. Further, current evidence indicates that B cells impede antitumor responses in many mouse models of cancer. Affara, N.I., et al. Cancer Cell, 2014, 25(6), 809-821; Shalapour, S., et al., Nature, 2017, 551, 340-345; Ammirante, M. et al., Nature, 2010, 464, 302-305.

T cells, particularly Chimeric Antigen Receptors for T cells (CAR-T) have proven effective against some hematopoietic tumors. CAR-Ts are engineered T cells designed to mimic elements of natural T cell receptors. In CAR-T cells, specific antigen recognition elements, often scFvs, are located in the extracellular domain of the CAR-T, anchored to the transmembrane domain followed by one or more signaling elements. Costimulatory molecules are frequently included in the CAR construct, typically in the extracellular, and/or transmembrane, and/or intracellular domains. Once the CAR-T cells is engaged by cognate antigen, a signal is transduced into the cell leading to activation of cytolytic programs and eventually target cell death. CAR-T cells must maintain in the host for long periods to eliminate tumor cells as well as maintain constant surveillance. Notwithstanding advances in hematologic malignancies, CAR-T cells have certain drawbacks, for example they have thus far proven relatively ineffective for solid tumors.

There exists a need for improved treatments utilizing T cell therapies, such as treatment in conjunction with engineered B cells, for the treatment of a variety of diseases and disorders, including cancer, heart disease, inflammatory disease, muscle wasting disease, neurological disease, and the like.

### SUMMARY OF THE INVENTION

It has now been found that B cells (including, but not limited to CAR-B cells herein) can be used to enhance T cell therapies. For example, in accordance with the invention, it has been found that modified CAR B cells present antigen and activate CD4⁺ and CD8⁺ T cells. Furthermore, it has been found that this effect can be enhanced by co-expression of CD80.

In certain embodiments, the invention relates to methods of treating a patient comprising administering to said patient an effective amount of (i) a plurality of isolated T cells and (ii) an effective amount of plurality of isolated modified B cells: wherein said isolated modified B cells are capable of expressing a chimeric receptor (CAR-B), and wherein said chimeric receptor comprises:
a) an extracellular domain, wherein the extracellular domain comprises an extracellular binding domain and a hinge domain;
b) a transmembrane domain; and
c) a cytoplasmic domain that comprises at least one signaling domain.

In certain embodiments, the isolated T cells and said CAR-B cells are administered sequentially or concurrently. In certain embodiments, the extracellular binding domain recognizes at least one antigen or protein expressed on the surface of a target cell.

In some embodiments, the extracellular binding domain(s) recognizes at least one antigen that is a secreted protein.

In certain embodiments, the target cell is selected from the group consisting of a tumor cell, a cardiac muscle cell, a skeletal muscle cell, a bone cell, a blood cell, a nerve cell, a fat cell, a skin cell, an endothelial cell, a hepatocyte, a pulmonary epithelial cell, and a fibroblast cell.

In some embodiments, the B cell expresses more than one CAR-B receptor construct.

In certain embodiments, the extracellular binding domain is a single chain variable fragment (scFv), or a full-length antibody or an antibody fragment, or the extracellular domain of a receptor or ligand. In some embodiments, the extracellular binding domain is capable of binding to an antigen or protein selected from the group consisting of: PSMA, GPC3, ASGR1, ASGR2, Sarcoglycan, Corin, FAP, MUC1, CEA153, JAM-1, LAF-1 and Her2.

In certain embodiments, the cytoplasmic domain comprises a domain that is selected from the group consisting of: CD79a (Immunoglobulin α), CD40, CD19, CD137, Fcγr2a, MyD88, CD21, Syk, FYN, LYN, PI3K, BTK, PLCγ2, CD3ζ and BLNK.

In some embodiments, the cytoplasmic domain comprises CD79a. In some embodiments, the isolated modified B cell is capable of expressing and secreting a payload, wherein the payload is either (i) not naturally expressed in a B cell or (ii) is expressed at higher levels than is naturally expressed in a B cell. In some aspects, the payload can be an antibody or fragment thereof. In certain aspects of the invention, the payload is at least one payload selected from cytokines, chemokines, T cell costimulatory molecules, and checkpoint molecules, the group consisting of: IL-1, IL-7, IL-8, IL-10, IL-12, IL-13, IL-17, IL-18, IL-21, interferon α, interferon β, interferon γ, TSLP, CCL21, FLT3L, XCL1, LIGHT(TNFSF14), OX40L, CD137L, CD40L, ICOSL, anti-CD3 antibody, CD47, TIM4-FC, CXCL13, CCL21, CD80, CD86, CD40L, IFNα A2, LIGHT , 4-1BBL, MDGF (C19orf10), FGF10, PDGF, agrin, TNF-α, GM-CSF, an anti-FAP antibody, an anti-TGF-β antibody; a TGF-β trap, decoy or other inhibitory molecule; an anti-BMP antibody; a BMP trap, decoy or other inhibitory molecule.

In certain embodiments, the isolated modified B cell is administered intra-tumorally, intravenously, subcutaneously, intradermally, or within an inflammatory lesion. In certain embodiments, the method further comprises administering to a patient one or more checkpoint inhibitors, with or without an additional chemotherapeutic agent.

In other aspects, the invention relates to a method of treating a patient comprising administering to said patient an effective amount of (i) a plurality of isolated non-B cell modified immune cells and (ii) an effective amount of plurality of isolated modified B cells; wherein said isolated modified B cells are capable of expressing a chimeric receptor (CAR-B), and wherein said chimeric receptor comprises:
a) an extracellular domain, wherein the extracellular domain comprises an extracellular binding domain and a hinge domain;
b) a transmembrane domain; and
c) a cytoplasmic domain that comprises at least one signaling domain.

In certain embodiments, the non-B cell modified immune cells are at least one of CAR-T cells, TILs, and TCR cells. In certain embodiments, the extracellular binding domain recognizes at least one antigen or protein expressed on the surface of a target cell.

In certain embodiments, the extracellular binding domain(s) recognizes at least one antigen that is a secreted protein. In certain aspects, the target cell is selected from the group consisting of a tumor cell, a cardiac muscle cell, a skeletal muscle cell, a bone cell, a blood cell, a nerve cell, a fat cell, a skin cell, an endothelial cell, a hepatocyte, a pulmonary epithelial cell, and a fibroblast cell.

In certain embodiments, the invention relates to a combination therapy comprising: An isolated modified non-B cell immune cell, and an isolated modified B cell capable of expressing a chimeric receptor, wherein said chimeric receptor comprises:
a) an extracellular domain, wherein the extracellular domain comprises an extracellular binding domain and a hinge domain;
b) a transmembrane domain; and
c) a cytoplasmic domain that comprises at least one signaling domain
wherein said modified B cell is optionally further capable of expressing a payload. In certain embodiments, the payload comprises at least one of CD80, or CD86.

In certain embodiments, the non-B cell modified immune cells are at least one of CAR-T cells, TILs, and TCR cells.

In accordance with the invention, a 3-component system was been developed comprising CAR B cells that were co-cultured with a source of specific antigen (antigen presenting cells, or APCs) and antigen-specific T cells.

As noted herein below, the consequence of B Cell Receptor (BCR) signaling in B cells leads not only to secretion of antibody but also to presentation of antigen fragments derived from the target antigen via processing within the B cells. Wennhold and colleagues as well as Kitamura presented data suggesting vaccine-induced antigen-specific B cells have antitumor benefit upon adoptive transfer to tumor-bearing mice. Indeed, antigen presentation may play a role in promoting antitumor T cell responses. It has now been found that antigen presentation is a mechanism-of-action for CAR B-mediated antitumor activity.

CAR-B cells can be co-transferred with other cell types bearing CAR’s to enhance their activity and persistence. Such cells can include T, NK, and myeloid (monocyte/macrophage) cells. Most CARs recognize antigen via scFv-like structures leading to signaling, facilitating cytolytic or phagocytic activity. These CAR-bearing cells can be potentiated by the provision of CAR-B cell-derived cytokines, leading to improved signaling and persistence. This can be demonstrated in an immune competent host bearing a syngeneic tumor co-treated with a murine CAR T cell, for example, along with a CAR B cell. The antigen specificity of the CAR B cell can be the same as the CAR T, for example. Alternatively, it can also be different although the greatest benefit is with a target expressed in the same tumor cell. Additionally, CAR cells targeting MHC/peptide complexes on the tumor cells can be activated by presentation of the same peptides derived from processing of antigen after uptake in CAR B cells.

CAR B cells can be engineered to express the CAR and payload constructs using, but not limited to, mRNA electroporation, recombinant adenovirus transduction, CRISPR editing methods or combinations thereof.

It has now also been found that engineered B cells can be efficacious in the treatment of various diseases and disorders as recited herein. The invention therefore relates to isolated modified B cells capable of activating or enhancing activity of T cells. Suitable T cell types include CAR-T, TILs, TCRs, and the like.

CD79 (also termed “Cluster of Differentiation 79”) is a transmembrane protein that forms a complex with the B-cell receptor and is capable of generating a signal following recognition of an antigen by the B-cell receptor.¹ CD79 is comprised of two different chains known as CD79a and CD79b (also termed Igα and Igβ). CD79a and CD79b are both members of the immunoglobulin superfamily. These form a heterodimer on the surface of B cells stabilized by disulfide bonding. Both CD79 chains contain an immunoreceptor tyrosine-based activation motif (“ITAM”) in their intracellular tail regions that propagate a signal in a B cell.²

It has also been found that CD79a (Immunoglobulin-α) when incorporated into the intracellular signaling domain of the CAR-B constructs of the invention exhibits superior qualities over CD79b (Immunoglobulin-β). Further, it has further been found that when used in the CAR-B constructs described herein, intracellular CD79b (Immunoglobulin-β) displays no (or even a negative effect) on efficacy. The invention thus relates to, inter alia, CAR-B constructs comprising the CD79a intracellular signaling domain.

In certain embodiments, the invention relates to an isolated modified B cell (“CAR-B cell), capable of expressing a chimeric receptor (“CAR-B receptor”), wherein said chimeric receptor comprises (a) an extracellular domain; (b) a transmembrane domain; and (c) a cytoplasmic domain that comprises at least one signaling domain. The cytoplasmic domain preferably comprises CD79a. In various embodiments, the extracellular domain comprises an extracellular binding domain and a hinge domain. In various embodiments, the extracellular binding domain(s) recognizes at least one antigen or protein expressed on the surface of a target cell. In various embodiments, the target cell is selected from the group consisting of a tumor cell, cardiac muscle cell, a skeletal muscle cell, a bone cell, a blood cell, a nerve cell, a fat cell, a skin cell, and an endothelial cell. In various embodiments, the B cell expresses more than one CAR-B receptor construct. In various embodiments, the CAR-B receptor comprises more than one extracellular binding domain. In various embodiments, the extracellular binding domain is a single chain variable fragment (scFv), or a full-length antibody, or the extracellular domain of a receptor or ligand. In various embodiments, the extracellular binding domain is capable of binding to an antigen or protein selected from the group consisting of: PSMA, GPC3, ASGR1, ASGR2, Sarcoglycan, Corin, FAP (fibroblast activation protein) and Her2. In various embodiments, the hinge domain is derived from the group consisting of IgG, CD28 and CD8. In various embodiments, the hinge domain is comprised of a nucleic acid sequence selected from the group consisting of SEQ ID Nos. 27, 29, 31. In various embodiments, the cytoplasmic domain comprises at least one signaling domain native to B cell receptors. In various embodiments, the cytoplasmic domain comprises a domain that is selected from the group consisting of: CD79a (Immunoglobulin α), CD79b (Immunoglobulin β), CD40, CD19, CD137, Fcγr2a, MyD88, CD21, Syk, FYN, LYN, PI3K, BTK, PLCγ2, CD3ζ and BLNK. In various embodiments, the cytoplasmic domain further comprises a costimulatory domain.

In various embodiments, the invention comprises an isolated modified B cell, wherein said B cell is capable of expressing and secreting a payload, wherein the payload is not naturally expressed in a B cell or is expressed at higher levels than is naturally expressed in a B cell. In various embodiments, the payload is an antibody or fragment thereof. In various embodiments, the antibody is a secreted antibody and can include blocking antibodies (eg anti-PD-1) or agonist antibodies (anti-CD137, GITR, OX40) engineered to contain native or engineered Fc regions and can be soluble or membrane-bound In various embodiments, the payload(s) can be immune modifiers such as chemokines or cytokines. In various embodiments, the payload is selected from the group consisting of: IL-1, IL-7, IL-8, IL-10, IL-12, IL-13, IL-17, IL18, IL-21, interferon α, interferon β, interferon γ, TSLP, CCL21, FLT3L, XCL1, LIGHT(TNFSF14), OX40L, CD137L, CD40L, ICOSL, anti-CD3 antibody, CD47, TIM4-FC, CXCL13, CCL21, CD80, CD86, CD40L, IFNα A2, LIGHT , 4-1BBL, MDGF (C19orf10), FGF10, PDGF, agrin, TNF-α, GM-CSF, an anti-FAP antibody, an anti-TGF-β antibody; a TGF-β trap, decoy or other inhibitory molecule; an anti-BMP antibody; a BMP trap, decoy or other inhibitory molecule. In various embodiments, the B cell is capable of expressing more than one payload. In various embodiments, the B cell is capable of expressing more than 1, 2, 3, 4, 5, 6, 7, 8, 9, 10, 11, or 12 payloads.

In various embodiments, the invention relates to a method of treating a patient comprising administering the modified B cell of the present invention. In various embodiments, the modified B cell is administered intra-tumorally, intravenously, subcutaneously, or intradermally. In various embodiments, the method further comprises administering a checkpoint inhibitor. In various embodiments, the checkpoint inhibitor to a checkpoint molecule that is selected from the group consisting of PD-1, PD-L1, CTLA-4, LAG3, TIM-3 and NKG2A proteins. In various embodiments, the checkpoint inhibitor is a monoclonal antibody.

In various embodiments, the invention relates to an isolated modified B cell, capable of expressing a chimeric receptor, wherein said chimeric receptor comprises (a) an extracellular domain, wherein the extracellular domain comprises an extracellular binding domain and a hinge domain; (b) a transmembrane domain; and (c) a cytoplasmic domain that comprises at least one signaling domain, wherein said modified B cell is further capable of expressing a payload, wherein the payload is not naturally expressed on the surface of a cell. In various embodiments, the extracellular binding domain recognizes an antigen or protein expressed on the surface of a target cell. In various embodiments, the target cell is selected from the group consisting of a tumor cell, a cardiac muscle cell, a skeletal muscle cell, a bone cell, a blood cell, a nerve cell, a fat cell, a skin cell and an endothelial cell. In various embodiments, the B cell expresses more than one CAR-B receptor construct. In various embodiments, the CAR-B receptor comprises more than one extracellular binding domain. In various embodiments, the extracellular binding domain is a single chain variable fragment (scFv), an antibody, or the extracellular domain of a receptor or ligand. In various embodiments, the extracellular binding domain is capable of binding to an antigen or protein selected from the group consisting of PSMA, GP3, ASGR1, ASGR2, Sarcoglycan, Corin, FAP and Her2. In various embodiments, the hinge domain is derived from the group consisting of IgG, CD28 and CD8. In various embodiments, the hinge domain is comprised of a nucleic acid sequence selected from the group consisting of SEQ ID Nos. 27, 29, and 31. In various embodiments, the cytoplasmic domain comprises at least one signaling domain native to B cells. In various embodiments, the cytoplasmic domain comprises a domain selected from the group consisting of: CD79a (Immunoglobulin α), CD79b (Immunoglobulin β), CD40, CD19, CD137, Fcγr2a, MyD88, CD21, Syk, FYN, LYN, PI3K, BTK, PLCγ2, CD3ζ and BLNK. In various embodiments, the cytoplasmic domain further comprises a costimulatory domain. In various embodiments, the payload is a secreted or membrane bound antibody or fragment thereof. In various embodiments, the payload is selected from the group consisting of: IL-1, IL-7, IL-8, IL-10, IL-12, IL-13, IL-17, IL-18, IL-21, interferon α, interferon β, interferon γ, TSLP, CCL21, FLT3L, XCL1, LIGHT(TNFSF14), OX40L, CD137L, CD40L, ICOSL, anti-CD3 antibody, CD47, TIM4-FC, CXCL13, CCL21, CD80, CD86, CD40L, IFNα A2, LIGHT , 4-1BBL, MDGF (C19orf10), FGF10, PDGF, agrin, TNF-α, GM-CSF, an anti-FAP antibody, an anti-TGF-β antibody; a TGF-β trap, decoy or other inhibitory molecule; an anti-BMP antibody; a BMP trap, decoy or other inhibitory molecule. In various embodiments, the B cell is capable of expressing more than one payload. In various embodiments, the B cell is capable of expressing more than 1, 2, 3, 4, 5, 6, 7, 8, 9, 10, 11, or 12 payloads. In various embodiments, the modified B cell further encodes at least one protein selected from the group consisting of: the cytoplasmic domains of CD79a, CD79b, CD40, CD19, CD137, Fcγr2a, CD3ζ and MyD88. In various embodiments, the intention relates to a method of treating a patient comprising administering the modified B cell. In various embodiments, the method further comprises administering a checkpoint inhibitor. In various embodiments, the checkpoint inhibitor is selected from inhibitors to one or more checkpoint molecules from the group consisting of: PD-1, PD-L1, CTLA-4, LAG3, TIM-3 and NKG2A. In various embodiments, the checkpoint inhibitor is a monoclonal antibody. In various embodiments, the present invention relates to an isolated modified B cell, capable of expressing a chimeric receptor, wherein said chimeric receptor comprises an extracellular domain, wherein said extracellular domain comprises a hinge domain and an extracellular binding domain, wherein said extracellular binding domain is not naturally expressed on a B cell; and wherein said extracellular binding domain is capable of recognizing a target of interest. In various embodiments, the binding domain is a single chain variable fragment (scFv), antibody, ligand or receptor. In various embodiments, the binding domain is an scFv. In various embodiments, the binding domain is a receptor, a ligand, or a fragment thereof. In various embodiments, the B cell is further capable of expressing a payload. In various embodiments, the invention comprises a method of treating a patient comprising administering the modified B cell to a patient.

In various embodiments, the present invention comprises a nucleic acid capable of expressing a chimeric B cell receptor, wherein said chimeric receptor comprises: (a) an extracellular domain, wherein said extracellular domain comprises an extracellular binding domain and a hinge domain; (b) a transmembrane domain; and (c) a cytoplasmic domain that comprises at least one signaling domain. In various embodiments, the extracellular binding domain, recognizes an antigen or protein expressed on the surface of a target cell. In various embodiments, the extracellular binding domain is a single chain variable fragment (scFv), antibody, receptor or ligand. In various embodiments, the target cell is selected from the group consisting of a tumor cell, a cardiac muscle cell, a skeletal muscle cell, a bone cell, a blood cell, a nerve cell, a fat cell, a skin cell and an endothelial cell. In various embodiments, the vector expresses more than one CAR-B receptor. In various embodiments, the CAR-B receptor expresses more than one extracellular binding domain. In various embodiments, the extracellular binding domain is capable of binding to an antigen or protein selected from the group consisting of: PSMA, GP3, ASGR1, ASGR2, Sarcoglycan, Corin, Her2, FAP, MUC1, CEA153, JAM-1, and LFA-1. In various embodiments, the hinge domain is derived from the group consisting of IgG, CD28 and CD8. In various embodiments, the hinge domain is comprised of a nucleic acid sequence selected from the group consisting of SEQ ID Nos. 27, 29, and 31. In various embodiments, the cytoplasmic domain comprises at least one signaling domain native to B cell receptors. In various embodiments, the cytoplasmic domain comprises a domain selected from the group consisting of: CD79a (Immunoglobulin α), CD79b (Immunoglobulin β), CD40, CD19, CD137, Fcγr2a, MyD88, CD21, Syk, FYN, LYN, PI3K, BTK, PLCγ2, CD3ζ and BLNK. In various embodiments, the cytoplasmic domain further comprises a costimulatory domain.

In various embodiments, the invention relates to a vector comprising a nucleic acid capable of expressing a chimeric B cell receptor, wherein said chimeric receptor comprises: (a) an extracellular domain, wherein said extracellular domain comprises an extracellular binding domain and a hinge domain; (b) a transmembrane domain; and (c) a cytoplasmic domain that comprises at least one signaling domain. In various embodiments, the extracellular binding domain recognizes an antigen or protein. In various embodiments, the target cell is selected from the group consisting of a tumor cell, a cardiac muscle cell, a skeletal muscle cell, a bone cell, a blood cell, a nerve cell, a fat cell, a skin cell and an endothelial cell. In various embodiments, the vector expresses more than one CAR-B receptor. In various embodiments, the CAR-B expresses more than one extracellular binding domain. In various embodiments, the extracellular binding domain is a single chain variable fragment (scFv), antibody, receptor or ligand. In various embodiments, the extracellular binding domain is capable of binding to an antigen or protein selected from the group consisting of: PSMA, GPC3, ASGR1, AGSR2, Sarcoglycan, Corin, Her2, FAP, MUC1, CEA153, JAM-1, and LFA-1. In various embodiments, the hinge domain is derived from the group consisting of IgG, CD28 and CD8. In various embodiments, the hinge domain is comprised of a nucleic acid sequence selected from the group consisting of SEQ ID Nos. 27, 29, and 31. In various embodiments, the cytoplasmic domain comprises at least one signaling domain native to B cells. In various embodiments, the cytoplasmic domain comprises a signaling domain selected from the group consisting of: CD79a (Immunoglobulin α), CD79b (Immunoglobulin β), CD40, CD19, CD137, Fcγr2a, MyD88, CD21, Syk, FYN, LYN, PI3K, BTK, PLCγ2, CD3ζ and BLNK. In various embodiments, the cytoplasmic domain further comprises a costimulatory domain. The various embodiments, the cytoplasmic region is comprised of multiple, 2 or more, domains, being either identical or unique.

In various embodiments, the invention relates to an isolated modified B cell, capable of expressing an integrin, a homing antibody, protein, a receptor, or combinations thereof, wherein said integrin, homing antibody, protein, or receptor is not naturally expressed in a B cell or is expressed at higher levels than is naturally expressed in a B cell; and wherein said integrin, homing antibody, protein, receptor, or combinations thereof is attracted to a site or target of interest. In various embodiments, the integrin, homing antibody, protein, and receptor is selected from CLA (PSGL-1 glycoform), CLA (PSGL-1 glycoform), CCR10, CCR3, CCR4, CCR5, CCR6, CCR9, CD43E, CD44, c-Met, CXCR3, CXCR4, LFA-1, LFA-1 (αLβ2), selectin ligands, VLA-4, VLA-4 (α4β1), and α4β7, or combinations thereof. In various embodiments, the site of interest is a homing or target tissue, an inflammatory site in a specific location or tissue, or a tumor or tumor microenvironment, where delivery of payloads is desirable. In various embodiments, the homing or target tissue is selected from skin, gut (intestine, colon, mesenteric lymph nodes (mLN), Peyer’s Patch (PP), small intestine), liver, lung, bone marrow, heart, peripheral lymph node (LN), CNS, thymus, and bone marrow. In various embodiments, the target of interest is selected from CXCL16, CCL17, CCL17(22), CCL20 (MIP-3α), CCL21, CCL25, CCL27, CCL28, CCL4, CCL5, CD62E, CD62P, CXCL10, CXCL12, CXCL13, CXCL16, CXCL9/CXCL10, CXCR3, E/P-selectin, E-selectin, GPR15L, HGF, Hyaluronate, ICAM-1, ligands for CCR1, 2, 5, MAdCAM, MAdCAM-1, PNAd, VAP-1, VCAM, and VCAM-1, or combinations thereof. In various embodiments, the method comprises treating a patient by administering the isolated modified B cell. In various embodiments, the method involves further administering a compound or a derivative thereof, wherein the compound or derivative thereof is capable of increasing the expression of the integrin, homing antibody, protein, and receptor, or combinations thereof. In various embodiments, the compound or a derivative thereof is capable of altering trafficking of B cells to a site or target of interest in the patient. In various embodiments, the compound is all-trans-retinoic acid (ATRA) or a derivative thereof.

In various embodiments, the invention relates to an isolated modified B cell, capable of expressing an immune inhibitory molecule, wherein said immune inhibitory molecule is not naturally expressed in a B cell or is expressed at higher levels than is naturally expressed in a B cell. In various embodiments, said immune inhibitory molecule is selected from IL-10, TGF-β, PD-L1, PD-L2, LAG-3, and TIM-3, or combinations thereof. In various embodiments, said immune inhibitory molecule is capable of decreasing inflammation and autoimmune activity of B cells at a site or target of interest in a patient. In various embodiments, the invention relates to a method of treating a patient comprising administering said isolated modified B cell. In various embodiments, said immune inhibitory molecule is selected from IL-10, TGF-β, PD-L1, PD-L2, LAG-3, and TIM-3, or combinations thereof. In various embodiments, said immune inhibitory molecule is capable of decreasing inflammation and autoimmune activity of B cells at a site or target of interest in the patient. In various embodiments, the invention relates to further administering a compound or a derivative thereof capable of increasing the expression of an integrin, a homing antibody, a protein, a receptor, or combinations thereof in the B cell. In various embodiments, said compound or derivative thereof is capable of altering trafficking of B cells to a site or target of interest in the patient. In various embodiments, said compound is all-trans-retinoic acid (ATRA) or a derivative thereof. In various embodiments, the invention relates to an isolated modified B cell, wherein the isolated modified B cell is treated with a compound or a derivative thereof, wherein said compound or derivative thereof is capable of increasing the expression of an integrin, a homing antibody, a protein, a receptor, or combinations thereof in B cells. In various embodiments, said compound or derivative thereof is capable of altering trafficking of B cells to a site or target of interest in the patient. In various embodiments, said compound is all-trans-retinoic acid (ATRA) or a derivative thereof. In various embodiments, said compound or derivative thereof is capable of (i) increasing the expression of an integrin, a homing antibody, a protein, a receptor, or combinations thereof in B cells, and (ii) altering trafficking of B cells to a site or target of interest in the patient. In various embodiments, said compound is all-trans-retinoic acid (ATRA) or a derivative thereof.

In various embodiments, the invention relates to an isolated modified B cell, capable of expressing at least one or more of a constitutively active Toll-like receptor (TLR), wherein said TLR is not naturally expressed in a B cell or is expressed at higher levels than is naturally expressed in a B cell. In various embodiments, said TLR is selected from TLR1, TLR2, TLR3, TLR4, TLR5, TLR6, TLR7, TLR8, TLR9, TLR10, TLR11, TLR12, and TLR13, or combinations thereof. In various embodiments, said TLR is capable of potentiating B cells for increasing immune responses in a patient. In various embodiments, said TLR is capable of producing potent effector B cells for increasing immune responses in a patient. In various embodiments, said immune inhibitory molecule is capable of decreasing inflammation and autoimmune activity of B cells at a site or target of interest in a patient. In various embodiments, said TLR is selected from TLR1, TLR2, TLR3, TLR4, TLR5, TLR6, TLR7, TLR8, TLR9, TLR10, TLR11, TLR12, and TLR13, or combinations thereof. In various embodiments, said TLR is capable of (i) potentiating B cells, and (ii) producing potent effector B cells, for increasing immune responses in a patient. In various embodiments, at least one or more of a TLR agonist is administered to the patient. In various embodiments, the isolated modified B cell is treated with at least one or more of a TLR agonist. In various embodiments, said TLR agonist is capable of (i) potentiating B cells, and (ii) producing potent effector B cells, for increasing immune responses in a patient. In various embodiments, said TLR agonist binds to one or more TLRs selected from TLR1, TLR2, TLR3, TLR4, TLR5, TLR6, TLR7, TLR8, TLR9, TLR10, TLR11, TLR12, and TLR13, or combinations thereof. In various embodiments, said TLR agonist is selected from CpG-rich oligonucleotides, double-stranded RNA mimic, polyinosinic acid:polycytidylic acid (poly-I:C). In various embodiments, said TLR agonist comprises CpG oligonucleotides. In various embodiments, said TLR agonist is capable of is capable of (i) potentiating B cells, and (ii) producing potent effector B cells, for increasing immune responses in the patient. In various embodiments, said TLR agonist binds to one or more TLRs selected from TLR1, TLR2, TLR3, TLR4, TLR5, TLR6, TLR7, TLR8, TLR9, TLR10, TLR11, TLR12, and TLR13, or combinations thereof. In various embodiments, said TLR agonist is selected from CpG-rich oligonucleotides, double-stranded RNA mimic, polyinosinic acid:polycytidylic acid (poly-I:C). In various embodiments, said TLR agonist comprises CpG oligonucleotides.

In various embodiments, the invention relates to an isolated modified B cell, wherein said B cell is electroporated with an mRNA encoding at least one or more of an antigen fused to a targeting signal. In various embodiments, said antigen is (i) not naturally presented by a B cell, (ii) not presented by a B cell simultaneously in both HLA class I and class II molecules naturally, or (iii) not presented by a B cell with high efficiencies in both HLA class I and class II molecules naturally. In various embodiments, said targeting signal is targeting signal of a lysosomal protein. In various embodiments, said targeting signal is a targeting signal of lysosome-associated membrane protein-1 (LAMP1). In various embodiments, said antigen is capable of being targeted to the lysosomes and presented simultaneously and efficiently in both HLA class I and class II molecules. In various embodiments, said B cells is capable of increasing antigen-specific immune responses in a patient. In various embodiments, said antigen is (i) not naturally presented by a B cell, (ii) not presented by a B cell simultaneously in both HLA class I and class II molecules naturally, or (iii) not presented by a B cell with high efficiencies in both HLA class I and class II molecules naturally. In various embodiments, said targeting signal is targeting signal of a lysosomal protein. In various embodiments, said targeting signal is a targeting signal of lysosome-associated membrane protein-1 (LAMP1). In various embodiments, said antigen is capable of being targeted to the lysosomes and presented simultaneously and efficiently in both HLA class I and class II molecules. In various embodiments, said B cells is capable of increasing antigen-specific immune responses in the patient.

### BRIEF DESCRIPTION OF THE FIGURES

FIG. 1 sets forth an example of a chimeric B Cell Receptor (CAR-B) of the present invention. In certain embodiments, the CAR-B construct will comprise an extracellular domain, a transmembrane domain, and a cytoplasmic domain. As depicted in FIG. 1, the extracellular domain may in certain embodiments comprise a binding domain and a hinge region. In certain embodiments, the binding region may be an scFv. CAR-B constructs are cloned into a vector for expression.

FIGS. 2A-2C show examples of engineered B cells with homing domains. In various embodiments, the engineered B cells may comprise (a) an scFv binding domain and optional hinge region; (b) an scFv binding domain directly linked to the cell through a transmembrane domain, or (c) a ligand/receptor binding domain directly linked to a cell through a transmembrane domain.

FIG. 3 shows examples of certain CAR-B constructs of the present invention. (A) CAR-B that binds GPC3. (B) CAR-B that binds PSMA.

FIG. 4 shows examples of CAR-B receptors of the present invention capable of binding (A) GPC3 and (B) PSMA. The “C” domain corresponds to the native BCR C-terminus.

FIG. 5 sets forth expression of various anti-PSMA CARs on the surface of HEK-293 cells.

FIGS. 6A-6C set forth a FACS Plot illustrating interrogation of binding of anti-PSMA CAR and of anti-sarcoglycan CAR to PSMA. B cells expressing anti-PSMA CAR-B constructs pWF396 and pWF397 bound PSMA whereas the B cells expressing pWF398 (anti-sarcoglycan CAR-B) did not bind PSMA.

FIG. 7 illustrates the ability of adenovirus F35 encoding GFP to transduce human B cells. Human B cells were isolated from peripheral blood. The B cells were infected with adenovirus encoding GFP. 0, 1, 3, 10 ul, representing the microliter volume of the adenovirus preparation used to infect human B cells. The titer of the adenovirus preparations were approximately 1 x e¹² particles/ml.

FIG. 8 describes an experiment where BALB/c mice were injected with CT26 bilateral tumors at day zero. At day 12 and day 16, tumor-bearing mice were injected intra-tumorally with payload-expressing cells at a volume of 10⁶ in 50 µL.

FIG. 9 illustrates the effect of 12 different combinations of payloads injected intra-tumorally on tumor volume over 30-35 days as compared to saline and 3T3 cells (without a payload).

FIG. 10 illustrates the effect of 12 different combinations of payloads injected intra-tumorally on tumor volume over 30-35 days as compared to saline and 3T3 cells (without a payload).

FIGS. 11A-11C illustrate the effect of the top three combinations of payloads injected intra-tumorally on tumor volume over 30 days as compared to saline and 3T3 cells (without a payload).

FIG. 12 illustrates the abscopal effect of intratumorally injected B cells. B cells were then injected either (i) fresh or (ii) first stimulated for 16-24 hours in growth media (RPMI, 10% FBS, 1% Pen/Strep, 5 ng/ml recombinant mouse IL-4, 100uM beta-mercaptoethanol) with 5 µg/ml Lipopolysaccharide. 5X10⁶ B cells were then intratumorally injected into the CT26 mouse model, and anti-tumor responses in the distal (abscopal) tumor where measured. Tumors were implanted at day 0, and at day 6 palpable tumor mass was observed. Treatment was initiated on day 6 intratumorally.

FIGS. 13A-13C illustrates expression of three CAR-B receptors (also referred to as CAR-B receptors) in mouse B cells 24 hours post transfection.

FIG. 14 illustrates the efficacy of PSMA-specific CAR engineered murine B cells on tumor volume and survival in BALB/c mice with CT26-PSMA tumors.

FIG. 15 illustrates the efficacy of PSMA-specific CAR engineered allogenic B cells on tumor volume and survival in BALB/c mice with CT26-PSMA tumors.

FIGS. 16A and 16B illustrate the efficacy of PSMA-specific CAR engineered murine B cells on immunocompromised BALB/c mice with CT26-PSMA tumors.

FIG. 17 illustrates the efficacy of murine B cells on tumor volume and survival in C57B1/6 mice with HEPA 1-6 GPC3 tumors.

FIG. 18 illustrates NFKb signaling in luciferase reporter cells in B cells engineered with four different CAR constructs that recognized GPC3, using GFP as a control.

FIG. 19 illustrates basal or tonic NFKb activity in the absence of cognate target antigen in CAR constructs expressed in human B cell reporter line.

FIG. 20 illustrates the efficacy of murine B cells electroporated with anti-GPC3CAR-CD79a and a CD80 payload mRNAs in syngeneic C57B1/6 mice with HEPA1-6GPC3 tumors.

FIGS. 21A-21C illustrate the responses of the saline control, anti-GPC3CAR-CD79a, and anti-GPC3CAR-CD79a plus CD80 combo B cell groups.

FIGS. 22A-22C illustrate the expression of the GPC3 CAR post-electroporation, using FACS plots.

FIG. 23 illustrates T cell activation at 24 hours. In this Figure, 293 cells are shown expressing HEL served as antigen-presenting cells. HEL-specific CAR B cell were co-cultured with the HEL antigen-presenting cells along with OTII cells at a 1:1:1 ratio. After ~24 hours, cells were recovered by centrifugation and subjected to flow cytometry using anti-CD69-PE (vendor) and gating on CD4 cells.

FIG. 24A shows IL-2 measurements with B cell alone and B cell with CAR (with CD79a and CD79b) for GPC3 and PSMA antigens.

FIG. 24B shows that antigen specific activation of CAR B cells stimulates immune enhancing cytokine production.

FIG. 25 shows tumor antigen processing by B cells and presentation potentiated by a CD80 payload.

FIG. 26 shows tumor antigen processing by B cells and presentation potentiated by a CD80 payload shown as percent of activated T cells.

FIG. 27 shows that HEL specific CAR-B cells can take up the HEL-OVA-antigen from co-cultured cells to specifically activate OVA-specific CD4 and CD8 T cells.

FIG. 28 is a diagrammatic representation of T Cell stimulation in vivo.

FIG. 29 is a diagrammatic representation of the modified B cells capturing antigen from tumors and activating T cells through antigen presentation.

FIG. 30 shows mouse B cells activated for 24 hours in presence of anti-CD40 and IL-4, and electroporated with wild-type (WT) Cas9 complex with I-A/I-E and b2m sgRNAs. Three or six days after targeting, expression of b2m and IA-IE was evaluated using flow cytometry.

FIG. 31 shows T cell activation (measured by OVA-specific CD4⁺/CD69⁺ and CD8⁺/CD69⁺) by GPC3 CAR B cells with CD80, with no knockout guide, Class I knockout guide, and Class II knockout guide.

FIG. 32 shows Study #2 related to FIG. 31 results which shows T cell activation (measured by OVA-specific CD4⁺/CD69⁺) by GPC3 CAR B cells with CD80, with no knockout guide, Class I knockout guide, and Class II knockout guide.

FIG. 33 shows T cell activation ((measured by OVA-specific CD8⁺/CD69⁺⁾ by GPC3 CAR B cells with CD80, with no knockout guide, Class I knockout guide, and Class II knockout guide.

### DETAILED DESCRIPTION

The invention relates to enhancing T cell therapies utilizing engineered B cells described herein. There T cell therapies can be autologous or allogeneic.

The engineered B cells may be used in conjunction (e.g., co-administration) with, or to enhance T cell function of a variety of T cell therapies. Such T cell therapies can target, for example at least one of (or combinations of) CD19, CD20, CD22, BCMA, CD123, PSMA, CLL-1, Her2, Her3, EGFR, ANG2, HGF, TNF, c-Met, IL-13, and the like.

Examples of such T cell therapies include, but are not limited to: tisagenlecleucel (tisa-cel, Kymriah®), axicabtagene ciloleucel (axi-cel, Yescarta®), brexucabtagene autoleucel (brexu-cel, Tecartus®), lisocabtagene maraleucel (liso-cel, Breyanzi®), idecabtagene vicleucel (ide-cel, Abecma®. It will be appreciated that the methods and combinations described herein can further be utilized in connection with T Cell Receptor (TCR) therapies.

A list of various CARs of various types are listed in the multiple tables below. These can be found at https://bluematterconsulting.com/2021-outlook-cell-based-therapies-in-oncology/), the contents of which are hereby incorporated by reference in their entirety. For example, a number of allogeneic cell therapies are currently in development, including, but not limited to:

| | |
|---|---|
| Product Name (Manufacturer) | Description |
| ALLO-501 / UCART19 (Allogene / Cellectis / Servier) | allogeneic, viral-transduced, safety edited |
| ALLO-501A (Allogene / Cellectis / Servier) | allogeneic, viral-transduced, rituximab recognition domain deleted |
| CTX110 (CRISPR Therapeutics) | allogeneic, enzymatic-modified (CRISPR), safety, persistence edited |
| PBCAR0191 (Precision Biosciences) | allogeneic, enzymatic-modified (ARCUS), safety edited |
| FT819 (Fate Therapeutics) | allogeneic (iSPC-derived), enzymatic-modified (CRISPR), safety edited |

Additional cell therapies targeting BCMA for e.g., multiple myeloma include but are not limited to:

| | |
|---|---|
| Product Name (Manufacturer) | Description |
| ide-cel (BMS / Celgene) | autologous, viral-transduced CAR-T |
| cilta-cel (J&J Janssen / Legend) | autologous, viral-transduced CAR-T |
| orva-cel (BMS / Celgene) | autologous, viral-transduced CAR-T |
| bb21217 (BMS / Celgene) | autologous, viral-transduced CAR-T (ide-cel plus P13K boost) |
| ALLO-715 (Allogene / Cellectis) | allogeneic, viral-transduced, safety edited CAR-T |
| CYAD-202 (celyad) | (allogeneic, viral-transduced, safety and add-ons) |
| CTX120 (CRISPR Therapeutics) | allogeneic, enzymatic-modified (CRISPR) safety edited CAR-T |
| Nex-T BMCA (BMS / Celgene) | autologous, optimized manufacturing |
| REGN5458 (Regeneron) | BMCA:CD3 T cell engager (TCE) |
| teclistamab (J&J Janssen) | BMCA:CD3 TCE |
| TNB-383B (Abbvie / Teneobio) | BCMA:CD3 TCE |

Additional cell therapies include:

| | |
|---|---|
| Product Name (Manufacturer) | Description |
| BPX-601 (Bellicum) | PSCA CAR-T (autologous, activation switch “GoCAR-T”) |
| BPX-603 (Bellicum) | HER2 CAR-T (autologous, dual safety / activation switches “Dual Switch GoCAR-T”) |
| PRGN-3005 (Precigen) | MUC16 CAR-T (UltraCAR-T: autologous, Sleeping Beauty, efficacy + safety switch) |
| PRGN-3006 (Precisgen) | CD33 CAR-T (UltraCAR-T: autologous, Sleeping Beauty, efficacy + safety switch) |
| CYAD-01 (Celyad) | NKG2D CAR-T (autologous) |
| CYAD-02 (Celyad) | NKG2D CAR-T (autologous, “next gen” design) |
| CYAD-101 (Celyad) | NKG2D CAR-T (allogeneic) |
| UCARTCS1A (Cellectis) | CS1/SLAMF7 CAR-T (allogeneic, safety edited) |
| UCART22 (Cellectis) | CD22 CAR-T (allogeneic, safety edited) |
| UCART123 (Cellectis) | CD123 CAR-T (allogeneic, safety edited) |
| PBCAR20A (Precision Biosciences) | CD20 CAR-T (allogeneic, safety edited) |

Additional T cell therapies include:

| | |
|---|---|
| Product Name (Manufacturer) | Description |
| BPX-601 (Bellicum) | PSCA CAR-T (autologous, activation switch “GoCAR-T”) |
| BPX-603 (Bellicum) | HER2 CAR-T (autologous, dual safety / activation switches “Dual Switch GoCAR-T”) |
| PRGN-3005 (Precigen) | MUC16 CAR-T (UltraCAR-T: autologous, Sleeping Beauty, efficacy + safety switch) |
| PRGN-3006 (Precisgen) | CD33 CAR-T (UltraCAR-T: autologous, Sleeping Beauty, efficacy + safety switch) |
| CYAD-01 (Celyad) | NKG2D CAR-T (autologous) |
| CYAD-02 (Celyad) | NKG2D CAR-T (autologous, “next gen” design) |
| CYAD-101 (Celyad) | NKG2D CAR-T (allogeneic) |
| UCARTCS1A (Cellectis) | CS1/SLAMF7 CAR-T (allogeneic, safety edited) |
| UCART22 (Cellectis) | CD22 CAR-T (allogeneic, safety edited) |
| UCART123 (Cellectis) | CD123 CAR-T (allogeneic, safety edited) |
| PBCAR20A (Precision Biosciences) | CD20 CAR-T (allogeneic, safety edited) |

| | |
|---|---|
| Product Name (Manufacturer) | Description |
| BPX-601 (Bellicum) | PSCA CAR-T (autologous, activation switch “GoCAR-T”) |
| BPX-603 (Bellicum) | HER2 CAR-T (autologous, dual safety / activation switches “Dual Switch GoCAR-T”) |
| PRGN-3005 (Precigen) | MUC16 CAR-T (UltraCAR-T: autologous, Sleeping Beauty, efficacy + safety switch) |
| PRGN-3006 (Precisgen) | CD33 CAR-T (UltraCAR-T: autologous, Sleeping Beauty, efficacy + safety switch) |
| CYAD-01 (Celyad) | NKG2D CAR-T (autologous) |
| CYAD-02 (Celyad) | NKG2D CAR-T (autologous, “next gen” design) |
| CYAD-101 (Celyad) | NKG2D CAR-T (allogeneic) |
| UCARTCS1A (Cellectis) | CS1/SLAMF7 CAR-T (allogeneic, safety edited) |
| UCART22 (Cellectis) | CD22 CAR-T (allogeneic, safety edited) |
| UCART123 (Cellectis) | CD123 CAR-T (allogeneic, safety edited) |
| PBCAR20A (Precision Biosciences) | CD20 CAR-T (allogeneic, safety edited) |

Additional CAR-T cell therapies for us in the current invention include:

| | |
|---|---|
| Product Name (Manufacturer) | Description |
| BPX-601 (Bellicum) | PSCA CAR-T (autologous, activation switch “GoCAR-T”) |
| BPX-603 (Bellicum) | HER2 CAR-T (autologous, dual safety / activation switches “Dual Switch GoCAR-T”) |
| PRGN-3005 (Precigen) | MUC16 CAR-T (UltraCAR-T: autologous, Sleeping Beauty, efficacy + safety switch) |
| PRGN-3006 (Precisgen) | CD33 CAR-T (UltraCAR-T: autologous, Sleeping Beauty, efficacy + safety switch) |
| CYAD-01 (Celyad) | NKG2D CAR-T (autologous) |
| CYAD-02 (Celyad) | NKG2D CAR-T (autologous, “next gen” design) |
| CYAD-101 (Celyad) | NKG2D CAR-T (allogeneic) |
| UCARTCS1A (Cellectis) | CS1/SLAMF7 CAR-T (allogeneic, safety edited) |
| UCART22 (Cellectis) | CD22 CAR-T (allogeneic, safety edited) |
| UCART123 (Cellectis) | CD123 CAR-T (allogeneic, safety edited) |
| PBCAR20A (Precision Biosciences) | CD20 CAR-T (allogeneic, safety edited) |

As noted, the above lists can be found in further detail at https://bluematterconsulting.com/2021-outlook-cell-based-therapies-in-oncology/).

The invention disclosed herein further relates to several embodiments of engineered or modified B cells:
1. B cells that have been modified to home to a site/target of interest, using, e.g., a binding domain such as an scFv, antibody, ligand, receptor, or fragments thereof;
2. B cells that have been modified with a homing domain, further comprising an activation, and optionally a costimulatory domain, such that the B cells can home and activate upon interaction with a desired target;
3. B cells engineered to be capable of making a desired protein payload, such as an antibody, therapeutic protein, polypeptide, nucleic acid sequence (such as RNAi) or the like;
4. Engineered B cells comprising a homing/binding domain, an activating domain, an optional costimulatory domain, and further engineered to express a desire protein payload, such as an antibody, therapeutic protein, polypeptide, nucleic acid sequence (such as RNAi) or the like;
5. B cells that have been modified to express an integrin, a homing antibody, protein, or a receptor, such that the B cells are attracted to specific ligands, chemokines, or attractants at a specific site/target of interest (e.g., a homing tissue) and can thereby home to the site/target of interest, for example, to deliver a desired payload;
6. B cells that have been modified to express an immune inhibitory molecule, such that the inflammation and autoimmune activity of B cells localized to a site/target of interest is decreased, thereby leading to a positive therapeutic response;
7. B cells that have been treated with a compound or derivatives thereof, such that trafficking of the B cells is altered by expression of specific B cell integrins and/or homing receptors;
8. B cells that have been (i) treated with a Toll-like receptor (TLR) agonist, and/or (ii) engineered to express a constitutively active TLR, for potentiating B cells and/or producing potent effector B cells for increasing immune responses in a subject;
9. B cells that have been electroporated with an mRNA encoding specific antigens of interest fused to a targeting signal of a lysosomal protein, such that the B cells can simultaneously and efficiently present the specific antigens and/or antigen-derived epitopes of interest in both HLA class I and class II molecules.
10. B cells that have been electroporated with a self-amplifying RNA that encodes any items noted heretofore in 1-9.

It is understood that the various embodiments of engineered or modified B cells of the present application are not mutually exclusive and can be combined with each other in any way and without any restriction unless explicitly indicated, for achieving of facilitating any of the results and/or therapeutic responses contemplated herein.

Tumor Antigen. In certain embodiments, the site/target of interest is a tumor antigen. The selection of the antigen-binding domain (moiety) of the invention will depend on the particular type of cancer to be treated. Some tumor antigens may be membrane bound, whereas other may be secreted. For example, a tumor antigen may be secreted and accumulate in the extracellular matrix, or the tumor antigen may be expressed as part of the MHC complex. Tumor antigens are well known in the art and may include, for example, CD19, KRAS, HGF, CLL, a glioma-associated antigen, carcinoembryonic antigen (CEA); β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, protein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, mesothelin, EGFR, BCMA, KIT and IL-13.

Infectious Disease Antigen. In certain embodiments, the site/target of interest is an infectious disease antigen against which an immune response may be desired. Infectious disease antigens are well known in the art and may include, but are not limited to, viruses, bacteria, protists, and parasitic antigens, such as parasites, fungi, yeasts, mycoplasma, viral proteins, bacterial proteins and carbohydrates, and fungal proteins and carbohydrates. In addition, the type of infectious disease of the infectious disease antigen is not particularly limited, and may include, but are not limited to, intractable diseases among viral infectious diseases such as AIDS, hepatitis B, Epstein Barr Virus (EBV) infection, HPV infection, HCV infection, SARS, SARS—CoV2, etc. Parasitic antigens may include, but are not limited to, the malaria parasite sporozoide protein.

In certain embodiments the modified B cells express an engineered B cell receptor (CAR-B) comprising an extracellular domain, a transmembrane domain and an intracellular domain. In certain embodiments, the extracellular domain comprises a binding domain and a hinge domain. In certain embodiments, the extracellular domain comprises a binding domain, such as an scFv, ligand, antibody, receptor, or fragment thereof which allows the modified B cell to target specific target cells by binding to proteins expressed on the surface of those cells. In certain embodiments, the modified tumor cells target and bind to proteins/antigens expressed on the surface of tumor cells. In certain embodiments, the modified B cell further expresses a payload. In certain embodiments, the payload is capable of increasing the number of cross-presenting antigen or antigenic fragments to dendritic cells (DC) in tumors or in lymph nodes. In certain embodiments, the payload is capable of activating and attracting T cells into tumors. In certain embodiments, the payload is capable of fomenting the formation of tertiary lymphoid structures (TLS) in tumors. In certain embodiments of the invention, the modified B cell expresses both a CAR-B and a payload. In certain embodiments, the CAR-B comprises stimulatory domains that activate expression of the payload when bound to an antigen or protein expressed on the surface of a tumor cell.

### Design and Domain Orientation of Chimeric Antigen Receptors in B Cells (CAR-Bs)

In various embodiments, the invention provides a chimeric B Cell Receptor (CAR-B). It will be appreciated that chimeric B cell receptors (CAR-Bs) are genetically engineered receptors. These engineered receptors can be readily inserted into and expressed by B cells in accordance with techniques known in the art. With a CAR-B, a single receptor can be programmed to both recognize a specific protein or antigen expressed on a tumor cell, and when bound to said protein or antigen elicit an anti-tumor response. In various embodiments, the CAR-Bs serve in part as a homing mechanism to deliver B cells to target tissue.

It will be appreciated that relative to the cell bearing the receptor, the chimeric B cell receptor of the invention will comprise an extracellular domain (which will comprise an antigen-binding domain and may comprise an extracellular signaling domain and/or a hinge domain), a transmembrane domain, and an intracellular domain. The intracellular domain comprises at least an activating domain, preferably comprised of CD79a (Immunoglobulin α), CD79b (Immunoglobulin β), CD40, CD19, CD137, CD3ζ Fcγr2a and/or MyD88. It will further be appreciated that the antigen-binding domain is engineered such that it is located in the extracellular protion of the molecule/construct, such that it is capable of recognizing and binding to its target or targets.

Structurally it will be appreciated that these domains correspond to locations relative to the immune cell. Exemplary CAR-B constructs in accordance with the invention are set forth in Table 1:

TABLE 1

| | | | | | |
|---|---|---|---|---|---|
| Construct Name | Extracellular Domain | Hinge | TM | Signal 1 | Signal 2 |
| pWF-82 | anti-PSMA | CD8 | CD28 | hCD19 | |
| pWF-83 | anti-PSMA | CD8 | CD28 | hCD40 | |
| pWF-84 | anti-PSMA | CD8 | CD28 | hCD40 | CD79b |
| pWF-85 | anti-PSMA | CD8 | CD28 | hCD40 | CD137 |
| pWF-86 | anti-PSMA | CD8 | CD28 | hCD40 | Fcγr2a |
| pWF-87 | anti-PSMA | CD8 | CD28 | hMyd88 | hCD40 |
| pWF-88 | anti-PSMA | CD8 | CD28 | CD79a | |
| pWF-89 | anti-PSMA | CD8 | CD28 | CD79b | |
| pWF-391 | anti-PSMA | 3x strep II tag | CD28 | CD79b | |
| pWF-394 | anti-Sarcoglycan | 3x strep II tag | CD28 | CD79b | |
| pWF-396 | anti-GPC-3 | CD8 | CD28 | CD79a | |
| pWF-397 | anti-GPC-3 | CD8 | CD28 | CD79b | |
| pWF-460 | anti-GPC-3 | Human IgG1 Fc | CD28 | CD79a | |
| pWF428 | anti-GPC-3 | Human Lambda Constant region | Human Lambda Constant region | | |
| pWF429 | anti-GPC-3 | Human IgG1 Fc | Human IgG1 Fc | | |
| pWF-521 | Anti-GPC3 vL-hclambda constant region-linker-vH-hcH1-cH2-cH3 | Human IgG1 Fc | Human IgG1 | Endogenous BCR complex | |
| pWF-533 | Anti-GPC3-vL-hcH1 | | Human IgG1 (complex with pWF534) | Endogenous BCR complex | |
| pWF-534 | Anti-GPC3-vH-hcKappa-hcH2-cH3 | Human IgG1 Fc | Human IgG1 | Endogenous BCR complex | |

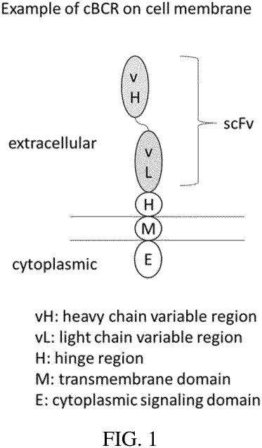

In various embodiments, chimeric B cell receptors are comprised of an extracellular domain, a transmembrane domain and a cytoplasmic domain. In various embodiments, the cytoplasmic domain comprises an activating domain. In various embodiments, the cytoplasmic domain may also comprise a co-stimulatory domain. In various embodiments, the extracellular domain comprises an antigen-binding domain. In various embodiments, the extracellular domain further comprises a hinge region between the antigen-binding domain and the transmembrane domain. FIG. 1 provides a schematic representation of a chimeric B cell receptor of various embodiments of the present invention.

Extracellular Domain. A number of extracellular domains may be used with the present invention. In various embodiments, the extracellular domain comprises an antigen-binding domain. In various embodiments, the extracellular domain may also comprise a hinge region and/or a signaling domain. In various embodiments, the extracellular domains containing IgG1constant domain may also comprise either IgG1(hole) or IgG1(knob) to facilitate directed CAR-B formation.

Antigen-Binding Domain and Binding Domain. As used herein, an “antigen binding domain,” “antigen-binding domain” or “binding domain” refers to a portion of the CAR-B capable of binding an antigen or protein expressed on the surface of a cell. In some embodiments, the antigen-binding domain binds to an antigen or protein on a cell involved in a hyperproliferative disease. In preferred embodiments, the antigen-binding domain binds to an antigen or protein expressed on the surface of a tumor cell. The antigen-binding molecules will be further understood in view of the definitions and descriptions below.

An antigen-binding domain is said to “specifically bind” its target antigen or protein when the dissociation constant (K_{d}) is 1x10⁻⁷ M. The antigen-binding domain specifically binds antigen with “high affinity” when the K_{d} is 1-5x10⁻⁹ M, and with “very high affinity” when the K_{d} is 1-5x10⁻¹⁰ M. In one embodiment, the antigen-binding domain has a K_{d} of 10⁻⁹ M. In one embodiment, the off-rate is <1x10⁻⁵. In other embodiments, the antigen-binding domain will bind to antigen or protein with a K_{d} of between about 10⁻⁷ M and 10⁻¹³ M, and in yet another embodiment the antigen-binding domain will bind with a K_{d} 1.0-5.0x¹⁰.

An antigen-binding domain is said to be “selective” when it binds to one target more tightly than it binds to a second target.

The term “neutralizing” refers to an antigen-binding domain that binds to a ligand and prevents or reduces the biological effect of that ligand. This can be done, for example, by directly blocking a binding site on the ligand or by binding to the ligand and altering the ligand’s ability to bind through indirect means (such as structural or energetic alterations in the ligand). In some embodiments, the term can also denote an antigen-binding domain that prevents the protein to which it is bound from performing a biological function.

The term “target” or “antigen” refers to a molecule or a portion of a molecule capable of being bound by an antigen-binding molecule. In certain embodiments, a target can have one or more epitopes.

The term “antibody” refers to what are known as immunoglobulins, Y-shaped proteins that are produced by the immune system to recognize a particular antigen. The term “antibody fragment” refers to antigen-binding fragments and Fc fragments of antibodies. Types of antigen-binding fragments include: F(ab')2, Fab, Fab' and scFv molecules. Fc fragments are generated entirely from the heavy chain constant region of an immunoglobulin.

Extracellular Signaling Domains. The extracellular domain is beneficial for signaling and for an efficient response of lymphocytes to an antigen. Extracellular domains of particular use in this invention may be derived from (i.e., comprise) CD28, CD28T (See e.g., U.S. Patent Application US2017/0283500A1), OX40, 4-1BB/CD137, CD2, CD7, CD27, CD30, CD40, programmed death-1 (PD-1), inducible T cell costimulator (ICOS), lymphocyte function-associated antigen-1 (LFA-1, CD1-1a/CD18), CD3 gamma, CD3 delta, CD3 epsilon, CD247, CD276 (B7-H3), LIGHT, (TNFSF14), NKG2C, CD79a (Immunoglobulin α), CD79b (Immunoglobulin β), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD1 la, LFA-1, ITGAM, CD1 lb, ITGAX, CD1lc, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof. The extracellular domain may be derived either from a natural or from a synthetic source.

Hinge Domains. As described herein, extracellular domains often comprise a hinge portion. This is a portion of the extracellular domain proximal to the cell membrane. The extracellular domain may further comprise a spacer region. A variety of hinges can be employed in accordance with the invention, including costimulatory molecules as discussed above, as well as immunoglobulin (Ig) sequences a 3X strep II spacer or other suitable molecules to achieve the desired special distance from the target cell. In some embodiments, the hinge region comprises the extracellular domain of CD28, or CD8 or a portion thereof as described herein.

Transmembrane Domains. The CAR-B can be designed to comprise a transmembrane domain that is fused or otherwise linked to the extracellular domain of the CAR-B-B. It can similarly be fused to the intracellular domain of the CAR-B. In one embodiment, the transmembrane domain that naturally is associated with one of the domains in a CAR-B is used. In some instances, the transmembrane domain can be selected or modified by amino acid substitution to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein. Transmembrane regions of particular use in this invention may be derived from (i.e. comprise) CD28, CD28T, OX-40, 4-1BB/CD137, CD2, CD7, CD27, CD30, CD40, programmed death-1 (PD-1), inducible T cell costimulator (ICOS), lymphocyte function-associated antigen-1 (LFA-1, CD1-1a/CD18), CD3 gamma, CD3 delta, CD3 epsilon, CD247, CD276 (B7-H3), LIGHT, (TNFSF14), NKG2C, CD79a (Immunoglobulin α), CD79b (Immunoglobulin β), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD1 la, LFA-1, ITGAM, CD1 lb, ITGAX, CD1 1c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

Optionally, short linkers may form linkages between any or some of the extracellular, transmembrane, and intracellular domains of the CAR-B.

In certain embodiments, the transmembrane domain in the CAR-B of the invention is the CD28 transmembrane domain. In one embodiment, the CD28 transmembrane domain comprises the nucleic acid sequence of SEQ ID NO: 1. In one embodiment, the CD28 transmembrane domain comprises the nucleic acid sequence that encodes the amino acid sequence of SEQ ID NO: 2. In one embodiment, the CD28 transmembrane domain comprises the nucleic acid sequence of SEQ ID NO: 3. In another embodiment, the CD28 transmembrane domain comprises the amino acid sequence of SEQ ID NO: 4.

In one embodiment, the transmembrane domain in the CAR-B of the invention is a CD8 transmembrane domain.

Intracellular (Cytoplasmic) Domains. The intracellular (IC, or cytoplasmic) domain of the CAR-B receptors of the invention can provide activation of at least one of the normal effector functions of the immune cell.

It will be appreciated that suitable intracellular molecules, include, but are not limited to CD79a (Immunoglobulin α), CD79b (Immunoglobulin β), CD40, CD19, CD137, Fcγr2a CD3ζ and MyD88. Intraceullar molecules may further include CD28, CD28T, OX-40, 4-1BB/CD137, CD2, CD7, CD27, CD30, CD40, programmed death-1 (PD-1), inducible T cell costimulator (ICOS), lymphocyte function-associated antigen-1 (LFA-1, CD1-1a/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD1 la, LFA-1, ITGAM, CD1 lb, ITGAX, CD1 1c, ITGB1, CD29, ITGB2, CD18, LFA-1, ITGB7, NKG2D, TNFR2, TRANCE/ RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof. The cytoplasmic signaling sequences within the cytoplasmic signaling portion of the CAR-B of the invention may be linked to each other in a random or specified order.

The term “co-stimulatory” domain or molecule as used herein refers to a heterogenous group of cell surface molecules that act to amplify or counteract initial activating signals of the cell.

In one preferred embodiment, the cytoplasmic domain is designed to comprise the signaling domain of hCD19, wherein the hCD19 domain comprises the nucleic acid sequence set forth in SEQ ID NO. 5. In another embodiment, the cytoplasmic domain is designed to comprise the signaling domain of hCD40, wherein the hCD40 domain comprises the nucleic acid sequence set forth in SEQ ID NO. 7. In another embodiment, the cytoplasmic domain is designed to comprise the signaling domain of hCD40 and hCD79b, wherein the hCD40 domain comprises the nucleic acid sequence set forth in SEQ ID NO. 7 and the hCD79b domain comprises the nucleic acid sequence set forth in SEQ ID NO. 25. In another embodiment, the cytoplasmic domain is designed to comprise the signaling domain of hCD40 and hCD137, wherein the hCD40 domain comprises the nucleic acid sequence set forth in SEQ ID NO. 7 and the hCD137 domain comprises the nucleic acid sequence set forth in SEQ ID NO. 13. In another embodiment, the cytoplasmic domain is designed to comprise the signaling domain of hCD40 and hFcγr2a, wherein the hCD40 domain comprises the nucleic acid sequence set forth in SEQ ID NO. 7 and the hFcγr2a domain comprises the nucleic acid sequence set forth in SEQ ID NO. 17. In another embodiment, the cytoplasmic domain is designed to comprise the signaling domain of hCD40 and hMyd88, wherein the hCD40 domain comprises the nucleic acid sequence set forth in SEQ ID NO. 7 and the hMyd88 domain comprises the nucleic acid sequence set forth in SEQ ID NO. 21. In another embodiment, the cytoplasmic domain is designed to comprise the signaling domain of hCD79a, wherein the hCD79a domain comprises the nucleic acid sequence set forth in SEQ ID NO. 23. In another embodiment, the cytoplasmic domain is designed to comprise the signaling domain of hCD79b, wherein the hCD79b domain comprises the nucleic acid sequence set forth in SEQ ID NO. 25. These embodiments are preferably of human origin but may be derived from other species. In various embodiments the signaling domain comprises both hCD79a in tandem with hCD79b or another hCD79a domain. In various embodiments the signaling domain comprises both hCD79b in tandem with hCD79a or another hCD79b domain.

### Modified B Cells

Modified B Cells that Express Payloads. In various embodiments of the present invention a modified B cell is provided that is capable of expressing a payload. As used herein the term “payload” refers to an amino acid sequence, a nucleic acid sequence encoding a peptide or protein, or an RNA molecule, for use as a therapeutic agent. In certain embodiments the payload is for delivery to the tumor or tumor microenvironment or the draining lymph node. In certain embodiments, it is desirable that the B cell deliver to the tumor or tumor microenvironment or draining lymph node a payload capable of, for example, increasing the number of cross-presenting dendritic cells (DCs) in tumors. Cross-presenting DCs will allow for improved presentation of tumor antigens. In various embodiments, the payload may be capable of activating and attracting T cells into tumors. Activating more T cells in tumors will complement the cross-presenting DCs to remold the tumor environment to have more potent antitumor immune capabilities. Payloads may also foment the formation of tertiary lymphoid structures (TLS) in tumors. Clinical studies have demonstrated that there is a relationship between B cells, TLS and responses to immune checkpoint blockade.

Nonexclusive examples of payloads of the present invention include: IL-1, IL-7, IL-8, IL-10, IL-12, IL-13, IL-17, IL-18, IL-21, interferon α, interferon β, interferon γ, TSLP, CCL21, FLT3L, XCL1, LIGHT(TNFSF14), OX40L, CD137L, CD40L, ICOSL, anti-CD3 antibody, CD47, TIM4-FC, CXCL13, CCL21, CD80, CD86, CD40L, IFNα A2, LIGHT , 4-1BBL, MDGF (C19orf10), FGF10, PDGF, agrin, TNF-α, GM-CSF, an anti-FAP antibody, an anti-TGF-β antibody; a TGF-β trap, decoy or other inhibitory molecule; an anti-BMP antibody; a BMP trap, decoy or other inhibitory molecule.

Signaling for Payload Expression. In various embodiments of the present invention, the payload is expressed in the modified B cell as a DNA construct under the control of an activated transcriptional pathway. In certain embodiments, the expression of the payload is controlled of the Nuclear Factor of Activated T cell (“NFAT”) pathway. The NFAT pathway is a transcription factor pathway activated during an immune response and is activated by the NFκB. In various embodiments, the modified B cell expresses both a payload and a CAR-B. In various embodiments, where the modified B cell expresses both a payload and a CAR-B, the CAR-B may further encode signaling molecules that induce activation of the NFκB pathway. Such molecules include but are not limited to: CD79a (Immunoglobulin α), CD79b (Immunoglobulin β), CD40, CD19, CD137, Fcγr2a, CD3ζ and MyD88.

In various embodiments, the invention relates to isolated B cells that express at least one payload. In various embodiments, the invention relates to isolated B cells that express more than one payload. In various embodiments, the invention relates to isolated B cells that express 1, 2, 3, 4, 5, 6, 7, 8, 9, 10, 11 or 12 different payloads.

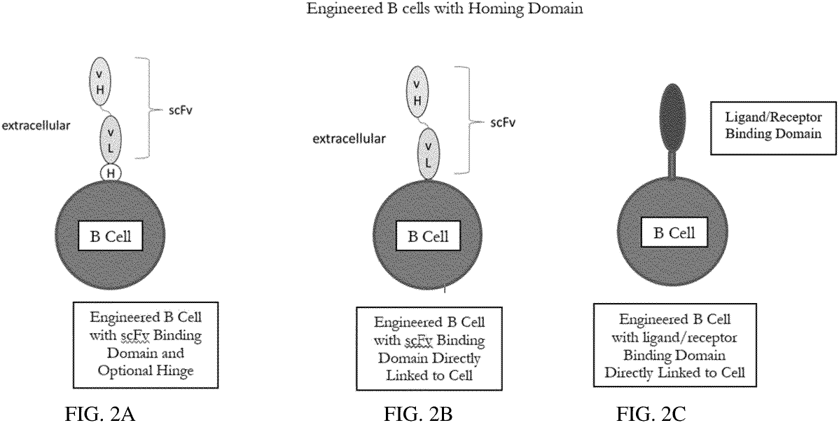

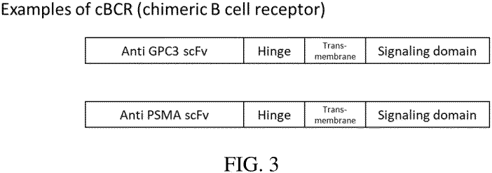

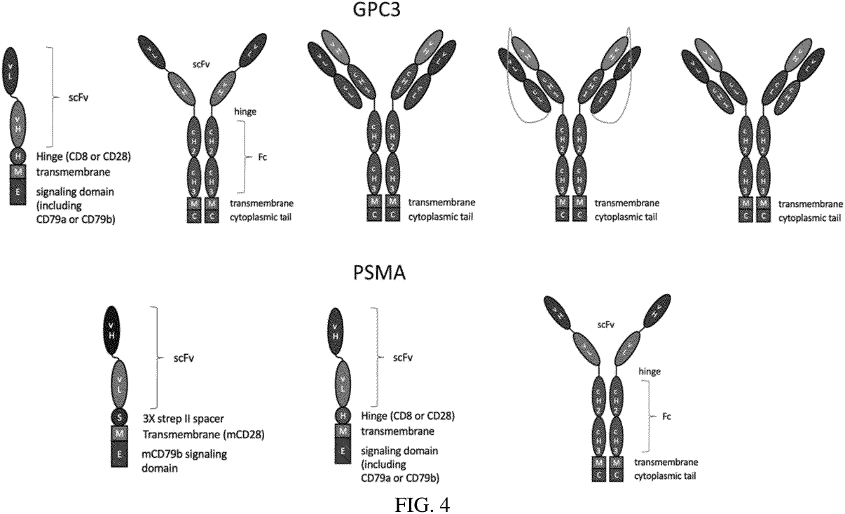

Modification of B Cells for homing. In various embodiments of the present invention, the engineered B cells can be modified with homing domains (e.g., as illustrated in FIG. 2) such that the B cells can home to a site/target of interest and activate upon interaction with the target. Additionally, B cell homing receptors expressed on B cell membranes that recognize addressins and ligands on target tissues, compound or derivatives thereof that alter the trafficking of B cells to a particular site, and inhibitory molecules inflammation and autoimmune activity of the B cells, can play a role in B cell homing and development of specialized immune responses.

Modified B cells that Express Integrin of Interest. The major homing receptors expressed by lymphocytes are the integrins, which are a large class of molecules characterized by a heterodimeric structure of α and β chains. In general, the pairing of specific α and β chains of the integrin determines the type of the homing receptor. For example, pairing of the α4 chain with β7 chain characterizes the major integrin molecule (α4β7) responsible for lymphocyte binding to Mucosal addressin cell adhesion molecule 1 (MAdCAM-1) expressed on high endothelial venules (HEVs) in Peyer’s patches (PP) and gastrointestinal (GI) tract lamina propria endothelial venules (LPVs). Similarly, pairing of the α4 chain with β1 chain characterizes the homing receptor (α4β1) for the skin.

In various embodiments of the present inventions, a B cell to be modified can be selected for in advance, with specific traits that mediate preferred localizations. For example, memory B cells expressing CXCR3 may be enriched for and then subjected to engineering. CXCR3 cells may be attracted to ligands expressed at sites of inflammation. As such, modified B cells can preferentially localize to such sites.

In various embodiments of the present invention, a modified B cell is provided that expresses the α4 and β7 chains of an integrin. It is desirable that expression of the α4β7 integrin will promote homing of the modified B cell to the colon. In various embodiments, a modified B cell is provided that expresses the α4 and β1 chains of an integrin. It is desirable that expression of the α4β1 integrin will promote homing of the modified B cell to the skin. In various embodiments, a modified B cell is provided that expresses a desired pairing of an α and a β chain of an integrin, such that the expressed integrin promotes homing of the modified B cell to a desired site/target of interest. Accordingly, in various embodiments, any desired combination of the α and β chains of an integrin is contemplated for expression in the B cells, such that the modified B cells expressing the specific integrin is targeted to a desired site/target of interest.

Modified B cells that Express Homing Receptors of Interest. B cells have an ability to home to inflammatory tissues and altering their homing receptor expression can complement their native homing tendencies. B cell localization is also driven by expression of attractant molecules (e.g., targets such as ligands and chemokines) at inflammatory sites in specific locations or tissues. Such molecules can also include antibodies, such as the MECA79 antibody that targets cells to peripheral node addressin (PNAd). Bahmani et al., J Clin Invest. 2018;128(11):4770-4786; Azzi et al., Cell Rep. 2016;15(6):1202-13. Accordingly, B cells can be engineered to express certain antibodies, proteins, and receptors that facilitate B cell homing to a site/target of interest and interactions of such B cells with the desired target. In certain instances, expression of such receptors redirects the B cells to the tissue of interest.

In various embodiments of the present invention, a modified B cell is provided that is capable of expressing a homing antibody, protein, or a receptor, expression of which is capable of directing the B cell to a specific site/target of interest. Exemplary homing of T cells to specific homing tissues (target tissues) using specific homing receptor/ligand pairs are set forth in Table 2. The same specific homing receptor/ligand pairs are also capable of facilitating homing of B cells to a specific homing tissue (target tissue). Accordingly, in various embodiments of the present invention, homing of the modified B cells to an exemplary homing tissue (target tissue) is facilitated using the corresponding homing receptor/ligand pairs as set forth in Table 2.

TABLE 2

T_{eff} cell homing receptors and their cognate ligands mediating organotropic targeting

| | | |
|---|---|---|
| Homing Tissue Type | T_{eff} Cell Homing Receptor | Cognate Ligand |
| Skin | CLA (PSGL-1 glycoform) | E/P-selectin |
| | CD43E | E-selectin |
| | VLA-4 (α₄β₁) | VCAM-1 |
| | LFA-1 (α_{L}β₂) | ICAM-1 |
| | CCR4 | CCL17 |
| | CCR10 | CCL27 |
| Gut (intestine, colon, mLN, PP) | α₄β₇ | MAdCAM-1 |
| | CCR9^{a} | CCL25^{a} |
| | CXCR4 | CXCL12 |
| | Selectin ligands^{b} | E/P-selectin^{b} |
| | VLA-4^{b} | VCAM-1^{b} |
| | LFA-1^{b} | ICAM-1^{b} |
| | CCR6^{b} | CCL20 (MIP-3α)^{b} |
| Liver | CD44 | Hyaluronate |
| | VLA-4 | VCAM-1 |
| | CCR5 | CCL5 |
| | | VAP-1 |
| | Selectin ligands^{b} | E/P-selectin |
| | α₄β₇ ^{b} | MAdCAM-1^{b} |
| Lung | LFA-1 | ICAM-1 |
| | CCR3 | CCL28 |
| | CCR4 | CCL17 |
| | CXCR4 | CXCL12 |
| | Selectin ligands^{b} | E/P-selectin^{b} |
| | VLA-4^{b} | VCAM-1^{b} |
| | LFA-1^{b} | ICAM-1^{b} |
| Bone Marrow | CLA (PSGL-1 glycoform) | E/P-selectin |
| | CD43E | E-selectin |
| | VLA-4 | VCAM-1 |
| | LFA-1 | ICAM-1 |
| | CXCR4 | CXCL12 |
| | α₄β₇ ^{b} | MAdCAM-1^{b} |
| Heart | CCR5 | CCL4, CCL5 |
| | CCR4 | ? |
| | CXCR3 | CXCL10 |
| | c-Met | HGF |
| Brain | VLA-4^{b} | VCAM-1^{b} |
| | LFA-1^{b} | ICAM-1^{b} |
| | CXCR3^{b} | CXCL9/CXCL 10 ^{b} |
| Peripheral LN^{c} | Selectin ligands^{b} | E/P-selectin^{b} |
| | LFA-1^{b} | ICAM-1^{b} |
| | CXCR3^{b} | CXCL9/CXCL 10 ^{b} |

^{a}Involved in T_{eff} cell homing to the intestine but not colon.
^{b}Inflammatory reactions, tissue injury.
^{c}Under non-inflamed, steady-state conditions, T_{eff} cells typically lose L-selectin and CCR7 expression and are largely restricted from LN access though may enter during inflammatory reactions (b) as shown. In contrast, both naive T cells and T_{cm} cells express L-selectin, CCR7, and CXCR4 and engage PNAd, CCL19/CCL21, and CXCL12, respectively, to undergo T-cell rolling and LFA-1/ICAM-1/2- mediated adhesion and transmigration into LNs.

Exemplary homing tissue (target tissue) type and ligand or chemokine that enables tissue-restricted B cell homing in accordance with the invention are set forth in Table 3.

TABLE 3

| | |
|---|---|
| Homing Tissue Type | Ligand/Chemokines |
| CNS | VCAM-1, CD62P, ligands for CCR1,2, 5, CXCR3 |
| Liver | CD62P, VAP-1, CXCL16 |
| Small Intestine | MAdCAM, CD62P, CCL25 |
| Colon | MAdCAM, CD62P, CCL20, GPR15L |
| Skin | CD62E, CD62P, CCL17(22), ICAM-1 |
| Thymus | VCAM, CD62P, CCL25 |
| Peripheral Lymph Node | PNAd, CCL21, ICAM-1 |
| Peyer’s Patch | MAdCAM, CCL21, CXCL13 |
| Bone Marrow | VCAM, CD62P, CXCL12, ICAM-1 |

In various embodiments of the present invention, a modified B cell is provided that expresses one or more of an antibody, a protein, or a receptor that facilitate homing of the modified B cell to the exemplary target/homing tissues using the specific homing receptor/ligand pairs as set forth in Table 2. In various embodiments of the present invention, a modified B cell is provided that expresses one or more of a homing receptor that facilitate homing of the modified B cell to the exemplary target/homing tissue using the ligand or chemokines are set forth in Tables 2 and/or 3. As used herein, the term “B cell homing” refers to localizing, targeting, trafficking, directing, or redirecting of the B cell of the present application to a site/target of interest, for example, a homing or target tissue, an inflammatory site in a specific location or tissue, or a tumor or tumor microenvironment, where delivery of therapeutic payloads is desirable. As used in the context of B cell homing, the term “antibody”, “protein” or a “receptor” refers to an amino acid sequence, a nucleic acid sequence encoding a peptide or protein, or an RNA molecule, for use as a therapeutic agent, which when expressed in a modified B cell of the present invention will direct the B cell to a site/target of interest.

In certain embodiments, the homing antibody, protein, or receptor molecule is for homing/targeting the modified B cell expressing such a molecule to a site/target of interest. In certain embodiments, the homing antibody, protein, or receptor molecule is for homing/targeting the modified B cell expressing such a molecule to inflammatory sites in specific locations or tissues. In certain embodiments, the homing antibody, protein or receptor is for targeting the B cell to a tumor or tumor microenvironment and to the tumor draining lymph node In certain embodiments, targeting B cells to particular locations is desirable so that the engineered or modified B cells of the present invention can deliver therapeutic payloads to desired locations of interest, for example, a homing or target tissue, an inflammatory site in a specific location or tissue, or a tumor or tumor microenvironment. Accordingly, in certain embodiments, it is desirable that the B cells home to a site/target of interest, for example, a tumor or tumor microenvironment and tumor-draining lymph node and deliver to the site/target of interest a payload capable of, for example, increasing the number of cross-presenting dendritic cells (DCs) at the site/target of interest (e.g., in tumors).

In various embodiments, the homing antibody, protein, or receptor is expressed in the modified or engineered B cell as a DNA construct. In various embodiments, the homing antibody, protein, or receptor is expressed in the modified B cell as a DNA construct under the control of a constitutively activated transcriptional pathway. In various embodiments, the homing antibody, protein, or receptor involved in the B cell homing/targeting is either not naturally expressed in a B cell or is expressed at higher levels than is naturally expressed in a B cell. Exemplary homing of the modified B cells to specific homing/target tissues using specific homing receptor/ligand pairs in accordance with the present invention is set forth in Table 4. It should be understood that, notwithstanding the exemplary homing tissues, homing receptor, and ligand pairs set forth in Table 4, a modified B cell of the present invention may be engineered to express any homing antibody, protein, or a receptor (e.g., any homing receptor set for in Table 2), such that the modified B cell can be directed to a specific site/target of interest.

TABLE 4

| | | |
|---|---|---|
| Homing Tissue Type | Homing Receptor | Ligand/Chemokine |
| Liver | CXCR6 | CXCL16 |
| Small Intestine | CCR9 | CCL25 |
| Large Intestine (Colon) | CCR6 | CCL20 |
| Lymph Node | CCR7 | CCL21 |
| Bone Marrow | CXCR4 | CXCL12 |
| Peyer’s Patch | CCR7 and CXCR5 | CCL21 and CXCL13, respectively |
| Skin | CCR4 | CCL17(22) |

Nonexclusive examples of homing (target) tissue types for the specific homing receptor/ligand pairs of the present invention include: skin, gut (intestine, colon, mesenteric lymph nodes (mLN), Peyer’s Patch (PP), small intestine), liver, lung, bone marrow, heart, peripheral lymph node (LN), CNS, thymus, and bone marrow.

Nonexclusive examples of homing receptors that can be paired with specific or corresponding attractants/ligands/chemokines of the present invention include: CLA (PSGL-1 glycoform), CLA (PSGL-1 glycoform), CCR10, CCR3, CCR4, CCR5, CCR6, CCR9, CD43E, CD44, c-Met, CXCR3, CXCR4, LFA-1, LFA-1 (αLβ2), Selectin ligands, VLA-4, VLA-4 (α4β1), and α4β7.

Nonexclusive examples of ligands/chemokines that can be paired with specific or corresponding homing receptors of the present invention include: CXCL16, CCL17, CCL17(22), CCL20 (MIP-3α), CCL21, CCL25, CCL27, CCL28, CCL4, CCL5, CD62E, CD62P, CXCL10, CXCL12, CXCL13, CXCL16, CXCL9/CXCL10, CXCR3, E/P-selectin, E-selectin, GPR15L, HGF, Hyaluronate, ICAM-1, ligands for CCR1,2, 5, MAdCAM, MAdCAM-1, PNAd, VAP-1, VCAM, and VCAM-1.

In certain embodiments of the present invention, a modified B cell is provided that express or have increased expression of the exemplary B cell homing receptors (e.g., as set forth in Table 2), such that the modified B cell is targeted to the corresponding homing tissue of interest that expresses the corresponding ligand/chemokines (e.g., as set forth in Tables 2 and/or 3). In certain embodiments of the present invention, a modified B cell is provided that co-expresses an integrin with a specific α and β chain pairing and a specific B cell homing receptor (e.g., as set forth in Tables 2 and/or 3), expression of which integrin and/or homing receptor promote or facilitate homing/targeting of the modified B cell to a site/target of interest. In some embodiments, a modified B cell is provided that co-expresses an α4β7 integrin and CCR9. It is desirable that co-expression of α4β7 and CCR9 will promote small intestine homing of the modified B cells of the present invention. In some embodiments, a modified B cell is provided that co-expresses an α4β1 integrin and CCR4. It is desirable that co-expression of α4β1 and CCR4 will promote small intestine homing of the modified B cells of the present invention.

Modified B cells that Express Immune Inhibitory Molecules. B cells are key contributors to many autoimmune diseases. However, B cells can be used therapeutically to antagonize autoimmunity. Specifically, B cells can be engineered to express at least one or more immune inhibitory molecules, which may decrease the autoimmune activity of the B cells, leading to decrease in an autoimmune disease. Immune inhibitory molecules are well known in the art. Such inhibitory molecules may include, but are not limited to, IL-10, TGF-β, PD-L1, PD-L2, LAG-3, and TIM-3. In certain embodiments of the present invention, a modified B cell is provided that is engineered to express at least one or more of an inhibitory molecule selected from IL-10, TGF-β, PD-L1, PD-L2, LAG-3, and TIM-3, or any combinations thereof, such that the inflammation at the site and autoimmune activity of the B cells localized to the site are decreased, thereby leading to a positive therapeutic response.

Compounds that alter B cell Trafficking. In certain embodiments of the present invention, a modified B cell is provided that is treated with at least one or more compound or derivatives thereof that alter the trafficking of B cells by inducing expression of a specific B cell integrin and/or a homing receptor. Compounds or derivatives thereof that alter the trafficking of B cells are well known in the art. In certain embodiments, a modified B cell is provided that is treated with all-trans-retinoic acid (ATRA) or derivatives thereof that promote homing of the B cells to gut (small intestine) due to the increased expression of α4β7 integrin and CCR9 homing receptor. As used herein, the term “compound” refers to a chemical, drug, a therapeutic agent, or derivatives thereof, that alter the trafficking of B cells in a desired manner.

In various embodiments of the present invention, a modified B cell engineered to co-express a specific integrin (e.g., with a specific α and β chain pairing) and a specific B cell homing receptor of interest is treated with at least one or more compounds or derivatives thereof that alter the trafficking of the modified B cells and promote homing of the cells to a specific site/target of interest due to the increased expression of the specific integrin and/or the homing receptor. In various embodiments, a B cell modified to co-express an integrin with a specific α and β chain pairings and a specific B cell homing receptor further expresses at least one or more immune inhibitory molecules, such that the autoimmune activity of the modified B cells targeted to a specific site of inflammation is decreased, leading to a decrease in the autoimmune disease. In some embodiments, a modified B cell engineered to express one or more immune inhibitory molecules, for example IL-10, TGF-β, PD-L1, PD-L2, LAG-3, and TIM-3, or combinations thereof, is treated with ATRA or derivatives thereof for a specified period of time, such that expression of the α4β7 integrin and CCR9 homing receptor is induced to promote B cell homing to a specific site/target of interest (e.g., the gut), but the inflammation at the site and autoimmune activity of B cells localized to the site are decreased, leading to a positive therapeutic response. In one embodiment, a modified B cell engineered to express one or more immune inhibitory molecules, for example IL-10, TGF-β, or combinations thereof, is treated with ATRA or derivatives thereof for a specified period of time, such that expression of the α4β7 integrin and CCR9 homing receptor is induced to promote B cell homing to a specific site/target of interest (e.g., the gut), but the inflammation at the site and autoimmune activity of B cells localized to the site are decreased, leading to a positive therapeutic response.

It is understood that, any B cell of the present invention modified to co-express a specific B cell integrin and homing receptor that targets the B cell to a particular homing/target tissue of interest, may be further engineered to express one or more immune inhibitory molecules for reducing inflammation and autoimmune activity of the B cells localized to the site, and/or treated with a compound that alter the homing/targeting of the modified B cells by inducing expression of the specific B cell integrin and/or the homing receptor.

Activation of B cells with TLR agonists and TLRs. B cells have a natural ability to uptake and present antigens recognized by their specific B cell receptors (BCRs). B cells activated by Toll-like receptors (TLRs) result in potent effector B cells in defending the body in an immune response. Expression of or increasing the expression of TLRs in B cells can provide a mechanism for potentiating B cells for innate signals regulating adaptive immune responses.

Activation of B cells with TLR agonists. In various embodiments of the present invention, a B cell is provided, where the B cell is treated in vitro with at least one TLR agonist. In various embodiments, the TLR can be a TLR1, TLR2, TLR3, TLR4, TLR5, TLR6, TLR7, TLR8, TLR9, TLR10, TLR11, TLR12, and/or a TLR13. In various embodiments, the TLR agonist preferentially binds to one or more TLR selected from the group consisting of TLR1, TLR2, TLR3, TLR4, TLR5, TLR6, TLR7, TLR8, TLR9, TLR10, TLR11, TLR12, and TLR13. TLR agonists are well known in the art and may include, but are not limited to, CpG-rich oligonucleotides and the double-stranded RNA mimic, polyinosinic acid:polycytidylic acid (poly-I:C). In various embodiments, the TLR agonist can be CpG oligonucleotides.

In various embodiments, each B cell may be treated with one TLR agonist. In various embodiments, each B cell may be treated with more than one TLR agonist. For example, each B cell may be treated 1, 2, 3, 4, 5, 6, 7, 8, 9, 10, 11, or 12 different TLR agonists. Alternatively, the patient may be administered a heterogeneous population of B cells, each B cell treated with a unique TLR agonist or a combination of TLR agonists. In some embodiments, the B cells for use a therapeutic agent is treated with one or more TLR agonists at the same time or in advance of the administration of the B cells to a subject or patient in need thereof. In certain embodiments, treatment with one or more TLR agonist is capable of producing more potent effector B cells for defending the body in an immune response. In certain embodiments, treatment with one or more TLR agonist is capable of potentiating B cells for immune responses. In some embodiments, treating a B cell of the present invention with at least one or more TLR agonists induces expression or activation of one or more TLRs.

Activation of B cells with TLR Expression. In various embodiments of the present invention, a modified B cell is provided that is capable of expressing a constitutively active TLR. In various embodiments, the TLR is expressed in the modified or engineered B cell as a DNA construct under the control of a constitutively activated transcriptional pathway. In various embodiments, the TLR is either not naturally expressed in a B cell or is expressed at higher levels than is naturally expressed in a B cell. In various embodiments, the TLR can be a TLR1, TLR2, TLR3, TLR4, TLR5, TLR6, TLR7, TLR8, TLR9, TLR10, TLR11, TLR12, and/or a TLR13.

In various embodiments, each B cell may express more than one constitutively active TLR. For example, each B cell may express 1, 2, 3, 4, 5, 6, 7, 8, 9, 10, 11, 12 or 13 different constitutively active TLRs. Alternatively, the patient may be administered a heterogeneous population of B cells, each B cell capable of expressing and/or secreting a unique TLR or combination of TLRs, which are constitutively active. In various embodiments, 1, 2, 3, 4, 5, 6, 7, 8, 9, 10, 11, 12 or 13 different constitutively active TLRs may be administered to the subject or patient through a heterogeneous population of B cells.

In certain embodiments of the present invention, the B cell is a modified B cell that expresses at least one constitutively active TLR. In certain embodiments, the modified B cell that expresses at least one constitutively active TLR is treated with one or more TLR agonist. In certain embodiments, the expression of the constitutively active TLR is capable of producing more potent effector B cells for defending the body in an immune response. In certain embodiments, the expression of the constitutively active TLR is capable of potentiating B cells for immune responses. In certain embodiments, the modified B cell expresses both a TLR that is constitutively active and any CAR-B of the present application. In various embodiments, the modified B cell expressing a TLR that is constitutively active and/or a CAR-B is further treated with one or more TLR agonist at the same time or in advance of the administration of the modified B cells to a subject or patient in need thereof. In certain embodiments, B cells may be engineered to express payloads and modifiers, such as TLRs, in the absence of CAR-B, for intratumoral administration.

Modified B Cells that Present Antigens Simultaneously in HLA Class I and Class II Molecules. B cells, in addition to their function in antibody production, also express high level of Human Leukocyte Antigen (HLA) class II molecules and can present antigens to CD4+ T cells(Hong et al., 2018, Immunity 49, 695-708). In various embodiments of the present invention, a modified B cell is provided that is capable of presenting specific antigens and/or antigen-derived epitopes of interest, such as tumor antigens or infectious disease antigens, simultaneously in both HLA class I and class II molecules. Tumor antigens and infectious disease antigens are well known in the art and are described in the foregoing sections. In certain embodiments, a specific antigen of interest, e.g., a tumor antigen or an infectious disease antigen, is fused to a targeting signal of a lysosomal protein that targets the antigen to the lysosomes and presents the antigen simultaneously and efficiently in both HLA class I and class II molecules. In some embodiments, the targeting signal is the targeting signal of lysosome-associated membrane protein-1 (LAMP1). In some embodiments, the targeting signal is capable of entering endosomal recycling compartments. The c-terminal sequence of Clec9A is such a targeting moiety. As used herein, a specific tumor antigen or an infectious disease antigen fused to a targeting signal refers to an amino acid sequence, a nucleic acid sequence encoding a peptide or protein, or an RNA molecule (e.g., an mRNA molecule), for use as a therapeutic agent. In one embodiment, a specific tumor antigen or an infectious disease antigen fused to a targeting signal refers to an mRNA molecule for use as a therapeutic agent. In certain embodiments, it is desirable that the specific tumor antigens and/or infectious disease antigens fused to a targeting signal, such as the targeting signal of LAMP1 or Clec9A, be targeted to the lysosomes or endosomes and presented simultaneously and efficiently in both HLA class I and class II molecules. In certain embodiments, it is desirable that electroporation of B cells (e.g., human B cells), before or after maturation, with an mRNA encoding specific tumor antigens and/or infectious disease antigens of interest fused to a targeting signal, such as the targeting signal of LAMP1 or Clec9A, be capable of simultaneously and efficiently presenting the specific antigens and/or antigen-derived epitopes in both HLA class I and class II molecules. In various embodiments, the specific tumor antigens and/or infectious disease antigens of interest is either not naturally presented by a B cell, is not presented by a B cell simultaneously in both HLA class I and class II molecules naturally, or is not presented by a B cell with high efficiencies in both HLA class I and class II molecules naturally. It is contemplated that, introduction of such electroporated B cells into a subject, e.g., a human host, will promote development of or potentiate antigen-specific immune responses by presenting specific antigens and/or antigen-derived epitopes of interest simultaneously and efficiently in both HLA class I and class II molecules.

In various embodiments, the invention relates to a nucleic acid sequence, e.g., an mRNA sequence, encoding at least one specific antigen of interest, e.g., a tumor antigen or an infectious disease antigen, fused to a targeting signal, such as the targeting signal of LAMP1, for use as a therapeutic agent in electroporation of B cells for simultaneously and efficiently presenting the specific antigen and/or antigen-derived epitopes in both HLA class I and class II molecules. In various embodiments, the invention relates to nucleic acid sequence, e.g., an mRNA sequence, encoding more than one (e.g., 1, 2, 3, 4, 5, or more) specific tumor antigen and/or an infectious disease antigen of interest fused to a targeting signal. In various embodiments, the invention relates to pools of different nucleic acid sequences, e.g., pools of different mRNA sequences, for use as a therapeutic agent in electroporation of B cells as described above, where each pool encodes at least one specific antigen of interest, e.g., a tumor antigen or an infectious disease antigen, fused to a targeting signal that is different from the other pools of the mRNA sequences. Accordingly, in some embodiments, the subject may be administered a homogeneous population of B cells, where each B cell is electroporated with an mRNA encoding at least one specific antigen of interest fused to a targeting signal. In some embodiments, the subject may be administered a homogeneous a population of B cells, where each B cell is electroporated with an mRNA encoding more than one specific antigen of interest fused to targeting signal. In some embodiments, the subject may be administered a heterogeneous population of B cells, where each B cell is electroporated with a combination of mRNAs each encoding at least one specific antigen of interest fused to a different targeting signal.

In some embodiments, the B cells for use in electroporation as described above may be any of the modified B cells of the present application. In some embodiments, the modified B cell comprises a chimeric antigen receptor for B cells (CAR-B). In various embodiments, the modified B cell can express a CAR-B and simultaneously and efficiently present specific antigen and/or antigen-derived epitopes of interest in both HLA class I and class II molecules.

In various embodiments, the invention relates to a method of administering an isolated B cell to a patient in need thereof. In various embodiments, a population of B cells may be administered to the patient. In various embodiments, each B cell may express more than one payload peptide or protein. For example, each B cell may express 1, 2, 3, 4, 5, 6, 7, 8, 9, 10, 11 or 12 different payloads. Alternatively, the patient may be administered a heterogeneous population of B cells, each B cell capable of expressing and/or secreting a unique payload or combination of payloads. In various embodiments, 1, 2, 3, 4, 5, 6, 7, 8, 9, 10, 11 or 12 different payloads may be administered to the patient through a heterogeneous population of B cells.

### Methods of Treatment

In some aspects, the invention therefore comprises a method for treating or preventing a tumor or cancerous tissue, comprising administering to a patient in need thereof an effective amount of at least one CAR-B disclosed herein.

Methods are provided for treating diseases or disorders, including cancer. In some embodiments, the invention relates to creating a B cell-mediated immune response in a subject, comprising administering an effective amount of the engineered immune cells of the present application to the subject. In some embodiments, the B cell-mediated immune response is directed against a target cell or cells. In some embodiments, the engineered immune cell comprises a chimeric antigen receptor for B cells (CAR-B). In some embodiments, the target cell is a tumor cell. In some aspects, the invention comprises a method for treating or preventing a malignancy, said method comprising administering to a subject in need thereof an effective amount of at least one isolated antigen-binding molecule described herein. In some aspects, the invention comprises a method for treating or preventing a malignancy, said method comprising administering to a subject in need thereof an effective amount of at least one immune cell, wherein the immune cell comprises at least one chimeric antigen receptor.

In some aspects, the invention comprises a pharmaceutical composition comprising at least one antigen-binding molecule as described herein and a pharmaceutically acceptable excipient. In some embodiments, the pharmaceutical composition further comprises an additional active agent.

In some embodiments, the subject is diagnosed with a metastatic disease localized to the liver. In other embodiments, the metastatic disease is a cancer. In still other embodiments, the cancer metastasized from a primary tumor in the breast, colon, rectum, esophagus, lung, pancreas and/or stomach. In still other embodiments, the subject is diagnosed with unresectable metastatic liver tumors. In yet other embodiments, the subject is diagnosed with unresectable metastatic liver tumors from primary colorectal cancer. In some embodiments, the subject is diagnosed with hepatocellular carcinoma.

It will be appreciated that target doses for modified B cells can range from 1 x10⁶-2x10¹⁰ cells/kg, preferably 2x10⁶ cells/kg, more preferably. It will be appreciated that doses above and below this range may be appropriate for certain subjects, and appropriate dose levels can be determined by the healthcare provider as needed. Additionally, multiple doses of cells can be provided in accordance with the invention.

Also provided are methods for reducing the size of a tumor in a subject, comprising administering to the subject a modified B cell of the present invention, wherein the cell comprises a CAR-B receptor comprising an antigen-binding domain that binds to an antigen on a tumor, a payload or both a CAR-B and a payload. In some embodiments, the subject has a solid tumor, or a blood malignancy such as lymphoma or leukemia. Lymphomas include, but are not limited to, Hodgkin’s and Non-Hodgkin’s lymphoma. Leukemias include, but are not limited to, ALL, CLL, AML and CML. Myelomas include, but are not limited to, multiple myeloma. Additional tumor types include, but are not limited to, tumors resulting lung cancer, breast cancer, colorectal cancer, pancreatic cancer, brain cancers (such as glioma and glioblastoma), melanoma, prostate cancer, bladder cancer, kidney cancer, renal cancers, endometrial cancer, thyroid cancers, and liver cancers.

In some embodiments, the modified B cell is delivered to a tumor bed. In some embodiments, the cancer is present in the bone marrow of the subject

Also provided are methods for homing B cells to a site/target of interest in a subject, comprising administering to the subject a modified B cell of the present invention, wherein the cell comprises an integrin, a homing antibody, protein, or a receptor that is attracted to a ligand, chemokine, or an attractant at the site/target of interest. In some embodiments, the site/target of interest is, for example, a homing or target tissue, an inflammatory site in a specific location or tissue, or a tumor or tumor microenvironment, where delivery of therapeutic payloads is desirable.

Also provided are methods for decreasing inflammation and autoimmune activity of B cells at a site/target of interest in a subject, comprising administering to the subject a modified B cell of the present invention, wherein the cell comprises an immune inhibitory molecule. In some embodiments, the site/target of interest is, for example, a homing or target tissue, an inflammatory site in a specific location or tissue, or a tumor or tumor microenvironment, where delivery of therapeutic payloads is desirable.

Also provided are methods for altering trafficking of B cells to a site/target of interest in a subject, comprising treating a B cell of the present invention with a compound or derivatives thereof suitable for altering B cell trafficking, and administering the treated B cell to the subject in need thereof. In some instances, the compound or derivatives thereof alters B cell trafficking by increasing the expression of an integrin, homing antibody, protein, receptor, or combinations thereof, expressed by the B cells.

Also provided are methods for potentiating B cells and/or producing potent effector B cells for increasing immune responses in a subject, comprising treating a B cell of the present invention with at least one or more TLR agonists, and administering the treated B cell to the subject in need thereof. In some embodiments, treating a B cell of the present invention with at least one or more TLR agonists induces expression or activation of one or more TLRs. In some embodiments, the method for potentiating B cells and/or producing potent effector B cells for increasing immune responses in a subject, further comprises administering to the subject a modified B cell of the present invention that expresses at least one or more constitutively active TLRs. Also provided are methods for potentiating B cells and/or producing potent effector B cells for increasing immune responses in a subject, comprising administering to the subject a modified B cell of the present invention, wherein the cell expresses a CAR-B receptor comprising an antigen-binding domain that binds to an antigen on a tumor, a constitutively active TLR or both a CAR-B and a constitutively active TLR, where the cell is treated with at least one or more TLR agonists at the same time or in advance of the administration of the cells to the subject.

Also provided are methods for increasing antigen-specific immune responses in a subject, comprising administering to the subject a modified B cell of the present invention, wherein the cell is electroporated with a nucleic acid sequence, e.g., an mRNA, encoding specific tumor antigens and/or infectious disease antigens fused to a targeting signal, such as the targeting signal of LAMP1 or Clec9A, for simultaneously and efficiently presenting the specific antigens and/or antigen-derived epitopes in both HLA class I and class II molecules. In some embodiments, the subject has a solid tumor, or a blood malignancy such as lymphoma or leukemia.

It is understood that the various embodiments of the methods of treatment using the engineered or modified B cells of the present application are not mutually exclusive and can be combined with each other in any way and without any restriction unless explicitly indicated, for achieving of facilitating any of the results and/or therapeutic responses contemplated herein.

In some embodiments, the modified B cells are autologous B cells. In some embodiments, the modified B cells are allogeneic B cells. In some embodiments, the modified B cells are heterologous B cells. In some embodiments, the modified B cells of the present application are transfected or transduced in vivo. In other embodiments, the engineered cells are transfected or transduced ex vivo.

As used herein, the term “subject” or “patient” means an individual. In some aspect, a subject is a mammal such as a human. In some aspect, a subject can be a non-human primate. Non-human primates include marmosets, monkeys, chimpanzees, gorillas, orangutans, and gibbons, to name a few. The term “subject” also includes domesticated animals, such as cats, dogs, etc., livestock (e.g., llama, horses, cows), wild animals (e.g., deer, elk, moose, etc.,), laboratory animals (e.g., mouse, rabbit, rat, gerbil, guinea pig, etc.) and avian species (e.g., chickens, turkeys, ducks, etc.). Preferably, the subject is a human subject. More preferably, the subject is a human patient.

The methods can further comprise administering one or more chemotherapeutic agents. In certain embodiments, the chemotherapeutic agent is a lymphodepleting (preconditioning) chemotherapeutic. Beneficial preconditioning treatment regimens, along with correlative beneficial biomarkers are described in e.g., U.S. Provisional Pat. Applications 62/262,143 and 62/167,750, as well as U.S. Pat. No. 9,855,298, which are hereby incorporated by reference in their entirety herein. These describe, e.g., methods of conditioning a patient in need of a T cell therapy comprising administering to the patient specified beneficial doses of cyclophosphamide (between 200 mg/m²/ day and 2000 mg/m²/day) and specified doses of fludarabine (between 20 mg/m²/day and 900 mg/m²/day). A preferred dose regimen involves treating a patient comprising administering daily to the patient about 500 mg/m²/day of cyclophosphamide and about 60 mg/m²/day of fludarabine for three days prior to administration of a therapeutically effective amount of engineered B cells to the patient.

In other embodiments, the antigen-binding molecule, transduced (or otherwise engineered) cells (such as CARs), and the chemotherapeutic agent are administered each in an amount effective to treat the disease or condition in the subject.

In certain embodiments, compositions comprising CAR-expressing immune effector cells disclosed herein may be administered in conjunction with any number of chemotherapeutic agents. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine resume; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, que-lamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2',2”-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (Taxol®, Bristol-Myers Squibb) and doxetaxel (Taxotere®, Rhone-Poulenc Rorer); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS2000; difluoromethylomithine (DMFO); retinoic acid derivatives such as Targretin™ (bexarotene), Panretin™, (alitretinoin); Ontak™ (denileukin diftitox); esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. Combinations of chemotherapeutic agents are also administered where appropriate, including, but not limited to CHOP, i.e., Cyclophosphamide (Cytoxan®) Doxorubicin (hydroxydoxorubicin), Fludarabine, Vincristine (Oncovin®), and Prednisone.

In some embodiments, the chemotherapeutic agent is administered at the same time or within one week after the administration of the engineered cell or nucleic acid. In other embodiments, the chemotherapeutic agent is administered from 1 to 4 weeks or from 1 week to 1 month, 1 week to 2 months, 1 week to 3 months, 1 week to 6 months, 1 week to 9 months, or 1 week to 12 months after the administration of the engineered cell or nucleic acid. In other embodiments, the chemotherapeutic agent is administered at least 1 month before administering the cell or nucleic acid. In some embodiments, the methods further comprise administering two or more chemotherapeutic agents.

A variety of additional therapeutic agents may be used in conjunction with the compositions described herein. For example, potentially useful additional therapeutic agents include PD-1 (or PD-L1) inhibitors such as nivolumab (Opdivo®), pembrolizumab (Keytruda®), pembrolizumab, pidilizumab, and atezolizumab (Tecentriq^{®}). Other additional therapeutics include anti-CTLA-4 antibodies (e.g., Ipilimumab®), anti-LAG-3 antibodies (e.g., Relatlimab, BMS), alone or in combination with PD-1 and/or PD-L1 inhibitors.

Additional therapeutic agents suitable for use in combination with the invention include, but are not limited to, ibrutinib (Imbruvica®), ofatumumab (Arzerra®), rituximab (Rituxan®), bevacizumab (Avastin®), trastuzumab (Herceptin®), trastuzumab emtansine (KADCYLA®), imatinib (Gleevec®), cetuximab (Erbitux®), panitumumab (Vectibix®), catumaxomab, ibritumomab, ofatumumab, tositumomab, brentuximab, alemtuzumab, gemtuzumab, erlotinib, gefitinib, vandetanib, afatinib, lapatinib, neratinib, axitinib, masitinib, pazopanib, sunitinib, sorafenib, toceranib, lestaurtinib, axitinib, cediranib, lenvatinib, nintedanib, pazopanib, regorafenib, semaxanib, sorafenib, sunitinib, tivozanib, toceranib, vandetanib, entrectinib, cabozantinib, imatinib, dasatinib, nilotinib, ponatinib, radotinib, bosutinib, lestaurtinib, ruxolitinib, pacritinib, cobimetinib, selumetinib, trametinib, binimetinib, alectinib, ceritinib, crizotinib, aflibercept, adipotide, denileukin diftitox, mTOR inhibitors such as Everolimus and Temsirolimus, hedgehog inhibitors such as sonidegib and vismodegib, CDK inhibitors such as CDK inhibitor (palbociclib).

In additional embodiments, the composition comprising CAR-containing B cells can be administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate. Exemplary NSAIDs include ibuprofen, naproxen, naproxen sodium, Cox-2 inhibitors, and sialylates. Exemplary analgesics include acetaminophen, oxycodone, tramadol of proporxyphene hydrochloride. Exemplary glucocorticoids include cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists, (e.g., etanercept (ENBREL®), adalimumab (HUMIRA®) and infliximab (REMICADE®)), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular) and minocycline.

In certain embodiments, the compositions described herein are administered in conjunction with a cytokine. “Cytokine” as used herein is meant to refer to proteins released by one cell population that act on another cell as intercellular mediators. Examples of cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor (HGF); fibroblast growth factor (FGF); prolactin; placental lactogen; mullerian-inhibiting substance; mouse gonadotropin-associated peptide; inhibin; activin; vascular endothelial growth factor; integrin; thrombopoietin (TPO); nerve growth factors (NGFs) such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1 alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

### Methods of Making

A variety of known techniques can be utilized in making the polynucleotides, polypeptides, vectors, antigen-binding molecules, immune cells, compositions, and the like according to the invention.

Prior to the in vitro manipulation or genetic modification of the immune cells described herein, the cells may be obtained from a subject. In some embodiments, the immune cells comprise B cells. B cells can be obtained from a number of sources, including peripheral blood mononuclear cells (PBMCs), bone marrow, lymph nodes tissue, cord blood, thymus tissue, tissue from a site of infection, ascites, pleural effusion, spleen tissue, and tumors. In certain embodiments, B cells can be obtained from a unit of blood collected from the subject using any number of techniques known to the skilled person, such as FICOLL™ separation. Cells may preferably be obtained from the circulating blood of an individual by apheresis. The apheresis product typically contains lymphocytes, including T cells, monocytes, granulocytes, B cells, other nucleated white blood cells, red blood cells, and platelets. In certain embodiments, the cells collected by apheresis may be washed to remove the plasma fraction, and placed in an appropriate buffer or media for subsequent processing. The cells may be washed with PBS. As will be appreciated, a washing step may be used, such as by using a semiautomated flowthrough centrifuge for example, the Cobe™ 2991 cell processor, the Baxter Cyto-Mate™, or the like. After washing, the cells may be resuspended in a variety of biocompatible buffers, or other saline solution with or without buffer. In certain embodiments, the undesired components of the apheresis sample may be removed.

The immune cells, such as B cells, can be genetically modified following isolation using known methods, or the immune cells can be activated and expanded (or differentiated in the case of progenitors) in vitro prior to being genetically modified. In another embodiment, the immune cells, such as B cells, are genetically modified with the chimeric B cell receptors described herein (e.g., transduced with a viral vector comprising one or more nucleotide sequences encoding a CAR-B) and then are activated and/or expanded in vitro. Methods for activating and expanding B cells are known in the art and are described, for example, in U.S. Pat. Nos. 6,905,874; 6,867,041; 6,797,514; and PCT WO 2012/079000, the contents of which are hereby incorporated by reference in their entirety. Generally, such methods include contacting PBMC or isolated B cells with a stimulatory agent and costimulatory agent generally attached to a bead or other surface, in a culture medium with appropriate cytokines, such as IL-2.

In other embodiments, the B cells may be activated and stimulated to proliferate with feeder cells and appropriate antibodies and cytokines using methods such as those described in U.S. Pat. Nos. 6,040,177; 5,827,642; and WO/2012129514, the contents of which are hereby incorporated by reference in their entirety.

Certain methods for making the constructs and engineered immune cells of the invention are described in PCT application PCT/US2015/14520, the contents of which are hereby incorporated by reference in their entirety. Additional methods of making the constructs and cells can be found in U.S. provisional pat. application No. 62/244,036 the contents of which are hereby incorporated by reference in their entirety.

For cloning of polynucleotides, the vector may be introduced into a host cell (an isolated host cell) to allow replication of the vector itself and thereby amplify the copies of the polynucleotide contained therein. The cloning vectors may contain sequence components generally include, without limitation, an origin of replication, promoter sequences, transcription initiation sequences, enhancer sequences, and selectable markers. These elements may be selected as appropriate by a person of ordinary skill in the art. For example, the origin of replication may be selected to promote autonomous replication of the vector in the host cell.

In certain embodiments, the present disclosure provides isolated host cells containing the vector provided herein. The host cells containing the vector may be useful in expression or cloning of the polynucleotide contained in the vector. Suitable host cells can include, without limitation, prokaryotic cells, fungal cells, yeast cells, or higher eukaryotic cells such as mammalian cells. Suitable prokaryotic cells for this purpose include, without limitation, eubacteria, such as Gram-negative or Gram-positive organisms, for example, Enterobactehaceae such as Escherichia, e.g., E. coli, Enterobacter, Erwinia, Klebsiella, Proteus, Salmonella, e.g., Salmonella typhimurium, Serratia, e.g., Serratia marc-escans, and Shigella, as well as Bacilli such as B. subtilis and B. licheniformis, Pseudomonas such as P. aeruginosa, and Streptomyces.

The vector can be introduced to the host cell using any suitable methods known in the art, including, without limitation, DEAE-dextran mediated delivery, calcium phosphate precipitate method, cationic lipids mediated delivery, liposome mediated transfection, electroporation, microprojectile bombardment, receptor-mediated gene delivery, delivery mediated by polylysine, histone, chitosan, and peptides. Standard methods for transfection and transformation of cells for expression of a vector of interest are well known in the art. In a further embodiment, a mixture of different expression vectors can be used in genetically modifying a donor population of immune effector cells wherein each vector encodes a different CAR-Bs as disclosed herein. The resulting transduced immune effector cells form a mixed population of engineered cells, with a proportion of the engineered cells expressing more than one different CAR-Bs.

In one embodiment, the invention provides a method of storing genetically engineered cells expressing CAR-Bs that target a protein. This involves cryopreserving the immune cells such that the cells remain viable upon thawing. A fraction of the immune cells expressing the CAR-B s can be cryopreserved by methods known in the art to provide a permanent source of such cells for the future treatment of patients afflicted with a malignancy. When needed, the cryopreserved transformed immune cells can be thawed, grown and expanded for more such cells.

As used herein, “cryopreserve” refers to the preservation of cells by cooling to sub-zero temperatures, such as (typically) 77 Kelvin or 196° C. (the boiling point of liquid nitrogen). Cryoprotective agents are often used at sub-zero temperatures to prevent the cells being preserved from damage due to freezing at low temperatures or warming to room temperature. Cryopreservative agents and optimal cooling rates can protect against cell injury. Cryoprotective agents which can be used in accordance with the invention include but are not limited to: dimethyl sulfoxide (DMSO) (Lovelock & Bishop, Nature, 1959, 183, 1394-1395; Ashwood-Smith, Nature, 1961, 190, 1204-1205), glycerol, polyvinylpyrrolidine (Rinfret, Ann. N.Y. Acad. Sci., 1960, 85, 576), and polyethylene glycol (Sloviter & Ravdin, Nature, 1962, 196, 48). The preferred cooling rate is 1 °-3° C./minute.

The term, “substantially pure,” is used to indicate that a given component is present at a high level. The component is desirably the predominant component present in a composition. Preferably it is present at a level of more than 30%, of more than 50%, of more than 75%, of more than 90%, or even of more than 95%, said level being determined on a dry weight/dry weight basis with respect to the total composition under consideration. At very high levels (e.g. at levels of more than 90%, of more than 95% or of more than 99%) the component can be regarded as being in “pure form.” Biologically active substances of the present invention (including polypeptides, nucleic acid molecules, antigen-binding molecules, moieties) can be provided in a form that is substantially free of one or more contaminants with which the substance might otherwise be associated. When a composition is substantially free of a given contaminant, the contaminant will be at a low level (e.g., at a level of less than 10%, less than 5%, or less than 1% on the dry weight/dry weight basis set out above).

In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion media can be any isotonic medium formulation, typically normal saline, Normosol™ R (Abbott) or Plasma-Lyte™ A (Baxter), but also 5% dextrose in water or Ringer’s lactate can be utilized. The infusion medium can be supplemented with human serum albumin.

Desired treatment amounts of cells in the composition is generally at least 2 cells or is more typically greater than 10² cells, and up to 10⁶, up to and including 10⁸ or 10⁹ cells and can be more than 10¹⁰ cells. The number of cells will depend upon the desired use for which the composition is intended, and the type of cells included therein. The density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells. In some aspects of the present invention, particularly since all the infused cells will be redirected to a particular target antigen, lower numbers of cells, in the range of 10⁶/kilogram (10⁶-10¹¹ per patient) may be administered. CAR-B treatments may be administered multiple times at dosages within these ranges. The cells may be autologous, allogeneic, or heterologous to the patient undergoing therapy. In some aspects, different CAR-B cells are found in a single product. The composition can be as few as 2, 3, 4, 5, 6, 7, 8, 9 or up to 10 different CAR-B cells. These can consist of cells expressing a chimeric CAR protein and B cells expressing other CARs and/or payloads.

The B cells of the present invention may be administered either alone, or as a pharmaceutical composition in combination with diluents and/or with other components such as IL-2 or other cytokines or cell populations. Pharmaceutical compositions of the present invention may comprise a CAR-B expressing cell population, such as B cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration. Treatment may also include one or more corticosteroid treatment, such as dexamethasone and/or methylprednisolone.

The compositions of the present application can comprise, consist essentially of, or consist of, the components disclosed.

The pharmaceutical compositions of the invention (solutions, suspensions or the like), may include one or more of the following: sterile diluents such as water for injection, saline solution, preferably physiological saline, Ringer’s solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfate; chelating agents such as ethylene-diaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

It will be appreciated that adverse events may be minimized by transducing the immune cells (containing one or more CAR-B) with a suicide gene. It may also be desired to incorporate an inducible “on” or “accelerator” switch into the immune cells. These techniques may employ the use of dimerization domains and optional activators of such domain dimerization. These techniques include, e.g., those described by Wu et al., Science 2014, 350(6258) utilizing FKBP/Rapalog dimerization systems in certain cells, the contents of which are incorporated by reference herein in their entirety. Additional dimerization technology is described in, e.g., Fegan et al. Chem. Rev. 2010, 110, 3315-3336 as well as U.S. Pat. Nos. 5,830,462; 5,834,266; 5,869,337; and 6,165,787, the contents of which are also incorporated by reference herein in their entirety. Additional dimerization pairs may include cyclosporine-A/cyclophilin, receptor, estrogen/estrogen receptor (optionally using tamoxifen), glucocorticoids/glucocorticoid receptor, tetracycline/tetracycline receptor, vitamin D/vitamin D receptor. Further examples of dimerization technology can be found in e.g., WO 2014/127261, WO 2015/090229, US 2014/0286987, US 2015/0266973, US 2016/0046700, U.S. Pat. No. 8,486,693, US 2014/0171649, and US 2012/0130076, the contents of which are further incorporated by reference herein in their entirety.

Suitable techniques include use of inducible caspase-9 (U.S. Appl. Pub. No. 2011/0286980) or a thymidine kinase, before, after or at the same time, as the cells are transduced with the CAR-B construct of the present invention. Additional methods for introducing suicide genes and/or “on” switches include CRISPR, TALENS, MEGATALEN, zinc fingers, RNAi, siRNA, shRNA, antisense technology, and other techniques known in the art.

Anti-CD20 or anti-CD19 represent additional means to reduce or eliminate engineered B cells if such cells are responsible for adverse events or pathologies.

It will be understood that descriptions herein are exemplary and explanatory only and are not restrictive of the invention as claimed. In this application, the use of the singular includes the plural unless specifically stated otherwise.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. As utilized in accordance with the present disclosure, the following terms, unless otherwise indicated, shall be understood to have the following meanings:

In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

The term “polynucleotide”, “nucleotide”, or “nucleic acid” includes both single-stranded and double-stranded nucleotide polymers. The nucleotides comprising the polynucleotide can be ribonucleotides or deoxyribonucleotides or a modified form of either type of nucleotide. Said modifications include base modifications such as bromouridine and inosine derivatives, ribose modifications such as 2', 3'-dideoxyribose, and internucleotide linkage modifications such as phosphorothioate, phosphorodithioate, phosphoroselenoate, phosphoro-diselenoate, phosphoro-anilothioate, phoshoraniladate and phosphoroamidate.

The term “oligonucleotide” refers to a polynucleotide comprising 200 or fewer nucleotides. Oligonucleotides can be single stranded or double stranded, e.g., for use in the construction of a mutant gene. Oligonucleotides can be sense or antisense oligonucleotides. An oligonucleotide can include a label, including a radiolabel, a fluorescent label, a hapten or an antigenic label, for detection assays. Oligonucleotides can be used, for example, as PCR primers, cloning primers or hybridization probes.

The term “control sequence” refers to a polynucleotide sequence that can affect the expression and processing of coding sequences to which it is ligated. The nature of such control sequences can depend upon the host organism. In particular embodiments, control sequences for prokaryotes can include a promoter, a ribosomal binding site, and a transcription termination sequence. For example, control sequences for eukaryotes can include promoters comprising one or a plurality of recognition sites for transcription factors, transcription enhancer sequences, and transcription termination sequence. “Control sequences” can include leader sequences (signal peptides) and/or fusion partner sequences.

As used herein, “operably linked” means that the components to which the term is applied are in a relationship that allows them to carry out their inherent functions under suitable conditions.

The term “vector” means any molecule or entity (e.g., nucleic acid, plasmid, bacteriophage or virus) used to transfer protein coding information into a host cell. The term “expression vector” or “expression construct” refers to a vector that is suitable for transformation of a host cell and contains nucleic acid sequences that direct and/or control (in conjunction with the host cell) expression of one or more heterologous coding regions operatively linked thereto. An expression construct can include, but is not limited to, sequences that affect or control transcription, translation, and, if introns are present, affect RNA splicing of a coding region operably linked thereto.

The term “host cell” refers to a cell that has been transformed, or is capable of being transformed, with a nucleic acid sequence and thereby expresses a gene of interest. The term includes the progeny of the parent cell, whether or not the progeny is identical in morphology or in genetic make-up to the original parent cell, so long as the gene of interest is present.

The term “transformation” refers to a change in a cell’s genetic characteristics, and a cell has been transformed when it has been modified to contain new DNA or RNA. For example, a cell is transformed where it is genetically modified from its native state by introducing new genetic material via transfection, transduction, or other techniques. Following transfection or transduction, the transforming DNA can recombine with that of the cell by physically integrating into a chromosome of the cell, or can be maintained transiently as an episomal element without being replicated, or can replicate independently as a plasmid. A cell is considered to have been “stably transformed” when the transforming DNA is replicated with the division of the cell.

The term “transfection” refers to the uptake of foreign or exogenous DNA by a cell. A number of transfection techniques are well known in the art and are disclosed herein. See, e.g., Graham et al., VIROLOGY, 1973, 52:456; Sambrook et al., Molecular Cloning: A Laboratory Manual, 2001, supra; Davis et al., Basic Methods in Molecular Biology, 1986, Elsevier; Chu et al., Gene, 1981, 13:197.

The term “transduction” refers to the process whereby foreign DNA is introduced into a cell via viral vector. See, e.g., Jones et al., Genetics: principles and analysis, 1998, Boston: Jones & Bartlett Publ.

The terms “polypeptide” or “protein” refer to a macromolecule having the amino acid sequence of a protein, including deletions from, additions to, and/or substitutions of one or more amino acids of the native sequence. The terms “polypeptide” and “protein” specifically encompass antigen-binding molecules, antibodies, or sequences that have deletions from, additions to, and/or substitutions of one or more amino acid of antigen-binding protein. The term “polypeptide fragment” refers to a polypeptide that has an amino-terminal deletion, a carboxyl-terminal deletion, and/or an internal deletion as compared with the full-length native protein. Such fragments can also contain modified amino acids as compared with the native protein. Useful polypeptide fragments include immunologically functional fragments of antigen-binding molecules.

The term “isolated” means (i) free of at least some other proteins with which it would normally be found, (ii) is essentially free of other proteins from the same source, e.g., from the same species, (iii) separated from at least about 50 percent of polynucleotides, lipids, carbohydrates, or other materials with which it is associated in nature, (iv) operably associated (by covalent or noncovalent interaction) with a polypeptide with which it is not associated in nature, or (v) does not occur in nature.

A “variant” of a polypeptide (e.g., an antigen-binding molecule) comprises an amino acid sequence wherein one or more amino acid residues are inserted into, deleted from and/or substituted into the amino acid sequence relative to another polypeptide sequence. Variants include fusion proteins.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”).

To calculate percent identity, the sequences being compared are typically aligned in a way that gives the largest match between the sequences. One example of a computer program that can be used to determine percent identity is the GCG program package, which includes GAP (Devereux et al., Nucl. Acid Res., 1984, 12, 387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). In certain embodiments, a standard comparison matrix (see, e.g., Dayhoff et al., 1978, Atlas of Protein Sequence and Structure, 1978, 5:345-352 for the PAM 250 comparison matrix; Henikoff et al., Proc. Natl. Acad. Sci. U.S.A. 1992, 89, 10915-10919 for the BLO-SUM 62 comparison matrix) is also used by the algorithm.

As used herein, the twenty conventional (e.g., naturally occurring) amino acids and their abbreviations follow conventional usage. See, e.g., Immunology A Synthesis (2nd Edition, Golub and Green, Eds., Sinauer Assoc., Sunderland, Mass. (1991)), which is incorporated herein by reference for any purpose. Stereoisomers (e.g., D-amino acids) of the twenty conventional amino acids, unnatural amino acids such as alpha-, alpha-disubstituted amino acids, N-alkyl amino acids, lactic acid, and other unconventional amino acids can also be suitable components for polypeptides of the present invention. Examples of unconventional amino acids include: 4-hydroxyproline, .gamma.-carboxy-glutamate, epsilon-N,N,N-trimethyllysine, e-N-acetyllysine, 0-phosphoserine, N-acetylserine, N-formylmethionine, 3-methylhistidine, 5-hydroxylysine, .sigma.-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Conservative amino acid substitutions can encompass non-naturally occurring amino acid residues, which are typically incorporated by chemical peptide synthesis rather than by synthesis in biological systems. These include peptidomimetics and other reversed or inverted forms of amino acid moieties. Naturally occurring residues can be divided into classes based on common side chain properties:
a) hydrophobic: norleucine, Met, Ala, Val, Leu, Ile;
b) neutral hydrophilic: Cys, Ser, Thr, Asn, Gln;
c) acidic: Asp, Glu;
d) basic: His, Lys, Arg;
e) residues that influence chain orientation: Gly, Pro; and
f) aromatic: Trp, Tyr, Phe.

For example, non-conservative substitutions can involve the exchange of a member of one of these classes for a member from another class.

In making changes to the antigen-binding molecule, the costimulatory or activating domains of the engineered T cell, according to certain embodiments, the hydropathic index of amino acids can be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (-0.4); threonine (-0.7); serine (-0.8); tryptophan (-0.9); tyrosine (-1.3); proline (-1.6); histidine (-3.2); glutamate (-3.5); glutamine (-3.5); aspartate (-3.5); asparagine (-3.5); lysine (-3.9); and arginine (-4.5). See, e.g., Kyte et al., J. Mol. Biol., 1982, 157, 105-131. It is known that certain amino acids can be substituted for other amino acids having a similar hydropathic index or score and still retain a similar biological activity. It is also understood in the art that the substitution of like amino acids can be made effectively on the basis of hydrophilicity, particularly where the biologically functional protein or peptide thereby created is intended for use in immunological embodiments, as in the present case. Exemplary amino acid substitutions are set forth in Table 5.

TABLE 5

| | | |
|---|---|---|
| Original Residues | Exemplary Substitutions | Preferred Substitutions |
| Ala | Val, Leu, Ile | Val |
| Arg | Lys, Gin, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Norleucine | Leu |
| Leu | Norleucine, Ile, Va, Met, Ala, Phe | Ile |
| Lys | Arg, 1, 4 Diamino-butyric Acid, Gin, Asn | Arg |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Norleucine | Leu |

The term “derivative” refers to a molecule that includes a chemical modification other than an insertion, deletion, or substitution of amino acids (or nucleic acids). In certain embodiments, derivatives comprise covalent modifications, including, but not limited to, chemical bonding with polymers, lipids, or other organic or inorganic moieties. In certain embodiments, a chemically modified antigen-binding molecule can have a greater circulating half-life than an antigen-binding molecule that is not chemically modified. In some embodiments, a derivative antigen-binding molecule is covalently modified to include one or more water soluble polymer attachments, including, but not limited to, polyethylene glycol, polyoxyethylene glycol, or polypropylene glycol.

Peptide analogs are commonly used in the pharmaceutical industry as non-peptide drugs with properties analogous to those of the template peptide. These types of non-peptide compound are termed “peptide mimetics” or “peptidomimetics.” Fauchere, J. L., Adv. Drug Res., 1986, 15, 29; Veber, D. F. & Freidinger, R. M., Trends in Neuroscience, 1985, 8, 392-396; and Evans, B. E., et al., J. Med. Chem., 1987, 30, 1229-1239, which are incorporated herein by reference for any purpose.

The term “therapeutically effective amount” refers to the amount of CAR-B cells determined to produce a therapeutic response in a mammal. Such therapeutically effective amounts are readily ascertained by one of ordinary skill in the art.

The terms “patient” and “subject” are used interchangeably and include human and non-human animal subjects as well as those with formally diagnosed disorders, those without formally recognized disorders, those receiving medical attention, those at risk of developing the disorders, etc.

The term “treat” and “treatment” includes therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. The term “prevent” does not require the 100% elimination of the possibility of an event. Rather, it denotes that the likelihood of the occurrence of the event has been reduced in the presence of the compound or method.

Standard techniques can be used for recombinant DNA, oligonucleotide synthesis, and tissue culture and transformation (e.g., electroporation, lipofection). Enzymatic reactions and purification techniques can be performed according to manufacturer’s specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

### Sequences

The following sequences will further exemplify the invention:

CD28 transmembrane domain - mouse

(SEQ ID NO: 1)
TTCTGGGCCCTTGTGGTGGTTGCCGGAGTGCTGTTTTGCTATGGGCTCCTGGTTA
CCGTTGCCCTTTGTGTGATTTGGACC

CD28 transmembrane domain - mouse

(SEQ ID NO: 2)
FWALVVVAGVLFCYGLLVTVALCVIWT

CD28 transmembrane domain - human

(SEQ ID NO: 3)
TTTTGGGTATTGGTAGTGGTGGGCGGAGTTTTAGCCTGCTACAGCCTCCTGGTAA
CAGTGGCTTTTATCATCTTTTGGGTG

CD28 transmembrane domain - human

(SEQ ID NO: 4)
FWVLVVVGGVLACYSLLVTVAFIIFWV

CD 19 cytoplasmic domain - human

(SEQ ID NO: 5)
CAGCGGGCTTTAGTCTTGCGGCGTAAACGTAAAAGAATGACAGATCCAACTCGC
AGGTTCTTCAAAGTGACCCCCCCACCTGGGTCCGGACCGCAGAACCAATATGGG
AATGTCCTGTCTCTGCCTACGCCTACAAGTGGACTGGGTAGGGCTCAGAGGTGG
GCTGCCGGTCTCGGCGGAACTGCGCCATCTTACGGAAATCCCTCCTCCGACGTTC
AGGCAGACGGGGCCCTGGGGTCTCGATCCCCGCCTGGTGTTGGACCAGAAGAGG
AAGAGGGCGAGGGCTACGAAGAGCCCGACTCCGAAGAGGACAGTGAGTTTTAC
GAGAACGACAGCAACCTGGGGCAGGATCAGCTGTCACAGGATGGCTCAGGATA
TGAAAACCCTGAGGACGAGCCTTTGGGGCCTGAAGATGAGGACTCCTTTTCTAA
TGCAGAGTCATATGAGAATGAGGACGAAGAATTGACTCAACCCGTGGCAAGAA
CAATGGATTTCCTCAGTCCACACGGGAGTGCATGGGACCCCTCCAGAGAGGCTA
CTAGCCTCGGTTCTCAAAGCTATGAGGACATGAGGGGTATTCTGTACGCAGCGC
CTCAGTTGAGGTCCATCCGCGGCCAGCCAGGCCCAAACCATGAGGAAGATGCCG
ATTCTTACGAAAACATGGACAACCCCGATGGTCCTGACCCCGCATGGGGGGGCG
GCGGGAGGATGGGCACCTGGTCTACTCGC

CD 19 cytoplasmic domain - human

(SEQ ID NO: 6)
QRALVLRRKRKRMTDPTRRFFKVTPPPGSGPQNQYGNVLSLPTPTSGLGRAQRWAA
GLGGTAPSYGNPSSDVQADGALGSRSPPGVGPEEEEGEGYEEPDSEEDSEFYENDSN
LGQDQLSQDGSGYENPEDEPLGPEDEDSFSNAESYENEDEELTQPVARTMDFLSPHG
SAWDPSREATSLGSQSYEDMRGILYAAPQLRSIRGQPGPNHEEDADSYENMDNPDG
PDPAWGGGGRMGTWSTR

CD40 cytoplasmic domain - human

(SEQ ID NO: 7)
AAGAAGGTTGCAAAAAAACCTACTAATAAGGCTCCCCATCCTAAGCAAGAGCCC
CAAGAAATTAACTTTCCCGATGATCTTCCGGGTTCTAACACGGCAGCCCCGGTG
CAGGAGACCCTGCATGGTTGTCAACCCGTCACTCAGGAGGACGGGAAAGAGTCT
CGTATCTCCGTCCAGGAGAGACAG

CD40 cytoplasmic domain - human

(SEQ ID NO: 8)
KKVAKKPTNKAPHPKQEPQEINFPDDLPGSNTAAPVQETLHGCQPVTQEDGKESRIS
VQERQ

CD40 + CD79b cytoplasmic domain - human

(SEQ ID NO: 9)
AAGAAGGTTGCAAAAAAACCTACTAATAAGGCTCCCCATCCTAAGCAAGAGCCC
CAAGAAATTAACTTTCCCGATGATCTTCCGGGTTCTAACACGGCAGCCCCGGTG
CAGGAGACCCTGCATGGTTGTCAACCCGTCACTCAGGAGGACGGGAAAGAGTCT
CGTATCTCCGTCCAGGAGAGACAGGACAAGGACGATAGTAAAGCAGGGATGGA
GGAGGACCATACATACGAGGGACTGGATATCGATCAGACAGCCACGTACGAAG
ACATTGTGACACTGAGAACTGGCGAGGTGAAGTGGTCAGTGGGAGAACATCCG
GGGCAGGAA

CD40 + CD79b cytoplasmic domain - human

(SEQ ID NO: 10)
KKVAKKPTNK APHPKQEPQE INFPDDLPGS NTAAPVQETL HGCQPVTQED
GKESRISVQE RQDKDDSKAG MEEDHTYEGL DIDQTATYED IVTLRTGEVK
WSVGEHPGQE

CD40 + CD137 cytoplasmic domain - human

(SEQ ID NO: 11)
AAGAAGGTTGCAAAAAAACCTACTAATAAGGCTCCCCATCCTAAGCAAGAGCCC
CAAGAAATTAACTTTCCCGATGATCTTCCGGGTTCTAACACGGCAGCCCCGGTG
CAGGAGACCCTGCATGGTTGTCAACCCGTCACTCAGGAGGACGGGAAAGAGTCT
CGTATCTCCGTCCAGGAGAGACAGAAAAGAGGCCGAAAAAAGCTGCTGTACAT
CTTCAAACAACCCTTCATGCGACCTGTTCAGACGACACAGGAGGAGGACGGCTG
CAGCTGTAGGTTTCCCGAAGAAGAGGAGGGAGGATGCGAACTT

CD40 + CD137 cytoplasmic domain - human

(SEQ ID NO: 12)
KKVAKKPTNKAPHPKQEPQEINFPDDLPGSNTAAPVQETLHGCQPVTQEDGKESRIS
VQERQKRGRKKLLYIFKQPFMRPVQTTQEEDGCSCRFPEEEEGGCEL

CD 137 cytoplasmic domain - human

(SEQ ID NO: 13)
AAAAGAGGCCGAAAAAAGCTGCTGTACATCTTCAAACAACCCTTCATGCGACCT
GTTCAGACGACACAGGAGGAGGACGGCTGCAGCTGTAGGTTTCCCGAAGAAGA
GGAGGGAGGATGCGAACTT

CD 137 cytoplasmic domain - human

(SEQ ID NO: 14)
KRGRKKLLYIFKQPFMRPVQTTQEEDGCSCRFPEEEEGGCEL

CD40 and Fc gamma receptor 2a cytoplasmic domain - human

(SEQ ID NO: 15)
AAGAAGGTTGCAAAAAAACCTACTAATAAGGCTCCCCATCCTAAGCAAGAGCCC
CAAGAAATTAACTTTCCCGATGATCTTCCGGGTTCTAACACGGCAGCCCCGGTG
CAGGAGACCCTGCATGGTTGTCAACCCGTCACTCAGGAGGACGGGAAAGAGTCT
CGTATCTCCGTCCAGGAGAGACAGCGCAAAAAACGTATAAGCGCAAACTCTACA
GATCCAGTAAAAGCCGCGCAATTCGAGCCTCCCGGCCGCCAGATGATTGCAATA
CGGAAACGTCAACTGGAGGAAACTAATAATGACTATGAGACGGCCGACGGTGG
ATACATGACCCTTAATCCCCGCGCGCCAACCGACGATGATAAGAACATATATCT
GACGCTCCCCCCTAACGATCACGTTAACAGTAATAAT

CD40 and Fc gamma receptor 2a cytoplasmic domain - human

(SEQ ID NO: 16)
KKVAKKPTNKAPHPKQEPQEINFPDDLPGSNTAAPVQETLHGCQPVTQEDGKESRIS
VQERQRKKRISANSTDPVKAAQFEPPGRQMIAIRKRQLEETNNDYETADGGYMTLN
PRAPTDDDKNIYLTLPPNDHVNSNN

Fc gamma receptor 2a cytoplasmic domain - human

(SEQ ID NO: 17)
CGCAAAAAACGTATAAGCGCAAACTCTACAGATCCAGTAAAAGCCGCGCAATTC
GAGCCTCCCGGCCGCCAGATGATTGCAATACGGAAACGTCAACTGGAGGAAACT
AATAATGACTATGAGACGGCCGACGGTGGATACATGACCCTTAATCCCCGCGCG
CCAACCGACGATGATAAGAACATATATCTGACGCTCCCCCCTAACGATCACGTT
AACAGTAATAAT

Fc gamma receptor 2a cytoplasmic domain - human

(SEQ ID NO: 18)
RKKRISANSTDPVKAAQFEPPGRQMIAIRKRQLEETNNDYETADGGYMTLNPRAPT
DDDKNIYLTLPPNDHVNSNN

Myd88 + CD40 cytoplasmic domain - human

(SEQ ID NO: 19)
ATGGCGGCGGGCGGGCCCGGCGCCGGAAGCGCCGCGCCAGTCTCATCTACGTCC
AGTCTGCCACTGGCTGCCCTGAACATGAGAGTGAGACGCCGTTTATCCCTCTTCC
TGAATGTGCGGACCCAGGTCGCCGCTGATTGGACCGCCCTGGCCGAAGAGATGG
ACTTTGAATACTTGGAAATCAGACAGCTGGAAACACAGGCAGACCCAACCGGG
AGACTGCTTGACGCCTGGCAGGGACGCCCAGGGGCAAGTGTTGGTCGGTTACTG
GAGCTTTTAACTAAGTTGGGCCGCGATGACGTGCTGTTGGAGTTAGGACCCAGT
ATCGAGGAGGATTGTCAGAAATACATCTTGAAACAGCAGCAGGAGGAGGCGGA
AAAGCCCCTGCAGGTGGCGGCCGTTGACAGCAGTGTACCCAGAACAGCTGAGCT
GGCCGGCATCACAACCCTGGATGATCCCCTGGGCCACATGCCTGAGAGGTTCGA
CGCTTTCATAAAGAAGGTTGCAAAAAAACCTACTAATAAGGCTCCCCATCCTAA
GCAAGAGCCCCAAGAAATTAACTTTCCCGATGATCTTCCGGGTTCTAACACGGC
AGCCCCGGTGCAGGAGACCCTGCATGGTTGTCAACCCGTCACTCAGGAGGACGG
GAAAGAGTCTCGTATCTCCGTCCAGGAGAGACAG

Myd88 + CD40 cytoplasmic domain - human

(SEQ ID NO: 20)
MAAGGPGAGSAAPVSSTSSLPLAALNMRVRRRLSLFLNVRTQVAADWTALAEEMD
FEYLEIRQLETQADPTGRLLDAWQGRPGASVGRLLELLTKLGRDDVLLELGPSIEED
CQKYILKQQQEEAEKPLQVAAVDSSVPRTAELAGITTLDDPLGHMPERFDAFIKKVA
KKPTNKAPHPKQEPQEINFPDDLPGSNTAAPVQETLHGCQPVTQEDGKESRISVQER
Q

Myd88 cytoplasmic domain - human

(SEQ ID NO: 21)
ATGGCGGCGGGCGGGCCCGGCGCCGGAAGCGCCGCGCCAGTCTCATCTACGTCC
AGTCTGCCACTGGCTGCCCTGAACATGAGAGTGAGACGCCGTTTATCCCTCTTCC
TGAATGTGCGGACCCAGGTCGCCGCTGATTGGACCGCCCTGGCCGAAGAGATGG
ACTTTGAATACTTGGAAATCAGACAGCTGGAAACACAGGCAGACCCAACCGGG
AGACTGCTTGACGCCTGGCAGGGACGCCCAGGGGCAAGTGTTGGTCGGTTACTG
GAGCTTTTAACTAAGTTGGGCCGCGATGACGTGCTGTTGGAGTTAGGACCCAGT
ATCGAGGAGGATTGTCAGAAATACATCTTGAAACAGCAGCAGGAGGAGGCGGA
AAAGCCCCTGCAGGTGGCGGCCGTTGACAGCAGTGTACCCAGAACAGCTGAGCT
GGCCGGCATCACAACCCTGGATGATCCCCTGGGCCACATGCCTGAGAGGTTCGA
CGCTTTCATA

Myd88 cytoplasmic domain - human

(SEQ ID NO: 22)
MAAGGPGAGSAAPVSSTSSLPLAALNMRVRRRLSLFLNVRTQVAADWTALAEEMD
FEYLEIRQLETQADPTGRLLDAWQGRPGASVGRLLELLTKLGRDDVLLELGPSIEED
CQKYILKQQQEEAEKPLQVAAVDSSVPRTAELAGITTLDDPLGHMPERFDAFI

CD79a cytoplasmic domain - human

(SEQ ID NO: 23)
AGGAAACGATGGCAGAACGAGAAGCTCGGGTTGGATGCCGGGGATGAATATGA
AGATGAAAACCTTTATGAAGGCCTGAACCTGGACGACTGCTCCATGTATGAGGA
CATCTCCCGGGGCCTCCAGGGCACCTACCAGGATGTGGGCAGCCTCAACATAGG
AGATGTCCAGCTGGAGAAGCCG

CD79a cytoplasmic domain - human

(SEQ ID NO: 24)
RKRWQNEKLGLDAGDEYEDENLYEGLNLDDCSMYEDISRGLQGTYQDVGSLNIGD
VQLEKP

CD79b cytoplasmic domain - human

(SEQ ID NO: 25)
CTGGACAAGGATGACAGCAAGGCTGGCATGGAGGAAGATCACACCTACGAGGG
CCTGGACATTGACCAGACAGCCACCTATGAGGACATAGTGACGCTGCGGACAGG
GGAAGTGAAGTGGTCTGTAGGTGAGCACCCAGGCCAGGAG

CD79b cytoplasmic domain - human

(SEQ ID NO: 26)
LDKDDSKAGMEEDHTYEGLDIDQTATYEDIVTLRTGEVKWSVGEHPGQE

CD8 hinge domain - human

(SEQ ID NO: 27)
TTCGTGCCTGTGTTCCTCCCAGCTAAGCCCACTACCACCCCCGCTCCAAGGCCGC
CCACGCCCGCTCCTACTATTGCTAGTCAGCCTTTAAGTTTACGACCCGAAGCTTG
CAGGCCCGCCGCCGGCGGCGCTGTGCACACCAGGGGGCTTGATTTTGCCTGCGA
C

CD8 hinge domain - human

(SEQ ID NO: 28)
FVPVFLPAKPTTTPAPRPPTPAPTIASQPLSLRPEACRPAAGGAVHTRGLDFACD

Spacer with 3X strep II tag

(SEQ ID NO: 29)
GGCGCTGGTAGTGGCGGTAACTGGAGCCACCCTCAATTTGAGAAGGGCGGGTCA
GGCGGATCAGGTGGTAGTGGTGGGTCCAACTGGAGCCATCCGCAATTTGAAAAG
GGCGGAAGCGGCGGTTCCGGCGGTTCAGGCGGTAGCAACTGGTCACATCCGCAA
TTTGAGAAAGGCGGGTCAGGCGGCGGG

Spacer with 3X strep II tag

(SEQ ID NO: 30)
GAGSGGNWSHPQFEKGGSGGSGGSGGSNWSHPQFEKGGSGGSGGSGGSNWSHPQF
EKGGSGGG

human IgG1Fc (transmembrane form)

(SEQ ID NO: 31)
CCCAAGAGCTGCGACAAGACCCACACCTGCCCCCCCTGCCCAGCCCCAGAGCTG
CTGGGCGGACCCTCCGTGTTCCTGTTCCCCCCCAAGCCCAAGGACACCCTGATG
ATCAGCAGGACCCCCGAGGTGACCTGCGTGGTGGTGGACGTGAGCCACGAGGA
CCCAGAGGTGAAGTTCAACTGGTACGTGGACGGCGTGGAGGTGCACAACGCCA
AGACCAAGCCCAGAGAGGAGCAGTACAACAGCACCTACAGGGTGGTGTCCGTG
CTGACCGTGCTGCACCAGGACTGGCTGAACGGCAAGGAATACAAGTGCAAGGT
CTCCAACAAGGCCCTGCCAGCCCCCATCGAAAAGACCATCAGCAAGGCCAAGG
GCCAGCCACGGGAGCCCCAGGTGTACACCCTGCCCCCCTCCCGGGAGGAGATGA
CCAAGAACCAGGTGTCCCTGACCTGTCTGGTGAAGGGCTTCTACCCCAGCGACA
TCGCCGTGGAGTGGGAGAGCAACGGCCAGCCCGAGAACAACTACAAGACCACC
CCCCCAGTGCTGGACAGCGACGGCAGCTTCTTCCTGTACAGCAAGCTGACCGTG
GACAAGTCCAGGTGGCAGCAGGGCAACGTGTTCAGCTGCAGCGTGATGCACGA
GGCCCTGCACAACCACTACACCCAGAAGAGCCTGAGCCTGTCCCCCGAGCTGCA
ACTGGAGGAGAGCTGTGCGGAGGCGCAGGACGGGGAGCTGGACGGGCTGTGGA
CGACCATCACCATCTTCATCACACTCTTCCTGTTAAGCGTGTGCTACAGTGCCAC
CGTCACCTTCTTCAAGGTGAAGTGGATCTTCTCCTCGGTGGTGGACCTGAAGCAG
ACCATCATCCCCGACTACAGGAACATGATCGGACAGGGGGCCTGA

human IgG1Fc (transmembrane form)

(SEQ ID NO: 32)
PKSCDKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQKS
LSLSPELQLEESCAEAQDGELDGLWTTITIFITLFLLSVCYSATVTFFKVKWIFSSVVD
LKQTIIPDYRNMIGQGA

anti-huPSMA scFv

(SEQ ID NO: 33)
GAGGTTCAACTTGTTCAATCTGGGGCAGAAGTGAAGAAGCCCGGGGCATCTGTG
AAAGTATCATGCAAAACATCCGGCTATACGTTTACCGAATACACCATTCACTGG
GTCAGACAGGCTCCCGGTCAAAGCCTCGAATGGATGGGAAATATTAACCCTAAC
AATGGCGGAACCACATATAATCAGAAATTCCAAGGCCGAGTGACGATAACTGTC
GATAAGAGTACGTCCACAGCTTACATGGAACTCAGCTCTTTGAGATCCGAAGAC
ACTGCAGTTTATTATTGTGCAGCTGGATGGAACTTCGACTATTGGGGACAAGGG
ACTCTTGTTACGGTGTCCAGTGGCAAACCAGGTAGTGGTAAACCCGGAAGCGGC
AAGCCCGGGAGCGGTAAACCTGGTAGCGACATCGTCATGACTCAAAGCCCTGAC
TCACTCGCCGTGAGCCTGGGAGAGCGTGCAACGCTATCTTGTCGGGCCTCTCAG
GATGTCGGAACTGCTGTAGACTGGTATCAACAGAAACCTGACCAATCACCAAAA
CTCCTGATTTATTGGGCCTCAACACGTCACACAGGAGTGCCAGATAGGTTCACA
GGTAGTGGCAGTGGAACTGATTTTACTTTGACAATTAGCAGCCTGCAAGCCGAA
GATGTAGCCGTTTACTTCTGTCAACAATATAACTCATACCCACTAACGTTCGGTG
CCGGGACGAAGGTAGAGATTAAA

anti-huPSMA scFv

(SEQ ID NO: 34)
EVQLVQSGAE VKKPGASVKV SCKTSGYTFT EYTIHWVRQA PGQSLEWMGN
INPNNGGTTY NQKFQGRVTI TVDKSTSTAY MELSSLRSED TAVYYCAAGW
NFDYWGQGTL VTVSSGKPGS GKPGSGKPGS GKPGSDIVMT QSPDSLAVSL
GERATLSCRA SQDVGTAVDW YQQKPDQSPK LLIYWASTRH TGVPDRFTGS
GSGTDFTLTI SSLQAEDVAV YFCQQYNSYP LTFGAGTKVE IK

anti-Sarcoglycan scFv

(SEQ ID NO: 35)
GAAGTCCAATTGGTTGAAAGCGGTGGTGGACTCGTCAAACCTGGCGGTAGCCTT
AAACTTTCATGTGCCGCAAGCGGCTTCACGTTTAGTAACTATGCTATGAGTTGGG
TCCGCCAAAGTCCAGAAAAGCGCCTCGAATGGGTGGCGGAGATCTCTGGAGGA
GGAACATATACATATTATCCAGACACCATGACCGGTAGGTTTACAATCTCAAGA
GACAACGCTAAGAACACCCTGTACCTGGAAATGTCAAGCCTGAGATCAGAAGAT
ACGGCCATGTATTATTGTACGCGCCTACTCGACTATTGGGGTCAAGGAACTTCCG
TGACGGTGTCAAGCGGAGGAGGTGGGAGCGGAGGAGGCGGAAGTGGCGGTGGT
GGCTCTGGTGGCGGTGGAAGTGATATAGTGATGACGCAAGCTGCCTTTTCAAAC
CCTGTTACTTTGGGGACTAGCGCATCAATCTCCTGTAGGTCCAGCAAATCTTTGC
TGCACAGTAATGGAATCACCTATCTTTTCTGGTATTTGCAAAAGCCTGGGCAGA
GCCCGCAACTGCTGATCTATCAAATGTCAAATCTTGCTTCCGGAGTTCCAGACCG
CTTCTCAAGTTCCGGGTCCGGCACTGATTTTACCTTGAGAATTTCTAGGGTCGAA
GCTGAAGACGTCGGTGTCTATTATTGCGCGCAAAACCTTGAGCTTCCATACACCT
TCGGGGGGGGCACAAAACTTGAGATCAAG

anti-Sarcoglycan scFv

(SEQ ID NO: 36)
EVQLVESGGGLVKPGGSLKLSCAASGFTFSNYAMSWVRQSPEKRLEWVAEISGGGT
YTYYPDTMTGRFTISRDNAKNTLYLEMSSLRSEDTAMYYCTRLLDYWGQGTSVTV
SSGGGGSGGGGSGGGGSGGGGSDIVMTQAAFSNPVTLGTSASISCRSSKSLLHSNGI
TYLFWYLQKPGQSPQLLIYQMSNLASGVPDRFSSSGSGTDFTLRISRVEAEDVGVYY
CAQNLELPYTFGGGTKLEIK

anti-hu GPC3 scFv

(SEQ ID NO: 37)
CAGTCTGTGTTGACGCAGCCGCCCTCAGTGTCTGCGGCCCCAGGACAGAGGGTC
ACCATCTCCTGCTCTGGAACCAGGTCCAACATTGGGAGTGATTATGTTTCCTGGT
ACCAACACCTCCCAGGAACAGCCCCCAAACTCCTCGTTTATGGCGATAATCTGC
GACCCTCAGGGATTCCTGACCGATTCTCTGCCTCCAAGTCTGGCACGTCAGCCAC
CCTGGGCATCACCGGACTCCAGACTGGGGACGAGGCCGATTATTACTGCGGCAC
ATGGGATTACACCCTGAATGGTGTGGTGTTCGGCGGAGGGACCAAGCTGACCGT
CCTAGGTTCTAGAGGTGGTGGTGGTAGCGGCGGCGGCGGCTCTGGTGGTGGTGG
ATCCCTCGAGATGGCCCAGGTGCAGCTGGTGGAGTCTGGGGGAGGCTTGGTACA
GCCTGGGGGGTCCCTGAGACTCTCCTGTGCAGCCTCTGGATTCACCTTTAGCAGC
TATGCCATGAGCTGGGTCCGCCAGGCTCCAGGGAAGGGGCTGGAGTGGGTCTCA
GTTATTTATAGCGGTGGTAGTAGCACATACTATGCAGACTCCGTGAAGGGCCGG
TTCACCATCTCCAGAGATAATTCCAAGAACACGCTGTATCTGCAAATGAACAGC
CTGAGAGCCGAGGACACGGCCGTATATTACTGTGCGCGCACTTCTTACCTGAAC
CATGGTGATTACTGGGGTCAAGGTACTCTGGTGACCGTGTCTAGCGCCGCTGCA

anti-hu GPC3 scFv

(SEQ ID NO: 38)
QSVLTQPPSVSAAPGQRVTISCSGTRSNIGSDYVSWYQHLPGTAPKLLVYGDNLRPS
GIPDRFSASKSGTSATLGITGLQTGDEADYYCGTWDYTLNGVVFGGGTKLTVLGSR
GGGGSGGGGSGGGGSLEMAQVQLVESGGGLVQPGGSLRLSCAASGFTFSSYAMSW
VRQAPGKGLEWVSVIYSGGSSTYYADSVKGRFTISRDNSKNTLYLQMNSLRAEDTA
VYYCARTSYLNHGDYWGQGTLVTVSSAAA

pWF-82

(SEQ ID NO: 39)
GAGGTTCAACTTGTTCAATCTGGGGCAGAAGTGAAGAAGCCCGGGGCATCTGTG
AAAGTATCATGCAAAACATCCGGCTATACGTTTACCGAATACACCATTCACTGG
GTCAGACAGGCTCCCGGTCAAAGCCTCGAATGGATGGGAAATATTAACCCTAAC
AATGGCGGAACCACATATAATCAGAAATTCCAAGGCCGAGTGACGATAACTGTC
GATAAGAGTACGTCCACAGCTTACATGGAACTCAGCTCTTTGAGATCCGAAGAC
ACTGCAGTTTATTATTGTGCAGCTGGATGGAACTTCGACTATTGGGGACAAGGG
ACTCTTGTTACGGTGTCCAGTGGCAAACCAGGTAGTGGTAAACCCGGAAGCGGC
AAGCCCGGGAGCGGTAAACCTGGTAGCGACATCGTCATGACTCAAAGCCCTGAC
TCACTCGCCGTGAGCCTGGGAGAGCGTGCAACGCTATCTTGTCGGGCCTCTCAG
GATGTCGGAACTGCTGTAGACTGGTATCAACAGAAACCTGACCAATCACCAAAA
CTCCTGATTTATTGGGCCTCAACACGTCACACAGGAGTGCCAGATAGGTTCACA
GGTAGTGGCAGTGGAACTGATTTTACTTTGACAATTAGCAGCCTGCAAGCCGAA
GATGTAGCCGTTTACTTCTGTCAACAATATAACTCATACCCACTAACGTTCGGTG
CCGGGACGAAGGTAGAGATTAAATTCGTGCCTGTGTTCCTCCCAGCTAAGCCCA
CTACCACCCCCGCTCCAAGGCCGCCCACGCCCGCTCCTACTATTGCTAGTCAGCC
TTTAAGTTTACGACCCGAAGCTTGCAGGCCCGCCGCCGGCGGCGCTGTGCACAC
CAGGGGGCTTGATTTTGCCTGCGACTTTTGGGTATTGGTAGTGGTGGGCGGAGTT
TTAGCCTGCTACAGCCTCCTGGTAACAGTGGCTTTTATCATCTTTTGGGTGCAGC
GGGCTTTAGTCTTGCGGCGTAAACGTAAAAGAATGACAGATCCAACTCGCAGGT
TCTTCAAAGTGACCCCCCCACCTGGGTCCGGACCGCAGAACCAATATGGGAATG
TCCTGTCTCTGCCTACGCCTACAAGTGGACTGGGTAGGGCTCAGAGGTGGGCTG
CCGGTCTCGGCGGAACTGCGCCATCTTACGGAAATCCCTCCTCCGACGTTCAGG
CAGACGGGGCCCTGGGGTCTCGATCCCCGCCTGGTGTTGGACCAGAAGAGGAAG
AGGGCGAGGGCTACGAAGAGCCCGACTCCGAAGAGGACAGTGAGTTTTACGAG
AACGACAGCAACCTGGGGCAGGATCAGCTGTCACAGGATGGCTCAGGATATGA
AAACCCTGAGGACGAGCCTTTGGGGCCTGAAGATGAGGACTCCTTTTCTAATGC
AGAGTCATATGAGAATGAGGACGAAGAATTGACTCAACCCGTGGCAAGAACAA
TGGATTTCCTCAGTCCACACGGGAGTGCATGGGACCCCTCCAGAGAGGCTACTA
GCCTCGGTTCTCAAAGCTATGAGGACATGAGGGGTATTCTGTACGCAGCGCCTC
AGTTGAGGTCCATCCGCGGCCAGCCAGGCCCAAACCATGAGGAAGATGCCGATT
CTTACGAAAACATGGACAACCCCGATGGTCCTGACCCCGCATGGGGGGGCGGCG
GGAGGATGGGCACCTGGTCTACTCGCTAG

pWF-82

(SEQ ID NO: 40)
EVQLVQSGAE VKKPGASVKV SCKTSGYTFT EYTIHWVRQA PGQSLEWMGN
INPNNGGTTY NQKFQGRVTI TVDKSTSTAY MELSSLRSED TAVYYCAAGW
NFDYWGQGTL VTVSSGKPGS GKPGSGKPGS GKPGSDIVMT QSPDSLAVSL
GERATLSCRA SQDVGTAVDW YQQKPDQSPK LLIYWASTRH TGVPDRFTGS
GSGTDFTLTI SSLQAEDVAV YFCQQYNSYP LTFGAGTKVE IKFVPVFLPA
KPTTTPAPRP PTPAPTIASQ PLSLRPEACR PAAGGAVHTR GLDFACDFWV
LVVVGGVLAC YSLLVTVAFI IFWVQRALVL RRKRKRMTDP TRRFFKVTPP
PGSGPQNQYG NVLSLPTPTS GLGRAQRWAA GLGGTAPSYG NPSSDVQADG
ALGSRSPPGV GPEEEEGEGY EEPDSEEDSE FYENDSNLGQ DQLSQDGSGY
ENPEDEPLGP EDEDSFSNAE SYENEDEELT QPVARTMDFL SPHGSAWDPS
REATSLGSQS YEDMRGILYA APQLRSIRGQ PGPNHEEDAD SYENMDNPDG
PDPAWGGGGR MGTWSTR-

pWF-83

(SEQ ID NO: 41)
GAGGTTCAACTTGTTCAATCTGGGGCAGAAGTGAAGAAGCCCGGGGCATCTGTG
AAAGTATCATGCAAAACATCCGGCTATACGTTTACCGAATACACCATTCACTGG
GTCAGACAGGCTCCCGGTCAAAGCCTCGAATGGATGGGAAATATTAACCCTAAC
AATGGCGGAACCACATATAATCAGAAATTCCAAGGCCGAGTGACGATAACTGTC
GATAAGAGTACGTCCACAGCTTACATGGAACTCAGCTCTTTGAGATCCGAAGAC
ACTGCAGTTTATTATTGTGCAGCTGGATGGAACTTCGACTATTGGGGACAAGGG
ACTCTTGTTACGGTGTCCAGTGGCAAACCAGGTAGTGGTAAACCCGGAAGCGGC
AAGCCCGGGAGCGGTAAACCTGGTAGCGACATCGTCATGACTCAAAGCCCTGAC
TCACTCGCCGTGAGCCTGGGAGAGCGTGCAACGCTATCTTGTCGGGCCTCTCAG
GATGTCGGAACTGCTGTAGACTGGTATCAACAGAAACCTGACCAATCACCAAAA
CTCCTGATTTATTGGGCCTCAACACGTCACACAGGAGTGCCAGATAGGTTCACA
GGTAGTGGCAGTGGAACTGATTTTACTTTGACAATTAGCAGCCTGCAAGCCGAA
GATGTAGCCGTTTACTTCTGTCAACAATATAACTCATACCCACTAACGTTCGGTG
CCGGGACGAAGGTAGAGATTAAATTCGTGCCTGTGTTCCTCCCAGCTAAGCCCA
CTACCACCCCCGCTCCAAGGCCGCCCACGCCCGCTCCTACTATTGCTAGTCAGCC
TTTAAGTTTACGACCCGAAGCTTGCAGGCCCGCCGCCGGCGGCGCTGTGCACAC
CAGGGGGCTTGATTTTGCCTGCGACTTTTGGGTATTGGTAGTGGTGGGCGGAGTT
TTAGCCTGCTACAGCCTCCTGGTAACAGTGGCTTTTATCATCTTTTGGGTGCTGG
ACAAGGATGACAGCAAGGCTGGCATGGAGGAAGATCACACCTACGAGGGCCTG
GACATTGACCAGACAGCCACCTATGAGGACATAGTGACGCTGCGGACAGGGGA
AGTGAAGTGGTCTGTAGGTGAGCACCCAGGCCAGGAGTGA

pWF-83

(SEQ ID NO: 42)
EVQLVQSGAE VKKPGASVKV SCKTSGYTFT EYTIHWVRQA PGQSLEWMGN
INPNNGGTTY NQKFQGRVTI TVDKSTSTAY MELSSLRSED TAVYYCAAGW
NFDYWGQGTL VTVSSGKPGS GKPGSGKPGS GKPGSDIVMT QSPDSLAVSL
GERATLSCRA SQDVGTAVDW YQQKPDQSPK LLIYWASTRH TGVPDRFTGS
GSGTDFTLTI SSLQAEDVAV YFCQQYNSYP LTFGAGTKVE IKFVPVFLPA
KPTTTPAPRP PTPAPTIASQ PLSLRPEACR PAAGGAVHTR GLDFACDFWV
LVVVGGVLAC YSLLVTVAFI IFWVLDKDDS KAGMEEDHTY EGLDIDQTAT
YEDIVTLRTG EVKWSVGEHP GQE-

pWF-84:

(SEQ ID NO: 43)
GAGGTTCAACTTGTTCAATCTGGGGCAGAAGTGAAGAAGCCCGGGGCATCTGTG
AAAGTATCATGCAAAACATCCGGCTATACGTTTACCGAATACACCATTCACTGG
GTCAGACAGGCTCCCGGTCAAAGCCTCGAATGGATGGGAAATATTAACCCTAAC
AATGGCGGAACCACATATAATCAGAAATTCCAAGGCCGAGTGACGATAACTGTC
GATAAGAGTACGTCCACAGCTTACATGGAACTCAGCTCTTTGAGATCCGAAGAC
ACTGCAGTTTATTATTGTGCAGCTGGATGGAACTTCGACTATTGGGGACAAGGG
ACTCTTGTTACGGTGTCCAGTGGCAAACCAGGTAGTGGTAAACCCGGAAGCGGC
AAGCCCGGGAGCGGTAAACCTGGTAGCGACATCGTCATGACTCAAAGCCCTGAC
TCACTCGCCGTGAGCCTGGGAGAGCGTGCAACGCTATCTTGTCGGGCCTCTCAG
GATGTCGGAACTGCTGTAGACTGGTATCAACAGAAACCTGACCAATCACCAAAA
CTCCTGATTTATTGGGCCTCAACACGTCACACAGGAGTGCCAGATAGGTTCACA
GGTAGTGGCAGTGGAACTGATTTTACTTTGACAATTAGCAGCCTGCAAGCCGAA
GATGTAGCCGTTTACTTCTGTCAACAATATAACTCATACCCACTAACGTTCGGTG
CCGGGACGAAGGTAGAGATTAAATTCGTGCCTGTGTTCCTCCCAGCTAAGCCCA
CTACCACCCCCGCTCCAAGGCCGCCCACGCCCGCTCCTACTATTGCTAGTCAGCC
TTTAAGTTTACGACCCGAAGCTTGCAGGCCCGCCGCCGGCGGCGCTGTGCACAC
CAGGGGGCTTGATTTTGCCTGCGACTTTTGGGTATTGGTAGTGGTGGGCGGAGTT
TTAGCCTGCTACAGCCTCCTGGTAACAGTGGCTTTTATCATCTTTTGGGTGAAGA
AGGTTGCAAAAAAACCTACTAATAAGGCTCCCCATCCTAAGCAAGAGCCCCAAG
AAATTAACTTTCCCGATGATCTTCCGGGTTCTAACACGGCAGCCCCGGTGCAGG
AGACCCTGCATGGTTGTCAACCCGTCACTCAGGAGGACGGGAAAGAGTCTCGTA
TCTCCGTCCAGGAGAGACAGGACAAGGACGATAGTAAAGCAGGGATGGAGGAG
GACCATACATACGAGGGACTGGATATCGATCAGACAGCCACGTACGAAGACATT
GTGACACTGAGAACTGGCGAGGTGAAGTGGTCAGTGGGAGAACATCCGGGGCA
GGAATAA

pWF-84:

(SEQ ID NO: 44)
EVQLVQSGAE VKKPGASVKV SCKTSGYTFT EYTIHWVRQA PGQSLEWMGN
INPNNGGTTY NQKFQGRVTI TVDKSTSTAY MELSSLRSED TAVYYCAAGW
NFDYWGQGTL VTVSSGKPGS GKPGSGKPGS GKPGSDIVMT QSPDSLAVSL
GERATLSCRA SQDVGTAVDW YQQKPDQSPK LLIYWASTRH TGVPDRFTGS
GSGTDFTLTI SSLQAEDVAV YFCQQYNSYP LTFGAGTKVE IKFVPVFLPA
KPTTTPAPRP PTPAPTIASQ PLSLRPEACR PAAGGAVHTR GLDFACDFWV
LVVVGGVLAC YSLLVTVAFI IFWVKKVAKK PTNKAPHPKQ EPQEINFPDD
LPGSNTAAPV QETLHGCQPV TQEDGKESRI SVQERQDKDD SKAGMEEDHT
YEGLDIDQTA TYEDIVTLRT GEVKWSVGEH PGQE-

pWF-85:

(SEQ ID NO: 45)
GAGGTTCAACTTGTTCAATCTGGGGCAGAAGTGAAGAAGCCCGGGGCATCTGTG
AAAGTATCATGCAAAACATCCGGCTATACGTTTACCGAATACACCATTCACTGG
GTCAGACAGGCTCCCGGTCAAAGCCTCGAATGGATGGGAAATATTAACCCTAAC
AATGGCGGAACCACATATAATCAGAAATTCCAAGGCCGAGTGACGATAACTGTC
GATAAGAGTACGTCCACAGCTTACATGGAACTCAGCTCTTTGAGATCCGAAGAC
ACTGCAGTTTATTATTGTGCAGCTGGATGGAACTTCGACTATTGGGGACAAGGG
ACTCTTGTTACGGTGTCCAGTGGCAAACCAGGTAGTGGTAAACCCGGAAGCGGC
AAGCCCGGGAGCGGTAAACCTGGTAGCGACATCGTCATGACTCAAAGCCCTGAC
TCACTCGCCGTGAGCCTGGGAGAGCGTGCAACGCTATCTTGTCGGGCCTCTCAG
GATGTCGGAACTGCTGTAGACTGGTATCAACAGAAACCTGACCAATCACCAAAA
CTCCTGATTTATTGGGCCTCAACACGTCACACAGGAGTGCCAGATAGGTTCACA
GGTAGTGGCAGTGGAACTGATTTTACTTTGACAATTAGCAGCCTGCAAGCCGAA
GATGTAGCCGTTTACTTCTGTCAACAATATAACTCATACCCACTAACGTTCGGTG
CCGGGACGAAGGTAGAGATTAAATTCGTGCCTGTGTTCCTCCCAGCTAAGCCCA
CTACCACCCCCGCTCCAAGGCCGCCCACGCCCGCTCCTACTATTGCTAGTCAGCC
TTTAAGTTTACGACCCGAAGCTTGCAGGCCCGCCGCCGGCGGCGCTGTGCACAC
CAGGGGGCTTGATTTTGCCTGCGACTTTTGGGTATTGGTAGTGGTGGGCGGAGTT
TTAGCCTGCTACAGCCTCCTGGTAACAGTGGCTTTTATCATCTTTTGGGTGAAGA
AGGTTGCAAAAAAACCTACTAATAAGGCTCCCCATCCTAAGCAAGAGCCCCAAG
AAATTAACTTTCCCGATGATCTTCCGGGTTCTAACACGGCAGCCCCGGTGCAGG
AGACCCTGCATGGTTGTCAACCCGTCACTCAGGAGGACGGGAAAGAGTCTCGTA
TCTCCGTCCAGGAGAGACAGAAAAGAGGCCGAAAAAAGCTGCTGTACATCTTCA
AACAACCCTTCATGCGACCTGTTCAGACGACACAGGAGGAGGACGGCTGCAGCT
GTAGGTTTCCCGAAGAAGAGGAGGGAGGATGCGAACTTTAA

pWF-85:

(SEQ ID NO: 46)
EVQLVQSGAE VKKPGASVKV SCKTSGYTFT EYTIHWVRQA PGQSLEWMGN
INPNNGGTTY NQKFQGRVTI TVDKSTSTAY MELSSLRSED TAVYYCAAGW
NFDYWGQGTL VTVSSGKPGS GKPGSGKPGS GKPGSDIVMT QSPDSLAVSL
GERATLSCRA SQDVGTAVDW YQQKPDQSPK LLIYWASTRH TGVPDRFTGS
GSGTDFTLTI SSLQAEDVAV YFCQQYNSYP LTFGAGTKVE IKFVPVFLPA
KPTTTPAPRP PTPAPTIASQ PLSLRPEACR PAAGGAVHTR GLDFACDFWV
LVVVGGVLAC YSLLVTVAFI IFWVKKVAKK PTNKAPHPKQ EPQEINFPDD
LPGSNTAAPV QETLHGCQPV TQEDGKESRI SVQERQKRGR KKLLYIFKQP
FMRPVQTTQE EDGCSCRFPE EEEGGCEL-

pWF-86

(SEQ ID NO: 150)
GAGGTTCAACTTGTTCAATCTGGGGCAGAAGTGAAGAAGCCCGGGGCATCTGTGAAA
GTATCATGCAAAACATCCGGCTATACGTTTACCGAATACACCATTCACTGGGTCAGAC
AGGCTCCCGGTCAAAGCCTCGAATGGATGGGAAATATTAACCCTAACAATGGCGGAA
CCACATATAATCAGAAATTCCAAGGCCGAGTGACGATAACTGTCGATAAGAGTACGT
CCACAGCTTACATGGAACTCAGCTCTTTGAGATCCGAAGACACTGCAGTTTATTATTG
TGCAGCTGGATGGAACTTCGACTATTGGGGACAAGGGACTCTTGTTACGGTGTCCAGT
GGCAAACCAGGTAGTGGTAAACCCGGAAGCGGCAAGCCCGGGAGCGGTAAACCTGG
TAGCGACATCGTCATGACTCAAAGCCCTGACTCACTCGCCGTGAGCCTGGGAGAGCG
TGCAACGCTATCTTGTCGGGCCTCTCAGGATGTCGGAACTGCTGTAGACTGGTATCAA
CAGAAACCTGACCAATCACCAAAACTCCTGATTTATTGGGCCTCAACACGTCACACAG
GAGTGCCAGATAGGTTCACAGGTAGTGGCAGTGGAACTGATTTTACTTTGACAATTAG
CAGCCTGCAAGCCGAAGATGTAGCCGTTTACTTCTGTCAACAATATAACTCATACCCA
CTAACGTTCGGTGCCGGGACGAAGGTAGAGATTAAATTCGTGCCTGTGTTCCTCCCAG
CTAAGCCCACTACCACCCCCGCTCCAAGGCCGCCCACGCCCGCTCCTACTATTGCTAG
TCAGCCTTTAAGTTTACGACCCGAAGCTTGCAGGCCCGCCGCCGGCGGCGCTGTGCAC
ACCAGGGGGCTTGATTTTGCCTGCGACTTTTGGGTATTGGTAGTGGTGGGCGGAGTTT
TAGCCTGCTACAGCCTCCTGGTAACAGTGGCTTTTATCATCTTTTGGGTGAAGAAGGT
TGCAAAAAAACCTACTAATAAGGCTCCCCATCCTAAGCAAGAGCCCCAAGAAATTAA
CTTTCCCGATGATCTTCCGGGTTCTAACACGGCAGCCCCGGTGCAGGAGACCCTGCAT
GGTTGTCAACCCGTCACTCAGGAGGACGGGAAAGAGTCTCGTATCTCCGTCCAGGAG
AGACAGCGCAAAAAACGTATAAGCGCAAACTCTACAGATCCAGTAAAAGCCGCGCA
ATTCGAGCCTCCCGGCCGCCAGATGATTGCAATACGGAAACGTCAACTGGAGGAAAC
TAATAATGACTATGAGACGGCCGACGGTGGATACATGACCCTTAATCCCCGCGCGCC
AACCGACGATGATAAGAACATATATCTGACGCTCCCCCCTAACGATCACGTTAACAGT
AATAATTAA

pWF-86:

(SEQ ID NO: 47)
EVQLVQSGAE VKKPGASVKV SCKTSGYTFT EYTIHWVRQA PGQSLEWMGN
INPNNGGTTY NQKFQGRVTI TVDKSTSTAY MELSSLRSED TAVYYCAAGW
NFDYWGQGTL VTVSSGKPGS GKPGSGKPGS GKPGSDIVMT QSPDSLAVSL
GERATLSCRA SQDVGTAVDW YQQKPDQSPK LLIYWASTRH TGVPDRFTGS
GSGTDFTLTI SSLQAEDVAV YFCQQYNSYP LTFGAGTKVE IKFVPVFLPA
KPTTTPAPRP PTPAPTIASQ PLSLRPEACR PAAGGAVHTR GLDFACDFWV
LVVVGGVLAC YSLLVTVAFI IFWVKKVAKK PTNKAPHPKQ EPQEINFPDD
LPGSNTAAPV QETLHGCQPV TQEDGKESRI SVQERQRKKR ISANSTDPVK
AAQFEPPGRQ MIAIRKRQLE ETNNDYETAD GGYMTLNPRA PTDDDKNIYL
TLPPNDHVNS NN-

pWF-87:

(SEQ ID NO: 48)
GAGGTTCAACTTGTTCAATCTGGGGCAGAAGTGAAGAAGCCCGGGGCATCTGTG
AAAGTATCATGCAAAACATCCGGCTATACGTTTACCGAATACACCATTCACTGG
GTCAGACAGGCTCCCGGTCAAAGCCTCGAATGGATGGGAAATATTAACCCTAAC
AATGGCGGAACCACATATAATCAGAAATTCCAAGGCCGAGTGACGATAACTGTC
GATAAGAGTACGTCCACAGCTTACATGGAACTCAGCTCTTTGAGATCCGAAGAC
ACTGCAGTTTATTATTGTGCAGCTGGATGGAACTTCGACTATTGGGGACAAGGG
ACTCTTGTTACGGTGTCCAGTGGCAAACCAGGTAGTGGTAAACCCGGAAGCGGC
AAGCCCGGGAGCGGTAAACCTGGTAGCGACATCGTCATGACTCAAAGCCCTGAC
TCACTCGCCGTGAGCCTGGGAGAGCGTGCAACGCTATCTTGTCGGGCCTCTCAG
GATGTCGGAACTGCTGTAGACTGGTATCAACAGAAACCTGACCAATCACCAAAA
CTCCTGATTTATTGGGCCTCAACACGTCACACAGGAGTGCCAGATAGGTTCACA
GGTAGTGGCAGTGGAACTGATTTTACTTTGACAATTAGCAGCCTGCAAGCCGAA
GATGTAGCCGTTTACTTCTGTCAACAATATAACTCATACCCACTAACGTTCGGTG
CCGGGACGAAGGTAGAGATTAAATTCGTGCCTGTGTTCCTCCCAGCTAAGCCCA
CTACCACCCCCGCTCCAAGGCCGCCCACGCCCGCTCCTACTATTGCTAGTCAGCC
TTTAAGTTTACGACCCGAAGCTTGCAGGCCCGCCGCCGGCGGCGCTGTGCACAC
CAGGGGGCTTGATTTTGCCTGCGACTTTTGGGTATTGGTAGTGGTGGGCGGAGTT
TTAGCCTGCTACAGCCTCCTGGTAACAGTGGCTTTTATCATCTTTTGGGTGATGG
CGGCGGGCGGGCCCGGCGCCGGAAGCGCCGCGCCAGTCTCATCTACGTCCAGTC
TGCCACTGGCTGCCCTGAACATGAGAGTGAGACGCCGTTTATCCCTCTTCCTGAA
TGTGCGGACCCAGGTCGCCGCTGATTGGACCGCCCTGGCCGAAGAGATGGACTT
TGAATACTTGGAAATCAGACAGCTGGAAACACAGGCAGACCCAACCGGGAGAC
TGCTTGACGCCTGGCAGGGACGCCCAGGGGCAAGTGTTGGTCGGTTACTGGAGC
TTTTAACTAAGTTGGGCCGCGATGACGTGCTGTTGGAGTTAGGACCCAGTATCG
AGGAGGATTGTCAGAAATACATCTTGAAACAGCAGCAGGAGGAGGCGGAAAAG
CCCCTGCAGGTGGCGGCCGTTGACAGCAGTGTACCCAGAACAGCTGAGCTGGCC
GGCATCACAACCCTGGATGATCCCCTGGGCCACATGCCTGAGAGGTTCGACGCT
TTCATAAAGAAGGTTGCAAAAAAACCTACTAATAAGGCTCCCCATCCTAAGCAA
GAGCCCCAAGAAATTAACTTTCCCGATGATCTTCCGGGTTCTAACACGGCAGCC
CCGGTGCAGGAGACCCTGCATGGTTGTCAACCCGTCACTCAGGAGGACGGGAAA
GAGTCTCGTATCTCCGTCCAGGAGAGACAGTGA

pWF-87:

(SEQ ID NO: 49)
EVQLVQSGAEVKKPGASVKVSCKTSGYTFTEYTIHWVRQAPGQSLEWMGNINPNN
GGTTYNQKFQGRVTITVDKSTSTAYMELSSLRSEDTAVYYCAAGWNFDYWGQGTL
VTVSSGKPGSGKPGSGKPGSGKPGSDIVMTQSPDSLAVSLGERATLSCRASQDVGTA
VDWYQQKPDQSPKLLIYWASTRHTGVPDRFTGSGSGTDFTLTISSLQAEDVAVYFC
QQYNSYPLTFGAGTKVEIKFVPVFLPAKPTTTPAPRPPTPAPTIASQPLSLRPEACRPA
AGGAVHTRGLDFACDFWVLVVVGGVLACYSLLVTVAFIIFWVMAAGGPGAGSAAP
VSSTSSLPLAALNMRVRRRLSLFLNVRTQVAADWTALAEEMDFEYLEIRQLETQAD
PTGRLLDAWQGRPGASVGRLLELLTKLGRDDVLLELGPSIEEDCQKYILKQQQEEAE
KPLQVAAVDSSVPRTAELAGITTLDDPLGHMPERFDAFIKKVAKKPTNKAPHPKQEP
QEINFPDDLPGSNTAAPVQETLHGCQPVTQEDGKESRISVQERQ-

pWF-88:

(SEQ ID NO: 50)
GAGGTTCAACTTGTTCAATCTGGGGCAGAAGTGAAGAAGCCCGGGGCATCTGTG
AAAGTATCATGCAAAACATCCGGCTATACGTTTACCGAATACACCATTCACTGG
GTCAGACAGGCTCCCGGTCAAAGCCTCGAATGGATGGGAAATATTAACCCTAAC
AATGGCGGAACCACATATAATCAGAAATTCCAAGGCCGAGTGACGATAACTGTC
GATAAGAGTACGTCCACAGCTTACATGGAACTCAGCTCTTTGAGATCCGAAGAC
ACTGCAGTTTATTATTGTGCAGCTGGATGGAACTTCGACTATTGGGGACAAGGG
ACTCTTGTTACGGTGTCCAGTGGCAAACCAGGTAGTGGTAAACCCGGAAGCGGC
AAGCCCGGGAGCGGTAAACCTGGTAGCGACATCGTCATGACTCAAAGCCCTGAC
TCACTCGCCGTGAGCCTGGGAGAGCGTGCAACGCTATCTTGTCGGGCCTCTCAG
GATGTCGGAACTGCTGTAGACTGGTATCAACAGAAACCTGACCAATCACCAAAA
CTCCTGATTTATTGGGCCTCAACACGTCACACAGGAGTGCCAGATAGGTTCACA
GGTAGTGGCAGTGGAACTGATTTTACTTTGACAATTAGCAGCCTGCAAGCCGAA
GATGTAGCCGTTTACTTCTGTCAACAATATAACTCATACCCACTAACGTTCGGTG
CCGGGACGAAGGTAGAGATTAAATTCGTGCCTGTGTTCCTCCCAGCTAAGCCCA
CTACCACCCCCGCTCCAAGGCCGCCCACGCCCGCTCCTACTATTGCTAGTCAGCC
TTTAAGTTTACGACCCGAAGCTTGCAGGCCCGCCGCCGGCGGCGCTGTGCACAC
CAGGGGGCTTGATTTTGCCTGCGACTTTTGGGTATTGGTAGTGGTGGGCGGAGTT
TTAGCCTGCTACAGCCTCCTGGTAACAGTGGCTTTTATCATCTTTTGGGTGAGGA
AACGATGGCAGAACGAGAAGCTCGGGTTGGATGCCGGGGATGAATATGAAGAT
GAAAACCTTTATGAAGGCCTGAACCTGGACGACTGCTCCATGTATGAGGACATC
TCCCGGGGCCTCCAGGGCACCTACCAGGATGTGGGCAGCCTCAACATAGGAGAT
GTCCAGCTGGAGAAGCCGTGA

pWF-88:

(SEQ ID NO: 51)
EVQLVQSGAEVKKPGASVKVSCKTSGYTFTEYTIHWVRQAPGQSLEWMGNINPNN
GGTTYNQKFQGRVTITVDKSTSTAYMELSSLRSEDTAVYYCAAGWNFDYWGQGTL
VTVSSGKPGSGKPGSGKPGSGKPGSDIVMTQSPDSLAVSLGERATLSCRASQDVGTA
VDWYQQKPDQSPKLLIYWASTRHTGVPDRFTGSGSGTDFTLTISSLQAEDVAVYFC
QQYNSYPLTFGAGTKVEIKFVPVFLPAKPTTTPAPRPPTPAPTIASQPLSLRPEACRPA
AGGAVHTRGLDFACDFWVLVVVGGVLACYSLLVTVAFIIFWVRKRWQNEKLGLD
AGDEYEDENLYEGLNLDDCSMYEDISRGLQGTYQDVGSLNIGDVQLEKP-

pWF-89:

(SEQ ID NO: 52)
GAGGTTCAACTTGTTCAATCTGGGGCAGAAGTGAAGAAGCCCGGGGCATCTGTG
AAAGTATCATGCAAAACATCCGGCTATACGTTTACCGAATACACCATTCACTGG
GTCAGACAGGCTCCCGGTCAAAGCCTCGAATGGATGGGAAATATTAACCCTAAC
AATGGCGGAACCACATATAATCAGAAATTCCAAGGCCGAGTGACGATAACTGTC
GATAAGAGTACGTCCACAGCTTACATGGAACTCAGCTCTTTGAGATCCGAAGAC
ACTGCAGTTTATTATTGTGCAGCTGGATGGAACTTCGACTATTGGGGACAAGGG
ACTCTTGTTACGGTGTCCAGTGGCAAACCAGGTAGTGGTAAACCCGGAAGCGGC
AAGCCCGGGAGCGGTAAACCTGGTAGCGACATCGTCATGACTCAAAGCCCTGAC
TCACTCGCCGTGAGCCTGGGAGAGCGTGCAACGCTATCTTGTCGGGCCTCTCAG
GATGTCGGAACTGCTGTAGACTGGTATCAACAGAAACCTGACCAATCACCAAAA
CTCCTGATTTATTGGGCCTCAACACGTCACACAGGAGTGCCAGATAGGTTCACA
GGTAGTGGCAGTGGAACTGATTTTACTTTGACAATTAGCAGCCTGCAAGCCGAA
GATGTAGCCGTTTACTTCTGTCAACAATATAACTCATACCCACTAACGTTCGGTG
CCGGGACGAAGGTAGAGATTAAATTCGTGCCTGTGTTCCTCCCAGCTAAGCCCA
CTACCACCCCCGCTCCAAGGCCGCCCACGCCCGCTCCTACTATTGCTAGTCAGCC
TTTAAGTTTACGACCCGAAGCTTGCAGGCCCGCCGCCGGCGGCGCTGTGCACAC
CAGGGGGCTTGATTTTGCCTGCGACTTTTGGGTATTGGTAGTGGTGGGCGGAGTT
TTAGCCTGCTACAGCCTCCTGGTAACAGTGGCTTTTATCATCTTTTGGGTGCTGG
ACAAGGATGACAGCAAGGCTGGCATGGAGGAAGATCACACCTACGAGGGCCTG
GACATTGACCAGACAGCCACCTATGAGGACATAGTGACGCTGCGGACAGGGGA
AGTGAAGTGGTCTGTAGGTGAGCACCCAGGCCAGGAGTGA

pWF-89:

(SEQ ID NO: 53)
EVQLVQSGAEVKKPGASVKVSCKTSGYTFTEYTIHWVRQAPGQSLEWMGNINPNN
GGTTYNQKFQGRVTITVDKSTSTAYMELSSLRSEDTAVYYCAAGWNFDYWGQGTL
VTVSSGKPGSGKPGSGKPGSGKPGSDIVMTQSPDSLAVSLGERATLSCRASQDVGTA
VDWYQQKPDQSPKLLIYWASTRHTGVPDRFTGSGSGTDFTLTISSLQAEDVAVYFC
QQYNSYPLTFGAGTKVEIKFVPVFLPAKPTTTPAPRPPTPAPTIASQPLSLRPEACRPA
AGGAVHTRGLDFACDFWVLVVVGGVLACYSLLVTVAFIIFWVLDKDDSKAGMEED
HTYEGLDIDQTATYEDIVTLRTGEVKWSVGEHPGQE

pWF-391:

(SEQ ID NO: 54)
GAGGTTCAACTTGTTCAATCTGGGGCAGAAGTGAAGAAGCCCGGGGCATCTGTG
AAAGTATCATGCAAAACATCCGGCTATACGTTTACCGAATACACCATTCACTGG
GTCAGACAGGCTCCCGGTCAAAGCCTCGAATGGATGGGAAATATTAACCCTAAC
AATGGCGGAACCACATATAATCAGAAATTCCAAGGCCGAGTGACGATAACTGTC
GATAAGAGTACGTCCACAGCTTACATGGAACTCAGCTCTTTGAGATCCGAAGAC
ACTGCAGTTTATTATTGTGCAGCTGGATGGAACTTCGACTATTGGGGACAAGGG
ACTCTTGTTACGGTGTCCAGTGGCAAACCAGGTAGTGGTAAACCCGGAAGCGGC
AAGCCCGGGAGCGGTAAACCTGGTAGCGACATCGTCATGACTCAAAGCCCTGAC
TCACTCGCCGTGAGCCTGGGAGAGCGTGCAACGCTATCTTGTCGGGCCTCTCAG
GATGTCGGAACTGCTGTAGACTGGTATCAACAGAAACCTGACCAATCACCAAAA
CTCCTGATTTATTGGGCCTCAACACGTCACACAGGAGTGCCAGATAGGTTCACA
GGTAGTGGCAGTGGAACTGATTTTACTTTGACAATTAGCAGCCTGCAAGCCGAA
GATGTAGCCGTTTACTTCTGTCAACAATATAACTCATACCCACTAACGTTCGGTG
CCGGGACGAAGGTAGAGATTAAAGGCGCTGGTAGTGGCGGTAACTGGAGCCAC
CCTCAATTTGAGAAGGGCGGGTCAGGCGGATCAGGTGGTAGTGGTGGGTCCAAC
TGGAGCCATCCGCAATTTGAAAAGGGCGGAAGCGGCGGTTCCGGCGGTTCAGGC
GGTAGCAACTGGTCACATCCGCAATTTGAGAAAGGCGGGTCAGGCGGCGGGTTT
TGGGCTCTCGTGGTGGTGGCTGGAGTGCTTTTCTGCTATGGCCTGCTGGTAACCG
TGGCCCTTTGTGTAATCTGGACCGATAAAGACGATGGAAAAGCCGGGATGGAAG
AAGACCATACCTACGAGGGGCTCAATATTGATCAAACCGCCACGTATGAAGACA
TTGTAACACTGCGCACAGGTGAGGTCAAGTGGTCCGTCGGTGAACACCCAGGAC
AAGAATAA

pWF-391:

(SEQ ID NO: 55)
EVQLVQSGAEVKKPGASVKVSCKTSGYTFTEYTIHWVRQAPGQSLEWMGNINPNN
GGTTYNQKFQGRVTITVDKSTSTAYMELSSLRSEDTAVYYCAAGWNFDYWGQGTL
VTVSSGKPGSGKPGSGKPGSGKPGSDIVMTQSPDSLAVSLGERATLSCRASQDVGTA
VDWYQQKPDQSPKLLIYWASTRHTGVPDRFTGSGSGTDFTLTISSLQAEDVAVYFC
QQYNSYPLTFGAGTKVEIKGAGSGGNWSHPQFEKGGSGGSGGSGGSNWSHPQFEK
GGSGGSGGSGGSNWSHPQFEKGGSGGGFWALVVVAGVLFCYGLLVTVALCVIWT
DKDDGKAGMEEDHTYEGLNIDQTATYEDIVTLRTGEVKWSVGEHPGQE

pWF-394:

(SEQ ID NO: 56)
GAAGTCCAATTGGTTGAAAGCGGTGGTGGACTCGTCAAACCTGGCGGTAGCCTT
AAACTTTCATGTGCCGCAAGCGGCTTCACGTTTAGTAACTATGCTATGAGTTGGG
TCCGCCAAAGTCCAGAAAAGCGCCTCGAATGGGTGGCGGAGATCTCTGGAGGA
GGAACATATACATATTATCCAGACACCATGACCGGTAGGTTTACAATCTCAAGA
GACAACGCTAAGAACACCCTGTACCTGGAAATGTCAAGCCTGAGATCAGAAGAT
ACGGCCATGTATTATTGTACGCGCCTACTCGACTATTGGGGTCAAGGAACTTCCG
TGACGGTGTCAAGCGGAGGAGGTGGGAGCGGAGGAGGCGGAAGTGGCGGTGGT
GGCTCTGGTGGCGGTGGAAGTGATATAGTGATGACGCAAGCTGCCTTTTCAAAC
CCTGTTACTTTGGGGACTAGCGCATCAATCTCCTGTAGGTCCAGCAAATCTTTGC
TGCACAGTAATGGAATCACCTATCTTTTCTGGTATTTGCAAAAGCCTGGGCAGA
GCCCGCAACTGCTGATCTATCAAATGTCAAATCTTGCTTCCGGAGTTCCAGACCG
CTTCTCAAGTTCCGGGTCCGGCACTGATTTTACCTTGAGAATTTCTAGGGTCGAA
GCTGAAGACGTCGGTGTCTATTATTGCGCGCAAAACCTTGAGCTTCCATACACCT
TCGGGGGGGGCACAAAACTTGAGATCAAGGGCGCTGGGAGCGGCGGGAATTGG
AGTCATCCACAATTCGAAAAGGGTGGGTCCGGCGGCAGTGGTGGAAGCGGCGG
GAGTAACTGGTCACATCCCCAGTTTGAGAAAGGCGGTAGTGGTGGCAGCGGCGG
TAGTGGTGGCAGTAATTGGAGCCATCCCCAATTCGAAAAGGGCGGTTCCGGCGG
CGGATTTTGGGCTCTTGTTGTGGTGGCCGGAGTATTGTTTTGCTATGGCCTGCTC
GTTACAGTGGCATTGTGCGTAATTTGGACTGATAAAGACGACGGCAAAGCCGGG
ATGGAAGAAGATCACACCTATGAGGGGCTTAATATAGATCAAACAGCCACATAT
GAAGATATTGTGACTCTAAGGACTGGAGAGGTTAAATGGAGTGTGGGTGAGCAT
CCAGGACAAGAATAA

pWF-394:

(SEQ ID NO: 57)
EVQLVESGGGLVKPGGSLKLSCAASGFTFSNYAMSWVRQSPEKRLEWVAEISGGGT
YTYYPDTMTGRFTISRDNAKNTLYLEMSSLRSEDTAMYYCTRLLDYWGQGTSVTV
SSGGGGSGGGGSGGGGSGGGGSDIVMTQAAFSNPVTLGTSASISCRSSKSLLHSNGI
TYLFWYLQKPGQSPQLLIYQMSNLASGVPDRFSSSGSGTDFTLRISRVEAEDVGVYY
CAQNLELPYTFGGGTKLEIKGAGSGGNWSHPQFEKGGSGGSGGSGGSNWSHPQFEK
GGSGGSGGSGGSNWSHPQFEKGGSGGGFWALVVVAGVLFCYGLLVTVALCVIWT
DKDDGKAGMEEDHTYEGLNIDQTATYEDIVTLRTGEVKWSVGEHPGQE

pWF-396:

(SEQ ID NO: 58)
CAGTCTGTGTTGACGCAGCCGCCCTCAGTGTCTGCGGCCCCAGGACAGAGGGTC
ACCATCTCCTGCTCTGGAACCAGGTCCAACATTGGGAGTGATTATGTTTCCTGGT
ACCAACACCTCCCAGGAACAGCCCCCAAACTCCTCGTTTATGGCGATAATCTGC
GACCCTCAGGGATTCCTGACCGATTCTCTGCCTCCAAGTCTGGCACGTCAGCCAC
CCTGGGCATCACCGGACTCCAGACTGGGGACGAGGCCGATTATTACTGCGGCAC
ATGGGATTACACCCTGAATGGTGTGGTGTTCGGCGGAGGGACCAAGCTGACCGT
CCTAGGTTCTAGAGGTGGTGGTGGTAGCGGCGGCGGCGGCTCTGGTGGTGGTGG
ATCCCTCGAGATGGCCCAGGTGCAGCTGGTGGAGTCTGGGGGAGGCTTGGTACA
GCCTGGGGGGTCCCTGAGACTCTCCTGTGCAGCCTCTGGATTCACCTTTAGCAGC
TATGCCATGAGCTGGGTCCGCCAGGCTCCAGGGAAGGGGCTGGAGTGGGTCTCA
GTTATTTATAGCGGTGGTAGTAGCACATACTATGCAGACTCCGTGAAGGGCCGG
TTCACCATCTCCAGAGATAATTCCAAGAACACGCTGTATCTGCAAATGAACAGC
CTGAGAGCCGAGGACACGGCCGTATATTACTGTGCGCGCACTTCTTACCTGAAC
CATGGTGATTACTGGGGTCAAGGTACTCTGGTGACCGTGTCTAGCGCCGCTGCA
TTCGTGCCTGTGTTCCTCCCAGCTAAGCCCACTACCACCCCCGCTCCAAGGCCGC
CCACGCCCGCTCCTACTATTGCTAGTCAGCCTTTAAGTTTACGACCCGAAGCTTG
CAGGCCCGCCGCCGGCGGCGCTGTGCACACCAGGGGGCTTGATTTTGCCTGCGA
CTTTTGGGTATTGGTAGTGGTGGGCGGAGTTTTAGCCTGCTACAGCCTCCTGGTA
ACAGTGGCTTTTATCATCTTTTGGGTGAGGAAACGATGGCAGAACGAGAAGCTC
GGGTTGGATGCCGGGGATGAATATGAAGATGAAAACCTTTATGAAGGCCTGAAC
CTGGACGACTGCTCCATGTATGAGGACATCTCCCGGGGCCTCCAGGGCACCTAC
CAGGATGTGGGCAGCCTCAACATAGGAGATGTCCAGCTGGAGAAGCCGTGA

pWF-396:

(SEQ ID NO: 59)
QSVLTQPPSVSAAPGQRVTISCSGTRSNIGSDYVSWYQHLPGTAPKLLVYGDNLRPS
GIPDRFSASKSGTSATLGITGLQTGDEADYYCGTWDYTLNGVVFGGGTKLTVLGSR
GGGGSGGGGSGGGGSLEMAQVQLVESGGGLVQPGGSLRLSCAASGFTFSSYAMSW
VRQAPGKGLEWVSVIYSGGSSTYYADSVKGRFTISRDNSKNTLYLQMNSLRAEDTA
VYYCARTSYLNHGDYWGQGTLVTVSSAAAFVPVFLPAKPTTTPAPRPPTPAPTIASQ
PLSLRPEACRPAAGGAVHTRGLDFACDFWVLVVVGGVLACYSLLVTVAFIIFWVRK
RWQNEKLGLDAGDEYEDENLYEGLNLDDCSMYEDISRGLQGTYQDVGSLNIGDVQ
LEKP

pWF-397:

(SEQ ID NO: 60)
CAGTCTGTGTTGACGCAGCCGCCCTCAGTGTCTGCGGCCCCAGGACAGAGGGTC
ACCATCTCCTGCTCTGGAACCAGGTCCAACATTGGGAGTGATTATGTTTCCTGGT
ACCAACACCTCCCAGGAACAGCCCCCAAACTCCTCGTTTATGGCGATAATCTGC
GACCCTCAGGGATTCCTGACCGATTCTCTGCCTCCAAGTCTGGCACGTCAGCCAC
CCTGGGCATCACCGGACTCCAGACTGGGGACGAGGCCGATTATTACTGCGGCAC
ATGGGATTACACCCTGAATGGTGTGGTGTTCGGCGGAGGGACCAAGCTGACCGT
CCTAGGTTCTAGAGGTGGTGGTGGTAGCGGCGGCGGCGGCTCTGGTGGTGGTGG
ATCCCTCGAGATGGCCCAGGTGCAGCTGGTGGAGTCTGGGGGAGGCTTGGTACA
GCCTGGGGGGTCCCTGAGACTCTCCTGTGCAGCCTCTGGATTCACCTTTAGCAGC
TATGCCATGAGCTGGGTCCGCCAGGCTCCAGGGAAGGGGCTGGAGTGGGTCTCA
GTTATTTATAGCGGTGGTAGTAGCACATACTATGCAGACTCCGTGAAGGGCCGG
TTCACCATCTCCAGAGATAATTCCAAGAACACGCTGTATCTGCAAATGAACAGC
CTGAGAGCCGAGGACACGGCCGTATATTACTGTGCGCGCACTTCTTACCTGAAC
CATGGTGATTACTGGGGTCAAGGTACTCTGGTGACCGTGTCTAGCGCCGCTGCA
TTCGTGCCTGTGTTCCTCCCAGCTAAGCCCACTACCACCCCCGCTCCAAGGCCGC
CCACGCCCGCTCCTACTATTGCTAGTCAGCCTTTAAGTTTACGACCCGAAGCTTG
CAGGCCCGCCGCCGGCGGCGCTGTGCACACCAGGGGGCTTGATTTTGCCTGCGA
CTTTTGGGTATTGGTAGTGGTGGGCGGAGTTTTAGCCTGCTACAGCCTCCTGGTA
ACAGTGGCTTTTATCATCTTTTGGGTGCTGGACAAGGATGACAGCAAGGCTGGC
ATGGAGGAAGATCACACCTACGAGGGCCTGGACATTGACCAGACAGCCACCTAT
GAGGACATAGTGACGCTGCGGACAGGGGAAGTGAAGTGGTCTGTAGGTGAGCA
CCCAGGCCAGGAGTGA

pWF-397:

(SEQ ID NO: 61)
QSVLTQPPSVSAAPGQRVTISCSGTRSNIGSDYVSWYQHLPGTAPKLLVYGDNLRPS
GIPDRFSASKSGTSATLGITGLQTGDEADYYCGTWDYTLNGVVFGGGTKLTVLGSR
GGGGSGGGGSGGGGSLEMAQVQLVESGGGLVQPGGSLRLSCAASGFTFSSYAMSW
VRQAPGKGLEWVSVIYSGGSSTYYADSVKGRFTISRDNSKNTLYLQMNSLRAEDTA
VYYCARTSYLNHGDYWGQGTLVTVSSAAAFVPVFLPAKPTTTPAPRPPTPAPTIASQ
PLSLRPEACRPAAGGAVHTRGLDFACDFWVLVVVGGVLACYSLLVTVAFIIFWVLD
KDDSKAGMEEDHTYEGLDIDQTATYEDIVTLRTGEVKWSVGEHPGQE

pWF-460:

(SEQ ID NO: 62)
CAGTCTGTGTTGACGCAGCCGCCCTCAGTGTCTGCGGCCCCAGGACAGAGGGTC
ACCATCTCCTGCTCTGGAACCAGGTCCAACATTGGGAGTGATTATGTTTCCTGGT
ACCAACACCTCCCAGGAACAGCCCCCAAACTCCTCGTTTATGGCGATAATCTGC
GACCCTCAGGGATTCCTGACCGATTCTCTGCCTCCAAGTCTGGCACGTCAGCCAC
CCTGGGCATCACCGGACTCCAGACTGGGGACGAGGCCGATTATTACTGCGGCAC
ATGGGATTACACCCTGAATGGTGTGGTGTTCGGCGGAGGGACCAAGCTGACCGT
CCTAGGTTCTAGAGGTGGTGGTGGTAGCGGCGGCGGCGGCTCTGGTGGTGGTGG
ATCCCTCGAGATGGCCCAGGTGCAGCTGGTGGAGTCTGGGGGAGGCTTGGTACA
GCCTGGGGGGTCCCTGAGACTCTCCTGTGCAGCCTCTGGATTCACCTTTAGCAGC
TATGCCATGAGCTGGGTCCGCCAGGCTCCAGGGAAGGGGCTGGAGTGGGTCTCA
GTTATTTATAGCGGTGGTAGTAGCACATACTATGCAGACTCCGTGAAGGGCCGG
TTCACCATCTCCAGAGATAATTCCAAGAACACGCTGTATCTGCAAATGAACAGC
CTGAGAGCCGAGGACACGGCCGTATATTACTGTGCGCGCACTTCTTACCTGAAC
CATGGTGATTACTGGGGTCAAGGTACTCTGGTGACCGTGTCTAGCCCCAAGAGC
TGCGACAAGACCCACACCTGCCCCCCCTGCCCAGCCCCAGAGCTGCTGGGCGGA
CCCTCCGTGTTCCTGTTCCCCCCCAAGCCCAAGGACACCCTGATGATCAGCAGG
ACCCCCGAGGTGACCTGCGTGGTGGTGGACGTGAGCCACGAGGACCCAGAGGT
GAAGTTCAACTGGTACGTGGACGGCGTGGAGGTGCACAACGCCAAGACCAAGC
CCAGAGAGGAGCAGTACAACAGCACCTACAGGGTGGTGTCCGTGCTGACCGTGC
TGCACCAGGACTGGCTGAACGGCAAGGAATACAAGTGCAAGGTCTCCAACAAG
GCCCTGCCAGCCCCCATCGAAAAGACCATCAGCAAGGCCAAGGGCCAGCCACG
GGAGCCCCAGGTGTACACCCTGCCCCCCTCCCGGGAGGAGATGACCAAGAACCA
GGTGTCCCTGACCTGTCTGGTGAAGGGCTTCTACCCCAGCGACATCGCCGTGGA
GTGGGAGAGCAACGGCCAGCCCGAGAACAACTACAAGACCACCCCCCCAGTGC
TGGACAGCGACGGCAGCTTCTTCCTGTACAGCAAGCTGACCGTGGACAAGTCCA
GGTGGCAGCAGGGCAACGTGTTCAGCTGCAGCGTGATGCACGAGGCCCTGCACA
ACCACTACACCCAGAAGAGCCTGAGCCTGTCCCCCGAGCTGCAACTGGAGGAGA
GCTGTGCGGAGGCGCAGGACGGGGAGCTGGACGGGCTGTGGACGACCATCACC
ATCTTCATCACACTCTTCCTGTTAAGCGTGTGCTACAGTGCCACCGTCACCTTCTT
CAAGGTGAAGTGGATCTTCTCCTCGGTGGTGGACCTGAAGCAGACCATCATCCC
CGACTACAGGAACATGATCGGACAGGGGGCCTGA

pWF-460:

(SEQ ID NO: 63)
QSVLTQPPSVSAAPGQRVTISCSGTRSNIGSDYVSWYQHLPGTAPKLLVYGDNLRPS
GIPDRFSASKSGTSATLGITGLQTGDEADYYCGTWDYTLNGVVFGGGTKLTVLGSR
GGGGSGGGGSGGGGSLEMAQVQLVESGGGLVQPGGSLRLSCAASGFTFSSYAMSW
VRQAPGKGLEWVSVIYSGGSSTYYADSVKGRFTISRDNSKNTLYLQMNSLRAEDTA
VYYCARTSYLNHGDYWGQGTLVTVSSPKSCDKTHTCPPCPAPELLGGPSVFLFPPKP
KDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVDKS
RWQQGNVFSCSVMHEALHNHYTQKSLSLSPELQLEESCAEAQDGELDGLWTTITIFI
TLFLLSVCYSATVTFFKVKWIFSSVVDLKQTIIPDYRNMIGQGA

pWF-428:

(SEQ ID NO: 64)
CAGTCTGTGTTGACGCAGCCGCCCTCAGTGTCTGCGGCCCCAGGACAGAGGGTC
ACCATCTCCTGCTCTGGAACCAGGTCCAACATTGGGAGTGATTATGTTTCCTGGT
ACCAACACCTCCCAGGAACAGCCCCCAAACTCCTCGTTTATGGCGATAATCTGC
GACCCTCAGGGATTCCTGACCGATTCTCTGCCTCCAAGTCTGGCACGTCAGCCAC
CCTGGGCATCACCGGACTCCAGACTGGGGACGAGGCCGATTATTACTGCGGCAC
ATGGGATTACACCCTGAATGGTGTGGTGTTCGGCGGAGGGACCAAGCTGACCGT
CCTAGGTCAGCCCAAGGCCAACCCCACTGTCACTCTGTTCCCGCCCTCCTCTGAG
GAGCTCCAAGCCAACAAGGCCACACTAGTGTGTCTGATCAGTGACTTCTACCCG
GGAGCTGTGACAGTGGCCTGGAAGGCAGATGGCAGCCCCGTCAAGGCGGGAGT
GGAGACCACCAAACCCTCCAAACAGAGCAACAACAAGTACGCGGCCAGCAGCT
ACCTGAGCCTGACGCCCGAGCAGTGGAAGTCCCACAGAAGCTACAGCTGCCAG
GTCACGCATGAAGGGAGCACCGTGGAGAAGACAGTGGCCCCTACAGAATGTTC
A

pWF-428:

(SEQ ID NO: 65)
QSVLTQPPSVSAAPGQRVTISCSGTRSNIGSDYVSWYQHLPGTAPKLLVYGDNLRPS
GIPDRFSASKSGTSATLGITGLQTGDEADYYCGTWDYTLNGVVFGGGTKLTVLGQP
KANPTVTLFPPSSEELQANKATLVCLISDFYPGAVTVAWKADGSPVKAGVETTKPS
KQSNNKYAASSYLSLTPEQWKSHRSYSCQVTHEGSTVEKTVAPTECS

pWF-429:

(SEQ ID NO: 66)
CAGGTGCAGCTGGTGGAGTCTGGGGGAGGCTTGGTACAGCCTGGGGGGTCCCTG
AGACTCTCCTGTGCAGCCTCTGGATTCACCTTTAGCAGCTATGCCATGAGCTGGG
TCCGCCAGGCTCCAGGGAAGGGGCTGGAGTGGGTCTCAGTTATTTATAGCGGTG
GTAGTAGCACATACTATGCAGACTCCGTGAAGGGCCGGTTCACCATCTCCAGAG
ATAATTCCAAGAACACGCTGTATCTGCAAATGAACAGCCTGAGAGCCGAGGACA
CGGCCGTATATTACTGTGCGCGCACTTCTTACCTGAACCATGGTGATTACTGGGG
TCAAGGTACTCTGGTGACCGTGTCTAGCGCCTCCACCAAGGGCCCATCGGTCTTC
CCCCTGGCACCCTCCTCCAAGAGCACCTCTGGGGGCACAGCGGCCCTGGGCTGC
CTGGTCAAGGACTACTTCCCCGAACCGGTGACGGTGTCGTGGAACTCAGGCGCC
CTGACCAGCGGCGTGCACACCTTCCCGGCTGTCCTACAGTCCTCAGGACTCTACT
CCCTCAGCAGCGTGGTGACCGTGCCCTCCAGCAGCTTGGGCACCCAGACCTACA
TCTGCAACGTGAACCACAAGCCCAGCAACACCAAGGTGGACAAGAGAGTGGAG
CCCAAGAGCTGCGACAAGACCCACACCTGCCCCCCCTGCCCAGCCCCAGAGCTG
CTGGGCGGACCCTCCGTGTTCCTGTTCCCCCCCAAGCCCAAGGACACCCTGATG
ATCAGCAGGACCCCCGAGGTGACCTGCGTGGTGGTGGACGTGAGCCACGAGGA
CCCAGAGGTGAAGTTCAACTGGTACGTGGACGGCGTGGAGGTGCACAACGCCA
AGACCAAGCCCAGAGAGGAGCAGTACAACAGCACCTACAGGGTGGTGTCCGTG
CTGACCGTGCTGCACCAGGACTGGCTGAACGGCAAGGAATACAAGTGCAAGGT
CTCCAACAAGGCCCTGCCAGCCCCCATCGAAAAGACCATCAGCAAGGCCAAGG
GCCAGCCACGGGAGCCCCAGGTGTACACCCTGCCCCCCTCCCGGGAGGAGATGA
CCAAGAACCAGGTGTCCCTGACCTGTCTGGTGAAGGGCTTCTACCCCAGCGACA
TCGCCGTGGAGTGGGAGAGCAACGGCCAGCCCGAGAACAACTACAAGACCACC
CCCCCAGTGCTGGACAGCGACGGCAGCTTCTTCCTGTACAGCAAGCTGACCGTG
GACAAGTCCAGGTGGCAGCAGGGCAACGTGTTCAGCTGCAGCGTGATGCACGA
GGCCCTGCACAACCACTACACCCAGAAGAGCCTGAGCCTGTCCCCCGAGCTGCA
ACTGGAGGAGAGCTGTGCGGAGGCGCAGGACGGGGAGCTGGACGGGCTGTGGA
CGACCATCACCATCTTCATCACACTCTTCCTGTTAAGCGTGTGCTACAGTGCCAC
CGTCACCTTCTTCAAGGTGAAGTGGATCTTCTCCTCGGTGGTGGACCTGAAGCAG
ACCATCATCCCCGACTACAGGAACATGATCGGACAGGGGGCCTGA

pWF-429:

(SEQ ID NO: 67)
QVQLVESGGGLVQPGGSLRLSCAASGFTFSSYAMSWVRQAPGKGLEWVSVIYSGGS
STYYADSVKGRFTISRDNSKNTLYLQMNSLRAEDTAVYYCARTSYLNHGDYWGQG
TLVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEPKSCDKTH
TCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPELQL
EESCAEAQDGELDGLWTTITIFITLFLLSVCYSATVTFFKVKWIFSSVVDLKQTIIPDY
RNMIGQGA-

mu CXCL13

(SEQ ID NO: 68)
ATGAGACTTTCAACAGCAACACTCCTCCTGTTGCTGGCTTCATGTCTGAGCCCTG
GTCATGGTATTTTGGAGGCCCACTATACAAATCTCAAATGTCGGTGTTCAGGCGT
AATATCCACCGTAGTCGGCCTGAACATTATCGATAGGATTCAGGTTACACCCCC
CGGGAACGGATGTCCTAAGACCGAGGTGGTGATTTGGACCAAGATGAAGAAGG
TCATTTGTGTGAACCCACGGGCTAAATGGCTGCAGCGTCTTTTGCGACACGTGCA
GTCCAAGAGCTTGTCCAGCACACCTCAGGCCCCAGTTAGCAAGCGACGTGCAGC
C

mu CXCL13

(SEQ ID NO: 69)
MRLSTATLLLLLASCLSPGHGILEAHYTNLKCRCSGVISTVVGLNIIDRIQVTPPGNG
CPKTEVVIWTKMKKVICVNPRAKWLQRLLRHVQSKSLSSTPQ APVSKRRAA

mu FLT3LG

(SEQ ID NO: 70)
ATGACAGTGCTGGCCCCCGCGTGGTCTCCCAATAGCTCACTCCTCCTCTTGCTGC
TACTGCTCAGCCCATGCCTCAGGGGCACCCCCGATTGTTACTTCAGCCACAGCCC
AATCTCCTCCAACTTCAAAGTGAAATTTAGGGAACTGACCGACCACCTGCTGAA
AGATTATCCTGTGACTGTGGCAGTGAACCTGCAAGACGAAAAGCATTGTAAGGC
GCTATGGAGCCTCTTTCTTGCCCAACGATGGATTGAGCAACTCAAAACTGTAGC
CGGAAGCAAAATGCAGACGCTACTGGAGGACGTGAATACTGAGATTCACTTCGT
TACCAGTTGTACTTTCCAGCCACTGCCAGAGTGTCTCAGGTTTGTGCAGACTAAT
ATCAGCCACCTGCTGAAGGATACTTGCACCCAGCTCCTGGCTCTCAAGCCTTGTA
TAGGCAAGGCTTGTCAAAATTTTAGCAGGTGTCTCGAAGTCCAGTGCCAGCCAG
ATTCATCCACACTGCTGCCGCCCCGAAGCCCTATCGCACTCGAAGCGACAGAGT
TGCCAGAGCCTCGTCCCAGACAGCTTCTGCTGCTGCTACTTCTGCTGCTGCCGCT
AACTCTGGTGCTACTTGCTGCCGCCTGGGGCCTCAGATGGCAACGCGCCAGACG
CCGAGGCGAACTCCACCCTGGGGTGCCACTGCCATCCCACCCA

mu FLT3LG

(SEQ ID NO: 71)
MTVLAPAWSPNSSLLLLLLLLSPCLRGTPDCYFSHSPISSNFKVKFRELTDHLLKDYP
VTVAVNLQDEKHCKALWSLFLAQRWIEQLKTVAGSKMQTLLEDVNTEIHFVTSCTF
QPLPECLRFVQTNISHLLKDTCTQLLALKPCIGKACQNFSRCLEVQCQPDSSTLLPPR
SPIALEATELPEPRPRQLLLLLLLLLPLTLVLLAAAWGLRWQRARRRGELHPGVPLPS
HP

mu XCL1

(SEQ ID NO: 72)
ATGCGACTCTTGTTGTTGACTTTTCTCGGAGTGTGCTGCCTGACACCCTGGGTCG
TAGAGGGAGTTGGCACTGAAGTACTAGAAGAGTCCTCCTGCGTTAACCTGCAGA
CACAGCGGCTCCCAGTCCAGAAAATTAAGACCTACATTATATGGGAAGGAGCAA
TGCGAGCGGTGATTTTTGTGACCAAGAGGGGTCTCAAGATTTGCGCGGACCCTG
AGGCCAAGTGGGTCAAAGCAGCTATTAAGACAGTAGACGGAAGAGCCTCCACC
AGGAAGAATATGGCAGAAACTGTACCGACCGGTGCGCAGCGGTCAACATCTAC
CGCAATCACACTCACCGGC

mu XCL1

(SEQ ID NO: 73)
MRLLLLTFLGVCCLTPWVVEGVGTEVLEESSCVNLQTQRLPVQKIKTYIIWEGAMR
AVIFVTKRGLKICADPEAKWVKAAIKTVDGRASTRKNMAETVPTGAQRSTSTAI
TLTG

mu Tim4(ECD)-muIgG2a Fc

(SEQ ID NO: 74)
ATGAGCAAGGGCCTTCTCCTGCTGTGGCTAGTAACTGAATTGTGGTGGTTGTACC
TGACACCTGCCGCTAGTGAGGACACCATCATTGGTTTCCTTGGGCAGCCCGTCAC
CCTCCCTTGCCATTACCTAAGCTGGAGCCAGTCACGGAACTCTATGTGCTGGGG
AAAGGGGTCATGCCCTAATTCCAAGTGCAACGCCGAGCTGTTGCGCACGGACGG
CACCAGAATAATCTCAAGAAAGTCCACCAAGTATACGCTGCTCGGCAAGGTGCA
ATTCGGTGAAGTGAGCTTGACCATAAGTAACACCAACCGCGGTGACTCCGGAGT
TTATTGTTGCAGGATCGAAGTGCCAGGCTGGTTTAACGACGTGAAGAAAAACGT
GCGGCTGGAACTGAGGAGGGCAACTACGACCAAGAAACCAACAACCACGACGA
GACCTACCACCACTCCTTACGTGACAACCACGACACCGGAGCTGTTGCCAACTA
CCGTCATGACAACATCTGTGTTGCCAACTACCACCCCCCCCCAAACGCTCGCGA
CAACTGCCTTTTCCACAGCCGTTACCACATGTCCTTCCACCACCCCAGGCTCTTT
TTCTCAAGAAACTACCAAGGGATCAGCTTTTACCACCGAGTCTGAAACTCTCCC
AGCAAGTAATCACTCACAGCGGTCAATGATGACCATCAGCACAGACATCGCTGT
CTTGAGACCTACTGGCAGCAATCCAGGCATTCTGCCCTCCACTTCACAGCTGACT
ACCCAAAAGACTACACTAACCACCAGCGAAAGTCTGCAGAAAACTACAAAGAG
CCATCAAATAAACTCCCGGCAGACTCCCAGAGGGCCCACAATCAAGCCCTGTCC
TCCATGCAAATGCCCAGCACCTAACCTCTTGGGTGGACCATCCGTCTTCATCTTC
CCTCCAAAGATCAAGGATGTACTCATGATCTCCCTGAGCCCCATAGTCACATGT
GTGGTGGTGGATGTGAGCGAGGATGACCCAGATGTCCAGATCAGCTGGTTTGTG
AACAACGTGGAAGTACACACAGCTCAGACACAAACCCATAGAGAGGATTACAA
CAGTACTCTCCGGGTGGTCAGTGCCCTCCCCATCCAGCACCAGGACTGGATGAG
TGGCAAGGAGTTCAAATGCAAGGTCAACAACAAAGACCTCCCAGCGCCCATCG
AGAGAACCATCTCAAAACCCAAAGGGTCAGTAAGAGCTCCACAGGTATATGTCT
TGCCTCCACCAGAAGAAGAGATGACTAAGAAACAGGTCACTCTGACCTGCATGG
TCACAGACTTCATGCCTGAAGACATTTACGTGGAGTGGACCAACAACGGGAAAA
CAGAGCTAAACTACAAGAACACTGAACCAGTCCTGGACTCTGATGGTTCTTACT
TCATGTACAGCAAGCTGAGAGTGGAAAAGAAGAACTGGGTGGAAAGAAATAGC
TACTCCTGTTCAGTGGTCCACGAGGGTCTGCACAATCACCACACGACTAAGAGC
TTCTCCCGGACTCCGGGTAAA

mu Tim4(ECD)-muIgG2a Fc

(SEQ ID NO: 75)
MSKGLLLLWLVTELWWLYLTPAASEDTIIGFLGQPVTLPCHYLSWSQSRNSMCWG
KGSCPNSKCNAELLRTDGTRIISRKSTKYTLLGKVQFGEVSLTISNTNRGDSGVYCCR
IEVPGWFNDVKKNVRLELRRATTTKKPTTTTRPTTTPYVTTTTPELLPTTVMTTSVLP
TTTPPQTLATTAFSTAVTTCPSTTPGSFSQETTKGSAFTTESETLPASNHSQRSMMTIS
TDIAVLRPTGSNPGILPSTSQLTTQKTTLTTSESLQKTTKSHQINSRQTPRGPTIKPCPP
CKCPAPNLLGGPSVFIFPPKIKDVLMISLSPIVTCVVVDVSEDDPDVQISWFVNNVEV
HTAQTQTHREDYNSTLRVVSALPIQHQDWMSGKEFKCKVNNKDLPAPIERTISKPK
GSVRAPQVYVLPPPEEEMTKKQVTLTCMVTDFMPEDIYVEWTNNGKTELNYKNTE
PVLDSDGSYFMYSKLRVEKKNWVERNSYSCSVVHEGLHNHHTTKSFSRTPGK

mu 4-1BB-L

(SEQ ID NO: 76)
ATGGATCAGCATACACTGGACGTGGAAGATACAGCCGATGCCAGACACCCTGCT
GGAACGTCCTGTCCCAGCGACGCTGCCCTGCTCAGAGACACCGGGCTGCTCGCA
GATGCTGCTCTGCTGAGTGATACCGTTCGGCCAACTAACGCGGCCCTACCCACA
GATGCCGCATATCCCGCGGTAAATGTCAGGGACCGGGAAGCTGCCTGGCCACCG
GCCCTCAATTTCTGCTCTAGACATCCGAAACTGTACGGTCTGGTCGCACTGGTAC
TGCTGCTACTTATAGCAGCTTGTGTTCCCATATTTACCCGCACTGAACCCAGACC
CGCTCTCACTATTACAACTTCACCAAACTTGGGCACACGTGAAAACAATGCAGA
TCAGGTTACCCCTGTAAGTCATATTGGATGCCCCAACACCACACAACAGGGAAG
TCCGGTGTTTGCAAAACTCCTTGCTAAGAATCAGGCTTCACTGTGTAACACTACT
CTTAATTGGCACTCACAAGACGGGGCCGGGAGTAGCTATCTCAGCCAAGGTCTC
CGCTATGAAGAAGATAAGAAAGAGTTGGTGGTGGACAGCCCAGGACTCTACTA
CGTCTTCCTGGAGCTAAAACTAAGCCCCACTTTTACTAACACTGGACATAAGGTC
CAAGGTTGGGTGTCCCTCGTACTTCAAGCTAAACCCCAGGTGGACGACTTCGAT
AACCTGGCGTTGACAGTTGAGCTCTTTCCTTGCTCTATGGAAAATAAGCTCGTGG
ATCGGAGCTGGTCTCAACTGTTGCTGCTTAAAGCCGGTCATCGTCTGTCTGTTGG
ACTACGCGCATACTTGCATGGAGCCCAGGACGCATATCGTGATTGGGAACTGAG
CTACCCGAATACCACTAGCTTTGGACTATTTCTTGTTAAACCAGATAATCCTTGG
GAG

mu 4-1BB-L

(SEQ ID NO: 77)
MDQHTLDVEDTADARHPAGTSCPSDAALLRDTGLLADAALLSDTVRPTNAALPTD
AAYPAVNVRDREAAWPPALNFCSRHPKLYGLVALVLLLLIAACVPIFTRTEPRPALT
ITTSPNLGTRENNADQVTPVSHIGCPNTTQQGSPVFAKLLAKNQASLCNTTLNWHSQ
DGAGSSYLSQGLRYEEDKKELVVDSPGLYYVFLELKLSPTFTNTGHKVQGWVSLVL
QAKPQVDDFDNLALTVELFPCSMENKLVDRSWSQLLLLKAGHRLSVGLRAYLHGA
QDAYRDWELSYPNTTSFGLFLVKPDNPWE

mu LIGHT (cleavage-deficient mutant)

(SEQ ID NO: 78)
ATGGAGAGCGTAGTGCAACCCAGCGTATTTGTGGTGGATGGACAGACCGACATC
CCATTCAGACGCTTGGAACAGAACCACCGAAGAAGGCGGTGCGGCACCGTCCA
GGTGTCCCTCGCTCTCGTGCTGCTGCTTGGTGCTGGCCTCGCAACACAAGGGTGG
TTTCTTTTGAGACTCCATCAACGCTTGGGAGACATAGTGGCCCACCTGCCTGATG
GTGGGAAGGGCTCTTGGCAGGACCAGCGATCACACCAGGCTAACCCCGCCGCTC
ACCTGACAGGGGCGAATGCCAGCTTGATCGGAATAGGTGGGCCGCTGCTGTGGG
AAACTAGGCTTGGACTTGCCTTTCTGAGAGGGCTTACATACCATGACGGAGCCC
TCGTAACAATGGAGCCTGGTTATTACTACGTGTACAGTAAGGTGCAGCTTTCTGG
AGTCGGGTGTCCCCAGGGGCTGGCTAACGGACTGCCCATCACTCATGGACTATA
CAAACGCACATCCAGATATCCTAAAGAGCTGGAACTGTTGGTGTCCCGTAGGAG
CCCGTGTGGCAGGGCCAACTCTTCCCGTGTGTGGTGGGACTCCTCTTTTCTGGGC
GGCGTGGTCCATCTGGAAGCTGGTGAGGAAGTCGTCGTAAGAGTACCTGGAAAC
CGTCTGGTTCGCCCCCGCGATGGCACCAGGTCCTACTTCGGAGCTTTCATGGTA

mu LIGHT (cleavage-deficient mutant)

(SEQ ID NO: 79)
MESVVQPSVFVVDGQTDIPFRRLEQNHRRRRCGTVQVSLALVLLLGAGLATQGWFL
LRLHQRLGDIVAHLPDGGKGSWQDQRSHQANPAAHLTGANASLIGIGGPLLWETRL
GLAFLRGLTYHDGALVTMEPGYYYVYSKVQLSGVGCPQGLANGLPITHGLYKRTS
RYPKELELLVSRRSPCGRANSSRVWWDSSFLGGVVHLEAGEEVVVRVPGNRLVRPR
DGTRSYFGAFMV

mu IL12 (transmembrane form)

(SEQ ID NO: 80)
ATGTGCCCACAGAAACTCACAATTTCTTGGTTCGCAATCGTCCTGCTGGTGTCAC
CCCTGATGGCAATGTGGGAGTTGGAAAAGGATGTATACGTCGTCGAGGTCGACT
GGACACCTGACGCTCCGGGTGAAACTGTCAACCTCACTTGCGATACTCCTGAAG
AGGACGACATCACGTGGACGAGCGACCAGCGACATGGAGTGATAGGGTCTGGC
AAGACGCTTACTATCACGGTTAAGGAATTTCTCGACGCAGGGCAGTACACATGT
CACAAGGGCGGCGAGACTCTGAGCCACTCCCATTTGCTGCTGCACAAGAAGGAG
AATGGTATCTGGTCTACCGAAATCCTGAAGAATTTTAAGAACAAGACTTTTCTG
AAATGCGAGGCCCCAAATTATTCCGGACGTTTCACTTGCAGTTGGCTCGTTCAAA
GAAATATGGACTTGAAATTTAACATTAAATCCAGCTCTTCATCTCCTGACAGCAG
GGCCGTAACTTGTGGAATGGCTTCATTGTCAGCTGAGAAAGTTACGCTTGACCA
AAGGGATTATGAGAAATACAGCGTGAGTTGCCAGGAAGATGTGACATGTCCAA
CGGCAGAGGAAACGTTGCCAATTGAGCTCGCTTTGGAAGCTCGTCAACAAAACA
AGTATGAAAACTATAGTACTAGCTTCTTCATACGGGACATCATCAAACCAGATC
CACCTAAGAATTTGCAGATGAAGCCTCTGAAGAATTCACAAGTCGAGGTATCCT
GGGAATACCCAGATTCATGGTCCACTCCTCATAGTTACTTTAGCCTGAAATTCTT
TGTACGCATACAGCGGAAGAAGGAGAAAATGAAGGAGACGGAAGAAGGCTGC
AATCAGAAAGGCGCTTTTCTTGTTGAAAAGACGAGCACTGAGGTTCAATGCAAA
GGCGGGAATGTATGTGTTCAAGCCCAAGATAGGTATTATAATAGCTCCTGCTCT
AAGTGGGCTTGCGTACCATGCAGAGTTAGAAGTGGCTCAACCTCAGGCTCCGGA
AAACCTGGTTCCGGTGAAGGTTCCACAAAAGGGCGTGTGATTCCTGTGTCCGGC
CCAGCTAGGTGTCTCTCCCAGTCACGGAATCTCCTGAAAACCACGGATGACATG
GTAAAGACAGCTAGGGAGAAACTCAAGCACTACTCCTGCACAGCTGAGGATATC
GATCATGAGGACATCACCAGGGACCAGACATCCACTCTGAAAACTTGCCTGCCT
TTGGAACTCCACAAGAACGAATCTTGTCTGGCAACGCGTGAAACGAGTTCTACT
ACAAGAGGGTCCTGTCTTCCCCCTCAAAAGACAAGCCTTATGATGACCTTGTGTC
TCGGTAGCATTTATGAGGACCTAAAGATGTATCAAACCGAGTTTCAGGCTATCA
ATGCAGCGCTCCAGAATCATAACCATCAGCAGATCATTCTTGACAAAGGAATGC
TCGTGGCCATTGATGAACTAATGCAGAGCCTAAACCACAATGGCGAGACTCTTC
GACAGAAACCGCCTGTGGGCGAGGCCGATCCATATAGAGTCAAAATGAAACTG
TGTATTCTCCTGCATGCATTTAGTACTCGTGTAGTGACTATTAACAGAGTGATGG
GTTACCTTTCCTCAGCTAATACACTTGTCCTCTTTGGCGCTGGGTTCGGCGCCGT
CATAACGGTTGTTGTCATCGTGGTAATAATCAAGTGCTTTTGCAAGCACAGGTCT
TGTTTTCGCAGGAATGAAGCCTCTAGAGAAACAAATAATTCACTGACCTTTGGC
CCCGAAGAAGCTCTTGCAGAGCAAACGGTGTTTCTC

mu IL12 (transmembrane form)

(SEQ ID NO: 81)
MCPQKLTISWFAIVLLVSPLMAMWELEKDVYVVEVDWTPDAPGETVNLTCDTPEE
DDITWTSDQRHGVIGSGKTLTITVKEFLDAGQYTCHKGGETLSHSHLLLHKKENGI
WSTEILKNFKNKTFLKCEAPNYSGRFTCSWLVQRNMDLKFNIKSSSSSPDSRAVTCG
MASLSAEKVTLDQRDYEKYSVSCQEDVTCPTAEETLPIELALEARQQNKYENYSTSF
FIRDIIKPDPPKNLQMKPLKNSQVEVSWEYPDSWSTPHSYFSLKFFVRIQRKKEKMK
ETEEGCNQKGAFLVEKTSTEVQCKGGNVCVQAQDRYYNSSCSKWACVPCRVRSGS
TSGSGKPGSGEGSTKGRVIPVSGPARCLSQSRNLLKTTDDMVKTAREKLKHYSCTA
EDIDHEDITRDQTSTLKTCLPLELHKNESCLATRETSSTTRGSCLPPQKTSLMMTLCL
GSIYEDLKMYQTEFQAINAALQNHNHQQIILDKGMLVAIDELMQSLNHNGETLRQK
PPVGEADPYRVKMKLCILLHAFSTRVVTINRVMGYLSSANTLVLFGAGFGAVITVV
VIVVIIKCFCKHRSCFRRNEASRETNNSLTFGPEEALAEQTVFL

mu IL12 (secreted form)

(SEQ ID NO: 82)
ATGTGTCAGTCACGCTATCTTCTCTTCCTTGCTACTCTGGCCTTGCTCAATCACTT
GTCCCTTGCTCGTGTGATTCCTGTGTCCGGCCCAGCTAGGTGTCTCTCCCAGTCA
CGGAATCTCCTGAAAACCACGGATGACATGGTAAAGACAGCTAGGGAGAAACT
CAAGCACTACTCCTGCACAGCTGAGGATATCGATCATGAGGACATCACCAGGGA
CCAGACATCCACTCTGAAAACTTGCCTGCCTTTGGAACTCCACAAGAACGAATC
TTGTCTGGCAACGCGTGAAACGAGTTCTACTACAAGAGGGTCCTGTCTTCCCCCT
CAAAAGACAAGCCTTATGATGACCTTGTGTCTCGGTAGCATTTATGAGGACCTA
AAGATGTATCAAACCGAGTTTCAGGCTATCAATGCAGCGCTCCAGAATCATAAC
CATCAGCAGATCATTCTTGACAAAGGAATGCTCGTGGCCATTGATGAACTAATG
CAGAGCCTAAACCACAATGGCGAGACTCTTCGACAGAAACCGCCTGTGGGCGA
GGCCGATCCATATAGAGTCAAAATGAAACTGTGTATTCTCCTGCATGCATTTAGT
ACTCGTGTAGTGACTATTAACAGAGTGATGGGTTACCTTTCCTCAGCTGGAAGC
GGCGCCACCAACTTCTCCCTGCTGAAGCAGGCCGGCGACGTGGAGGAGAACCCC
GGCCCCATGTGCCCACAGAAACTCACAATTTCTTGGTTCGCAATCGTCCTGCTGG
TGTCACCCCTGATGGCAATGTGGGAGTTGGAAAAGGATGTATACGTCGTCGAGG
TCGACTGGACACCTGACGCTCCGGGTGAAACTGTCAACCTCACTTGCGATACTC
CTGAAGAGGACGACATCACGTGGACGAGCGACCAGCGACATGGAGTGATAGGG
TCTGGCAAGACGCTTACTATCACGGTTAAGGAATTTCTCGACGCAGGGCAGTAC
ACATGTCACAAGGGCGGCGAGACTCTGAGCCACTCCCATTTGCTGCTGCACAAG
AAGGAGAATGGTATCTGGTCTACCGAAATCCTGAAGAATTTTAAGAACAAGACT
TTTCTGAAATGCGAGGCCCCAAATTATTCCGGACGTTTCACTTGCAGTTGGCTCG
TTCAAAGAAATATGGACTTGAAATTTAACATTAAATCCAGCTCTTCATCTCCTGA
CAGCAGGGCCGTAACTTGTGGAATGGCTTCATTGTCAGCTGAGAAAGTTACGCT
TGACCAAAGGGATTATGAGAAATACAGCGTGAGTTGCCAGGAAGATGTGACAT
GTCCAACGGCAGAGGAAACGTTGCCAATTGAGCTCGCTTTGGAAGCTCGTCAAC
AAAACAAGTATGAAAACTATAGTACTAGCTTCTTCATACGGGACATCATCAAAC
CAGATCCACCTAAGAATTTGCAGATGAAGCCTCTGAAGAATTCACAAGTCGAGG
TATCCTGGGAATACCCAGATTCATGGTCCACTCCTCATAGTTACTTTAGCCTGAA
ATTCTTTGTACGCATACAGCGGAAGAAGGAGAAAATGAAGGAGACGGAAGAAG
GCTGCAATCAGAAAGGCGCTTTTCTTGTTGAAAAGACGAGCACTGAGGTTCAAT
GCAAAGGCGGGAATGTATGTGTTCAAGCCCAAGATAGGTATTATAATAGCTCCT
GCTCTAAGTGGGCTTGCGTACCATGCAGAGTTAGAAGT

mu IL12 (secreted form)

(SEQ ID NO: 83)
MCQSRYLLFLATLALLNHLSLARVIPVSGPARCLSQSRNLLKTTDDMVKTAREKLK
HYSCTAEDIDHEDITRDQTSTLKTCLPLELHKNESCLATRETSSTTRGSCLPPQKTSL
MMTLCLGSIYEDLKMYQTEFQAINAALQNHNHQQIILDKGMLVAIDELMQSLNHN
GETLRQKPPVGEADPYRVKMKLCILLHAFSTRVVTINRVMGYLSSAGSGATNFSLL
KQAGDVEENPGPMCPQKLTISWFAIVLLVSPLMAMWELEKDVYVVEVDWTPDAPG
ETVNLTCDTPEEDDITWTSDQRHGVIGSGKTLTITVKEFLDAGQYTCHKGGETLSHS
HLLLHKKENGIWSTEILKNFKNKTFLKCEAPNYSGRFTCSWLVQRNMDLKFNIKSSS
SSPDSRAVTCGMASLSAEKVTLDQRDYEKYSVSCQEDVTCPTAEETLPIELALEARQ
QNKYENYSTSFFIRDIIKPDPPKNLQMKPLKNSQVEVSWEYPDSWSTPHSYFSLKFFV
RIQRKKEKMKETEEGCNQKGAFLVEKTSTEVQCKGGNVCVQ
AQDRYYNSSCSKWACVPCRV RS

mu IFN alpha A2

(SEQ ID NO: 84)
ATGGCCAGGCTTTGCGCTTTTCTCGTCATGCTGATCGTCATGAGTTACTGGTCCA
TTTGCAGCCTCGGATGTGATCTGCCCCACACCTACAACCTGCGCAACAAACGAG
CTCTCAAAGTGTTGGCCCAAATGAGGCGGTTGCCCTTCCTTTCCTGTCTCAAAGA
CAGGCAAGATTTTGGATTTCCACTAGAGAAAGTAGACAATCAACAGATACAGAA
AGCTCAAGCTATCCCCGTGTTGAGGGACTTGACTCAACAGACGTTGAATCTATTT
ACTAGCAAGGCCAGCTCTGCTGCTTGGAATGCCACCCTTCTTGACTCATTTTGCA
ATGACCTACATCAACAACTGAATGATCTCCAAACATGTTTGATGCAGCAGGTAG
GTGTCCAAGAACCCCCGCTTACTCAGGAAGACGCCCTTCTGGCTGTCCGCAAGT
ACTTTCACAGAATCACAGTGTACCTGCGCGAAAAGAAACACTCCCCCTGCGCTT
GGGAAGTGGTCAGGGCCGAGGTTTGGCGAGCCCTGAGTAGCTCCGTCAATCTCC
TTCCTCGGTTGTCCGAGGAGAAAGAG

mu IFN alpha A2

(SEQ ID NO: 85)
MARLCAFLVMLIVMSYWSICSLGCDLPHTYNLRNKRALKVLAQMRRLPFLSCLKD
RQDFGFPLEKVDNQQIQKAQAIPVLRDLTQQTLNLFTSKASSAAWNATLLDSFCND
LHQQLNDLQTCLMQQVGVQEPPLTQEDALLAVRKYFHRITVYLREKKHSPCAWEV
VRAEVWRALSSSVNLLPRLSEEKE

mu CD80

(SEQ ID NO: 86)
ATGGCTTGCAACTGTCAGCTCATGCAAGATACTCCCCTGCTTAAGTTTCCCTGCC
CTAGACTCATTCTCCTCTTCGTCCTTCTCATTCGCCTAAGCCAGGTGAGTTCCGAT
GTGGATGAACAACTGAGTAAATCTGTCAAGGATAAAGTTCTGCTCCCATGCCGC
TACAATAGCCCCCATGAGGACGAGTCCGAAGATAGGATTTACTGGCAGAAACAT
GATAAGGTGGTGCTATCCGTCATTGCCGGTAAATTGAAGGTGTGGCCCGAATAT
AAGAATAGAACCCTGTATGACAACACAACTTATAGCCTAATCATCCTCGGTCTC
GTACTGAGCGACCGAGGTACTTACTCATGCGTTGTGCAGAAGAAGGAGCGCGGA
ACATACGAAGTCAAGCACCTTGCATTGGTGAAATTGTCAATAAAAGCTGACTTT
TCAACTCCTAATATTACTGAATCAGGTAACCCTTCCGCAGACACTAAAAGAATT
ACATGCTTCGCCTCTGGCGGGTTTCCCAAACCACGGTTCTCTTGGCTAGAGAATG
GGAGAGAACTTCCAGGTATCAATACAACCATCTCTCAAGACCCAGAATCAGAAC
TGTACACCATCTCCAGCCAACTCGATTTCAATACCACAAGAAATCATACAATAA
AATGTCTGATAAAGTACGGAGATGCACATGTCTCTGAAGATTTCACATGGGAGA
AACCACCAGAGGACCCGCCAGACAGCAAGAATACACTTGTCCTCTTTGGCGCTG
GGTTCGGCGCCGTCATAACGGTTGTTGTCATCGTGGTAATAATCAAGTGCTTTTG
CAAGCACAGGTCTTGTTTTCGCAGGAATGAAGCCTCTAGAGAAACAAATAATTC
ACTGACCTTTGGCCCCGAAGAAGCTCTTGCAGAGCAAACGGTGTTTCTC

mu CD80

(SEQ ID NO: 87)
MACNCQLMQDTPLLKFPCPRLILLFVLLIRLSQVSSDVDEQLSKSVKDKVLLPCRYN
SPHEDESEDRIYWQKHDKVVLSVIAGKLKVWPEYKNRTLYDNTTYSLIILGLVLSDR
GTYSCVVQKKERGTYEVKHLALVKLSIKADFSTPNITESGNPSADTKRITCFASGGFP
KPRFSWLENGRELPGINTTISQDPESELYTISSQLDFNTTRNHTIKCLIKYGDAHVSED
FTWEKPPEDPPDSKNTLVLFGAGFGAVITVVVIVVIIKCFCKHRSCFRR
NEASRETNNSLTFGPEEALAEQTVFL

mu CD40-L

(SEQ ID NO: 88)
ATGATCGAAACTTATTCCCAACCCTCACCGCGCTCAGTAGCAACTGGCCTACCA
GCCAGCATGAAGATATTCATGTACCTCTTGACTGTATTCTTGATCACGCAAATGA
TTGGTAGTGTTTTGTTCGCCGTTTATCTCCACAGGCGCCTGGATAAAGTTGAAGA
AGAGGTTAATCTCCATGAAGACTTCGTGTTCATTAAGAAACTCAAAAGATGTAA
CAAAGGTGAGGGATCTCTGTCTCTTCTGAACTGTGAGGAGATGCGACGGCAATT
CGAGGACCTCGTAAAAGACATAACTCTCAACAAAGAAGAGAAGAAAGAAAACT
CTTTCGAGATGCAACGGGGCGACGAGGACCCTCAAATTGCCGCACATGTCGTTT
CTGAAGCGAATTCCAATGCCGCGTCCGTGCTCCAGTGGGCGAAGAAGGGATACT
ACACGATGAAGAGCAACCTTGTGATGCTTGAAAATGGCAAGCAGCTCACAGTTA
AACGCGAGGGACTCTACTATGTATACACCCAAGTGACCTTTTGTTCCAACCGGG
AGCCAAGTAGCCAACGCCCGTTCATCGTTGGGCTGTGGCTCAAGCCTTCTTCAG
GGAGTGAACGAATCCTTCTCAAGGCAGCCAACACGCATTCCAGCAGCCAACTGT
GTGAGCAACAATCCGTGCATCTTGGCGGGGTCTTTGAGCTGCAAGCGGGCGCCT
CTGTGTTCGTGAATGTTACCGAAGCCAGCCAGGTTATCCACCGCGTGGGTTTCAG
TAGTTTTGGCCTGCTCAAGCTG

mu CD40-L

(SEQ ID NO: 89)
MIETYSQPSPRSVATGLPASMKIFMYLLTVFLITQMIGSVLFAVYLHRRLDKVEEEV
NLHEDFVFIKKLKRCNKGEGSLSLLNCEEMRRQFEDLVKDITLNKEEKKENSFEMQ
RGDEDPQIAAHVVSEANSNAASVLQWAKKGYYTMKSNLVMLENGKQLTVKREGL
YYVYTQVTFCSNREPSSQRPFIVGLWLKPSSGSERILLKAANTHSSSQLCEQQSVHLG
GVFELQAGASVFVNVTEASQVIHRVGFSSFGLLKL

mu IL21

(SEQ ID NO: 90)
ATGGAGCGTACTCTGGTCTGCCTTGTTGTGATATTCTTGGGGACAGTTGCACACA
AATCATCACCCCAAGGACCGGATAGACTCCTCATACGCCTGCGCCATCTGATTG
ACATTGTCGAGCAGTTGAAGATTTATGAGAACGACCTGGACCCTGAACTATTGA
GCGCGCCTCAAGACGTCAAAGGGCATTGCGAGCATGCTGCATTTGCATGTTTTC
AGAAAGCTAAGCTCAAACCAAGTAATCCCGGTAACAATAAAACATTCATCATCG
ACCTGGTGGCCCAACTAAGACGCCGGTTGCCGGCGCGCCGGGGTGGTAAGAAA
CAGAAACATATTGCTAAATGCCCCTCTTGCGACTCTTACGAGAAAAGGACACCT
AAGGAATTCCTCGAACGATTGAAATGGTTGTTGCAGAAGATGATCCATCAACAT
CTGAGC

mu IL21

(SEQ ID NO: 91)
MERTLVCLVVIFLGTVAHKSSPQGPDRLLIRLRHLIDIVEQLKIYENDLDPELLSAPQ
DVKGHCEHAAFACFQKAKLKPSNPGNNKTFIIDLVAQLRRRLPARRGGKKQK
HIAKCPSCDSYEKRTPKEFLERLKWLLQKM IHQHLS

mu CCL21

(SEQ ID NO: 92)
ATGGCACAAATGATGACACTGTCCCTACTTAGTCTAGTTCTAGCTTTGTGTATTC
CCTGGACTCAAGGCAGTGACGGAGGAGGACAAGACTGCTGCCTCAAATATTCTC
AAAAGAAAATCCCTTATTCTATAGTCCGAGGTTACCGTAAGCAAGAACCGAGTC
TAGGTTGTCCTATCCCCGCAATCCTCTTTCTACCACGGAAACATAGCAAACCAGA
ATTGTGCGCCAACCCAGAAGAGGGTTGGGTCCAAAATTTGATGAGGCGCCTTGA
CCAACCACCGGCCCCGGGTAAACAATCACCGGGGTGTCGGAAGAATAGGGGTA
CATCCAAATCCGGGAAGAAAGGGAAGGGGAGTAAGGGCTGTAAGAGAACGGA
ACAAACTCAACCTAGCAGAGGT

mu CCL21

(SEQ ID NO: 93)
MAQMMTLSLLSLVLALCIPWTQGSDGGGQDCCLKYSQKKIPYSIVRGYRKQEPSLG
CPIPAILFLPRKHSKPELCANPEEGWVQNLMRRLDQPPAPGKQSPGCRKNRGTSKSG
KKGKGSKGCKRTEQTQPSRG

anti-mu CD3 scFv-transmembrane

(SEQ ID NO: 94)
ATGGAAACCGACACATTGCTCCTCTGGGTTCTCCTTCTATGGGTCCCCGGTTCCA
CCGGAGATATCCAAATGACACAATCACCCAGCAGCCTGCCTGCCTCTCTGGGCG
ACCGCGTTACCATCAATTGTCAAGCTTCCCAAGATATAAGTAATTATCTCAACTG
GTACCAGCAAAAGCCCGGTAAAGCGCCTAAATTGCTGATTTATTATACTAATAA
ACTCGCAGATGGAGTTCCTAGTAGATTTTCTGGTTCAGGGAGTGGACGGGACTC
CAGTTTTACCATATCAAGTCTGGAATCCGAGGATATCGGCAGCTACTATTGCCA
GCAATATTATAATTACCCTTGGACTTTTGGACCCGGGACTAAACTTGAGATCAA
AAGAGGCGGAGGAGGCAGTGGTGGTGGTGGATCAGGCGGCGGTGGTAGTGAGG
TACAACTCGTGGAATCAGGCGGCGGACTGGTCCAACCCGGCAAGAGCCTTAAAC
TCTCTTGTGAGGCCAGTGGATTTACATTCAGCGGTTATGGAATGCACTGGGTGA
GACAAGCTCCCGGCAGGGGCCTAGAATCAGTGGCGTACATCACCAGCTCATCAA
TAAACATTAAATACGCTGATGCAGTCAAGGGCCGGTTTACTGTATCCCGCGACA
ACGCTAAGAATCTTCTCTTTCTGCAAATGAACATACTTAAGAGCGAGGATACTG
CCATGTATTATTGTGCCCGCTTCGATTGGGATAAGAATTATTGGGGACAAGGCA
CCATGGTTACCGTTAGTAGTCCAAACATCACATCAAATAATAGCAACCCCGTGG
AAGGGGACGACTCTGTTTCACTCACCTGTGATTCCTATACCGATCCTGATAATAT
CAACTATCTATGGTCTCGTAACGGTGAAAGTCTCAGCGAAGGCGACCGGTTGAA
ACTCTCCGAAGGTAACAGAACCCTTACGCTTCTGAACGTCACCCGGAACGATAC
CGGGCCCTATGTTTGCGAAACTAGGAACCCTGTTAGCGTGAATCGTAGCGACCC
TTTCTCCCTAAATAATACTCTAGTGCTATTCGGAGCGGGATTCGGTGCCGTCATC
ACAGTAGTCGTTATTGTAGTCATTATTAAATGCTTTTGTAAACATAGGTCTTGCT
TCAGAAGAAATGAGGCCAGCCGTGAAACTAATAATTCCCTGACCTTTGGGCCCG
AAGAAGCTTTGGCTGAACAGACTGTGTTTCTC

anti-mu CD3 scFv-transmembrane

(SEQ ID NO: 95)
METDTLLLWVLLLWVPGSTGDIQMTQSPSSLPASLGDRVTINCQASQDISNYLNWY
QQKPGKAPKLLIYYTNKLADGVPSRFSGSGSGRDSSFTISSLESEDIGSYYCQQYYNY
PWTFGPGTKLEIKRGGGGSGGGGSGGGGSEVQLVESGGGLVQPGKSLKLSCEASGF
TFSGYGMHWVRQAPGRGLESVAYITSSSINIKYADAVKGRFTVSRDNAKNLLFLQM
NILKSEDTAMYYCARFDWDKNYWGQGTMVTVSSPNITSNNSNPVEGDDSVSLTCD
SYTDPDNINYLWSRNGESLSEGDRLKLSEGNRTLTLLNVTRNDTGPYVCETRNPVSV
NRSDPFSLNNTLVLFGAGFGAVITVVVIVVIIKCFCKHRSCFRRNEASRETNNSLTFG
PEEALAEQTVFL

mu TSLP

(SEQ ID NO: 96)
ATGGTTCTTCTCAGGAGCCTCTTCATCCTGCAAGTACTAGTACGGATGGGGCTAA
CTTACAACTTTTCTAACTGCAACTTCACGTCAATTACGAAAATATATTGTAACAT
AATTTTTCATGACCTGACTGGAGATTTGAAAGGGGCTAAGTTCGAGCAAATCGA
GGACTGTGAGAGCAAGCCAGCTTGTCTCCTGAAAATCGAGTACTATACTCTCAA
TCCTATCCCTGGCTGCCCTTCACTCCCCGACAAAACATTTGCCCGGAGAACAAG
AGAAGCCCTCAATGACCACTGCCCAGGCTACCCTGAAACTGAGAGAAATGACG
GTACTCAGGAAATGGCACAAGAAGTCCAAAACATCTGCCTGAATCAAACCTCAC
AAATTCTAAGATTGTGGTATTCCTTCATGCAATCTCCAGAA

mu TSLP

(SEQ ID NO: 97)
MVLLRSLFILQVLVRMGLTYNFSNCNFTSITKIYCNIIFHDLTGDLKGAKFEQIEDCES
KPACLLKIEYYTLNPIPGCPSLPDKTFARRTREALNDHCPGYPETERNDGTQEMAQE
VQNICLNQTSQILRLWYSFMQSPE

mu GM-CSF

(SEQ ID NO: 98)
ATGTGGCTGCAGAATTTACTTTTCCTGGGCATTGTGGTCTACAGCCTCTCAGCAC
CCACCCGCTCACCCATCACTGTCACCCGGCCTTGGAAGCATGTAGAGGCCATCA
AAGAAGCCCTGAACCTCCTGGATGACATGCCTGTCACGTTGAATGAAGAGGTAG
AAGTCGTCTCTAACGAGTTCTCCTTCAAGAAGCTAACATGTGTGCAGACCCGCCT
GAAGATATTCGAGCAGGGTCTACGGGGCAATTTCACCAAACTCAAGGGCGCCTT
GAACATGACAGCCAGCTACTACCAGACATACTGCCCCCCAACTCCGGAAACGGA
CTGTGAAACACAAGTTACCACCTATGCGGATTTCATAGACAGCCTTAAAACCTTT
CTGACTGATATCCCCTTTGAATGCAAAAAACCAGGCCAAAAA

mu GM-CSF

(SEQ ID NO: 99)
MWLQNLLFLGIVVYSLSAPTRSPITVTRPWKHVEAIKEALNLLDDMPVTLNEEVEV
VSNEFSFKKLTCVQTRLKIFEQGLRGNFTKLKGALNMTASYYQTYCPPTPETDCETQ
VTTYADFIDSLKTFLTDIPFECKKPGQK

mu IFN gamma

(SEQ ID NO: 100)
ATGAACGCTACACACTGCATCTTGGCTTTGCAGCTCTTCCTCATGGCTGTTTCTG
GCTGTTACTGCCACGGCACAGTCATTGAAAGCCTAGAAAGTCTGAATAACTATT
TTAACTCAAGTGGCATAGATGTGGAAGAAAAGAGTCTCTTCTTGGATATCTGGA
GGAACTGGCAAAAGGATGGTGACATGAAAATCCTGCAGAGCCAGATTATCTCTT
TCTACCTCAGACTCTTTGAAGTCTTGAAAGACAATCAGGCCATCAGCAACAACA
TAAGCGTCATTGAATCACACCTGATTACTACCTTCTTCAGCAACAGCAAGGCGA
AAAAGGATGCATTCATGAGTATTGCCAAGTTTGAGGTCAACAACCCACAGGTCC
AGCGCCAAGCATTCAATGAGCTCATCCGAGTGGTCCACCAGCTGTTGCCGGAAT
CCAGCCTCAGGAAGCGGAAAAGGAGTCGCTGC

mu IFN gamma

(SEQ ID NO: 101)
MNATHCILALQLFLMAVSGCYCHGTVIESLESLNNYFNSSGIDVEEKSLFLDIWRNW
QKDGDMKILQSQIISFYLRLFEVLKDNQAISNNISVIESHLITTFFSNSKAKKDAFMSI
AKFEVNNPQVQRQAFNELIRVVHQLLPESSLRKRKRSRC

mu IL7

(SEQ ID NO: 102)
ATGTTCCATGTTTCTTTTAGATATATCTTTGGAATTCCTCCACTGATCCTTGTTCT
GCTGCCTGTCACATCATCTGAGTGCCACATTAAAGACAAAGAAGGTAAAGCATA
TGAGAGTGTACTGATGATCAGCATCGATGAATTGGACAAAATGACAGGAACTGA
TAGTAATTGCCCGAATAATGAACCAAACTTTTTTAGAAAACATGTATGTGATGA
TACAAAGGAAGCTGCTTTTCTAAATCGTGCTGCTCGCAAGTTGAAGCAATTTCTT
AAAATGAATATCAGTGAAGAATTCAATGTCCACTTACTAACAGTATCACAAGGC
ACACAAACACTGGTGAACTGCACAAGTAAGGAAGAAAAAAACGTAAAGGAACA
GAAAAAGAATGATGCATGTTTCCTAAAGAGACTACTGAGAGAAATAAAAACTT
GTTGGAATAAAATTTTGAAGGGCAGTATA

mu IL7

(SEQ ID NO: 103)
MFHVSFRYIFGIPPLILVLLPVTSSECHIKDKEGKAYESVLMISIDELDKMTGTDSNCP
NNEPNFFRKHVCDDTKEAAFLNRAARKLKQFLKMNISEEFNVHLLTVSQGTQTLVN
CTSKEEKNVKEQKKNDACFLKRLLREIKTCWNKILKGSI

mu ICOS-L

(SEQ ID NO: 104)
ATGCAGCTAAAGTGTCCCTGTTTTGTGTCCTTGGGAACCAGGCAGCCTGTTTGGA
AGAAGCTCCATGTTTCTAGCGGGTTCTTTTCTGGTCTTGGTCTGTTCTTGCTGCTG
TTGAGCAGCCTCTGTGCTGCCTCTGCAGAGACTGAAGTCGGTGCAATGGTGGGC
AGCAATGTGGTGCTCAGCTGCATTGACCCCCACAGACGCCATTTCAACTTGAGT
GGTCTGTATGTCTATTGGCAAATCGAAAACCCAGAAGTTTCGGTGACTTACTACC
TGCCTTACAAGTCTCCAGGGATCAATGTGGACAGTTCCTACAAGAACAGGGGCC
ATCTGTCCCTGGACTCCATGAAGCAGGGTAACTTCTCTCTGTACCTGAAGAATGT
CACCCCTCAGGATACCCAGGAGTTCACATGCCGGGTATTTATGAATACAGCCAC
AGAGTTAGTCAAGATCTTGGAAGAGGTGGTCAGGCTGCGTGTGGCAGCAAACTT
CAGTACACCTGTCATCAGCACCTCTGATAGCTCCAACCCGGGCCAGGAACGTAC
CTACACCTGCATGTCCAAGAATGGCTACCCAGAGCCCAACCTGTATTGGATCAA
CACAACGGACAATAGCCTAATAGACACGGCTCTGCAGAATAACACTGTCTACTT
GAACAAGTTGGGCCTGTATGATGTAATCAGCACATTAAGGCTCCCTTGGACATC
TCGTGGGGATGTTCTGTGCTGCGTAGAGAATGTGGCTCTCCACCAGAACATCAC
TAGCATTAGCCAGGCAGAAAGTTTCACTGGAAATAACACAAAGAACCCACAGG
AAACCCACAATAATGAGTTAAAAGTCCTTGTCCCCGTCCTTGCTGTACTGGCGGC
AGCGGCATTCGTTTCCTTCATCATATACAGACGCACGCGTCCCCACCGAAGCTAT
ACAGGACCCAAGACTGTACAGCTTGAACTTACAGACCACGCC

mu ICOS-L

(SEQ ID NO: 105)
MQLKCPCFVSLGTRQPVWKKLHVSSGFFSGLGLFLLLLSSLCAASAETEVGAMVGS
NVVLSCIDPHRRHFNLSGLYVYWQIENPEVSVTYYLPYKSPGINVDSSYKNRGHLSL
DSMKQGNFSLYLKNVTPQDTQEFTCRVFMNTATELVKILEEVVRLRVAANFSTPVIS
TSDSSNPGQERTYTCMSKNGYPEPNLYWINTTDNSLIDTALQNNTVYLNKLGLYDVI
STLRLPWTSRGDVLCCVENVALHQNITSISQAESFTGNNTKNPQETHNNELKVLVPV
LAVLAAAAFVSFIIYRRTRPHRSYTGPKTVQLELTD HA

mu CD47

(SEQ ID NO: 106)
ATGTGGCCCTTGGCGGCGGCGCTGTTGCTGGGCTCCTGCTGCTGCGGTTCAGCTC
AACTACTGTTTAGTAACGTCAACTCCATAGAGTTCACTTCATGCAATGAAACTGT
GGTCATCCCTTGCATCGTCCGTAATGTGGAGGCGCAAAGCACCGAAGAAATGTT
TGTGAAGTGGAAGTTGAACAAATCGTATATTTTCATCTATGATGGAAATAAAAA
TAGCACTACTACAGATCAAAACTTTACCAGTGCAAAAATCTCAGTCTCAGACTT
AATCAATGGCATTGCCTCTTTGAAAATGGATAAGCGCGATGCCATGGTGGGAAA
CTACACTTGCGAAGTGACAGAGTTATCCAGAGAAGGCAAAACAGTTATAGAGCT
GAAAAACCGCACGGTTTCGTGGTTTTCTCCAAATGAAAAGATCCTCATTGTTATT
TTCCCAATTTTGGCTATACTCCTGTTCTGGGGAAAGTTTGGTATTTTAACACTCA
AATATAAATCCAGCCATACGAATAAGAGAATCATTCTGCTGCTCGTTGCCGGGC
TGGTGCTCACAGTCATCGTGGTTGTTGGAGCCATCCTTCTCATCCCAGGAGAAAA
GCCCGTGAAGAATGCTTCTGGACTTGGCCTCATTGTAATCTCTACGGGGATATTA
ATACTACTTCAGTACAATGTGTTTATGACAGCTTTTGGAATGACCTCTTTCACCA
TTGCCATATTGATCACTCAAGTGCTGGGCTACGTCCTTGCTTTGGTCGGGCTGTG
TCTCTGCATCATGGCATGTGAGCCAGTGCACGGCCCCCTTTTGATTTCAGGTTTG
GGGATCATAGCTCTAGCAGAACTACTTGGATTAGTTTATATGAAGTTTGTCGCTT
CCAACCAGAGGACTATCCAACCTCCTAGGAATAGG

mu CD47

(SEQ ID NO: 107)
MWPLAAALLLGSCCCGSAQLLFSNVNSIEFTSCNETVVIPCIVRNVEAQSTEEMFVK
WKLNKSYIFIYDGNKNSTTTDQNFTSAKISVSDLINGIASLKMDKRDAMVGNYTCE
VTELSREGKTVIELKNRTVSWFSPNEKILIVIFPILAILLFWGKFGILTLKYKSSHTNKR
IILLLVAGLVLTVIVVVGAILLIPGEKPVKNASGLGLIVISTGILILLQYNVFMTAFGMT
SFTIAILITQVLGYVLALVGLCLCIMACEPVHGPLLISGLGIIALAELLGLVYMKFVAS
NQRTIQPPRNR

Mu Sarcoglycan alpha:

(SEQ ID NO: 108)
ATGGCAGCAGCAGTAACTTGGATACCTCTCCTGGCAGGTCTCCTGGCAGGACTG
AGGGACACCAAGGCCCAGCAGACAACTTTACACCTACTTGTGGGTCGTGTGTTT
GTGCATCCTTTGGAACATGCCACCTTCCTGCGCCTTCCAGAACACGTTGCGGTGC
CACCCACTGTCCGACTCACCTACCACGCTCACCTCCAGGGACATCCAGACCTGC
CCAGGTGGCTGCACTACACACAGCGCAGTCCCTATAACCCTGGCTTCCTCTACG
GCTCCCCCACTCCAGAAGATCGTGGGTACCAAGTCATCGAGGTCACAGCCTACA
ATCGAGACAGTTTTGACACCACTAGACAGAGGCTGCTGCTGCTGATTGGGGACC
CCGAAGGTCCCCGGTTGCCATACCAAGCTGAGTTCCTGGTGCGCAGCCATGATG
TGGAGGAGGTGCTGCCCACCACACCTGCCAACCGCTTCCTCACCGCCTTGGGGG
GACTGTGGGAGCCAGGAGAGCTTCAGCTGCTCAACATCACTTCCGCCTTGGACC
GGGGAGGCCGAGTCCCTCTTCCTATTGAGGGACGGAAGGAAGGGGTATACATTA
AGGTAGGCTCTGCCACACCCTTCTCCACCTGCCTGAAGATGGTGGCGTCGCCCG
ACAGCTATGCCCGTTGTGCCCAGGGACAGCCTCCACTACTGTCCTGCTACGACA
CTTTGGCACCCCACTTCCGCGTTGACTGGTGCAATGTGTCTCTGGTAGACAAGTC
AGTACCCGAGCCCCTGGATGAGGTACCTACTCCAGGCGATGGGATCTTGGAGCA
CGACCCGTTCTTCTGCCCACCCACTGAAGCCACAGACCGAGACTTCCTGACAGA
TGCCTTGGTGACCCTCTTGGTGCCTTTGTTGGTGGCTCTGCTGCTTACTCTGTTGC
TGGCTTACATCATGTGCTTTCGGCGTGAAGGACGGCTGAAGAGAGACATGGCCA
CCTCTGACATCCAGATGTTTCACCACTGTTCCATCCATGGGAATACAGAAGAGCT
TCGGCAGATGGCAGCCAGCCGAGAGGTGCCCCGGCCTCTTTCCACCTTGCCCAT
GTTTAATGTTCGTACAGGAGAGCGGTTACCTCCCCGAGTAGACAGCGCACAGAT
GCCTCTTATCCTGGACCAGCAC

Mu Sarcoglycan alpha:

(SEQ ID NO: 109)
MAAAVTWIPLLAGLLAGLRDTKAQQTTLHLLVGRVFVHPLEHATFLRLPEHVAVPP
TVRLTYHAHLQGHPDLPRWLHYTQRSPYNPGFLYGSPTPEDRGYQVIEVTAYNRDS
FDTTRQRLLLLIGDPEGPRLPYQAEFLVRSHDVEEVLPTTPANRFLTALGGLWEPGE
LQLLNITSALDRGGRVPLPIEGRKEGVYIKVGSATPFSTCLKMVASPDSYARCAQGQ
PPLLSCYDTLAPHFRVDWCNVSLVDKSVPEPLDEVPTPGDGILEHDPFFCPPTEATDR
DFLTDALVTLLVPLLVALLLTLLLAYIMCFRREGRLKRDMATSDIQMFHHCSIHGNT
EELRQMAASREVPRPLSTLPMFNVRTGERLPPRVDSAQM PLILDQH

Mu FGF10

(SEQ ID NO: 110)
ATGTGGAAATGGATACTGACACATTGTGCCTCAGCCTTTCCCCACCTGCCGGGCT
GCTGTTGCTGCTTCTTGTTGCTCTTTTTGGTGTCTTCGTTCCCTGTCACCTGCCAA
GCTCTTGGTCAGGACATGGTGTCACAGGAGGCCACCAACTGCTCTTCTTCCTCCT
CGTCCTTCTCCTCTCCTTCCAGTGCGGGAAGGCATGTGCGGAGCTACAATCACCT
CCAAGGAGATGTCCGCTGGAGAAGGCTGTTCTCCTTCACCAAGTACTTTCTCACG
ATTGAGAAGAACGGCAAGGTCAGCGGGACCAAGAATGAAGACTGTCCGTACAG
TGTCCTGGAGATAACATCAGTGGAAATCGGAGTTGTTGCCGTCAAAGCCATCAA
CAGCAACTATTACTTAGCCATGAACAAGAAGGGGAAACTCTATGGCTCAAAAGA
GTTTAACAACGACTGTAAGCTGAAAGAGAGAATAGAGGAAAATGGATACAACA
CCTATGCATCTTTTAACTGGCAGCACAATGGCAGGCAAATGTATGTGGCATTGA
ATGGAAAAGGAGCTCCCAGGAGAGGACAAAAAACAAGAAGGAAAAACACCTCT
GCTCACTTCCTCCCCATGACGATCCAAACA

Mu FGF10

(SEQ ID NO: 111)
MWKWILTHCASAFPHLPGCCCCFLLLFLVSSFPVTCQALGQDMVSQEATNCSSSSSS
FSSPSSAGRHVRSYNHLQGDVRWRRLFSFTKYFLTIEKNGKVSGTKNEDCPYSVLEI
TSVEIGVVAVKAINSNYYLAMNKKGKLYGSKEFNNDCKLKERIEENGYNTYASFN
WQHNGRQMYVALNGKGAPRRGQKTRRKNTSAHFLPMTIQT

Mu Agrin

(SEQ ID NO: 112)
ATGCCTCCTCTGCCACTGGAACACAGACCCAGGCAGCAGCCTGGTGCCTCCGTG
CTGGTTCGGTACTTCATGATCCCCTGCAACATCTGCTTGATCCTCTTGGCTACTTC
TACGTTGGGCTTTGCGGTGCTGCTTTTCCTCAGCAACTACAAACCTGGGATCCAC
TTCACAGCAGCGCCTTCTATGCCTCCTGATGTGTGCAGGGGAATGTTATGTGGCT
TTGGTGCTGTGTGTGAACCTAGTGTTGAGGATCCAGGCCGGGCCTCCTGTGTGTG
CAAGAAGAATGTCTGCCCTGCTATGGTAGCTCCTGTGTGTGGCTCAGATGCTTCC
ACCTATAGCAACGAGTGTGAGCTACAGCGTGCACAGTGCAACCAGCAACGGCG
CATCCGCCTACTCCGCCAAGGGCCATGTGGGTCCCGGGACCCCTGTGCCAATGT
GACCTGCAGTTTCGGTAGTACCTGTGTACCTTCAGCCGATGGACAGACCGCCTC
GTGTCTGTGTCCTACAACCTGCTTTGGGGCCCCTGATGGCACAGTGTGTGGCAGT
GATGGTGTTGACTACCCTAGTGAGTGCCAGCTGCTCCGTCATGCCTGTGCCAACC
AGGAGCACATCTTTAAGAAGTTCGATGGTCCTTGTGACCCCTGCCAGGGCAGCA
TGTCAGACCTGAATCATATTTGCCGGGTGAACCCACGTACACGGCACCCAGAAA
TGCTTCTGCGGCCTGAGAACTGCCCCGCCCAACACACACCTATCTGTGGAGATG
ATGGGGTCACCTATGAAAACGACTGTGTCATGAGCCGTATAGGTGCAGCCCGTG
GCCTGCTTCTCCAGAAAGTGCGCTCTGGTCAATGCCAGACTCGAGACCAGTGCC
CGGAGACCTGCCAGTTTAACTCTGTATGCCTGTCCCGCCGCGGCCGTCCCCACTG
TTCCTGCGATCGCGTCACCTGTGATGGGGCTTACAGGCCAGTGTGTGCCCAGGA
TGGGCACACGTATGACAATGACTGTTGGCGCCAACAGGCCGAGTGTCGACAACA
GCAGACCATTCCCCCCAAGCACCAGGGCCCGTGTGACCAGACCCCATCCCCGTG
CCGTGGAGCGCAGTGTGCATTTGGGGCAACATGCACAGTGAAGAATGGGAAAG
CTGTGTGCGAGTGCCAGCGGGTGTGCTCGGGCGGCTACGATCCTGTGTGCGGCA
GTGATGGTGTCACTTACGGCAGTGTGTGCGAGCTGGAATCCATGGCCTGTACCC
TTGGGCGGGAAATCCGAGTGGCCCGCAGAGGACCGTGTGACCGATGTGGGCAG
TGCCGGTTTGGATCCTTGTGCGAGGTGGAGACTGGACGCTGTGTGTGCCCCTCTG
AGTGTGTGGAGTCAGCCCAGCCCGTATGTGGCTCTGACGGACACACATATGCTA
GTGAATGTGAGCTGCATGTCCACGCCTGTACACACCAGATCAGCCTATACGTGG
CCTCAGCCGGACACTGCCAGACCTGTGGAGAAACAGTTTGTACCTTTGGGGCTG
TGTGCTCAGCTGGACAGTGTGTATGTCCCCGTTGTGAGCACCCCCCACCTGGCCC
TGTGTGCGGCAGTGATGGCGTCACCTACCTCAGTGCCTGTGAGCTCCGAGAGGC
TGCCTGTCAGCAGCAGGTACAAATTGAGGAGGCCCGTGCAGGGCCGTGTGAGCC
GGCTGAGTGTGGCTCAGGGGGCTCTGGGTCTGGGGAAGACAATGCGTGTGAGCA
GGAGCTGTGTCGGCAGCATGGTGGTGTCTGGGATGAGGACTCAGAAGACGGGC
CGTGTGTCTGTGACTTTAGTTGCCAGAGTGTCCTTAAAAGCCCAGTGTGTGGCTC
AGATGGAGTCACCTATAGCACGGAGTGCCATCTGAAGAAGGCCAGATGTGAAG
CGCGGCAAGAGCTGTACGTCGCTGCTCAGGGAGCCTGCCGGGGCCCTACCTTGG
CTCCACTGCTACCTATGGCCTCCCCACACTGTGCCCAAACCCCCTATGGCTGCTG
CCAGGACAATGTCACTGCTGCCCAGGGTGTGGGCTTGGCTGGCTGTCCCAGCAC
CTGCCATTGCAACCCACACGGCTCCTATAGCGGCACTTGTGACCCAGTCACAGG
GCAGTGCTCCTGCCGACCAGGTGTAGGAGGCCTCAGGTGTGATCGCTGTGAGCC
TGGCTTCTGGAACTTCCGTGGCATTGTCACCGATGGACATAGTGGTTGCACTCCC
TGCAGCTGTGACCCTCGGGGTGCTGTAAGAGATGACTGTGAGCAGATGACTGGA
TTGTGTTCCTGTAGACCTGGTGTGGCTGGTCCCAAGTGTGGGCAGTGTCCAGATG
GTCAAGCCCTGGGCCATCTTGGCTGTGAAGCAGATCCCACAACACCAGTGACTT
GTGTGGAAATGCACTGTGAGTTTGGCGCCTCCTGCGTAGAGGAGGCTGGTTTTG
CCCAGTGTGTCTGCCCAACTCTCACATGTCCAGAGGCTAACTCTACCAAGGTCTG
TGGATCAGATGGTGTCACATACGGCAATGAATGCCAGCTGAAGACCATTGCCTG
CCGCCAGCGTCTGGACATCTCCATTCAGAGTCTTGGTCCATGCCGGGAGAGTGTT
GCTCCTGGGGTTTCCCCTACATCTGCATCTATGACCACCCCAAGGCATATCCTGA
GCAGGACACTGGCGTCTCCCCACAGCAGCCTTCCTCTGTCTCCCAGCACTACTGC
CCATGATTGGCCCACCCCATTACCCACATCACCTCAGACCGTAGTCGGCACCCCC
AGGAGCACTGCAGCCACACCCTCTGATGTGGCCAGTCTTGCTACAGCGATCTTC
AGGGAATCTGGCAGCACCAACGGCAGTGGCGATGAGGAGCTCAGTGGCGATGA
GGAGGCCAGTGGGGGCGGGTCTGGGGGACTTGAGCCCCCGGTGGGCAGCGTTG
TGGTGACCCACGGGCCACCCATCGAGAGGGCTTCCTGTTACAACTCACCTTTGG
GCTGCTGCTCAGATGGCAAGACACCCTCACTGGACTCAGAAGGCTCCAACTGTC
CAGCTACCAAGGCATTCCAGGGCGTGCTGGAGCTTGAGGGGGTCGAGGGACAG
GAACTGTTCTACACACCAGAGATGGCTGACCCCAAGTCAGAGTTGTTTGGGGAG
ACTGCAAGGAGCATTGAGAGCACGCTGGACGACCTGTTCCGGAATTCGGATGTT
AAGAAGGACTTCTGGAGCATCCGCCTACGGGAACTGGGGCCTGGCAAATTAGTC
CGTGCCATTGTGGATGTTCACTTTGACCCCACCACAGCCTTCCAGGCACCAGATG
TGGGTCAGGCCTTGCTCCAACAGATCCAGGTATCCAGGCCGTGGGCCCTGGCAG
TGAGGAGGCCTCTGCGGGAGCATGTGCGATTCTTGGACTTTGACTGGTTTCCCAC
TTTTTTTACGGGAGCTGCAACAGGAACCACAGCTGCTGTGGCCACAGCCAGAGC
CACCACTGTGAGCCGACTGTCTGCCTCTTCTGTCACCCCACGAGTCTACCCCAGT
TACACCAGCCGGCCTGTTGGCAGAACTACGGCACCGCTAACCACTCGCCGGCCA
CCAACCACTACCGCCAGTATTGACCGACCTCGGACTCCAGGCCCGCAACGGCCC
CCAAAGTCCTGTGATTCCCAGCCTTGCCTCCACGGAGGTACCTGCCAGGACCTG
GATTCTGGCAAGGGTTTCAGCTGCAGCTGTACTGCAGGCAGGGCTGGCACTGTC
TGTGAGAAAGTGCAGCTCCCCTCTGTGCCAGCTTTTAAGGGCCACTCCTTCTTGG
CCTTCCCCACCCTCCGAGCCTACCACACGCTGCGTCTGGCACTAGAATTCCGGGC
GCTGGAGACAGAGGGACTGCTGCTCTACAATGGCAATGCACGTGGCAAAGATTT
CCTGGCTCTGGCTCTGTTGGATGGTCATGTACAGTTCAGGTTCGACACGGGCTCA
GGGCCGGCGGTGCTAACAAGCTTAGTGCCAGTGGAACCGGGACGGTGGCACCG
CCTCGAGTTGTCACGGCATTGGCGGCAGGGCACACTTTCTGTGGATGGCGAGGC
TCCTGTTGTAGGTGAAAGTCCGAGTGGCACTGATGGCCTCAACTTGGACACGAA
GCTCTATGTGGGTGGTCTCCCAGAAGAACAAGTTGCCACGGTGCTTGATCGGAC
CTCTGTGGGCATCGGCCTGAAAGGATGCATTCGTATGTTGGACATCAACAACCA
GCAGCTGGAGCTGAGCGATTGGCAGAGGGCTGTGGTTCAAAGCTCTGGTGTGGG
GGAATGCGGAGACCATCCCTGCTCACCTAACCCCTGCCATGGCGGGGCCCTCTG
CCAGGCCCTGGAGGCTGGCGTGTTCCTCTGTCAGTGCCCACCTGGCCGCTTTGGC
CCAACTTGTGCAGATGAAAAGAACCCCTGCCAACCGAACCCCTGCCACGGGTCA
GCCCCCTGCCATGTGCTTTCCAGGGGTGGGGCCAAGTGTGCGTGCCCCCTGGGA
CGCAGTGGTTCCTTCTGTGAGACAGTCCTGGAGAATGCTGGCTCCCGGCCCTTCC
TGGCTGACTTTAATGGCTTCTCCTACCTGGAACTGAAAGGCTTGCACACCTTCGA
GAGAGACCTAGGGGAGAAGATGGCGCTGGAGATGGTGTTCTTGGCTCGTGGGCC
CAGTGGCTTACTCCTCTACAATGGGCAGAAGACGGATGGCAAGGGGGACTTTGT
ATCCCTGGCCCTGCATAACCGGCACCTAGAGTTCCGCTATGACCTTGGCAAGGG
GGCTGCAATCATCAGGAGCAAAGAGCCCATAGCCCTGGGCACCTGGGTTAGGGT
ATTCCTGGAACGAAATGGCCGCAAGGGTGCCCTTCAAGTGGGTGATGGGCCCCG
TGTGCTAGGGGAATCTCCGAAATCCCGCAAGGTCCCGCACACCATGCTCAACCT
CAAGGAGCCCCTCTATGTGGGGGGAGCTCCTGACTTCAGCAAGCTGGCTCGGGG
CGCTGCAGTGGCCTCCGGCTTTGATGGTGCCATCCAGCTGGTGTCTCTAAGAGGC
CATCAGCTGCTGACTCAGGAGCATGTGTTGCGGGCAGTAGATGTAGCGCCTTTT
GCAGGCCACCCTTGTACCCAGGCCGTGGACAACCCCTGCCTTAATGGGGGCTCC
TGTATCCCGAGGGAAGCCACTTATGAGTGCCTGTGTCCTGGGGGCTTCTCTGGGC
TGCACTGCGAGAAGGGGATAGTTGAGAAGTCAGTGGGGGACCTAGAAACACTG
GCCTTTGATGGGCGGACCTACATCGAGTACCTCAATGCTGTGACTGAGAGCGAG
CTGACCAATGAGATCCCAGCCCCCGAAACTCTGGATTCCCGGGCCCTTTTCAGTG
AGAAAGCGCTGCAGAGCAACCACTTTGAGCTGAGCTTACGCACTGAGGCCACGC
AGGGGCTGGTGCTGTGGATTGGAAAGGTTGGAGAACGTGCAGACTACATGGCTC
TGGCCATTGTGGATGGGCACCTACAACTGAGCTATGACCTAGGCTCCCAGCCAG
TTGTGCTGCGCTCCACTGTGAAGGTCAACACCAACCGCTGGCTTCGAGTCAGGG
CTCACAGGGAGCACAGGGAAGGTTCCCTTCAGGTGGGCAATGAAGCCCCTGTGA
CTGGCTCTTCCCCGCTGGGTGCCACACAATTGGACACAGATGGAGCCCTGTGGC
TTGGAGGCCTACAGAAGCTTCCTGTGGGGCAGGCTCTCCCCAAGGCCTATGGCA
CGGGTTTTGTGGGCTGTCTGCGGGACGTGGTAGTGGGCCATCGCCAGCTGCATC
TGCTGGAGGACGCTGTCACCAAACCAGAGCTAAGACCCTGCCCCACTCTCTGA

Mu Agrin

(SEQ ID NO: 113)
MPPLPLEHRPRQQPGASVLVRYFMIPCNICLILLATSTLGFAVLLFLSNYKPGIHFTAA
PSMPPDVCRGMLCGFGAVCEPSVEDPGRASCVCKKNVCPAMVAPVCGSDASTYSN
ECELQRAQCNQQRRIRLLRQGPCGSRDPCANVTCSFGSTCVPSADGQTASCLCPTTC
FGAPDGTVCGSDGVDYPSECQLLRHACANQEHIFKKFDGPCDPCQGSMSDLNHICR
VNPRTRHPEMLLRPENCPAQHTPICGDDGVTYENDCVMSRIGAARGLLLQKVRSGQ
CQTRDQCPETCQFNSVCLSRRGRPHCSCDRVTCDGAYRPVCAQDGHTYDNDCWRQ
QAECRQQQTIPPKHQGPCDQTPSPCRGAQCAFGATCTVKNGKAVCECQRVCSGGY
DPVCGSDGVTYGSVCELESMACTLGREIRVARRGPCDRCGQCRFGSLCEVETGRCV
CPSECVESAQPVCGSDGHTYASECELHVHACTHQISLYVASAGHCQTCGETVCTFG
AVCSAGQCVCPRCEHPPPGPVCGSDGVTYLSACELREAACQQQVQIEEARAGPCEP
AECGSGGSGSGEDNACEQELCRQHGGVWDEDSEDGPCVCDFSCQSVLKSPVCGSD
GVTYSTECHLKKARCEARQELYVAAQGACRGPTLAPLLPMASPHCAQTPYGCCQD
NVTAAQGVGLAGCPSTCHCNPHGSYSGTCDPVTGQCSCRPGVGGLRCDRCEPGFW
NFRGIVTDGHSGCTPCSCDPRGAVRDDCEQMTGLCSCRPGVAGPKCGQCPDGQAL
GHLGCEADPTTPVTCVEMHCEFGASCVEEAGFAQCVCPTLTCPEANSTKVCGSDGV
TYGNECQLKTIACRQRLDISIQSLGPCRESVAPGVSPTSASMTTPRHILSRTLASPHSS
LPLSPSTTAHDWPTPLPTSPQTVVGTPRSTAATPSDVASLATAIFRESGSTNGSGDEE
LSGDEEASGGGSGGLEPPVGSVVVTHGPPIERASCYNSPLGCCSDGKTPSLDSEGSN
CPATKAFQGVLELEGVEGQELFYTPEMADPKSELFGETARSIESTLDDLFRNSDVKK
DFWSIRLRELGPGKLVRAIVDVHFDPTTAFQAPDVGQALLQQIQVSRPWALAVRRP
LREHVRFLDFDWFPTFFTGAATGTTAAVATARATTVSRLSASSVTPRVYPSYTSRPV
GRTTAPLTTRRPPTTTASIDRPRTPGPQRPPKSCDSQPCLHGGTCQDLDSGKGFSCSC
TAGRAGTVCEKVQLPSVPAFKGHSFLAFPTLRAYHTLRLALEFRALETEGLLLYNGN
ARGKDFLALALLDGHVQFRFDTGSGPAVLTSLVPVEPGRWHRLELSRHWRQGTLS
VDGEAPVVGESPSGTDGLNLDTKLYVGGLPEEQVATVLDRTSVGIGLKGCIRMLDI
NNQQLELSDWQRAVVQSSGVGECGDHPCSPNPCHGGALCQALEAGVFLCQCPPGR
FGPTCADEKNPCQPNPCHGSAPCHVLSRGGAKCACPLGRSGSFCETVLENAGSRPFA
DFNGFSYLELKGLHTFERDLGEKMALEMVFLARGPSGLLLYNGQKTDGKGDFVSL
ALHNRHLEFRYDLGKGAAIIRSKEPIALGTWVRVFLERNGRKGALQVGDGPRVLGE
SPKSRKVPHTMLNLKEPLYVGGAPDFSKLARGAAVASGFDGAIQLVSLRGHQLLTQ
EHVLRAVDVAPFAGHPCTQAVDNPCLNGGSCIPREATYECLCPGGFSGLHCEKGIVE
KSVGDLETLAFDGRTYIEYLNAVTESELTNEIPAPETLDSRALFSEKALQSNHFELSL
RTEATQGLVLWIGKVGERADYMALAIVDGHLQLSYDLGSQPVVLRSTVKVNTNRW
LRVRAHREHREGSLQVGNEAPVTGSSPLGATQLDTDGALWLGGLQKLPVGQALPK
AYGTGFVGCLRDVVVGHRQLHLLEDAVTKPELRPCPTL

Mu IL10

(SEQ ID NO: 114)
ATGCCTGGCTCAGCACTGCTATGCTGCCTGCTCTTACTGACTGGCATGAGGATCA
GCAGGGGCCAGTACAGCCGGGAAGACAATAACTGCACCCACTTCCCAGTCGGCC
AGAGCCACATGCTCCTAGAGCTGCGGACTGCCTTCAGCCAGGTGAAGACTTTCT
TTCAAACAAAGGACCAGCTGGACAACATACTGCTAACCGACTCCTTAATGCAGG
ACTTTAAGGGTTACTTGGGTTGCCAAGCCTTATCGGAAATGATCCAGTTTTACCT
GGTAGAAGTGATGCCCCAGGCAGAGAAGCATGGCCCAGAAATCAAGGAGCATT
TGAATTCCCTGGGTGAGAAGCTGAAGACCCTCAGGATGCGGCTGAGGCGCTGTC
ATCGATTTCTCCCCTGTGAAAATAAGAGCAAGGCAGTGGAGCAGGTGAAGAGTG
ATTTTAATAAGCTCCAAGACCAAGGTGTCTACAAGGCCATGAATGAATTTGACA
TCTTCATCAACTGCATAGAAGCATACATGATGATCAAAATGAAAAGCTAA

Mu IL10

(SEQ ID NO: 115)
MPGSALLCCLLLLTGMRISRGQYSREDNNCTHFPVGQSHMLLELRTAFSQVKTFFQT
KDQLDNILLTDSLMQDFKGYLGCQALSEMIQFYLVEVMPQAEKHGPEIKEHLNSLG
EKLKTLRMRLRRCHRFLPCENKSKAVEQVKSDFNKLQDQGVYKAMNEFDIFINCIE
AYMMIKMKS

Mu MYDGF (C19orf10)

(SEQ ID NO: 116)
ATGGCAGCCCCCAGCGGAGGCTTCTGGACTGCGGTGGTCCTGGCGGCCGCAGCG
CTGAAATTGGCCGCCGCTGTGTCCGAGCCCACCACCGTGCCATTTGACGTGAGG
CCCGGAGGGGTCGTGCATTCGTTCTCCCAGGACGTAGGACCCGGGAACAAGTTT
ACATGTACATTCACCTACGCTTCCCAAGGAGGGACCAACGAGCAATGGCAGATG
AGCCTGGGGACAAGTGAAGACAGCCAGCACTTTACCTGTACCATCTGGAGGCCC
CAGGGGAAATCCTACCTCTACTTCACACAGTTCAAGGCTGAGTTGCGAGGTGCT
GAGATCGAGTATGCCATGGCCTACTCCAAAGCCGCATTTGAGAGAGAGAGTGAT
GTCCCCCTGAAAAGTGAGGAGTTTGAAGTGACCAAGACAGCAGTGTCTCACAGG
CCTGGGGCCTTCAAAGCTGAGCTCTCCAAGCTGGTGATCGTAGCCAAGGCGGCA
CGCTCGGAGCTGTGA

Mu MYDGF (C19orf10)

(SEQ ID NO: 117)
MAAPSGGFWTAVVLAAAALKLAAAVSEPTTVPFDVRPGGVVHSFSQDVGPGNKFT
CTFTYASQGGTNEQWQMSLGTSEDSQHFTCTIWRPQGKSYLYFTQFKAELRGAEIE
YAMAYSKAAFERESDVPLKSEEFEVTKTAVSHRPGAFKAELSKLVIVAKAARSEL

pWF-521

(SEQ ID NO: 118)
CAGTCTGTGTTGACGCAGCCGCCCTCAGTGTCTGCGGCCCCAGGACAGAGGGTC
ACCATCTCCTGCTCTGGAACCAGGTCCAACATTGGGAGTGATTATGTTTCCTGGT
ACCAACACCTCCCAGGAACAGCCCCCAAACTCCTCGTTTATGGCGATAATCTGC
GACCCTCAGGGATTCCTGACCGATTCTCTGCCTCCAAGTCTGGCACGTCAGCCAC
CCTGGGCATCACCGGACTCCAGACTGGGGACGAGGCCGATTATTACTGCGGCAC
ATGGGATTACACCCTGAATGGTGTGGTGTTCGGCGGAGGGACCAAGCTGACCGT
CCTAGGTCAGCCCAAGGCCAACCCCACTGTCACTCTGTTCCCGCCCTCCTCTGAG
GAGCTCCAAGCCAACAAGGCCACACTAGTGTGTCTGATCAGTGACTTCTACCCG
GGAGCTGTGACAGTGGCCTGGAAGGCAGATGGCAGCCCCGTCAAGGCGGGAGT
GGAGACCACCAAACCCTCCAAACAGAGCAACAACAAGTACGCGGCCAGCAGCT
ACCTGAGCCTGACGCCCGAGCAGTGGAAGTCCCACAGAAGCTACAGCTGCCAG
GTCACGCATGAAGGGAGCACCGTGGAGAAGACAGTGGCCCCTACAGAATGTTC
AGGCGCCGGATCTGGTGGAAACTGGAGTCATCCCCAATTCGAGAAGGGCGGAA
GCGGTGGGAGTGGCGGGTCCGGTGGAAGCAACTGGTCACACCCACAATTCGAG
AAAGGCGGTTCTGGCGGATCTGGTGGATCTGGCGGAAGTAACTGGTCTCATCCT
CAATTCGAAAAGGGCGGAAGCGGTGGCGGCAGGCTAGGTGGAGGCTCAGTGCA
GGTGCAGCTGGTGGAGTCTGGGGGAGGCTTGGTACAGCCTGGGGGGTCCCTGAG
ACTCTCCTGTGCAGCCTCTGGATTCACCTTTAGCAGCTATGCCATGAGCTGGGTC
CGCCAGGCTCCAGGGAAGGGGCTGGAGTGGGTCTCAGTTATTTATAGCGGTGGT
AGTAGCACATACTATGCAGACTCCGTGAAGGGCCGGTTCACCATCTCCAGAGAT
AATTCCAAGAACACGCTGTATCTGCAAATGAACAGCCTGAGAGCCGAGGACAC
GGCCGTATATTACTGTGCGCGCACTTCTTACCTGAACCATGGTGATTACTGGGGT
CAAGGTACTCTGGTGACCGTGTCTAGCGCCTCCACCAAGGGCCCATCGGTCTTCC
CCCTGGCACCCTCCTCCAAGAGCACCTCTGGGGGCACAGCGGCCCTGGGCTGCC
TGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTC
CCTCAGCAGCGTGGTGACCGTGCCCTCCAGCAGCTTGGGCACCCAGACCTACAT
CTGCAACGTGAACCACAAGCCCAGCAACACCAAGGTGGACAAGAGAGTGGAGC
CCAAGAGCTGCGACAAGACCCACACCTGCCCCCCCTGCCCAGCCCCAGAGCTGC
TGGGCGGACCCTCCGTGTTCCTGTTCCCCCCCAAGCCCAAGGACACCCTGATGAT
CAGCAGGACCCCCGAGGTGACCTGCGTGGTGGTGGACGTGAGCCACGAGGACC
CAGAGGTGAAGTTCAACTGGTACGTGGACGGCGTGGAGGTGCACAACGCCAAG
ACCAAGCCCAGAGAGGAGCAGTACAACAGCACCTACAGGGTGGTGTCCGTGCT
GACCGTGCTGCACCAGGACTGGCTGAACGGCAAGGAATACAAGTGCAAGGTCT
CCAACAAGGCCCTGCCAGCCCCCATCGAAAAGACCATCAGCAAGGCCAAGGGC
CAGCCACGGGAGCCCCAGGTGTACACCCTGCCCCCCTCCCGGGAGGAGATGACC
AAGAACCAGGTGTCCCTGACCTGTCTGGTGAAGGGCTTCTACCCCAGCGACATC
GCCGTGGAGTGGGAGAGCAACGGCCAGCCCGAGAACAACTACAAGACCACCCC
CCCAGTGCTGGACAGCGACGGCAGCTTCTTCCTGTACAGCAAGCTGACCGTGGA
CAAGTCCAGGTGGCAGCAGGGCAACGTGTTCAGCTGCAGCGTGATGCACGAGG
CCCTGCACAACCACTACACCCAGAAGAGCCTGAGCCTGTCCCCCGAGCTGCAAC
TGGAGGAGAGCTGTGCGGAGGCGCAGGACGGGGAGCTGGACGGGCTGTGGACG
ACCATCACCATCTTCATCACACTCTTCCTGTTAAGCGTGTGCTACAGTGCCACCG
TCACCTTCTTCAAGGTGAAGTGGATCTTCTCCTCGGTGGTGGACCTGAAGCAGAC
CATCATCCCCGACTACAGGAACATGATCGGACAGGGGGCCTGA

pWF-521

(SEQ ID NO: 119)
QSVLTQPPSVSAAPGQRVTISCSGTRSNIGSDYVSWYQHLPGTAPKLLVYGDNLRPS
GIPDRFSASKSGTSATLGITGLQTGDEADYYCGTWDYTLNGVVFGGGTKLTVLGQP
KANPTVTLFPPSSEELQANKATLVCLISDFYPGAVTVAWKADGSPVKAGVETTKPS
KQSNNKYAASSYLSLTPEQWKSHRSYSCQVTHEGSTVEKTVAPTECSGAGSGGNW
SHPQFEKGGSGGSGGSGGSNWSHPQFEKGGSGGSGGSGGSNWSHPQFEKGGSGGG
RLGGGSVQVQLVESGGGLVQPGGSLRLSCAASGFTFSSYAMSWVRQAPGKGLEWV
SVIYSGGSSTYYADSVKGRFTISRDNSKNTLYLQMNSLRAEDTAVYYCARTSYLNH
GDYWGQGTLVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNTKVDKRVE
PKSCDKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQKS
LSLSPELQLEESCAEAQDGELDGLWTTITIFITLFLLSVCYSATVTFFKVKWIFSSVVD
LKQTIIPDYRNMIGQGA

pWF-533

(SEQ ID NO: 120)
CAGTCTGTGTTGACGCAGCCGCCCTCAGTGTCTGCGGCCCCAGGACAGAGGGTC
ACCATCTCCTGCTCTGGAACCAGGTCCAACATTGGGAGTGATTATGTTTCCTGGT
ACCAACACCTCCCAGGAACAGCCCCCAAACTCCTCGTTTATGGCGATAATCTGC
GACCCTCAGGGATTCCTGACCGATTCTCTGCCTCCAAGTCTGGCACGTCAGCCAC
CCTGGGCATCACCGGACTCCAGACTGGGGACGAGGCCGATTATTACTGCGGCAC
ATGGGATTACACCCTGAATGGTGTGGTGTTCGGCGGAGGGACCAAGCTGACCGT
CCTAtcttcaGCCTCCACCAAGGGCCCATCGGTCTTCCCCCTGGCACCCTCCTCCAAG
AGCACCTCTGGGGGCACAGCGGCCCTGGGCTGCCTGGTCAAGGACTACTTCCCC
GAACCGGTGACGGTGTCGTGGAACTCAGGCGCCCTGACCAGCGGCGTGCACACC
TTCCCGGCTGTCCTACAGTCCTCAGGACTCTACTCCCTCAGCAGCGTGGTGACCG
TGCCCTCCAGCAGCTTGGGCACCCAGACCTACATCTGCAACGTGAACCACAAGC
CCAGCAACACCAAGGTGGACAAGAGAGTGGAGCCCAAGAGCTGC

pWF-533

(SEQ ID NO: 121)
QSVLTQPPSVSAAPGQRVTISCSGTRSNIGSDYVSWYQHLPGTAPKLLVYGDNLRPS
GIPDRFSASKSGTSATLGITGLQTGDEADYYCGTWDYTLNGVVFGGGTKLTVLSSAS
TKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKRVEPKSC

pWF-534

(SEQ ID NO: 122)
CAGGTGCAGCTGGTGGAGTCTGGGGGAGGCTTGGTACAGCCTGGGGGGTCCCTG
AGACTCTCCTGTGCAGCCTCTGGATTCACCTTTAGCAGCTATGCCATGAGCTGGG
TCCGCCAGGCTCCAGGGAAGGGGCTGGAGTGGGTCTCAGTTATTTATAGCGGTG
GTAGTAGCACATACTATGCAGACTCCGTGAAGGGCCGGTTCACCATCTCCAGAG
ATAATTCCAAGAACACGCTGTATCTGCAAATGAACAGCCTGAGAGCCGAGGACA
CGGCCGTATATTACTGTGCGCGCACTTCTTACCTGAACCATGGTGATTACTGGGG
TCAAGGTACTCTGGTGACCGTGTCTAGCGCCTCCGTGGCTGCACCATCTGTCTTC
ATCTTCCCGCCATCTGATGAGCAGTTGAAATCTGGAACTGCCTCTGTTGTGTGCC
TGCTGAATAACTTCTATCCCAGAGAGGCCAAAGTACAGTGGAAGGTGGATAACG
CCCTCCAATCGGGTAACTCCCAGGAGAGTGTCACAGAGCAGGACAGCAAGGAC
AGCACCTACAGCCTCAGCAGCACCCTGACGCTGAGCAAAGCAGACTACGAGAA
ACACAAAGTCTACGCCTGCGAAGTCACCCATCAGGGCCTGAGCTCGCCCGTCAC
AAAGAGCTTCAACAGGGGAGAGTGTGACAAGACCCACACCTGCCCCCCCTGCCC
AGCCCCAGAGCTGCTGGGCGGACCCTCCGTGTTCCTGTTCCCCCCCAAGCCCAA
GGACACCCTGATGATCAGCAGGACCCCCGAGGTGACCTGCGTGGTGGTGGACGT
GAGCCACGAGGACCCAGAGGTGAAGTTCAACTGGTACGTGGACGGCGTGGAGG
TGCACAACGCCAAGACCAAGCCCAGAGAGGAGCAGTACAACAGCACCTACAGG
GTGGTGTCCGTGCTGACCGTGCTGCACCAGGACTGGCTGAACGGCAAGGAATAC
AAGTGCAAGGTCTCCAACAAGGCCCTGCCAGCCCCCATCGAAAAGACCATCAGC
AAGGCCAAGGGCCAGCCACGGGAGCCCCAGGTGTACACCCTGCCCCCCTCCCGG
GAGGAGATGACCAAGAACCAGGTGTCCCTGACCTGTCTGGTGAAGGGCTTCTAC
CCCAGCGACATCGCCGTGGAGTGGGAGAGCAACGGCCAGCCCGAGAACAACTA
CAAGACCACCCCCCCAGTGCTGGACAGCGACGGCAGCTTCTTCCTGTACAGCAA
GCTGACCGTGGACAAGTCCAGGTGGCAGCAGGGCAACGTGTTCAGCTGCAGCGT
GATGCACGAGGCCCTGCACAACCACTACACCCAGAAGAGCCTGAGCCTGTCCCC
CGAGCTGCAACTGGAGGAGAGCTGTGCGGAGGCGCAGGACGGGGAGCTGGACG
GGCTGTGGACGACCATCACCATCTTCATCACACTCTTCCTGTTAAGCGTGTGCTA
CAGTGCCACCGTCACCTTCTTCAAGGTGAAGTGGATCTTCTCCTCGGTGGTGGAC
CTGAAGCAGACCATCATCCCCGACTACAGGAACATGATCGGACAGGGGGCCTG
A

pWF-534

(SEQ ID NO: 123)
QVQLVESGGGLVQPGGSLRLSCAASGFTFSSYAMSWVRQAPGKGLEWVSVIYSGGS
STYYADSVKGRFTISRDNSKNTEYEQMNSERAEDTAVYYCARTSYENHGDYWGQG
TLVTVSSASVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSFNRGEC
DKTHTCPPCPAPEEEGGPSVFEFPPKPKDTEMISRTPEVTCVVVDVSHEDPEVKFNW
YVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQKSLSLSP
ELQLEESCAEAQDGELDGLWTTITIFITLFLLSVCYSATVTFFKVKWIFSSVVDLKQTI
IPDYRNMIGQGA

mu IL15

(SEQ ID NO: 124)
ATGAAAATTTTGAAACCATATATGAGGAATACATCCATCTCGTGCTACTTGTGTT
TCCTTCTAAACAGTCACTTTTTAACTGAGGCTGGCATTCATGTCTTCATTTTGGGC
TGTGTCAGTGTAGGTCTCCCTAAAACAGAGGCCAACTGGATAGATGTAAGATAT
GACCTGGAGAAAATTGAAAGCCTTATTCAATCTATTCATATTGACACCACTTTAT
ACACTGACAGTGACTTTCATCCCAGTTGCAAAGTTACTGCAATGAACTGCTTTCT
CCTGGAATTGCAGGTTATTTTACATGAGTACAGTAACATGACTCTTAATGAAAC
AGTAAGAAACGTGCTCTACCTTGCAAACAGCACTCTGTCTTCTAACAAGAATGT
AGCAGAATCTGGCTGCAAGGAATGTGAGGAGCTGGAGGAGAAAACCTTCACAG
AGTTTTTGCAAAGCTTTATACGCATTGTCCAAATGTTCATCAACACGTCC

mu IL15

(SEQ ID NO: 125)
MKILKPYMRNTSISCYLCFLLNSHFLTEAGIHVFILGCVSVGLPKTEANWIDVRYDLE
KIESLIQSIHIDTTLYTDSDFHPSCKVTAMNCFLLELQVILHEYSNMTLNETVRNVLY
LANSTLSSNKNVAESGCKECEELEEKTFTEFLQSFIRIVQMFINTS

anti-human GPC3 CAR (79a)

(SEQ ID NO: 126)
CAGTCTGTGTTGACGCAGCCGCCCTCAGTGTCTGCGGCCCCAGGACAGAGGGTC
ACCATCTCCTGCTCTGGAACCAGGTCCAACATTGGGAGTGATTATGTTTCCTGG
TACCAACACCTCCCAGGAACAGCCCCCAAACTCCTCGTTTATGGCGATAATCTG
CGACCCTCAGGGATTCCTGACCGATTCTCTGCCTCCAAGTCTGGCACGTCAGCC
ACCCTGGGCATCACCGGACTCCAGACTGGGGACGAGGCCGATTATTACTGCGG
CACATGGGATTACACCCTGAATGGTGTGGTGTTCGGCGGAGGGACCAAGCTGA
CCGTCCTAGGTTCTAGAGGTGGTGGTGGTAGCGGCGGCGGCGGCTCTGGTGGT
GGTGGATCCCTCGAGATGGCCCAGGTGCAGCTGGTGGAGTCTGGGGGAGGCTT
GGTACAGCCTGGGGGGTCCCTGAGACTCTCCTGTGCAGCCTCTGGATTCACCTT
TAGCAGCTATGCCATGAGCTGGGTCCGCCAGGCTCCAGGGAAGGGGCTGGAGT
GGGTCTCAGTTATTTATAGCGGTGGTAGTAGCACATACTATGCAGACTCCGTGA
AGGGCCGGTTCACCATCTCCAGAGATAATTCCAAGAACACGCTGTATCTGCAA
ATGAACAGCCTGAGAGCCGAGGACACGGCCGTATATTACTGTGCGCGCACTTC
TTACCTGAACCATGGTGATTACTGGGGTCAAGGTACTCTGGTGACCGTGTCTAG
CGCCGCTGCAGTGGTCCCCGTGCTGCAGAAAGTTAATAGCACCACCACTAAACCTGT
CCTGAGGACTCCTAGTCCAGTGCACCCAACAGGGACCAGTCAGCCACAGAGACCGG
AAGACTGCAGACCAAGAGGTTCAGTGAAGGGAACCGGCCTGGATTTCGCCTGCGAT
TTTTGGGCCCTGGTCGTCGTCGCAGGAGTTTTGTTTTGCTATGGACTGCTCGTCA
CAGTTGCTTTGTGTGTTATCTGGACAAGGAAACGGTGGCAAAATGAGAAGTTTGGG
GTGGACATGCCAGATGACTATGAAGATGAAAATCTCTATGAGGGCCTGAACCTTGAT
GACTGTTCTATGTATGAGGACATCTCCAGGGGACTCCAGGGCACCTACCAGGATGTG
GGCAACCTCCACATTGGAGATGCCCAGCTGGAAAAGCCATGA

anti-human GPC3 CAR (79a)

(SEQ ID NO: 127)
QSVLTQPPSVSAAPGQRVTISCSGTRSNIGSDYVSWYQHLPGTAPKLLVYGDNLRPS
GIPDRFSASKSGTSATLGITGLQTGDEADYYCGTWDYTLNGVVFGGGTKLTVLGSR
GGGGSGGGGSGGGGSLEMAQVQLVESGGGLVQPGGSLRLSCAASGFTFSSYAMS
WVRQAPGKGLEWVSVIYSGGSSTYYADSVKGRFTISRDNSKNTLYLQMNSLRAED
TAVYYCARTSYLNHGDYWGQGTLVTVSSAAAVVPVLQKVNSTTTKPVLRTPSPVHP
TGTSQPQRPEDCRPRGSVKGTGLDFACDFWALVVVAGVLFCYGLLVTVALCVIWTR
KRWQNEKFGVDMPDDYEDENLYEGLNLDDCSMYEDISRGLQGTYQDVGNLHIGDAQL
EKP

anti-human PSMA(XENP14484) CAR 79a

(SEQ ID NO: 128)
GAGGTTCAACTTGTTCAATCTGGGGCAGAAGTGAAGAAGCCCGGGGCATCTGT
GAAAGTATCATGCAAAACATCCGGCTATACGTTTACCGAATACACCATTCACTG
GGTCAGACAGGCTCCCGGTCAAAGCCTCGAATGGATGGGAAATATTAACCCTA
ACAATGGCGGAACCACATATAATCAGAAATTCCAAGGCCGAGTGACGATAACT
GTCGATAAGAGTACGTCCACAGCTTACATGGAACTCAGCTCTTTGAGATCCGAA
GACACTGCAGTTTATTATTGTGCAGCTGGATGGAACTTCGACTATTGGGGACAA
GGGACTCTTGTTACGGTGTCCAGTGGCAAACCAGGTAGTGGTAAACCCGGAAG
CGGCAAGCCCGGGAGCGGTAAACCTGGTAGCGACATCGTCATGACTCAAAGCC
CTGACTCACTCGCCGTGAGCCTGGGAGAGCGTGCAACGCTATCTTGTCGGGCCT
CTCAGGATGTCGGAACTGCTGTAGACTGGTATCAACAGAAACCTGACCAATCA
CCAAAACTCCTGATTTATTGGGCCTCAACACGTCACACAGGAGTGCCAGATAG
GTTCACAGGTAGTGGCAGTGGAACTGATTTTACTTTGACAATTAGCAGCCTGCA
AGCCGAAGATGTAGCCGTTTACTTCTGTCAACAATATAACTCATACCCACTAAC
GTTCGGTGCCGGGACGAAGGTAGAGATTAAAGTGGTCCCCGTGCTGCAGAAAGT
TAATAGCACCACCACTAAACCTGTCCTGAGGACTCCTAGTCCAGTGCACCCAACAGG
GACCAGTCAGCCACAGAGACCGGAAGACTGCAGACCAAGAGGTTCAGTGAAGGGAA
CCGGCCTGGATTTCGCCTGCGATTTTTGGGCCCTGGTCGTCGTCGCAGGAGTTTT
GTTTTGCTATGGACTGCTCGTCACAGTTGCTTTGTGTGTTATCTGGACAAGGAAA
CGGTGGCAAAATGAGAAGTTTGGGGTGGACATGCCAGATGACTATGAAGATGAAAAT
CTCTATGAGGGCCTGAACCTTGATGACTGTTCTATGTATGAGGACATCTCCAGGGGA
CTCCAGGGCACCTACCAGGATGTGGGCAACCTCCACATTGGAGATGCCCAGCTGGA
AAAGCCATGA

anti-human PSMA(XENP14484) CAR 79a

(SEQ ID NO: 129)
EVQLVQSGAEVKKPGASVKVSCKTSGYTFTEYTIHWVRQAPGQSLEWMGNINPNN
GGTTYNQKFQGRVTITVDKSTSTAYMELSSLRSEDTAVYYCAAGWNFDYWGQGT
LVTVSSGKPGSGKPGSGKPGSGKPGSDIVMTQSPDSLAVSLGERATLSCRASQDVG
TAVDWYQQKPDQSPKLLIYWASTRHTGVPDRFTGSGSGTDFTLTISSLQAEDVAVY
FCQQYNSYPLTFGAGTKVEIKVVPVLQKVNSTTTKPVLRTPSPVHPTGTSQPQRPEDC
RPRGSVKGTGLDFACDFWALVVVAGVLFCYGLLVTVALCVIWTRKRWQNEKFGVD
MPDDYEDENLYEGLNLDDCSMYEDISRGLQGTYQDVGNLHIGDAQLEKP

mouse IL 12a-mouse IgG2a Fc

(SEQ ID NO: 130)
ATGTGTCAGTCACGCTATCTTCTCTTCCTTGCTACTCTGGCCTTGCTCAATCACT
TGTCCCTTGCTCGTGTGATTCCTGTGTCCGGCCCAGCTAGGTGTCTCTCCCAGTC
ACGGAATCTCCTGAAAACCACGGATGACATGGTAAAGACAGCTAGGGAGAAAC
TCAAGCACTACTCCTGCACAGCTGAGGATATCGATCATGAGGACATCACCAGG
GACCAGACATCCACTCTGAAAACTTGCCTGCCTTTGGAACTCCACAAGAACGA
ATCTTGTCTGGCAACGCGTGAAACGAGTTCTACTACAAGAGGGTCCTGTCTTCC
CCCTCAAAAGACAAGCCTTATGATGACCTTGTGTCTCGGTAGCATTTATGAGGA
CCTAAAGATGTATCAAACCGAGTTTCAGGCTATCAATGCAGCGCTCCAGAATCA
TAACCATCAGCAGATCATTCTTGACAAAGGAATGCTCGTGGCCATTGATGAACT
AATGCAGAGCCTAAACCACAATGGCGAGACTCTTCGACAGAAACCGCCTGTGG
GCGAGGCCGATCCATATAGAGTCAAAATGAAACTGTGTATTCTCCTGCATGCAT
TTAGTACTCGTGTAGTGACTATTAACAGAGTGATGGGTTACCTTTCCTCAGCTC
CCAGAGGGCCCACAATCAAGCCCTGTCCTCCATGCAAATGCCCAGCACCTAAC
CTCTTGGGTGGACCATCCGTCTTCATCTTCCCTCCAAAGATCAAGGATGTACTC
ATGATCTCCCTGAGCCCCATAGTCACATGTGTGGTGGTGGATGTGAGCGAGGAT
GACCCAGATGTCCAGATCAGCTGGTTTGTGAACAACGTGGAAGTACACACAGC
TCAGACACAAACCCATAGAGAGGATTACAACAGTACTCTCCGGGTGGTCAGTG
CCCTCCCCATCCAGCACCAGGACTGGATGAGTGGCAAGGAGTTCAAATGCAAG
GTCAACAACAAAGACCTCCCAGCGCCCATCGAGAGAACCATCTCAAAACCCAA
AGGGTCAGTAAGAGCTCCACAGGTATATGTCTTGCCTCCACCAGAAGAAGAGA
TGACTAAGAAACAGGTCACTCTGACCTGCATGGTCACAGACTTCATGCCTGAAG
ACATTTACGTGGAGTGGACCAACAACGGGAAAACAGAGCTAAACTACAAGAAC
ACTGAACCAGTCCTGGACTCTGATGGTTCTTACTTCATGTACAGCAAGCTGAGA
GTGGAAAAGAAGAACTGGGTGGAAAGAAATAGCTACTCCTGTTCAGTGGTCCA
CGAGGGTCTGCACAATCACCACACGACTAAGAGCTTCTCCCGGACTCCGGGTA
AATAG

mouse IL 12a-mouse IgG2a Fc

(SEQ ID NO: 131)
MCQSRYLLFLATLALLNHLSLARVIPVSGPARCLSQSRNLLKTTDDMVKTAREKLK
HYSCTAEDIDHEDITRDQTSTLKTCLPLELHKNESCLATRETSSTTRGSCLPPQKTSL
MMTLCLGSIYEDLKMYQTEFQAINAALQNHNHQQIILDKGMLVAIDELMQSLNHN
GETLRQKPPVGEADPYRVKMKLCILLHAFSTRVVTINRVMGYLSSAPRGPTIKPCPP
CKCPAPNLLGGPSVFIFPPKIKDVLMISLSPIVTCVVVDVSEDDPDVQISWFVNNVEV
HTAQTQTHREDYNSTLRVVSALPIQHQDWMSGKEFKCKVNNKDLPAPIERTISKPK
GSVRAPQVYVLPPPEEEMTKKQVTLTCMVTDFMPEDIYVEWTNNGKTELNYKNTE
PVLDSDGSYFMYSKLRVEKKNWVERNSYSCSVVHEGLHNHHTTKSFSRTPGK*

mouse IL 12b-mouse IgG2a Fc

(SEQ ID NO: 132)
ATGTGCCCACAGAAACTCACAATTTCTTGGTTCGCAATCGTCCTGCTGGTGTCA
CCCCTGATGGCAATGTGGGAGTTGGAAAAGGATGTATACGTCGTCGAGGTCGA
CTGGACACCTGACGCTCCGGGTGAAACTGTCAACCTCACTTGCGATACTCCTGA
AGAGGACGACATCACGTGGACGAGCGACCAGCGACATGGAGTGATAGGGTCTG
GCAAGACGCTTACTATCACGGTTAAGGAATTTCTCGACGCAGGGCAGTACACA
TGTCACAAGGGCGGCGAGACTCTGAGCCACTCCCATTTGCTGCTGCACAAGAA
GGAGAATGGTATCTGGTCTACCGAAATCCTGAAGAATTTTAAGAACAAGACTTT
TCTGAAATGCGAGGCCCCAAATTATTCCGGACGTTTCACTTGCAGTTGGCTCGT
TCAAAGAAATATGGACTTGAAATTTAACATTAAATCCAGCTCTTCATCTCCTGA
CAGCAGGGCCGTAACTTGTGGAATGGCTTCATTGTCAGCTGAGAAAGTTACGCT
TGACCAAAGGGATTATGAGAAATACAGCGTGAGTTGCCAGGAAGATGTGACAT
GTCCAACGGCAGAGGAAACGTTGCCAATTGAGCTCGCTTTGGAAGCTCGTCAA
CAAAACAAGTATGAAAACTATAGTACTAGCTTCTTCATACGGGACATCATCAA
ACCAGATCCACCTAAGAATTTGCAGATGAAGCCTCTGAAGAATTCACAAGTCG
AGGTATCCTGGGAATACCCAGATTCATGGTCCACTCCTCATAGTTACTTTAGCC
TGAAATTCTTTGTACGCATACAGCGGAAGAAGGAGAAAATGAAGGAGACGGA
AGAAGGCTGCAATCAGAAAGGCGCTTTTCTTGTTGAAAAGACGAGCACTGAGG
TTCAATGCAAAGGCGGGAATGTATGTGTTCAAGCCCAAGATAGGTATTATAAT
AGCTCCTGCTCTAAGTGGGCTTGCGTACCATGCAGAGTTAGAAGTCCCAGAGG
GCCCACAATCAAGCCCTGTCCTCCATGCAAATGCCCAGCACCTAACCTCTTGGG
TGGACCATCCGTCTTCATCTTCCCTCCAAAGATCAAGGATGTACTCATGATCTC
CCTGAGCCCCATAGTCACATGTGTGGTGGTGGATGTGAGCGAGGATGACCCAG
ATGTCCAGATCAGCTGGTTTGTGAACAACGTGGAAGTACACACAGCTCAGACA
CAAACCCATAGAGAGGATTACAACAGTACTCTCCGGGTGGTCAGTGCCCTCCCC
ATCCAGCACCAGGACTGGATGAGTGGCAAGGAGTTCAAATGCAAGGTCAACAA
CAAAGACCTCCCAGCGCCCATCGAGAGAACCATCTCAAAACCCAAAGGGTCAG
TAAGAGCTCCACAGGTATATGTCTTGCCTCCACCAGAAGAAGAGATGACTAAG
AAACAGGTCACTCTGACCTGCATGGTCACAGACTTCATGCCTGAAGACATTTAC
GTGGAGTGGACCAACAACGGGAAAACAGAGCTAAACTACAAGAACACTGAAC
CAGTCCTGGACTCTGATGGTTCTTACTTCATGTACAGCAAGCTGAGAGTGGAAA
AGAAGAACTGGGTGGAAAGAAATAGCTACTCCTGTTCAGTGGTCCACGAGGGT
CTGCACAATCACCACACGACTAAGAGCTTCTCCCGGACTCCGGGTAAATAG

mouse IL 12b-mouse IgG2a Fc

(SEQ ID NO: 133)
MCPQKLTISWFAIVLLVSPLMAMWELEKDVYVVEVDWTPDAPGETVNLTCDTPEE
DDITWTSDQRHGVIGSGKTLTITVKEFLDAGQYTCHKGGETLSHSHLLLHKKENGI
WSTEILKNFKNKTFLKCEAPNYSGRFTCSWLVQRNMDLKFNIKSSSSSPDSRAVTC
GMASLSAEKVTLDQRDYEKYSVSCQEDVTCPTAEETLPIELALEARQQNKYENYST
SFFIRDIIKPDPPKNLQMKPLKNSQVEVSWEYPDSWSTPHSYFSLKFFVRIQRKKEK
MKETEEGCNQKGAFLVEKTSTEVQCKGGNVCVQAQDRYYNSSCSKWACVPCRVR
SPRGPTIKPCPPCKCPAPNLLGGPSVFIFPPKIKDVLMISLSPIVTCVVVDVSEDDPDV
QISWFVNNVEVHTAQTQTHREDYNSTLRVVSALPIQHQDWMSGKEFKCKVNNKD
LPAPIERTISKPKGSVRAPQVYVLPPPEEEMTKKQVTLTCMVTDFMPEDIYVEWTN
NGKTELNYKNTEPVLDSDGSYFMYSKLRVEKKNWVERNSYSCSVVHEGLHNHHT
TKSFSRTPGK

mouse IL 12a-mouse IgG2a Fc (Silent)

(SEQ ID NO: 134)
ATGTGTCAGTCACGCTATCTTCTCTTCCTTGCTACTCTGGCCTTGCTCAATCACT
TGTCCCTTGCTCGTGTGATTCCTGTGTCCGGCCCAGCTAGGTGTCTCTCCCAGTC
ACGGAATCTCCTGAAAACCACGGATGACATGGTAAAGACAGCTAGGGAGAAAC
TCAAGCACTACTCCTGCACAGCTGAGGATATCGATCATGAGGACATCACCAGG
GACCAGACATCCACTCTGAAAACTTGCCTGCCTTTGGAACTCCACAAGAACGA
ATCTTGTCTGGCAACGCGTGAAACGAGTTCTACTACAAGAGGGTCCTGTCTTCC
CCCTCAAAAGACAAGCCTTATGATGACCTTGTGTCTCGGTAGCATTTATGAGGA
CCTAAAGATGTATCAAACCGAGTTTCAGGCTATCAATGCAGCGCTCCAGAATCA
TAACCATCAGCAGATCATTCTTGACAAAGGAATGCTCGTGGCCATTGATGAACT
AATGCAGAGCCTAAACCACAATGGCGAGACTCTTCGACAGAAACCGCCTGTGG
GCGAGGCCGATCCATATAGAGTCAAAATGAAACTGTGTATTCTCCTGCATGCAT
TTAGTACTCGTGTAGTGACTATTAACAGAGTGATGGGTTACCTTTCCTCAGCTC
CCAGAGGGCCCACAATCAAGCCCTGTCCTCCATGCAAATGCCCAGCACCTAAC
GCTGCCGGTGGACCATCCGTCTTCATCTTCCCTCCAAAGATCAAGGATGTACTC
ATGATCTCCCTGAGCCCCATAGTCACATGTGTGGTGGTGGATGTGAGCGAGGAT
GACCCAGATGTCCAGATCAGCTGGTTTGTGAACAACGTGGAAGTACACACAGC
TCAGACACAAACCCATAGAGAGGATTACAACAGTACTCTCCGGGTGGTCAGTG
CCCTCCCCATCCAGCACCAGGACTGGATGAGTGGCAAGGAGTTCAAATGCAAG
GTCAACAACAAAGACCTCGGAGCGCCCATCGAGAGAACCATCTCAAAACCCAA
AGGGTCAGTAAGAGCTCCACAGGTATATGTCTTGCCTCCACCAGAAGAAGAGA
TGACTAAGAAACAGGTCACTCTGACCTGCATGGTCACAGACTTCATGCCTGAAG
ACATTTACGTGGAGTGGACCAACAACGGGAAAACAGAGCTAAACTACAAGAAC
ACTGAACCAGTCCTGGACTCTGATGGTTCTTACTTCATGTACAGCAAGCTGAGA
GTGGAAAAGAAGAACTGGGTGGAAAGAAATAGCTACTCCTGTTCAGTGGTCCA
CGAGGGTCTGCACAATCACCACACGACTAAGAGCTTCTCCCGGACTCCGGGTA
AATGA

mouse IL 12a-mouse IgG2a Fc (Silent)

(SEQ ID NO: 135)
MCQSRYLLFLATLALLNHLSLARVIPVSGPARCLSQSRNLLKTTDDMVKTAREKLK
HYSCTAEDIDHEDITRDQTSTLKTCLPLELHKNESCLATRETSSTTRGSCLPPQKTSL
MMTLCLGSIYEDLKMYQTEFQAINAALQNHNHQQIILDKGMLVAIDELMQSLNHN
GETLRQKPPVGEADPYRVKMKLCILLHAFSTRVVTINRVMGYLSSAPRGPTIKPCPP
CKCPAPNAAGGPSVFIFPPKIKDVLMISLSPIVTCVVVDVSEDDPDVQISWFVNNVE
VHTAQTQTHREDYNSTLRVVSALPIQHQDWMSGKEFKCKVNNKDLGAPIERTISKP
KGSVRAPQVYVLPPPEEEMTKKQVTLTCMVTDFMPEDIYVEWTNNGKTELNYKN
TEPVLDSDGSYFMYSKLRVEKKNWVERNSYSCSVVHEGLHNHHTTKSFSRTPGK*

mouse IL 12b-mouse IgG2a Fc (Silent)

(SEQ ID NO: 136)
ATGTGCCCACAGAAACTCACAATTTCTTGGTTCGCAATCGTCCTGCTGGTGTCA
CCCCTGATGGCAATGTGGGAGTTGGAAAAGGATGTATACGTCGTCGAGGTCGA
CTGGACACCTGACGCTCCGGGTGAAACTGTCAACCTCACTTGCGATACTCCTGA
AGAGGACGACATCACGTGGACGAGCGACCAGCGACATGGAGTGATAGGGTCTG
GCAAGACGCTTACTATCACGGTTAAGGAATTTCTCGACGCAGGGCAGTACACA
TGTCACAAGGGCGGCGAGACTCTGAGCCACTCCCATTTGCTGCTGCACAAGAA
GGAGAATGGTATCTGGTCTACCGAAATCCTGAAGAATTTTAAGAACAAGACTTT
TCTGAAATGCGAGGCCCCAAATTATTCCGGACGTTTCACTTGCAGTTGGCTCGT
TCAAAGAAATATGGACTTGAAATTTAACATTAAATCCAGCTCTTCATCTCCTGA
CAGCAGGGCCGTAACTTGTGGAATGGCTTCATTGTCAGCTGAGAAAGTTACGCT
TGACCAAAGGGATTATGAGAAATACAGCGTGAGTTGCCAGGAAGATGTGACAT
GTCCAACGGCAGAGGAAACGTTGCCAATTGAGCTCGCTTTGGAAGCTCGTCAA
CAAAACAAGTATGAAAACTATAGTACTAGCTTCTTCATACGGGACATCATCAA
ACCAGATCCACCTAAGAATTTGCAGATGAAGCCTCTGAAGAATTCACAAGTCG
AGGTATCCTGGGAATACCCAGATTCATGGTCCACTCCTCATAGTTACTTTAGCC
TGAAATTCTTTGTACGCATACAGCGGAAGAAGGAGAAAATGAAGGAGACGGA
AGAAGGCTGCAATCAGAAAGGCGCTTTTCTTGTTGAAAAGACGAGCACTGAGG
TTCAATGCAAAGGCGGGAATGTATGTGTTCAAGCCCAAGATAGGTATTATAAT
AGCTCCTGCTCTAAGTGGGCTTGCGTACCATGCAGAGTTAGAAGTCCCAGAGG
GCCCACAATCAAGCCCTGTCCTCCATGCAAATGCCCAGCACCTAACGCTGCCGG
TGGACCATCCGTCTTCATCTTCCCTCCAAAGATCAAGGATGTACTCATGATCTC
CCTGAGCCCCATAGTCACATGTGTGGTGGTGGATGTGAGCGAGGATGACCCAG
ATGTCCAGATCAGCTGGTTTGTGAACAACGTGGAAGTACACACAGCTCAGACA
CAAACCCATAGAGAGGATTACAACAGTACTCTCCGGGTGGTCAGTGCCCTCCCC
ATCCAGCACCAGGACTGGATGAGTGGCAAGGAGTTCAAATGCAAGGTCAACAA
CAAAGACCTCGGAGCGCCCATCGAGAGAACCATCTCAAAACCCAAAGGGTCAG
TAAGAGCTCCACAGGTATATGTCTTGCCTCCACCAGAAGAAGAGATGACTAAG
AAACAGGTCACTCTGACCTGCATGGTCACAGACTTCATGCCTGAAGACATTTAC
GTGGAGTGGACCAACAACGGGAAAACAGAGCTAAACTACAAGAACACTGAAC
CAGTCCTGGACTCTGATGGTTCTTACTTCATGTACAGCAAGCTGAGAGTGGAAA
AGAAGAACTGGGTGGAAAGAAATAGCTACTCCTGTTCAGTGGTCCACGAGGGT
CTGCACAATCACCACACGACTAAGAGCTTCTCCCGGACTCCGGGTAAATGA

mouse IL 12b-mouse IgG2a Fc (Silent)

(SEQ ID NO: 137)
MCPQKLTISWFAIVLLVSPLMAMWELEKDVYVVEVDWTPDAPGETVNLTCDTPEE
DDITWTSDQRHGVIGSGKTLTITVKEFLDAGQYTCHKGGETLSHSHLLLHKKENGI
WSTEILKNFKNKTFLKCEAPNYSGRFTCSWLVQRNMDLKFNIKSSSSSPDSRAVTC
GMASLSAEKVTLDQRDYEKYSVSCQEDVTCPTAEETLPIELALEARQQNKYENYST
SFFIRDIIKPDPPKNLQMKPLKNSQVEVSWEYPDSWSTPHSYFSLKFFVRIQRKKEK
MKETEEGCNQKGAFLVEKTSTEVQCKGGNVCVQAQDRYYNSSCSKWACVPCRVR
SPRGPTIKPCPPCKCPAPNAAGGPSVFIFPPKIKDVLMISLSPIVTCVVVDVSEDDPDV
QISWFVNNVEVHTAQTQTHREDYNSTLRVVSALPIQHQDWMSGKEFKCKVNNKD
LGAPIERTISKPKGSVRAPQVYVLPPPEEEMTKKQVTLTCMVTDFMPEDIYVEWTN
NGKTELNYKNTEPVLDSDGSYFMYSKLRVEKKNWVERNSYSCSVVHEGLHNHHT
TKSFSRTPGK

CD3 zeta cytoplasmic domain-human

(SEQ ID NO: 138)
RVKFSRSADAPAYQQGQNQLYNELNLGRREEYDVLDKRRGRDPEMGGKPQRRKN
PQEGLYNELQKDKMAEAYSEIGMKGERRRGKGHDGLYQGLSTATKDTYDALHM
QALPPR

CD3 zeta cytoplasmic domain-human

(SEQ ID NO: 139)
AGAGTGAAGTTCAGCAGGAGCGCAGACGCCCCCGCGTACCAGCAGGGCCAGA
ACCAGCTCTATAACGAGCTCAATCTAGGACGAAGAGAGGAGTACGATGTTTTG
GACAAGAGACGTGGCCGGGACCCTGAGATGGGGGGAAAGCCGCAGAGAAGGA
AGAACCCTCAGGAAGGCCTGTACAATGAACTGCAGAAAGATAAGATGGCGGA
GGCCTACAGTGAGATTGGGATGAAAGGCGAGCGCCGGAGGGGCAAGGGGCAC
GATGGCCTTTACCAGGGTCTCAGTACAGCCACCAAGGACACCTACGACGCCCTT
CACATGCAGGCCCTGCCCCCTCGCTAA

pWF-506

(SEQ ID NO: 140)
METDTLLLWVLLLWVPGSTGQSVLTQPPSVSAAPGQRVTISCSGTRSNIGSDYVSW
YQHLPGTAPKLLVYGDNLRPSGIPDRFSASKSGTSATLGITGLQTGDEADYYCGTW
DYTLNGVVFGGGTKLTVLGSRGGGGSGGGGSGGGGSLEMAQVQLVESGGGLVQP
GGSLRLSCAASGFTFSSYAMSWVRQAPGKGLEWVSVIYSGGSSTYYADSVKGRFTI
SRDNSKNTLYLQMNSLRAEDTAVYYCARTSYLNHGDYWGQGTLVTVSSPKSCDK
THTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQKSLSLS
PELQLEESCAEAQDGELDGLWTTITIFITLFLLSVCYSATVTFFKVKWIFSSVVDLKQ
TIIPDYRNMIGQGA

pWF-506

(SEQ ID NO: 141)
ATGGAAACCGATACACTGCTGCTGTGGGTGCTGCTGCTGTGGGTGCCAGGATCT
ACCGGTCAGTCTGTGTTGACGCAGCCGCCCTCAGTGTCTGCGGCCCCAGGACAG
AGGGTCACCATCTCCTGCTCTGGAACCAGGTCCAACATTGGGAGTGATTATGTT
TCCTGGTACCAACACCTCCCAGGAACAGCCCCCAAACTCCTCGTTTATGGCGAT
AATCTGCGACCCTCAGGGATTCCTGACCGATTCTCTGCCTCCAAGTCTGGCACG
TCAGCCACCCTGGGCATCACCGGACTCCAGACTGGGGACGAGGCCGATTATTA
CTGCGGCACATGGGATTACACCCTGAATGGTGTGGTGTTCGGCGGAGGGACCA
AGCTGACCGTCCTAGGTTCTAGAGGTGGTGGTGGTAGCGGCGGCGGCGGCTCT
GGTGGTGGTGGATCCCTCGAGATGGCCCAGGTGCAGCTGGTGGAGTCTGGGGG
AGGCTTGGTACAGCCTGGGGGGTCCCTGAGACTCTCCTGTGCAGCCTCTGGATT
CACCTTTAGCAGCTATGCCATGAGCTGGGTCCGCCAGGCTCCAGGGAAGGGGC
TGGAGTGGGTCTCAGTTATTTATAGCGGTGGTAGTAGCACATACTATGCAGACT
CCGTGAAGGGCCGGTTCACCATCTCCAGAGATAATTCCAAGAACACGCTGTATC
TGCAAATGAACAGCCTGAGAGCCGAGGACACGGCCGTATATTACTGTGCGCGC
ACTTCTTACCTGAACCATGGTGATTACTGGGGTCAAGGTACTCTGGTGACCGTG
TCTAGCCCCAAGAGCTGCGACAAGACCCACACCTGCCCCCCCTGCCCAGCCCCA
GAGCTGCTGGGCGGACCCTCCGTGTTCCTGTTCCCCCCCAAGCCCAAGGACACC
CTGATGATCAGCAGGACCCCCGAGGTGACCTGCGTGGTGGTGGACGTGAGCCA
CGAGGACCCAGAGGTGAAGTTCAACTGGTACGTGGACGGCGTGGAGGTGCACA
ACGCCAAGACCAAGCCCAGAGAGGAGCAGTACAACAGCACCTACAGGGTGGT
GTCCGTGCTGACCGTGCTGCACCAGGACTGGCTGAACGGCAAGGAATACAAGT
GCAAGGTCTCCAACAAGGCCCTGCCAGCCCCCATCGAAAAGACCATCAGCAAG
GCCAAGGGCCAGCCACGGGAGCCCCAGGTGTACACCCTGCCCCCCTCCCGGGA
GGAGATGACCAAGAACCAGGTGTCCCTGACCTGTCTGGTGAAGGGCTTCTACC
CCAGCGACATCGCCGTGGAGTGGGAGAGCAACGGCCAGCCCGAGAACAACTAC
AAGACCACCCCCCCAGTGCTGGACAGCGACGGCAGCTTCTTCCTGTACAGCAA
GCTGACCGTGGACAAGTCCAGGTGGCAGCAGGGCAACGTGTTCAGCTGCAGCG
TGATGCACGAGGCCCTGCACAACCACTACACCCAGAAGAGCCTGAGCCTGTCC
CCCGAGCTGCAACTGGAGGAGAGCTGTGCGGAGGCGCAGGACGGGGAGCTGG
ACGGGCTGTGGACGACCATCACCATCTTCATCACACTCTTCCTGTTAAGCGTGT
GCTACAGTGCCACCGTCACCTTCTTCAAGGTGAAGTGGATCTTCTCCTCGGTGG
TGGACCTGAAGCAGACCATCATCCCCGACTACAGGAACATGATCGGACAGGGG
GCCTGA

pWF-507:

(SEQ ID NO: 142)
METDTLLLWVLLLWVPGSTGQSVLTQPPSVSAAPGQRVTISCSGTRSNIGSDYVSW
YQHLPGTAPKLLVYGDNLRPSGIPDRFSASKSGTSATLGITGLQTGDEADYYCGTW
DYTLNGVVFGGGTKLTVLGQPKANPTVTLFPPSSEELQANKATLVCLISDFYPGAV
TVAWKADGSPVKAGVETTKPSKQSNNKYAASSYLSLTPEQWKSHRSYSCQVTHE
GSTVEKTVAPTECS

pWF-507:

(SEQ ID NO: 143)
ATGGAAACCGATACACTGCTGCTGTGGGTGCTGCTGCTGTGGGTGCCAGGATCT
ACCGGTCAGTCTGTGTTGACGCAGCCGCCCTCAGTGTCTGCGGCCCCAGGACAG
AGGGTCACCATCTCCTGCTCTGGAACCAGGTCCAACATTGGGAGTGATTATGTT
TCCTGGTACCAACACCTCCCAGGAACAGCCCCCAAACTCCTCGTTTATGGCGAT
AATCTGCGACCCTCAGGGATTCCTGACCGATTCTCTGCCTCCAAGTCTGGCACG
TCAGCCACCCTGGGCATCACCGGACTCCAGACTGGGGACGAGGCCGATTATTA
CTGCGGCACATGGGATTACACCCTGAATGGTGTGGTGTTCGGCGGAGGGACCA
AGCTGACCGTCCTAGGTCAGCCCAAGGCCAACCCCACTGTCACTCTGTTCCCGC
CCTCCTCTGAGGAGCTCCAAGCCAACAAGGCCACACTAGTGTGTCTGATCAGTG
ACTTCTACCCGGGAGCTGTGACAGTGGCCTGGAAGGCAGATGGCAGCCCCGTC
AAGGCGGGAGTGGAGACCACCAAACCCTCCAAACAGAGCAACAACAAGTACG
CGGCCAGCAGCTACCTGAGCCTGACGCCCGAGCAGTGGAAGTCCCACAGAAGC
TACAGCTGCCAGGTCACGCATGAAGGGAGCACCGTGGAGAAGACAGTGGCCCC
TACAGAATGTTCATAG

pWF-508:

(SEQ ID NO: 144)
MVFTPQILGLMLFWISASRGQVQLVESGGGLVQPGGSLRLSCAASGFTFSSYAMSW
VRQAPGKGLEWVSVIYSGGSSTYYADSVKGRFTISRDNSKNTLYLQMNSLRAEDT
AVYYCARTSYLNHGDYWGQGTLVTVSSASTKGPSVFPLAPSSKSTSGGTAALGCL
VKDYFPEPVTVSWNSGALTSGVHTFPAVLQSSGLYSLSSVVTVPSSSLGTQTYICNV
NHKPSNTKVDKRVEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGNVF
SCSVMHEALHNHYTQKSLSLSPELQLEESCAEAQDGELDGLWTTITIFITLFLLSVC
YSATVTFFKVKWIFSSVVDLKQTIIPDYRNMIGQGA

pWF-508:

(SEQ ID NO: 145)
ATGGTGTTTACACCGCAAATATTGGGGCTCATGCTTTTCTGGATCAGTGCAAGC
AGGGGACAGGTGCAGCTGGTGGAGTCTGGGGGAGGCTTGGTACAGCCTGGGGG
GTCCCTGAGACTCTCCTGTGCAGCCTCTGGATTCACCTTTAGCAGCTATGCCAT
GAGCTGGGTCCGCCAGGCTCCAGGGAAGGGGCTGGAGTGGGTCTCAGTTATTT
ATAGCGGTGGTAGTAGCACATACTATGCAGACTCCGTGAAGGGCCGGTTCACC
ATCTCCAGAGATAATTCCAAGAACACGCTGTATCTGCAAATGAACAGCCTGAG
AGCCGAGGACACGGCCGTATATTACTGTGCGCGCACTTCTTACCTGAACCATGG
TGATTACTGGGGTCAAGGTACTCTGGTGACCGTGTCTAGCGCCTCCACCAAGGG
CCCATCGGTCTTCCCCCTGGCACCCTCCTCCAAGAGCACCTCTGGGGGCACAGC
GGCCCTGGGCTGCCTGGTCAAGGACTACTTCCCCGAACCGGTGACGGTGTCGTG
GAACTCAGGCGCCCTGACCAGCGGCGTGCACACCTTCCCGGCTGTCCTACAGTC
CTCAGGACTCTACTCCCTCAGCAGCGTGGTGACCGTGCCCTCCAGCAGCTTGGG
CACCCAGACCTACATCTGCAACGTGAACCACAAGCCCAGCAACACCAAGGTGG
ACAAGAGAGTGGAGCCCAAGAGCTGCGACAAGACCCACACCTGCCCCCCCTGC
CCAGCCCCAGAGCTGCTGGGCGGACCCTCCGTGTTCCTGTTCCCCCCCAAGCCC
AAGGACACCCTGATGATCAGCAGGACCCCCGAGGTGACCTGCGTGGTGGTGGA
CGTGAGCCACGAGGACCCAGAGGTGAAGTTCAACTGGTACGTGGACGGCGTGG
AGGTGCACAACGCCAAGACCAAGCCCAGAGAGGAGCAGTACAACAGCACCTA
CAGGGTGGTGTCCGTGCTGACCGTGCTGCACCAGGACTGGCTGAACGGCAAGG
AATACAAGTGCAAGGTCTCCAACAAGGCCCTGCCAGCCCCCATCGAAAAGACC
ATCAGCAAGGCCAAGGGCCAGCCACGGGAGCCCCAGGTGTACACCCTGCCCCC
CTCCCGGGAGGAGATGACCAAGAACCAGGTGTCCCTGACCTGTCTGGTGAAGG
GCTTCTACCCCAGCGACATCGCCGTGGAGTGGGAGAGCAACGGCCAGCCCGAG
AACAACTACAAGACCACCCCCCCAGTGCTGGACAGCGACGGCAGCTTCTTCCT
GTACAGCAAGCTGACCGTGGACAAGTCCAGGTGGCAGCAGGGCAACGTGTTCA
GCTGCAGCGTGATGCACGAGGCCCTGCACAACCACTACACCCAGAAGAGCCTG
AGCCTGTCCCCCGAGCTGCAACTGGAGGAGAGCTGTGCGGAGGCGCAGGACGG
GGAGCTGGACGGGCTGTGGACGACCATCACCATCTTCATCACACTCTTCCTGTT
AAGCGTGTGCTACAGTGCCACCGTCACCTTCTTCAAGGTGAAGTGGATCTTCTC
CTCGGTGGTGGACCTGAAGCAGACCATCATCCCCGACTACAGGAACATGATCG
GACAGGGGGCCTGA

pWF-509:

(SEQ ID NO: 146)
METDTLLLWVLLLWVPGSTGQSVLTQPPSVSAAPGQRVTISCSGTRSNIGSDYVSW
YQHLPGTAPKLLVYGDNLRPSGIPDRFSASKSGTSATLGITGLQTGDEADYYCGTW
DYTLNGVVFGGGTKLTVLGSRGGGGSGGGGSGGGGSLEMAQVQLVESGGGLVQP
GGSLRLSCAASGFTFSSYAMSWVRQAPGKGLEWVSVIYSGGSSTYYADSVKGRFTI
SRDNSKNTLYLQMNSLRAEDTAVYYCARTSYLNHGDYWGQGTLVTVSSAAAFVP
VFLPAKPTTTPAPRPPTPAPTIASQPLSLRPEACRPAAGGAVHTRGLDFACDFWVLV
VVGGVLACYSLLVTVAFIIFWVLDKDDSKAGMEEDHTYEGLDIDQTATYEDIVTLR
TGEVKWSVGEHPGQE

pWF-509:

(SEQ ID NO: 147)
ATGGAAACCGATACACTGCTGCTGTGGGTGCTGCTGCTGTGGGTGCCAGGATCT
ACCGGTCAGTCTGTGTTGACGCAGCCGCCCTCAGTGTCTGCGGCCCCAGGACAG
AGGGTCACCATCTCCTGCTCTGGAACCAGGTCCAACATTGGGAGTGATTATGTT
TCCTGGTACCAACACCTCCCAGGAACAGCCCCCAAACTCCTCGTTTATGGCGAT
AATCTGCGACCCTCAGGGATTCCTGACCGATTCTCTGCCTCCAAGTCTGGCACG
TCAGCCACCCTGGGCATCACCGGACTCCAGACTGGGGACGAGGCCGATTATTA
CTGCGGCACATGGGATTACACCCTGAATGGTGTGGTGTTCGGCGGAGGGACCA
AGCTGACCGTCCTAGGTTCTAGAGGTGGTGGTGGTAGCGGCGGCGGCGGCTCT
GGTGGTGGTGGATCCCTCGAGATGGCCCAGGTGCAGCTGGTGGAGTCTGGGGG
AGGCTTGGTACAGCCTGGGGGGTCCCTGAGACTCTCCTGTGCAGCCTCTGGATT
CACCTTTAGCAGCTATGCCATGAGCTGGGTCCGCCAGGCTCCAGGGAAGGGGC
TGGAGTGGGTCTCAGTTATTTATAGCGGTGGTAGTAGCACATACTATGCAGACT
CCGTGAAGGGCCGGTTCACCATCTCCAGAGATAATTCCAAGAACACGCTGTATC
TGCAAATGAACAGCCTGAGAGCCGAGGACACGGCCGTATATTACTGTGCGCGC
ACTTCTTACCTGAACCATGGTGATTACTGGGGTCAAGGTACTCTGGTGACCGTG
TCTAGCGCCGCTGCATTCGTGCCTGTGTTCCTCCCAGCTAAGCCCACTACCACC
CCCGCTCCAAGGCCGCCCACGCCCGCTCCTACTATTGCTAGTCAGCCTTTAAGT
TTACGACCCGAAGCTTGCAGGCCCGCCGCCGGCGGCGCTGTGCACACCAGGGG
GCTTGATTTTGCCTGCGACTTTTGGGTATTGGTAGTGGTGGGCGGAGTTTTAGC
CTGCTACAGCCTCCTGGTAACAGTGGCTTTTATCATCTTTTGGGTGCTGGACAA
GGATGACAGCAAGGCTGGCATGGAGGAAGATCACACCTACGAGGGCCTGGAC
ATTGACCAGACAGCCACCTATGAGGACATAGTGACGCTGCGGACAGGGGAAGT
GAAGTGGTCTGTAGGTGAGCACCCAGGCCAGGAGTGA

pWF-510:

(SEQ ID NO: 148)
METDTLLLWVLLLWVPGSTGQSVLTQPPSVSAAPGQRVTISCSGTRSNIGSDYVSW
YQHLPGTAPKLLVYGDNLRPSGIPDRFSASKSGTSATLGITGLQTGDEADYYCGTW
DYTLNGVVFGGGTKLTVLGSRGGGGSGGGGSGGGGSLEMAQVQLVESGGGLVQP
GGSLRLSCAASGFTFSSYAMSWVRQAPGKGLEWVSVIYSGGSSTYYADSVKGRFTI
SRDNSKNTLYLQMNSLRAEDTAVYYCARTSYLNHGDYWGQGTLVTVSSAAAFVP
VFLPAKPTTTPAPRPPTPAPTIASQPLSLRPEACRPAAGGAVHTRGLDFACDFWVLV
VVGGVLACYSLLVTVAFIIFWVRKRWQNEKLGLDAGDEYEDENLYEGLNLDDCS
MYEDISRGLQGTYQDVGSLNIGDVQLEKP

pWF-510:

(SEQ ID NO: 149)
ATGGAAACCGATACACTGCTGCTGTGGGTGCTGCTGCTGTGGGTGCCAGGATCT
ACCGGTCAGTCTGTGTTGACGCAGCCGCCCTCAGTGTCTGCGGCCCCAGGACAG
AGGGTCACCATCTCCTGCTCTGGAACCAGGTCCAACATTGGGAGTGATTATGTT
TCCTGGTACCAACACCTCCCAGGAACAGCCCCCAAACTCCTCGTTTATGGCGAT
AATCTGCGACCCTCAGGGATTCCTGACCGATTCTCTGCCTCCAAGTCTGGCACG
TCAGCCACCCTGGGCATCACCGGACTCCAGACTGGGGACGAGGCCGATTATTA
CTGCGGCACATGGGATTACACCCTGAATGGTGTGGTGTTCGGCGGAGGGACCA
AGCTGACCGTCCTAGGTTCTAGAGGTGGTGGTGGTAGCGGCGGCGGCGGCTCT
GGTGGTGGTGGATCCCTCGAGATGGCCCAGGTGCAGCTGGTGGAGTCTGGGGG
AGGCTTGGTACAGCCTGGGGGGTCCCTGAGACTCTCCTGTGCAGCCTCTGGATT
CACCTTTAGCAGCTATGCCATGAGCTGGGTCCGCCAGGCTCCAGGGAAGGGGC
TGGAGTGGGTCTCAGTTATTTATAGCGGTGGTAGTAGCACATACTATGCAGACT
CCGTGAAGGGCCGGTTCACCATCTCCAGAGATAATTCCAAGAACACGCTGTATC
TGCAAATGAACAGCCTGAGAGCCGAGGACACGGCCGTATATTACTGTGCGCGC
ACTTCTTACCTGAACCATGGTGATTACTGGGGTCAAGGTACTCTGGTGACCGTG
TCTAGCGCCGCTGCATTCGTGCCTGTGTTCCTCCCAGCTAAGCCCACTACCACC
CCCGCTCCAAGGCCGCCCACGCCCGCTCCTACTATTGCTAGTCAGCCTTTAAGT
TTACGACCCGAAGCTTGCAGGCCCGCCGCCGGCGGCGCTGTGCACACCAGGGG
GCTTGATTTTGCCTGCGACTTTTGGGTATTGGTAGTGGTGGGCGGAGTTTTAGCC
TGCTACAGCCTCCTGGTAACAGTGGCTTTTATCATCTTTTGGGTGAGGAAACGA
TGGCAGAACGAGAAGCTCGGGTTGGATGCCGGGGATGAATATGAAGATGAAA
ACCTTTATGAAGGCCTGAACCTGGACGACTGCTCCATGTATGAGGACATCTCCC
GGGGCCTCCAGGGCACCTACCAGGATGTGGGCAGCCTCAACATAGGAGATGTC
CAGCTGGAGAAGCCGTGA

The nucleic acid and amino acid sequences for the clone GPC3-TM-OVA used herein are set forth below.

huGPC3 extra-cellular domain-mouse CD80 (transmembrane + cytoplasmic domain) + OVA (MHC I, MHC II) epitopes

Nucleotide Sequence: (Seq Id No: 151)
ATGGCCGGGACCGTGCGCACCGCGTGCTTGGTGGTGGCGATGCTGCTCAGCTTGGACT
TCCCGGGACAGGCGCAGCCCCCGCCGCCGCCGCCGGACGCCACCTGTCACCAAGTCC
GCTCCTTCTTCCAGAGACTGCAGCCCGGACTCAAGTGGGTGCCAGAAACTCCCGTGCC
AGGATCAGATTTGCAAGTATGTCTCCCTAAGGGCCCAACATGCTGCTCAAGAAAGAT
GGAAGAAAAATACCAACTAACAGCACGATTGAACATGGAACAGCTGCTTCAGTCTGC
AAGTATGGAGCTCAAGTTCTTAATTATTCAGAATGCTGCGGTTTTCCAAGAGGCCTTT
GAAATTGTTGTTCGCCATGCCAAGAACTACACCAATGCCATGTTCAAGAACAACTACC
CAAGCCTGACTCCACAAGCTTTTGAGTTTGTGGGTGAATTTTTCACAGATGTGTCTCTC
TACATCTTGGGTTCTGACATCAATGTAGATGACATGGTCAATGAATTGTTTGACAGCC
TGTTTCCAGTCATCTATACCCAGCTAATGAACCCAGGCCTGCCTGATTCAGCCTTGGA
CATCAATGAGTGCCTCCGAGGAGCAAGACGTGACCTGAAAGTATTTGGGAATTTCCC
CAAGCTTATTATGACCCAGGTTTCCAAGTCACTGCAAGTCACTAGGATCTTCCTTCAG
GCTCTGAATCTTGGAATTGAAGTGATCAACACAACTGATCACCTGAAGTTCAGTAAGG
ACTGTGGCCGAATGCTCACCAGAATGTGGTACTGCTCTTACTGCCAGGGACTGATGAT
GGTTAAACCCTGTGGCGGTTACTGCAATGTGGTCATGCAAGGCTGTATGGCAGGTGTG
GTGGAGATTGACAAGTACTGGAGAGAATACATTCTGTCCCTTGAAGAACTTGTGAAT
GGCATGTACAGAATCTATGACATGGAGAACGTACTGCTTGGTCTCTTTTCAACAATCC
ATGATTCTATCCAGTATGTCCAGAAGAATGCAGGAAAGCTGACCACCACTATTGGCA
AGTTATGTGCCCATTCTCAACAACGCCAATATAGATCTGCTTATTATCCTGAAGATCT
CTTTATTGACAAGAAAGTATTAAAAGTTGCTCATGTAGAACATGAAGAAACCTTATCC
AGCCGAAGAAGGGAACTAATTCAGAAGTTGAAGTCTTTCATCAGCTTCTATAGTGCTT
TGCCTGGCTACATCTGCAGCCATAGCCCTGTGGCGGAAAACGACACCCTTTGCTGGAA
TGGACAAGAACTCGTGGAGAGATACAGCCAAAAGGCAGCAAGGAATGGAATGAAAA
ACCAGTTCAATCTCCATGAGCTGAAAATGAAGGGCCCTGAGCCAGTGGTCAGTCAAA
TTATTGACAAACTGAAGCACATTAACCAGCTCCTGAGAACCATGTCTATGCCCAAAGG
TAGAGTTCTGGATAAAAACCTGGATGAGGAAGGGTTTGAAAGTGGAGACTGCGGTGA
TGATGAAGATGAGTGCATTGGAGGCTCTGGTGATGGAATGATAAAAGTGAAGAATCA
GCTCCGCTTCCTTGCAGAACTGGCCTATGATCTGGATGTGGATGATGCGCCTGGAAAC
AGTCAGCAGGCAACTCCGAAGGACAACGAGATAAGCACCTTTCACAACGAGAAACCA
CCAGAGGACCCGCCAGACAGCAAGAATACACTTGTCCTCTTTGGCGCTGGGTTCGGC
GCCGTCATAACGGTTGTTGTCATCGTGGTAATAATCAAGTGCTTTTGCAAGCACAGGT
CTTGTTTTCGCAGGAATGAAGCCTCTAGAGAAACAAATAATTCACTGACCTTTGGCCC
CGAAGAAGCTCTTGCAGAGCAAACGGTGTTTCTCATGCTCGTGTTGTTGCCTGACGAA
GTGTCAGGCCTTGAACAGTTGGAATCAATCATCAATTTTGAAAAACTGACCGAGTGG
ACATCTAGCAACGTTATGGAGGAGCGAAAGATAAAAGTGTATCTGCCTAGAATGAAG
ATGGAGGAAAAGTATAACCTGACTTCCGTCCTGATGGCCATGGGGATCACCGATGTTT
TCAGCAGCTCCGCTAACCTTAGCGGCATTTCCAGCGCTGAGAGTCTGAAGATTTCTCA
AGCAGTGCACGCTGCCCACGCCGAGATTAATGAGGCTGGGCGCGAAGTAGTCGGCTC
AGCAGAGGCAGGCGTCGATGCAGCATAG

Amino Acid Sequence: (Seq Id No: 152)
MAGTVRTACLVVAMLLSLDFPGQAQPPPPPPDATCHQVRSFFQRLQPGLKWVPETPVPGS
DLQVCLPKGPTCCSRKMEEKYQLTARLNMEQLLQSASMELKFLIIQNAAVFQEAFEIVVR
HAKNYTNAMFKNNYPSLTPQAFEFVGEFFTDVSLYILGSDINVDDMVNELFDSLFPVIYTQ
LMNPGLPDSALDINECLRGARRDLKVFGNFPKLIMTQVSKSLQVTRIFLQALNLGIEVINTT
DHLKFSKDCGRMLTRMWYCSYCQGLMMVKPCGGYCNVVMQGCMAGVVEIDKYWRE
YILSLEELVNGMYRIYDMENVLLGLFSTIHDSIQYVQKNAGKLTTTIGKLCAHSQQRQYRS
AYYPEDLFIDKKVLKVAHVEHEETLSSRRRELIQKLKSFISFYSALPGYICSHSPVAENDTL
CWNGQELVERYSQKAARNGMKNQFNLHELKMKGPEPVVSQIIDKLKHINQLLRTMSMP
KGRVLDKNLDEEGFESGDCGDDEDECIGGSGDGMIKVKNQLRFLAELAYDLDVDDAPGN
SQQATPKDNEISTFHNEKPPEDPPDSKNTLVLFGAGFGAVITVVVIVVIIKCFCKHRSCFRR
NEASRETNNSLTFGPEEALAEQTVFLMLVLLPDEVSGLEQLESIINFEKLTEWTSSNVMEE
RKIKVYLPRMKMEEKYNLTSVLMAMGITDVFSSSANLSGISSAESLKISQAVHAAHAEINE
AGREVVGSAEAGVDAA

### INCORPORATION BY REFERENCE

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present invention. To the extent that any of the definitions or terms provided in the references incorporated by reference differ from the terms and discussion provided herein, the present terms and definitions control.

### EQUIVALENTS

The foregoing written specification is considered to be sufficient to enable one skilled in the art to practice the invention. The foregoing description and examples detail certain preferred embodiments of the invention and describe the best mode contemplated by the inventors. It will be appreciated, however, that no matter how detailed the foregoing may appear in text, the invention may be practiced in many ways and the invention should be construed in accordance with the appended claims and any equivalents thereof.

The following examples, including the experiments conducted and results achieved, are provided for illustrative purposes only and are not to be construed as limiting the present invention.

### EXAMPLES

### Example 1 - Chimeric Antigen Receptor for B Cells (CAR-B) Constructs to Bind PSMA

DNA Constructs. Exemplary CAR-B constructs were designed to recognize Prostate Specific Membrane Antigen (“PSMA”). PSMA is an antigen that is expressed more highly on prostate cancer cells than on other non-cancerous cells. Various construct were made comprising an extracellular domain that comprised an scFv specific for PSMA, an extracellular hinge region from CD8, a CD28 transmembrane domain, and various intracellular signaling domains. A list of the constructs is provided in Table 6:

TABLE 6

| | |
|---|---|
| Construct | Description |
| pWF-82 | pTLPW-SFFV-XENP14484 scFv-hCD8H-hCD28M-hCD19E (SEQ ID NOS. 39 and 40) |
| pWF-83 | pTLPW-SFFV-XENP14484 scFv-hCD8H-hCD28M-hCD40E (SEQ ID NOS. 41 and 42) |
| pWF-84 | pTLPW-SFFV-XENP14484 scFv-hCD8H-hCD28M-h(CD40+CD79b)E (SEQ ID NOS. 43 and 44) |
| pWF-85 | pTLPW-SFFV-XENP14484 scFv-hCD8H-hCD28M-h(CD40+CD137)E (SEQ ID NOS. 45 and 46) |
| pWF-86 | pTLPW-SFFV-XENP14484 scFv-hCD8H-hCD28M-h(CD40+Fcγr2a)E (SEQ ID NOS. 47) |
| pWF-87 | pTLPW-SFFV-XENP14484 scFv-hCD8H-hCD28M-h(hMyd88+CD40)E (SEQ ID NOS. 48 and 49) |
| pWF-88 | pTLPW-SFFV-XENP14484 scFv-hCD8H-hCD28M-hCD79aE (SEQ ID NOS. 50 and 51) |
| pWF-89 | pTLPW-SFFV-XENP14484 scFv-hCD8H-hCD28M-hCD79bE (SEQ ID NOS. 52 and 53) |

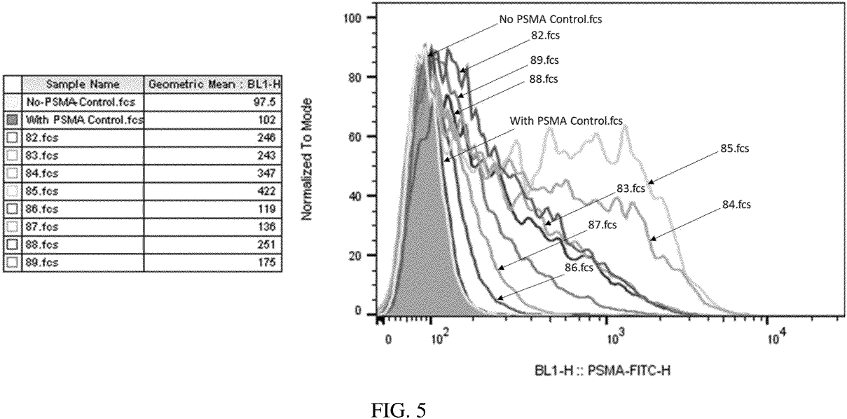

Expression of anti-PSMA CAR-B on HEK-293 Cells. The constructs encoding pWF82 to pWF89 were used to prepare lentivirus in Lentix cells using the Takara lentivirus preparation kit. Expression of the various CAR-B constructs was measured using flow cytometry using antibodies specific for PSMA (biotin-PSMA, Sinobiological and is depicted in FIG. 5.

Expression of anti-PSMA CAR-B in Human B Cells. To measure expression and binding of anti-PSMA CAR-B's in B cells, two additional constructs were made:

TABLE 7

| | |
|---|---|
| Construct | Description |
| pWF-391 | pMMLV(LTR)-hEF1a promoter-anti hPSMA(XENP14484)-CBCR (SEQ ID NOS. 54 and 55) |
| pWF-394 | pMMLV(LTR)-hEF1a promoter-anti sarcoglycan CBCR1 (SEQ ID NOS. 56 and 57) |

A MMLV based vector was used for the preparation of the retrovirus. The retrovirus was used to infect mouse B cells isolated from the spleen. After transduction, B cells were further expanded on feeder cells expressing CD40L and soluble IL-4. The expression of anti-PSMA CAR-B was detected by using recombinant biotin-PSMA. PE-labeled streptavidin was used to detect PSMA binding in HEK-293 cells.

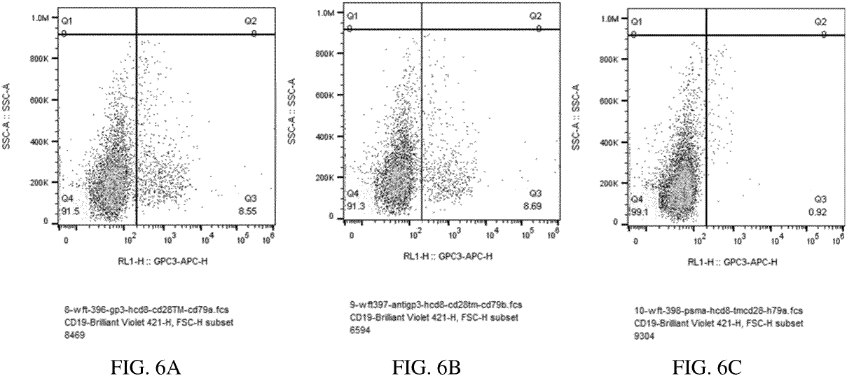

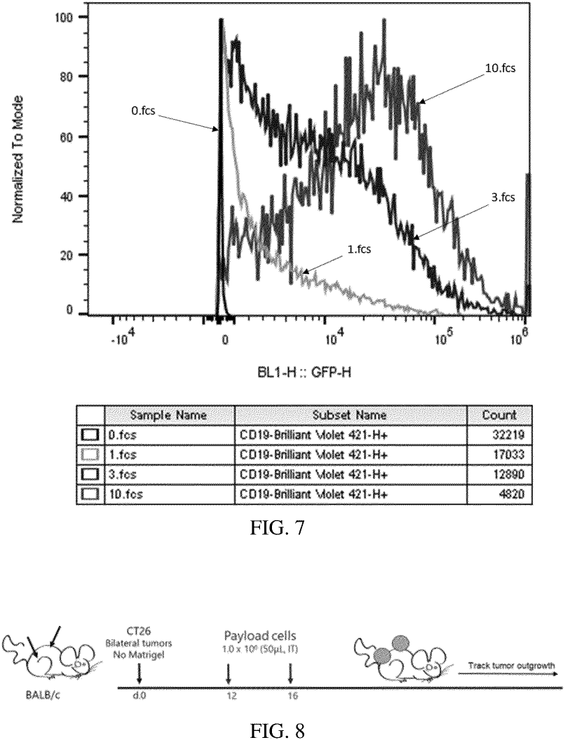

Results. The results of this experiment are depicted in FIG. 6 and demonstrate that it is possible to create mouse B cell that expresses a CAR-B that can bind with specificity to an antigen. For example, B cells expressing pWF396 or pWF397 bound to PSMA whereas the B cells expressing pWF394 did not bind PSMA pWF398 was designed to bind sarcoglycan not PSMA).

### Example 2 - Chimeric Antigen Receptor on B Cells (CAR-B) Constructs to Bind GPC3

DNA Construct. Exemplary CAR constructs were designed to recognize glypican-3 (GPC-3). Glypican-3 is expressed on hepatocellular carcinoma cells among other tumor types, but not on most non-cancer cells. GPC3 can be used to target an anti-GPC3 CAR to hepatocellular carcinoma, as well as other cancers in which GPC3 is expressed (e.g. ovarian clear cell carcinoma, pediatric cancers, lung cancers (i.e. lung adenocarcinoma and lung squamous cell carcinoma), urothelial carcinoma, thyroid cancer, gastric cancer, and others). Various construct were made comprising an extracellular domain that comprised an scFv specific for GPC-3, an extracellular hinge region from CD8, a CD28 transmembrane domain, and various intracellular signaling domains. An additional anti-PSMA CAR-B was constructed as a control for these experiments. A list of the constructs is provided in Table 8.

TABLE 8

| | |
|---|---|
| Construct | Description |
| pWF-396 | pMMLV(LTR)-hEF1a promoter-anti-GPC3 scFv-hCD8H-hCD28M-hCD79aE (SEQ ID NOS. 58 and 59) |
| pWF-397 | pMMLV(LTR)-hEF1a promoter-anti-GPC3 scFv-hCD8H-hCD28M-hCD79bE (SEQ ID NOS. 60 and 61) |
| pWF-398 | pMMLV(LTR)-hEF1a promoter-anti-hPSMA(XENP14484) scFv-hCD8H-hCD28M-hCD79aE (SEQ ID NOS. 62 and 63) |

Expression of anti-GPC-3 on HEK-293 Cell. Lentiviral transductions were used to express GPC3 CAR-B proteins on the surface of HEK293 cells. Expression was determined by flow cytometry with an anti-idiotype antibody specific for GPC-3 (Eureka Therapeutics).

Expression of anti-GPC-3 CAR-B in Human B Cells. pWF 396, 397 and 398 encoding CAR constructs were used to prepare MMLV retrovirus. This retrovirus was used to transduce mouse B cells isolated by negative selection (Stem Cell Technologies) and activated for 24 hours by co-culture with HeLa cells expressing CD40L and the addition of soluble IL-4. 48 hours post-transduction, expression was confirmed using flow cytometry. The expression of the CAR-B was detected using an anti-idiotype antibody against human GPC3. The anti-idiotype antibody was obtained from Eureka Therapeutics.

Results. Mouse B cells expressing anti-GPC-3 CAR-Bs, pWF-396 and 397, were expressed and specifically bound by anti-GPC3 idiotype antibody.

### Example 3 - Adenovirus Variant F35 Expressing GFP

Adenovirus variant F35 expressing GFP was demonstrated to efficiently infect human B cells. Human B cells were isolated from the peripheral blood. The B cells were infected with adenovirus encoding GFP at volumes of 0, 1, 3, 10 µL. The titer of the adenovirus preparations were approximately 1 x e¹² particles/ml.

### Example 4 - Delivering Payloads to Tumor Cells

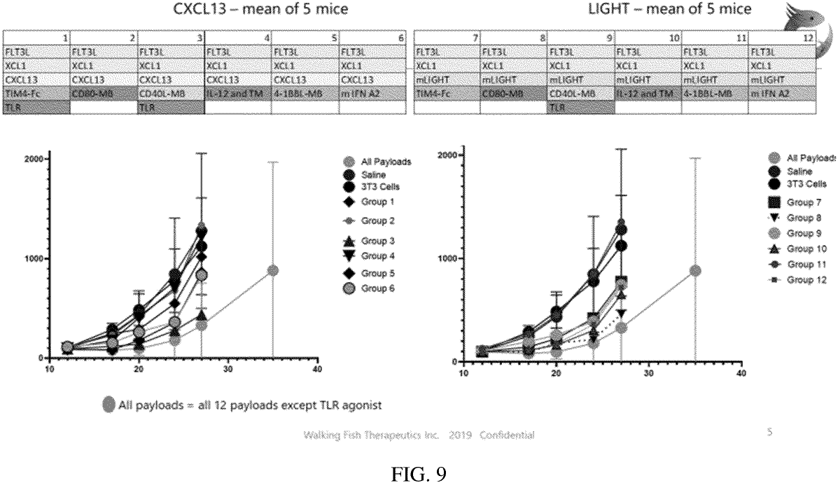

A large screening study was conducted to examine the effect of payloads on NIH3T3 fibroblasts in a CT26 Model. Payloads included various immunomodulators, including cytokines and chemokines. First, BALB/c mice were injected with CT26 tumors into their left and right flank. See FIG. 8. Twelve and sixteen days later, mice were injected into the right flank tumor, with various combinations of 4-5 payloads. Tumor volume was measured for up to 35 days.

Generation of the BALB/C CT26 tumor model. A total of 139 mice were injected with CT26 tumors into their left and right flanks.

Selection of Payload. Twelve peptides were identified for their potential to (i) recruit and activate dendritic cells; (ii) initiate homing and guidance of dendritic cells and T cells into the tumor site; and (iii) activate effector T cells. The payloads screened are listed in Table 9.

TABLE 9

| Payload | SEQ ID NO. |
|---|---|
| FLT3L | 70, 71 |
| XCL1 | 72, 73 |
| TIM4-Fc | 74, 75 |
| CXCL13 | 68, 69 |
| mCCL21 | 92, 93 |
| mCD80 - membrane bound | 86, 87 |
| mCD40L - membrane bound | 88, 89 |
| mlFNa A2 | 84, 85 |
| mlL-12 | 80, 81 |
| mlL-21 | 90, 91 |
| mLIGHT mutant | 78, 79 |
| M4-1BBL-membrane bound | 76, 77 |
| mIL-15 | 124, 125 |

Each was either given a combination of 4-5 payloads, all 12 payloads, or 3T3 cells (without payload) or saline as a control. In total, there were twenty-seven groups (n = 5 mice/group). The experimental groups are identified in Table 10.

TABLE 10

| Group # | Treatment |
|---|---|
| 1 | FLT3L, XCL1, CXCL13, TIM4-Fc, TLR |
| 2 | FLT3L, XCL1, CXCL13, CD80-MB |
| 3 | FLT3L, XCL1, CXCL13, CD40L-MB, TLR |
| 4 | FLT3L, XCL1, CXCL13, IL-12 and TM |
| 5 | FLT3L, XCL1, CXCL13, 4-1BBL-MB |
| 6 | FLT3L, XCL1, CXCL13, IFNa A2 |
| 7 | FLT3L, XCL1, LIGHT, TIM4-Fc |
| 8 | FLT3L, XCL1, LIGHT, CD80-MB |
| 9 | FLT3L, XCL1, LIGHT, CD40L-MB, TLR |
| 10 | FLT3L, XCL1, LIGHT, IL-12 and TM |
| 11 | FLT3L, XCL1, LIGHT, 4-1BBL-MB |
| 12 | FLT3L, XCL1, LIGHT, IFNa A2 |
| 13 | FLT3L, XCL1, IL-21, TIM4-Fc |
| 14 | FLT3L, XCL1, IL-21, CD80-MB |
| 15 | FLT3L, XCL1, IL-21, CD40L-MB |
| 16 | FLT3L, XCL1, IL-21, IL-12 and TM |
| 17 | FLT3L, XCL1, IL-21, 4-1BBL-MB |
| 18 | FLT3L, XCL1, IL-21, IFNa A2 |
| 19 | FLT3L, XCL1, CCL21, TIM4-Fc |
| 20 | FLT3L, XCL1, CCL21, CD80-MB |
| 21 | FLT3L, XCL1, CCL21, CD40L-MB |
| 22 | FLT3L, XCL1, CCL21, IL-12 and TM |
| 23 | FLT3L, XCL1, CCL21, 4-1BBL-MB |
| 24 | FLT3L, XCL1, CCL21, IFNa A2 |
| 25 | All Payloads |
| 26 | Saline |
| 27 | 3t3 cells (no payload) |

Dosing. Tumor volume was between 100 mm³ and 150 mm³ at the time of the first injection. For the groups receiving 4 payloads, each pay load was delivered at 2.5 x 10⁵ cells per injection for a total of 10⁶ cells. For the groups receiving 5 payloads, each pay load was delivered at [x] cells per injection for a total of 3 x 10⁶ cells. The fifth payload was co-administered with Poly(I:C), which is a ds-RNA analog. Payloads were administered by intra-tumor injection. The volume of administration was 50 µL for all groups except the poly (I—C) group and the large 12-way group, where the volume was 150 µL.

Payload Administration Procedures. Cells were harvested with versene (not in the presences of trypsin). Once collected, the cells were counted, spun and resuspended in a volume that could be adjusted to 20 x 10⁶ /ml after the cells are recounted. TLR agonist (Invivogen Cat# ODN:1826) by resuspending lyophilized powder in water provided. TLR agonist was resuspended at 10 mg/ml and heated to 70degC and then let to sit at RT for 1 hour prior to using. The dose of TLR agonist is 50 µg in 50 µl.

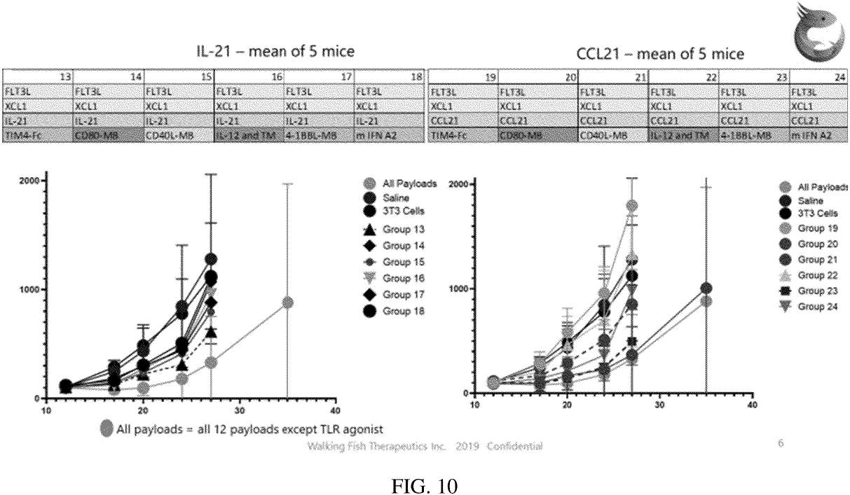

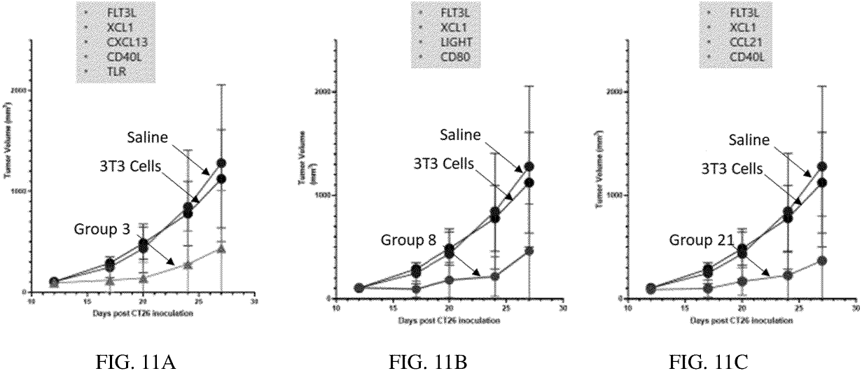

Results. The results are depicted in FIGS. 9-11. Several combinations of payloads injected ipsilaterally demonstrated antitumor activity in the contralateral tumors manifested as delayed tumor growth in this model. Groups 3, 8 and 21 showed the most significant impairment of tumor growth over 30 days.

### Example 5 - Modified B Cells That Express and Secrete Payloads

Experimental Design. A BALB/c mouse CT26 tumor model was used to evaluate the efficacy of modified B cells expressing various payload on tumor volume and survival. Mice were injected with tumor cells at a volume of 100 µL. On day 6 once tumors had reached a volume of 175 mm³, mice were injected with modified B cells expressing various payloads as described below. Tumor volume and survival were measured for 17 days.

Isolation of Mouse PBMCs. Mouse PBMCs or splenocytes are isolated from blood or spleen, respectively. PBMCs are isolated using Lympholyte-M (CedarLane, Cat#CL5030). Splenocytes are isolated by manual cell separation through a 70 micron nylon cell strainer. B cells are then isolated from PBMCs or splenocytes via immunomagnetic negative selection using EasySep® Mouse B cell Isolation Kit (Stem Cell Technologies, Cat #19854).

Selection of Payloads. Nucleic acid sequences expressing payload peptides or proteins are transfected or transduced into isolated B cells. The following twelve peptides were identified for their potential to (i) recruit and activate dendritic cells; (ii) initiate homing and guidance of dendritic cells and T cells into the tumor site; and (iii) activate effector T cells. The payloads screened are listed in Table 9.

Each mouse was either given a combination of 4-5 payloads, or isolated B cells (without payload) or saline as a control. In total, there were twenty-seven groups (n = 5 mice/group). The experimental groups are identified in Table 11.

TABLE 11

| Group # | Treatment |
|---|---|
| 3 | FLT3L, XCL1, CXCL13, CD40L-MB, TLR |
| 8 | FLT3L, XCL1, mLIGHT, CD80-MB |
| 21 | FLT3L, XCL1, CCL21, CD40L-MB |
| 26 | Saline |
| 27 | B cells (no payload) |

Generation of Payload Expressing B Cells. For transfection, purified or cultured B cells are washed and suspended in Cytoporation Medium T (BTX, Cat # 47-0002) at 5 x 10⁶ to 25 x 10⁶ cells per ml and mixed with 7.5 µg to 50 µg RNA (RNA constructs are designed and prepped in house or purchased from TriLink using CleanCap® and fully substituted with Pseudo-U). 200 µL cell/RNA suspension electroporated using BTX Agilpulse® Electroporation System.

Dosing. Tumor volume was between 100 mm³ and 150 mm³ at the time of the first injection. For the groups receiving 4 payloads, each payload was delivered at 2.5 x 10⁵ cells per injection for a total of 10⁶ cells delivered. For the groups receiving 5 payloads, each pay load was delivered at 2.5 x 10⁵ cells per injection for a total of 1.25 x 10⁶ cells delivered. Payloads were injected intra-tumor. The volume of administration was 50 µL for groups receiving 4 payloads, the volume of administration was 100 µL for groups receiving 5 payloads.

Payload Administration Procedures. Cells were harvested with versene (not in the presence of trypsin). Once collected, the cells were counted, spun and resuspended in a volume that could be adjusted to 20 x 10⁶ /ml. TLR agonist (InvivoGen Cat# ODN:1826) by resuspending lyophilized powder in water provided. TLR agonist was resuspended at 10 mg/ml and heated to 70° C. and then let to sit at RT for 1 hour prior to using. The dose of TLR agonist is 50 µg in 50 µl.

### Example 6 - Anti-Tumor Activity of Intratumorally Injected B Cells

Mouse splenocytes were obtained and isolated via manual cell separation utilizing a 70 micron nylon cell strainer. Autologous (BALB/c) or allogeneic (C57B1/6) donor mice were used (data shown utilized allogeneic B cells). B cells were isolated from the splenocytes above using immunomagnetic negative selection via the EasySep® Mouse B Cell Isolation Kit (Stem Cell Technologies®, Cat #19854).

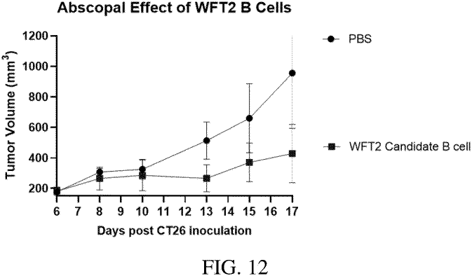

B cells were then injected either (i) fresh or (ii) first stimulated for 16-24 hours in growth media (RPMI, 10% FBS, 1% Pen/Strep, 5 ng/ml recombinant mouse IL-4, 100uM beta-mercaptoethanol) with 5 µg/ml Lipopolysaccharide. 5X10⁶ B cells were then intratumorally injected into the CT26 mouse model, and anti-tumor responses in the distal (abscopal) tumor where measured. Tumors were implanted at day 0, and at day 6 palpable tumor mass was observed. Treatment was initiated on day 6 intratumorally. The results are set forth in FIG. 12.

### Example 7 - Expression of Chimeric Antigen Receptor (CAR) in B Cells Using RNA Electroporation to Make CAR B Cells

Mouse PBMCs or splenocytes were isolated from blood or spleen as follows. Mouse PBMCs were isolated using Lympholyte-M (CedarLane, Cat #CL5030), and splenocytes were isolated by manual cell separation via passage through a 70 micron nylon cell strainer. B cells were then isolated from PBMCs or splenocytes, respectively, via immunomagnetic negative selection using the EasySep® Mouse B Cell Isolation Kit (Stem Cell Technologies, Cat #19854).

B cells were then stimulated for 16-24 hours in growth media (RPMI, 10% FBS, 1% Pen/Strep, 5 ng/ml recombinant mouse IL-4, and 100 uM beta-mercaptoethanol) with 5-15 ug/ml lipopolysaccharide. B cells were then transduced or transfected using known techniques (viral transfection or electroporation) to achieve either stable or transient expression of CAR-B. A strep II tag was incorporated for post-translational detection. Representative CAR-Bs depicted are as follows:
1. XENP PSMA CBCR (3X strep II tag)
2. HyHEL10 CBCR (3X strep II tag)
3. D1.3-M3 HEL CBCR (3X strep II tag)

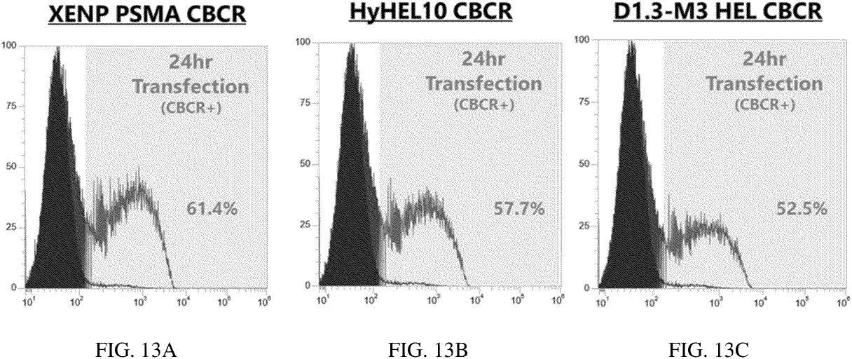

For transfection, purified or cultured B cells were washed and suspended in Cytoporation Medium T (BTX, Cat #47-0002) at 5x10⁶ to 25x10⁶ cells per ml and mixed with 7.5 ug to 50 ug RNA (RNA constructs were designed and prepped either in-house or purchased from TriLink using CleanCap® and fully substituted with Pseudo-U). A 200ul cell/RNA suspension was obtained and electroporated using the BTX AgilePulse® Electroporation System. Cells were then washed in PBS and prepped for IV injection into immune-incompetent mice with established HepG2 tumor cells that express respective antigen (e.g. GPC3, HEL, PSMA). Translation and expression of protein of interest was then measured using an anti-Strep II tag antibody. The results are set forth in FIG. 13. In FIG. 13, the X axis shows strength of expression signal as measured by flow cytometry, and the Y axis sets forth percent of cells expressing the desired protein of interest (PSMA, HEL).

This experiment demonstrates that the desired RNA sequence/s are successfully transfected or transduced (accordingly), the RNA is successfully translated, and the desired protein of interest is expressed on the cell surface.

### Example 8 - Modified B Cells Expressing Integrins and Homing Receptors

Nucleic acid constructs expressing an integrin, a homing receptor, or both are constructed using known techniques. Mouse and Human B cells are transfected or transduced (accordingly) with the nucleic acid constructs to express the integrin, the homing receptor, or both. These modified cells are administered intravenously into mice or a human host. Time-lapse imaging will measure accumulation of the modified B cells at the site/target of interest, such as a homing or target tissue, an inflammatory site in a specific location or tissue, or a tumor or tumor microenvironment, to establish that expression of an integrin and/or a homing receptor of defined homing specificity endows the B cells with the ability to home to and accumulate at the site/target of interest where delivery of therapeutic payloads is desirable. A screening study is conducted according to the techniques of Example 5 to examine delivery and effect of payloads at the site/target of interest.

### Example 9 - Altering B Cell Trafficking

Isolated B cells are cultured with a specific concentration of all-trans-retinoic acid (ATRA) or derivatives thereof that induce expression of α4β7 integrin and the homing receptor CCR9. Thereafter, the B cells are harvested and administered intravenously into mice. There are two experimental groups of the recipient mice. The first group of mice are pre-treated with DSS or TNBS to induce gut inflammation. The second group of mice are not treated with DSS or TNBS. Inflammation similar to that observed in human intestinal bowl diseases is induced by pretreatment with DSS or TNBS. Administered B cells treated with ATRA or derivative thereof will home to areas of inflammation consistent with their homing potential due to increased expression of α4β7 integrin and the homing receptor CCR9.

### Example 10 - Modified B Cells Expressing Immune Inhibitory Molecules

Nucleic acid constructs expressing an immune inhibitory molecule selected from IL-10, TGF-β, PD-L1, PD-L2, LAG-3, and TIM-3, or any combinations thereof, are constructed using known techniques. Mouse and Human B cells are transfected or transduced (accordingly) with the nucleic acid constructs to express one or more of the immune inhibitory molecules listed above. These modified cells are administered intravenously into mice or a human host or elsewhere near or at sites of inflammation. Time-lapse imaging will measure accumulation of the modified B cells at a site/target of interest, such as a homing or target tissue, an inflammatory site in a specific location or tissue, or a tumor or tumor microenvironment, to establish that inflammation at the site and autoimmune activity of the B cells localized to the site are decreased, thereby leading to a positive therapeutic response.

### Example 11 - Activation of B Cells With TLRs

B cells are treated with TLR agonists and/or modified to express a constitutively active TLR for use in potentiating B cells for immune responses and producing potent effector B cells to increase antigen-specific immune responses in a subject. Isolated mouse or human B cells are treated in vitro with a TLR agonist at the same time or in advance of the administration of the B cells. In some instances, the mouse or human B cells are treated with more than one TLR agonists.

A modified B cell, transfected or transduced with or without a CAR-B construct of the foregoing examples, is engineered to express one or more constitutively active TLRs. Each TLR is introduced into the modified B cell (transduced or transfected using known techniques) as a DNA construct under the control of a constitutively activated transcriptional pathway. A modified B cell, expressing one or more constitutively active TLRs (with or without a CAR-B construct), is also treated with one or more TLR agonists at the same time or in advance of the administration of the modified B cells to a subject or patient in need thereof. Time-lapse imaging and other known techniques will measure accumulation of the modified B cells in the desired location and confirm expression of the TLR(s) and any expressed CAR-B of a defined specificity.

This experiment will demonstrate that the desired DNA sequence/s encoding specific TLR(s) of interest are successfully transfected or transduced (accordingly) into B cells with or without a CAR-B construct and treated with or without TLR agonist(s), the RNA is successfully translated, the desired TLR(s) are expressed in the B cells for producing potent effector B cells potentiating B cells for immune responses.

### Example 12 - Antigen Presentation Both in HLA Class I and Class II Molecules Using RNA Electroporated B cells

mRNA Constructs. Exemplary mRNA constructs are designed by fusing a specific antigen, e.g., a tumor antigen or an infectious disease antigen, to the targeting signal of a the lysosomal protein LAMP1, to target the specific antigen to the lysosomes and present the antigen simultaneously and efficiently in both HLA class I and class II molecules. Tumor antigens and infectious disease antigens are well known in the art and can include any antigen of interest against which an immune response is desired. Various mRNA constructs are made encoding at least one specific antigen of interest fused to the targeting signal of LAMP1 that is capable of presenting the specific antigen simultaneously and efficiently by both HLA class I and class II molecules when transfected into a suitable immune cell.

Experimental Design. Isolated mouse or human B cells are electroporated in vitro with an mRNA construct described above (i.e., encoding a specific antigen of interest fused to the targeting signal of LAMP1) using known mRNA electroporation techniques. In some instances, the mouse or human B cells are also transduced or transfected using known techniques with a CAR-B construct according to any of the foregoing examples. The mRNA electroporated B cells, transduced with or without a CAR-B construct of interest, are introduced intravenously into mice or a human host. Time-lapse imaging will measure accumulation of the modified B cells in the desired location and also confirm expression of CAR-B of a defined specificity. Translation and expression of the specific tumor antigens or infectious disease antigens of interest are measured using known techniques to establish that the antigens of interest are targeted to the lysosomes and presented simultaneously and efficiently by both HLA class I and class II molecules.

This experiment will demonstrate that the desired mRNA sequence/s encoding specific antigens of interest fused to a targeting signal are successfully transfected into B cells (which, if desired, are also transduced with a CAR-B construct), the mRNA is successfully translated, and the electroporated and modified B cells simultaneously and efficiently present the specific antigen of interest by both HLA class I and class II molecules for increasing antigen-specific immune responses in a subject.

### Example 13 - B Cells Expressing a PSMA-Specific CAR Reduce Tumor Growth in CT26-PSMA Tumors

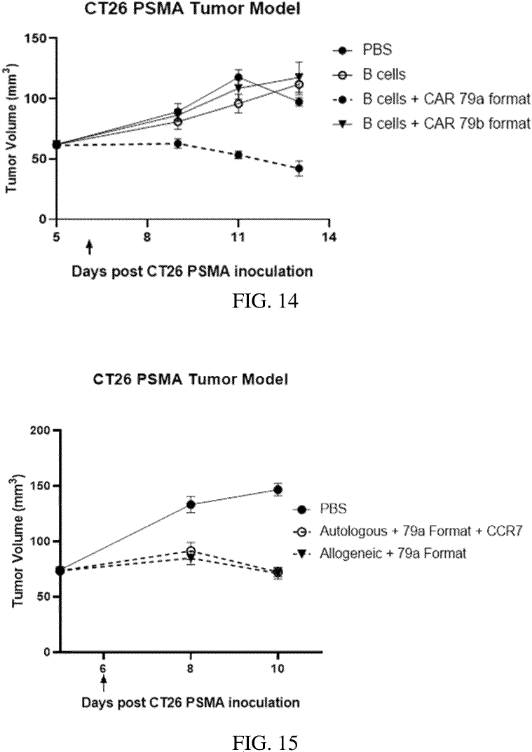

Mouse Tumor Model. A BALB/c CT26-PSMA tumor model engineered to express human PSMA was used to evaluate the efficacy of PSMA-specific CAR engineered murine B cells on tumor volume and survival. Eight-week-old BALB/c mice were injected on one hind flank with 1.0x10⁶ CT26-PSMA tumor cells in a volume of 50 µl. On day 5 when the tumor volume reached approximately 60 mm³ the mice were distributed equally into 3 groups of 10 mice. Treatment of mice was started on day 6 using murine B cells engineered with mRNA encoding two different PSMA-specific CAR formats or un-engineered B cells administered intravenously at a dose of 1.5x10⁶ cells in 100µl, or saline on day 6. Tumor volume was measured using calipers on day 5, 9, 11, and 13. There was a statistically significant tumor reduction of 57% in the PSMA-CAR group (format 79a) relative to saline on day 13. There was not a significant reduction of tumor volume on day 13 in the PSMA-CAR treatment group (format 79b) relative to saline (FIG. 14).

Engineering of Murine B Cells. Mouse splenocytes were isolated from BALB/c donor spleens by manual cell separation through a 70 micron nylon cell strainer. B cells were then isolated from splenocytes via immunomagnetic negative selection using EasySep Mouse B cell Isolation Kit (Stem Cell Technologies, Cat #19854). B cells were stimulated for 24 hours in growth media (RPMI, 10% FBS, 25 mM HEPES, 1% Pen/Strep, 5 ng/ml recombinant mouse IL-4, 100µM beta-mercaptoethanol) with anti-CD40 (250 ng/ml). Cells were then electroporated with 20 µg CAR mRNA construct per 3.6x10⁶ B cells using BTX AgilePulse electroporation system set at 280V for 1 ms. Cells were washed and resuspended in PBS at a concentration of 15x10⁶ B cells /ml. 100 µl of cell suspension were used per dose.
PSMA construct CD79a: pmRNA_d7_13_anti hPSMA(XENP14484) scFv-mCD8H-mCD28M-mCD79aE #ab-1
PSMA construct CD79b: pmRNA_d7_13_anti hPSMA(XENP14484) scFv-mCD8H-mCD28M-mCD79bE #ac-1

### Example 14 - Allogenic B Cells Expressing a PSMA-Specific CAR Reduce Tumor Growth in CT26-PSMA Tumors

Mouse Tumor Model. A BALB/c CT26-PSMA tumor model engineered to express human PSMA was used to evaluate the efficacy of PSMA-specific CAR engineered allogeneic murine B cells on tumor volume and survival. Eight-week-old BALB/c mice were injected on one hind flank with 1.0x10⁶ CT26-PSMA tumor cells in a volume of 50 µl. On day 5 when the tumor volume reached approximately 70 mm³ the mice were distributed equally into 3 groups of 10 mice. Treatment of mice was started on day 6 using autologous murine B cells engineered with mRNA encoding a PSMA-specific CAR and an mRNA encoding CCR7 or allogeneic murine B cells engineered with mRNA encoding a PSMA-specific CAR administered intravenously at a dose of 1.5x10⁶ cells in 100µl, or saline. Tumor volume was measured using calipers on day 5, 8, and 10. There was a statistically significant tumor reduction of 51% in the allogeneic and autologous engineered B cell groups relative to saline on day 10 (FIG. 15). (p<0.005).

Engineering of Murine B Cells. Mouse splenocytes were isolated from autologous BALB/c and allogeneic C57B1/6 donor spleens by manual cell separation through a 70 micron nylon cell strainer. B cells were then isolated from splenocytes via immunomagnetic negative selection using EasySep Mouse B cell Isolation Kit (Stem Cell Technologies, Cat #19854). B cells were stimulated for 24 hours in growth media (RPMI, 10% FBS, 25 mM HEPES, 1% Pen/Strep, 5 ng/ml recombinant mouse IL-4, 100µM beta-mercaptoethanol) with anti-CD40 (250 ng/ml). Cells were then electroporated with 20 ug CAR mRNA construct per 3.6x10⁶ B cells using BTX AgilePulse electroporation system set at 280V for 1 ms. Cells were washed and resuspended in PBS at a concentration of 15x10⁶ B cells /ml. 100 µl of cell suspension were used per dose. Example 15 - The Antitumor Activity of PSMA-CAR-Engineered B Cells Depends on an Intact Host Immune System

Mouse Tumor Models. The effect of antitumor PSMA-CAR B cells was studied in WT and immunocompromised NSG mice.

WT Mice. A BALB/c CT26-PSMA tumor model engineered to express human PSMA was used to evaluate the efficacy of PSMA-specific CAR engineered murine B cells on tumor volume and survival in WT mice. Eight-week-old BALB/c mice were injected on one hind flank with 1.0x10⁶ CT26-PSMA tumor cells in a volume of 50 µl. On day 5 when the tumor volume reached approximately 60 mm³ the mice were distributed equally into 4 groups of 10 mice. Treatment of mice was started on day 6 using murine B cells engineered with mRNA encoding two different PSMA-specific CAR formats or un-engineered B cells administered intravenously at a dose of 1.5x10⁶ cells in 100µl, or saline on day 6. Tumor volume was measured using calipers on day 5, 9, 11, and 13. There was a statistically significant tumor reduction of 57% in the PSMA-CAR group (format 79a) relative to saline on day 13. There was not a significant reduction of tumor volume on day 13 in the PSMA-CAR treatment group (format 79b) or un-engineered B cells, relative to saline (FIG. 14).

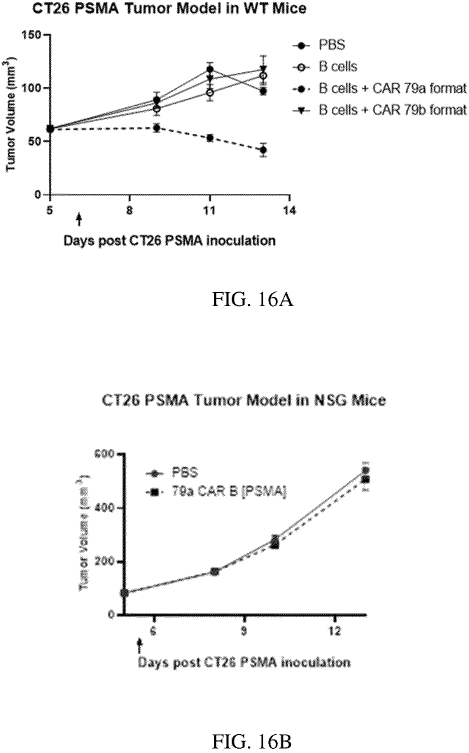

NSG Mice. A BALB/c CT26-PSMA tumor model engineered to express human PSMA was used to evaluate the efficacy of PSMA-specific CAR engineered murine B cells on tumor volume and survival in immunocompromised mice. Eight-week-old NSG mice were injected on one hind flank with 1.0x10⁶ CT26-PSMA tumor cells in a volume of 50 µl. On day 5 when the tumor volume reached approximately 60 mm³ the mice were distributed equally into 2 groups of 10 mice. Treatment of mice was started on day 6 using murine B cells engineered with mRNA encoding a PSMA-specific CAR format administered intravenously at a dose of 1.5x10⁶ cells in 100µl, or saline on day 6. Tumor volume was measured using calipers on day 5, 8, 10, and 13. There was no significant reduction in tumor volume in the PSMA-CAR group (format 79a) relative to saline on day 13 (FIG. 16B).

Engineering of Murine B Cells. Mouse splenocytes were isolated from autologous BALB/c and allogeneic C57B1/6 donor spleens by manual cell separation through a 70 micron nylon cell strainer. B cells were then isolated from splenocytes via immunomagnetic negative selection using EasySep Mouse B cell Isolation Kit (Stem Cell Technologies, Cat #19854). B cells were stimulated for 24 hours in growth media (RPMI, 10% FBS, 25 mM HEPES, 1% Pen/Strep, 5 ng/ml recombinant mouse IL-4, 100µM beta-mercaptoethanol) with anti-CD40 (250 ng/ml). Cells were then electroporated with 20 ug CAR mRNA construct per 3.6x10⁶ B cells using BTX AgilePulse electroporation system set at 280V for 1 ms. Cells were washed and resuspended in PBS at a concentration of 15x10⁶ B cells / ml. 100 µl of cell suspension were used per dose.
PSMA construct CD79a: pmRNA_d7_13_anti hPSMA(XENP14484) scFv-mCD8H-mCD28M-mCD79aE #ab-1
PSMA construct CD79b: pmRNA_d7_13_anti hPSMA(XENP14484) scFv-mCD8H-mCD28M-mCD79bE #ac-1

### Example 16 - B Cells Expressing a GPC3-Specific CAR Reduce Tumor Growth in HEPA 1-6 GPC3 Tumors

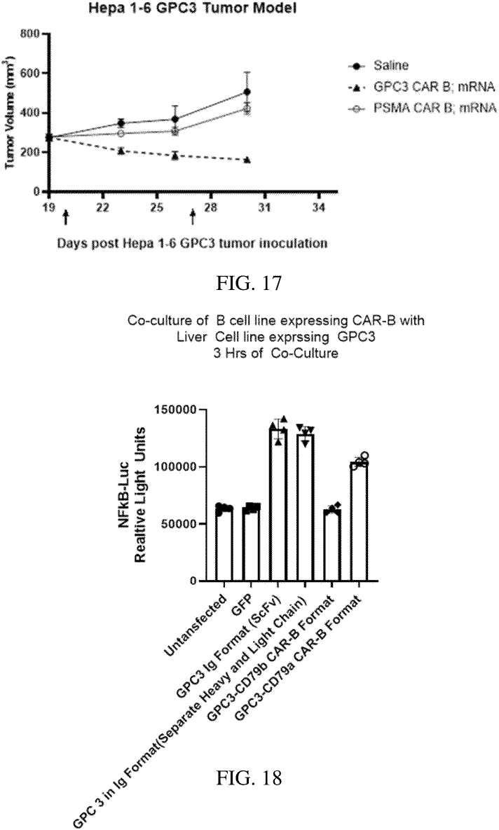

Mouse Tumor Model. A C57B1/6 HEPA 1-6 tumor model engineered to express human GPC3 (HEPA 1-6-GPC3) was used to evaluate the efficacy of murine B cells on tumor volume and survival. Eight-week-old C57B1/6 mice were injected on one hind flank with 5.0x10⁶ HEPA 1-6-GPC3 tumor cells at a volume of 200 µl. On day 19 when the tumors volume reached approximately 250 mm³ the mice were distributed equally into 3 groups of 10 mice. Treatment of mice was started on day 20 using murine B cells engineered with mRNA encoding a GPC3-specific CAR or a PSMA-specific CAR administered intravenously at a dose of 1.5x10⁶ cells in 100µl, or saline on day 20 and day 27. Tumor volume was measured using calipers on day 19, 23, 26, and 30. There was a statistically significant tumor reduction of 68% in the GPC3-CAR group relative to saline on day 30. There was not a significant reduction of tumor volume on day 30 in the PSMA-CAR treatment group relative to saline. (note: this study is still in progress on 1-19-2021) (FIG. 17).

Engineering of Murine B Cells. Mouse splenocytes were isolated from C57B1/6 donor spleens by manual cell separation through a 70 micron nylon cell strainer. B cells were then isolated from splenocytes via immunomagnetic negative selection using EasySep Mouse B cell Isolation Kit (Stem Cell Technologies, Cat #19854). B cells were stimulated for 24 hours in growth media (RPMI, 10% FBS, 25 mM HEPES, 1% Pen/Strep, 5 ng/ml recombinant mouse IL-4, 100µM beta-mercaptoethanol) with anti-CD40 (250 ng/ml). Cells were then electroporated with 20 µg CAR mRNA construct per 3.6x10⁶ B cells using BTX AgilePulse electroporation system set at 280V for 1 ms. Cells were washed and resuspended in PBS at a concentration of 15x106 B cells /ml. 100 µl of cell suspension were used per dose.
GPC3 mRNA construct: pmRNA_d7_13_anti-hGPC3 scFv-mCD8H-mCD28M-mCD79aE #15-1
PSMA construct: pmRNA_d7_13_anti hPSMA(XENP14484) scFv-mCD8H-mCD28M-mCD79aE #ab-1

### Example 17 - Multimerized GPC3 Can Activate NFκB Expression of Luciferase in Cells Expressing a GPC3 CAR in a Dose-Responsive Manner

CAR-B Construct Design. Five CAR-B constructs were designed using three basic formats (i) CAR 2 (an scFv, a hinge domain, a transmembrane domain and a signaling domain (see FIG XA)); (ii) CAR 3 (a multimerized receptor complex with 2 of each of the following: an scFv, a hinge domain, an FC domain, a transmembrane domain and a cytoplasmic tail (see FIG XB)); (iii) CAR 4 (a multimerized receptor complex with 2 of each of the following: (a FAB domain, a hinge domain, an FC domain, a transmembrane domain and a cytoplasmic tail (see FIG XC). The five CAR-B constructs are as follows:

TABLE 12

| | |
|---|---|
| pWF-506 (SEQ ID NO. 140/141) | pmRNA_d7_13_anti-hGPC3 scFv-hIgG1 Fc [TM + cyto] A-1 (CAR 3) |
| pWF-507 (SEQ ID NO. 142/143) / pWF-508 | pmRNA_d7_13_anti-hGPC3 vl-hcLamda / pmRNA_d7_13_anti-hGPC3 vH-hlgHg1 [TM+ cyto] (CAR 4) |
| (SEQ ID NO. 144/145) | |
| pWF-509 (SEQ ID NO. 146/147) | pmRNA d7_13 anti-hGPC3 scFv-hCD8H-hCD28M-hCD79bE (CAR 2) |
| pWF-510 (SEQ ID NO. 148/149) | pmRNA_d7_13_anti-hGPC3 scFv-hCD8H-hCD28M-hCD79aE (CAR 2) |

NFKκB Reporter Assay: Antigen induced signaling. Ramos NFκB-luciferase reporter cells were transduced with mRNA coding for one of the CAR-B constructs listed above. Ramos NFκB-luciferase reporter cells were transfected at 280 V and 1 msec with 10 µg of RNA in 200 µL of electroporation buffer followed by culturing overnight in growth medium. The cells were left at room temperature for 4 hours to quiesce the cells to reduce background. 30,000 of the transfected cells were transferred to each well in a multi-well plate in a volume of 30 µL per well. The transfected Ramos cells were then incubated with GPC3 protein multimerized with streptavidin, streptavidin control or GPC3-Fc protein for 3 hours in growth medium. 30 µL of Bioglo® substrate (Promega) was added to each well and the plate was read within 5 minutes using a luminometer. As demonstrated in FIG. 18, multimerized GPC3 was capable of activating NFκB expression of luciferase in cells expressing three of the four GPC3 CAR-Bs except pWF-509 (GPC3-CD79b). All four constructs displayed good binding to GPC3 in FACS assays. Therefore, CD79b was an example where a CAR, which had good binding affinity, did not signal.

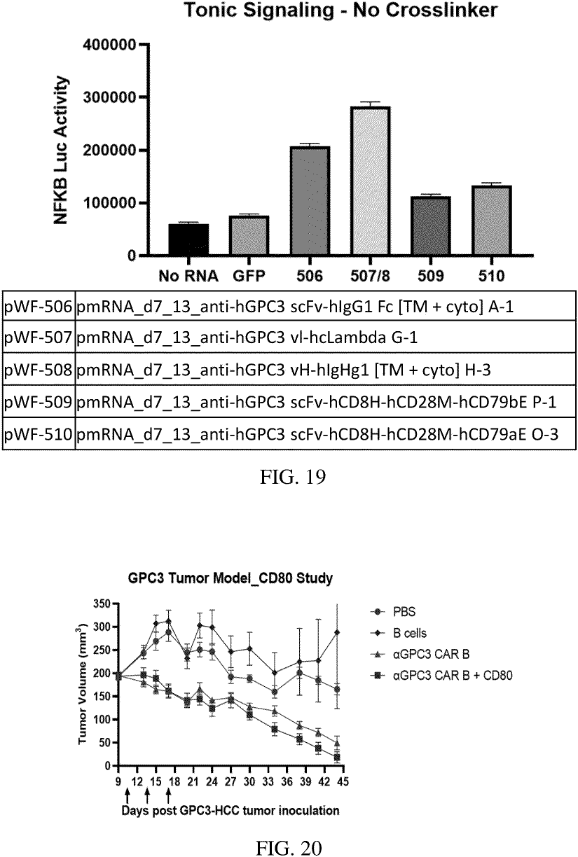

NFκB Reporter Assay: Tonic Signaling. Tonic signaling was also assessed, using the NFκB Reporter Assay. CAR constructs, which induced elevated tonic signaling in the absence of cognate antigen binding, were generated. FIG. 19 shows that the four CAR-B constructs were expressed in a human B cell reporter line and NFκB luciferase activity was measured in the absence of cognate target antigen. Each construct displayed significant tonic signaling activity. Engineered B cells with tonic signaling CAR Bs remained at a high number in vivo and led to high and durable expression of replacement factors or other payloads.

### Example 18 - A CD80 Payload Enhances the Antitumor Activity of Anti-GPC3CAR-CD79a B Cells

Experimental Design. A syngeneic C57B1/6 mouse HEPA1-6GPC3 tumor is a model of human HCC engineered to express human GPC3. This model was used to evaluate the efficacy of murine B cells electroporated with anti-GPC3CAR-CD79a and a CD80 payload mRNAs. Mice were injected on one hind flank with 5.0 X 10⁶ HEPA1-6GPC3 tumor cells at a volume of 200 ul in matrigel. On day 11, 14, and 17 the mice were administered a 200 ul IV dose of 1.5x10⁶ B cells, B cells engineered with anti-GPC3CAR-CD79a, B cells engineered with anti-GPC3CAR-CD79a and CD80, or saline as indicated in FIG. 20. The B cells were engineered with mRNA as described below. Tumor volume was monitored on multiple days as indicated in FIG. 20.

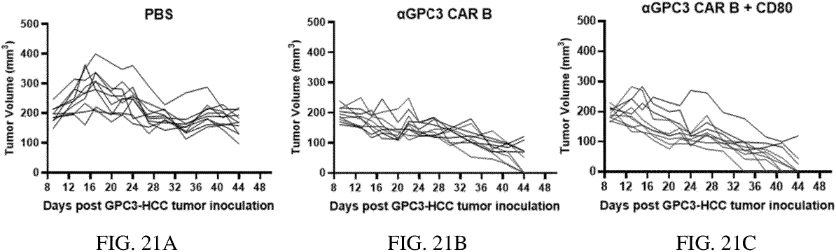

As can be seen in FIG. 20, both the anti-GPC3 CAR-CD79a, and anti-GPC3CAR-CD79a plus CD80 combo displayed a statistically significant effect relative to saline or un-engineered B cells on day 44 and at multiple earlier time points. Additionally, by day 44 there were no complete responses in the saline control or B cell control groups but the anti-GPC3CAR-CD79a, and anti-GPC3CAR-CD79a plus CD80 combo resulted in 4 and 7 complete responses, respectively, as indicated in FIGS. 21A-21C. These data demonstrate that inclusion of the CD80 payload as mRNA potentiated the antitumor activity of B cells co-electroporated with an antigen-specific GPC3 CAR.

B Cell preparation. Mouse splenocytes were isolated from C57B1/6 donor spleens by mechanical cell separation through a 70 micron nylon cell strainer. B cells were then isolated from splenocytes via immunomagnetic negative selection using EasySep Mouse B cell Isolation Kit (Stem Cell Technologies, Cat #19854). B cells were stimulated for 24 hours in growth media (RPMI, 10% FBS, 1% Pen/Strep, 5 ng/ml recombinant mouse IL-4, 100uM beta-mercaptoethanol) with 250 ng/ml CD40 antibody (anti-murine CD40 Ab). Cells were then electroporated with 20ug mRNA per 1.0x10⁷ B cells using BTX AgilePulse electroporation system set at 400V for 1 ms, 2 ms interval for 5 pulses. When two mRNA’s were cotransfected, 20ug of each mRNA was used. Immediately after electroporation, the cells were washed in PBS and prepared for IV administration at a dose of 1.0x10⁷ per 200ul. The electroporated cells were administered to mice within 90 minutes after electroporation.

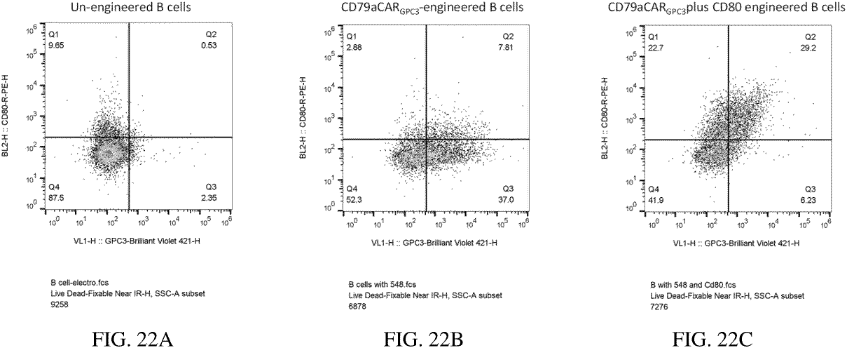

Twelve hours after electroporation, a small aliquot of the cells were stained for expression of anti-GPC3CAR-CD79a and CD80 expression. For detection of anti-GPC3CAR-CD79a expression, GPC3-Avitag and Streptavidin-BV421 were used. CD80 expression was measured with an anti-CD80-PE FACS antibody. The FACS plots in FIGS. 22A-22C show expression of the GPC3 CAR post-electroporation. CD80 was expressed at a basal level in un-engineered B cells, thus accounting for the ~10% positivity. This level remained in the CAR sample, but was increased dramatically in the CAR + CD80 sample. The latter suggested efficient expression of CD80.

### Example 19 - T Cell Activation by B Cells

In accordance with the invention, a 3-component system was developed comprising CAR B cells that are co-cultured with a source of specific antigen (antigen presenting cells or APC) and antigen-specific T cells. The method was as follows.

T cell activation was measured by determining the expression of CD69 on CD4 and CD8 T cell after co-culture with the B cells. Briefly the CAR-B cells were prepared by transfecting B cells with mRNA encoding the CAR-B gene. The B cells were electroporated at 280 V for 1 msec in 300 uL of electroporation buffer with 10ug of CAR-B mRNA and 9 ug of CD80 mRNA when included. The concentration of B cells in the electroporation buffer was 4.5 million B cells per ml. After electroporation the B cell were cultured in growth medium for 4 to 5 hrs. The B cells were then co-cultured with target cells that express the antigen. The target cells were either 293 cells expressing GPC3-linked to ovalbumin or CHO cells expressing GPC3-linked to ovalbumin. The target cells (CHO or 293) were plated at 250,000 cells per well in a 24 well plate and cultured for 24 hours. The next day the medium was removed and 250,000 B cells expressing CAR-B were added. After incubation for 1 to 2 hours, CD4 or CD8 T cells purified by negative selection using T cell isolation kits (Stemcell Inc.) were added. Polyclonal T cells were purified from wildtype C57 mice and OVA-specific CD4 and CD8 T cells were purified from OT-2 and OT-1 mice, respectively. The co-culture of CAR-B cells, target cells and T cells were incubated at 37° C. for 12 to 18 hrs.

To test and measure T cell activation, the mixture of cells was harvested and stained with BV421 anti-mouse CD4 or CD8 marker (BV421) and anti-CD69 (FITC), followed by flow cytometry.

The cell surface target antigen is chimeric where the extracellular domain is recognized by the CAR. The C-terminal sequence contains amino acid sequences recognized by cocultured T cells. Recognition of the target antigen by T cells requires antigen transfer from APC to MHC-matched B cells for processing and presentation via MHC II. This 3-component method leads to presentation of antigen (HEL=hen egg lysozyme) to T cells in an antigen-specific manner, resulting in upregulation of CD69, an activation marker. Controls using unmodified B cells, as well as employing B cells expressing a CAR with a different antigen specificity, resulted in background expression of CD69, further supporting the finding that CAR B cells in accordance with the invention behave in a manner similar to native BCRs. Further, it has been determined that for potency of activation of CD4 T cells in co-culture, CD79a is a superior signaling element than CD79b.

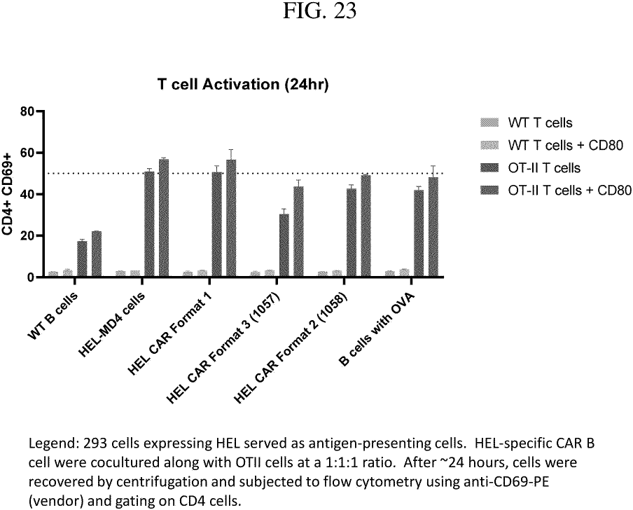

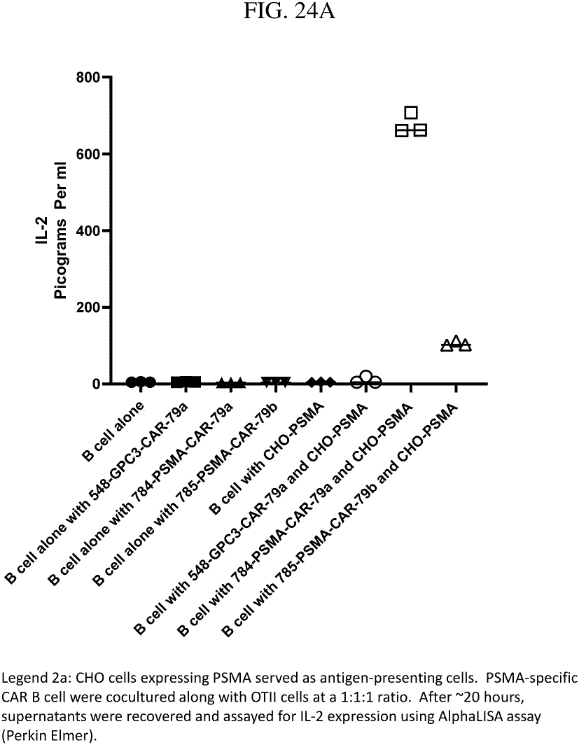

Here, HEL-specific CAR B cell were co-cultured along with OTII cells at a 1:1:1 ratio. After ~24 hours, cells were recovered by centrifugation and subjected to flow cytometry using anti-CD69-PE (vendor) and gating on CD4 cells. The results are set forth in FIG. 23 as percent CD4⁺/CD69⁺ T cells.

### Example 20 - Antigen Specific Activation of CAR B Cells Stimulates Immune Enhancing Cytokine Production

It has now been demonstrated that CAR engagement also leads to secretion of proteins including (but not limited to) IL-2, GM-CSF, and TNF-alpha in an antigen-specific manner. In vivo, IL-2 and other proteins are capable of promoting survival and activation of local antigen-specific T cells. Indeed, this could be an effective complement to antigen presentation by B cells. One or both mechanisms may account for the antitumor activity of adoptively transferred CAR B cells to tumor-bearing mice.

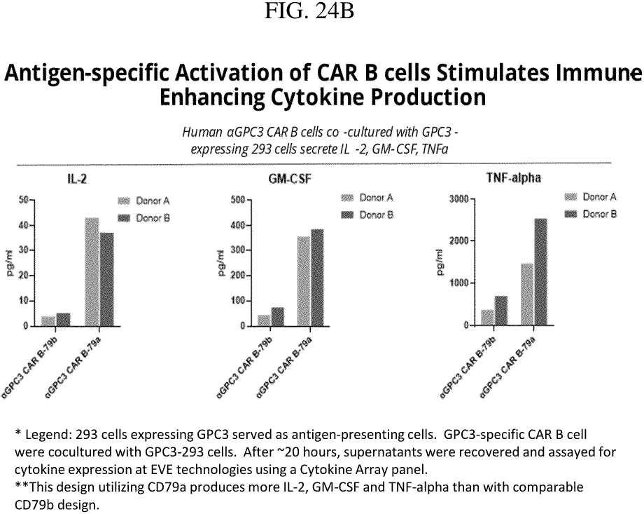

Here, 293 cells expressing GPC3-TM-OVA served as antigen-presenting cells. GPC3-specific CAR B cells were cocultured with GPC3-TM-OVA-293 cells. After ~20 hours, supernatants were recovered and assayed for cytokine expression at EVE technologies using a Cytokine Array panel. This design utilized the CD79a costimulatory domain. As seen in the Figures, use of CD79a as the costimulatory domain in the CAR-B produces more IL-2, GM-CSF and TNF-alpha than with use of a comparable CD79b costim design. The results are set forth in FIGS. 24A and 24B.

### Example 21 - Tumor Antigen Processing by B Cells and Presentation

Here we tested tumor antigen processing by B cells with and without the CD80 payload. Antigen specific T cell activation was measured for control (polyclonal B cells +/-CD80) as compared to antigen B specific B cells +/- CD80.

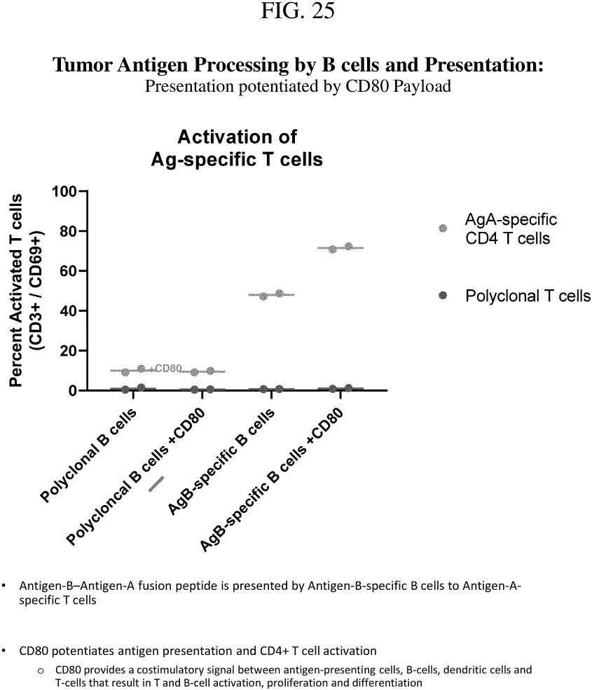

The results of this experiment demonstrate antigen specific activation by B cells. Antigen-B-Antigen-A fusion peptide is presented by Antigen-B-specific B cells to Antigen-A-specific T cells. It can be seen that CD80 potentiates antigen presentation and CD4+ T cell activation. CD80 provides a costimulatory signal between antigen-presenting cells, B-cells, dendritic cells and T-cells that result in T and B-cell activation, proliferation and differentiation. The results are set forth in FIG. 25.

### Example 22 - Tumor Antigen Processing by B Cells and Presentation

Here we tested tumor antigen processing by B cells with and without the CD80 payload. Antigen specific T cell activation was measured for control (polyclonal B cells +/-CD80) as compared to HEL antigen-specific B cells +/- CD80.

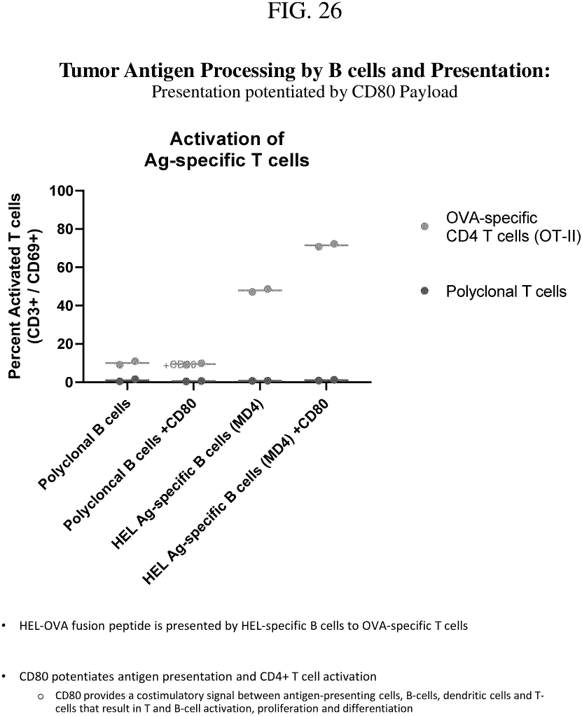

The results of this experiment demonstrate antigen specific activation by B cells. HEL-OVA fusion peptide is presented by HEL-specific B cells to OVA-specific T cells. It can be seen that CD80 potentiates antigen presentation and CD4+ T cell activation. CD80 provides a costimulatory signal between antigen-presenting cells, B-cells, dendritic cells and T-cells that result in T and B-cell activation, proliferation and differentiation. The results are set forth in FIG. 26.

### Example 23 - T Cell Activation by CAR-B

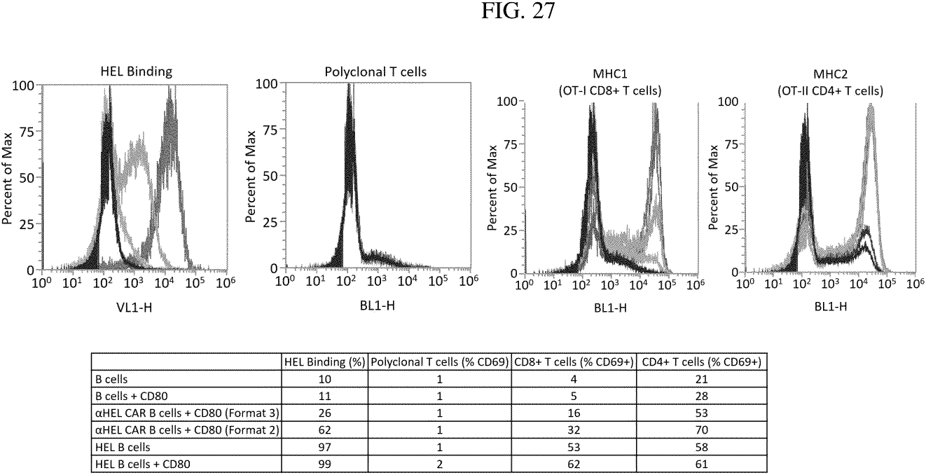

T cell activation was measured by determining CD69 abundance on CD4 and CD8 T cells after co-culture with B cells. Briefly, CAR-B cells were prepared by transfecting B cells with mRNA encoding the CAR-B gene. The B cells were then electroporated at 280V for 1 msec in 300 uL of electroporation buffer along with 10ug of CAR-B mRNA and 9 ug of CD80 mRNA. The concentration of B cells in the electroporation buffer was 4.5 million B cells per ml. After electroporation, the B cells were cultured in growth medium for 4-5 hrs. The B cells were then cocultured with target cells that express the antigen of interest. The target cells were either 293 cells expressing GPC3-linked to ovalbumin or CHO cells expressing GPC3-linked to ovalbumin. The target cells (CHO or 293) were plated at 250,000 cells per well in a 24 well plate and cultured for 24 hours. The next day, the medium was removed and 250,000 B cells expressing CAR-B were added. After incubation of 1-2 hrs. purified CD4 or CD8 T cells were then added. The T cells were purified by negative selection using T cell isolation kits from Stemcell Inc. Polyclonal T cells were then purified from wildtype C57 mice and OVA specific CD4 and CD8 T cells were purified from OT-2 and OT-1 mice respectively. The co-culture of CAR-B cells, target cells and T cells were then incubated at 37C for 12-18 hrs. To measure T cell activation, the mixture of cells was stained with anti-mouse CD4 or CD8 marker (BV421) and anti-CD69 (FITC). In certain experiments, the CAR-B were prepared via adenovirus transduction. Here, freshly isolated B cells were transduced with adenovirus encoding CAR gene. An anti-mouse CD40 adaptor was then used to increase viral transduction efficiency. Results are set forth in FIG. 27.

### Example 24 - In Vivo T Cell Stimulation

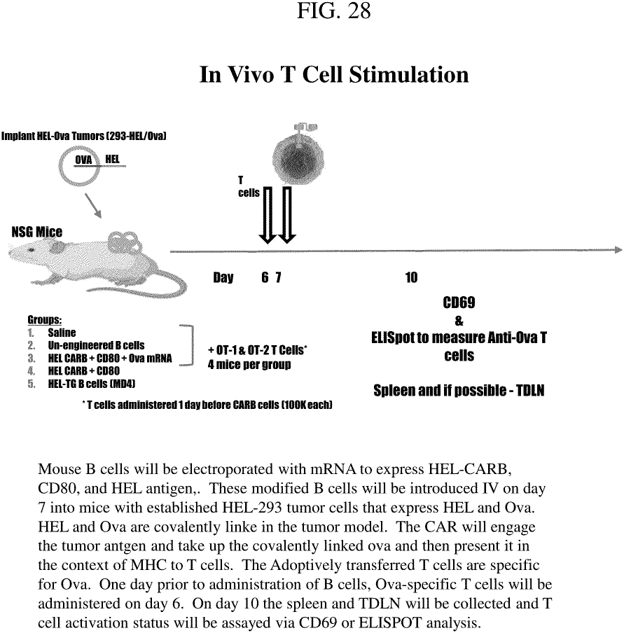

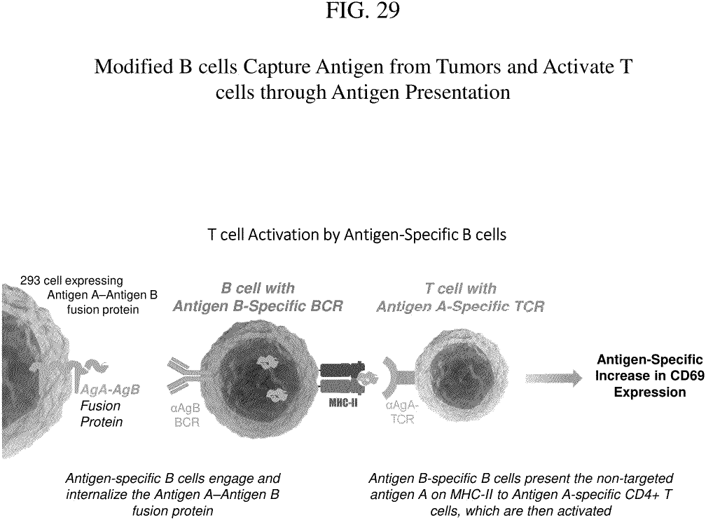

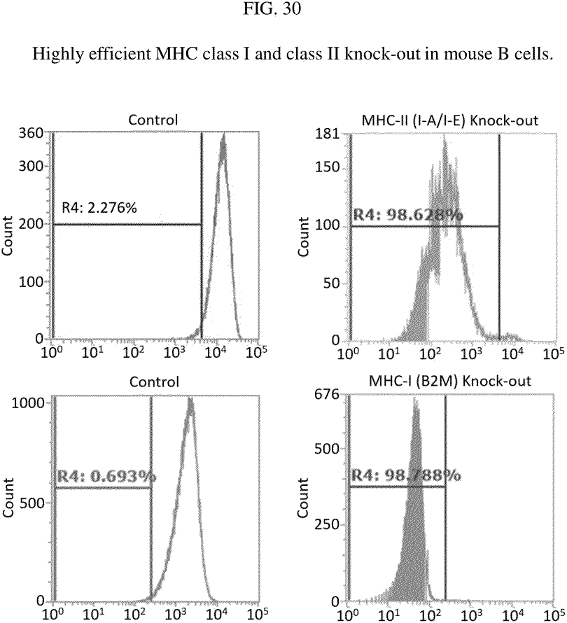

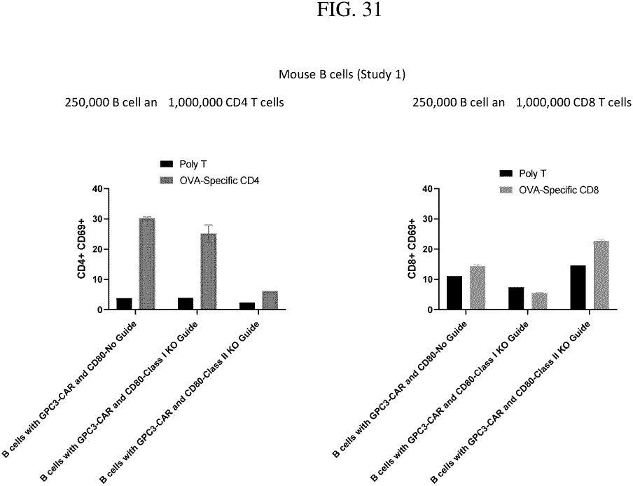

Mouse B cells will be electroporated with mRNA to express HEL-CARB, CD80, and HEL antigen. These modified B cells will be introduced IV on day 7 into mice with established HEL-293 tumor cells that express HEL and Ova. HEL and Ova are covalently linked in the tumor model. The CAR will engage the tumor antigen, take up the covalently linked ova, and then present it in the context of MHC to T cells. The adoptively transferred T cells are specific for Ova. One day prior to administration of B cells, Ova-specific T cells will be administered on day 6. On day 10 the spleen and TDLN will be collected and T cell activation status will be assayed via CD69 or ELISPOT analysis. Exemplary results are set forth in FIG. 28.

### Example 25 - MHCI and MHCII Knock-Out (KO) Using CRISPR—Cas9

Chemically modified sgRNAs oligomer targeting mouse β2m and I-A/I-E genes were manufactured by IDT (Integrated DNA Technologies, Coralville, Iowa, USA). Recombinant S. pyogenes Cas9 enzyme was purchased from IDT (Integrated DNA Technologies, Coralville, Iowa, USA). Cas9 was incubated with sgRNAs at room temperature for 10 minutes prior to mixing with B cells. Engineering of mouse B cells was carried out using an Amaxa™ 4D-Nucleofector™ in P4 nucleofection solution with program DI-100 (Lonza, Basel, Switzerland). 100 pmol RNP was used for electroporation with 1 million or 500000 cells activated mouse B cells in 20 µl volume.

### ASSESSMENT OF MHCI AND MHCII KNOCK-OUT EFFICIENCY

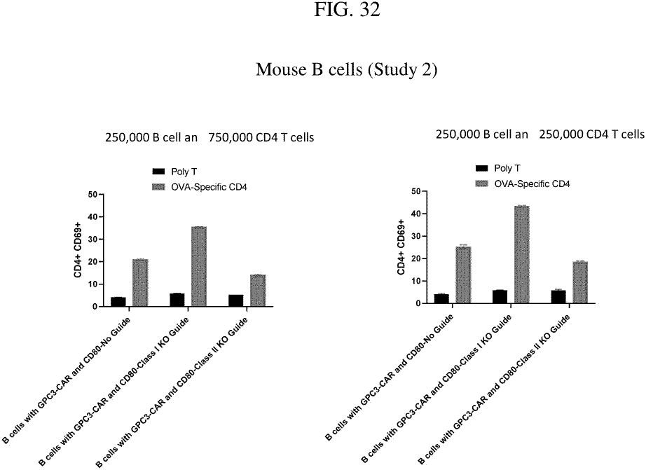

The expression of cell surface MHC I and MHC II was measured by flow cytometry using Attune NxT Flow Cytometer (Invitrogen, Carlsbad, CA, USA) to assess knock-out efficiency. Surface markers were detected using PE-MHC I (H2) (Biolegend, Cat # 125506) and PE-MHC II (I-A/I-E) (Biolegend, Cat# 107608). Additionally, cells were stained with LIVE/DEAD™ Fixable Near-IR (Invitrogen, Carlsbad, CA, USA) to discriminate live and dead cells according to manufacturer’s instructions. The results are set forth in FIG. 30.

### Example 26 - Assessment of Anti-GPC3 CAR B and Effect on T Cell Counts

Control Cells were MHC Class I null (beta 2 micro globin deletion) and MHC Class II null (CTIIA deletion). Here, we compared the activity of the above cells with respect to antigen presentation via BCR mediated uptake to stimulate antigen specific T cells (Cd69 upregulation is the measure of T cell activation).

The antigen was GPC3-OVA (transmembrane protein); the BCR was Anti-GPC3-CAR-B (79a format) and cd80; and amounts for B and T cells measured was as follows: B cells = 0.25 million cells; T cells = 1 million cells. The results are shown in FIG. 31.

### Example 27 - Murine B Cells

Here, the control cells utilized were as follows: MHC Class I null (beta 2 micro globin deletion); MHC Class II null (CTIIA deletion).

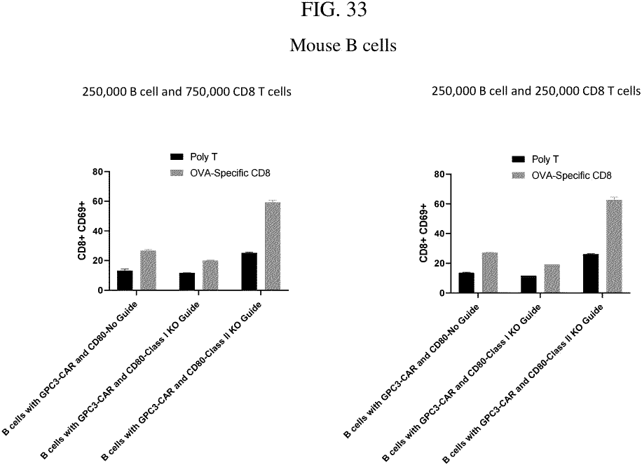

The activity of the above cells was compared with respect to antigen presentation via BCR-mediated uptake to stimulate antigen specific T cells. CD69 upregulation is a measure of T cell activation. The antigen utilized was GPC3-OVA (transmembrane protein in CHO cells). The CAR-B used was anti-GPC3-CAR-B (CD79a format) and CD80. B and T cells were measured as follows: B cells: 0.25 million; T cells = 0.25 million. B and T cells were further measured as follows: B cells: 0.25 million; T cells = 0.75 million. The results are set forth in FIGS. 32 and 33.

What is claimed:

1. A method of treating a patient comprising administering to said patient an effective amount of (i) a plurality of isolated T cells and (ii) an effective amount of plurality of isolated modified B cells:
wherein said isolated modified B cells are capable of expressing a chimeric receptor (CAR-B), and wherein said chimeric receptor comprises
d) an extracellular domain, wherein the extracellular domain comprises an extracellular binding domain and a hinge domain;
e) a transmembrane domain; and
f) a cytoplasmic domain that comprises at least one signaling domain.

2. The method of claim 1 wherein said isolated T cells and said CAR-B cells are administered sequentially or concurrently.

3. The method of claim 1, wherein said extracellular binding domain recognizes at least one antigen or protein expressed on the surface of a target cell.

4. The method of claim 1, wherein the extracellular binding domain(s) recognizes at least one antigen that is a secreted protein.

5. The isolated modified B cell of claim 4, wherein said target cell is selected from the group consisting of a tumor cell, a cardiac muscle cell, a skeletal muscle cell, a bone cell, a blood cell, a nerve cell, a fat cell, a skin cell, an endothelial cell, a hepatocyte, a pulmonary epithelial cell, and a fibroblast cell.

6. The method of claim 1 wherein said B cell expresses more than one CAR-B receptor construct.

7. The method of claim 1 wherein said extracellular binding domain is a single chain variable fragment (scFv), or a full-length antibody or an antibody fragment, or the extracellular domain of a receptor or ligand.

8. The method of claim 1, wherein said extracellular binding domain is capable of binding to an antigen or protein selected from the group consisting of: PSMA, GPC3, ASGR1, ASGR2, SGCA, Corin, FAP, MUC1, CEA153, JAM-1, LAF-1, Her2; AFP, and MAGE.

9. The method of claim 1 wherein said cytoplasmic domain comprises a domain that is selected from the group consisting of: CD79a (Immunoglobulin α), CD40, CD19, CD137, Fcγr2a, MyD88, CD21, Syk, FYN, LYN, PI3K, BTK, PLCγ2, CD3ζ and BLNK.

10. The method of claim 1 wherein said cytoplasmic domain comprises CD79a.

11. The method of claim 1 wherein said isolated modified B cell is capable of expressing and secreting a payload, wherein the payload is not naturally expressed in a B cell or is expressed at higher levels than is naturally expressed in a B cell.

12. The method of claim 1 wherein the payload is an antibody or fragment thereof.

13. The method of claim 1 wherein said payload is at least one payload selected from cytokines, chemokines, T cell costimulatory molecules, and checkpoint molecules, the group consisting of: IL-1, IL-7, IL-8, IL-10, IL-12, IL-13, IL-17, IL-18, IL-21, interferon α, interferon β, interferon γ, TSLP, CCL21, FLT3L, XCL1, LIGHT(TNFSF14), OX40L, CD137L, CD40L, ICOSL, anti-CD3 antibody, CD47, TIM4-FC, CXCL13, CCL21, CD80, CD86, CD40L, IFNα A2, LIGHT , 4-1BBL, MDGF (C19orf10), FGF10, PDGF, agrin, TNF-α, GM-CSF, an anti-FAP antibody, an anti-TGF-β antibody; a TGF-β trap, decoy or other inhibitory molecule; an anti-BMP antibody; a BMP trap, decoy or other inhibitory molecule.

14. The method of claim 1, wherein the isolated modified B cell is administered intra-tumorally, intravenously, subcutaneously, intradermally, or within an inflammatory lesion.

15. The method of claim 1, further comprising administering to said patient one or more checkpoint inhibitors, with or without an additional chemotherapeutic agent.

16. A method of treating a patient comprising administering to said patient an effective amount of (i) a plurality of isolated non-B cell modified immune cells and (ii) an effective amount of plurality of isolated modified B cells;
wherein said isolated modified B cells are capable of expressing a chimeric receptor (CAR-B), and wherein said chimeric receptor comprises
g) an extracellular domain, wherein the extracellular domain comprises an extracellular binding domain and a hinge domain;
h) a transmembrane domain; and
i) a cytoplasmic domain that comprises at least one signaling domain.

17. The method of claim 1 wherein said isolated T cells and said CAR-B cells are administered sequentially or concurrently.

18. The method according to claim 16 wherein said non-B cell modified immune cells are at least one of CAR-T cells, TILs, and TCR cells.

19. The method of claim 16 wherein said extracellular binding domain recognizes at least one antigen or protein expressed on the surface of a target cell.

20. The method of claim 16 wherein the extracellular binding domain(s) recognizes at least one antigen that is a secreted protein.

21. The isolated modified B cell of claim 19, wherein said target cell is selected from the group consisting of a tumor cell, a cardiac muscle cell, a skeletal muscle cell, a bone cell, a blood cell, a nerve cell, a fat cell, a skin cell, an endothelial cell, a hepatocyte, a pulmonary epithelial cell, and a fibroblast cell.

22. A combination therapy comprising:
a) An isolated modified non-B cell immune cell, and
b) an isolated modified B cell, capable of expressing a chimeric receptor, wherein said chimeric receptor comprises:
j) an extracellular domain, wherein the extracellular domain comprises an extracellular binding domain and a hinge domain;
k) a transmembrane domain; and
1) a cytoplasmic domain that comprises at least one signaling domain
wherein said modified B cell is optionally further capable of expressing a payload.

23. The therapy of claim 22 wherein said payload comprises at least one of CD80 or CD86.

24. The therapy of claim 22 wherein said non-B cell modified immune cells are at least one of CAR-T cells, TILs, andTCR cells.
